(19) United States
(12) Patent Application Publication
(10) Pub. No.: US 2025/0145600 A1
(43) Pub. Date: May 8, 2025

(54) 2-((4-((S)-2-(4-CHLORO-2-FLUOROPHENYL)-2-METHYLBENZO[D][1,3]DIOXOL-4-YL)PIPERIDIN-1-YL)METHYL)-1-(((S)-OXETAN-2-YL)METHYL)-1H-IMIDAZOLE DERIVATIVES AS ACTIVATORS OF THE GLP1 RECEPTOR FOR THE TREATMENT OF OBESITY

(71) Applicant: Novartis AG, Basel (CH)

(72) Inventors: Martin ALLAN, Worcester, MA (US); Matthew CARSON, Harvard, MA (US); Thomas CAYA, Somerville, MA (US); Tanzina FAZAL, Burlington, MA (US); Ming QIAN, Watertown, MA (US); Troy SMITH, Nashua, NH (US); Liansheng SU, Winchester, MA (US); Lihua YANG, Westford, MA (US); Ping ZHANG, Somerville, MA (US)

(21) Appl. No.: 18/836,800

(22) PCT Filed: Feb. 10, 2023
(86) PCT No.: PCT/IB2023/051207

§ 371 Date: Aug. 8, 2024

Related U.S. Application Data
(60) Provisional application No. 63/308,843, filed on Feb. 10, 2022.
(60) Provisional application No. 63/323,401, filed on Mar. 24, 2022.
(60) Provisional application No. 63/378,749, filed on Oct. 7, 2022.

(51) Int. Cl. C07D 413/14; A61K 31/454; A61K 31/4545; A61K 45/06; C07D 405/14; C07D 417/14

(52) U.S. Cl. CPC C07D 413/14; A61K 31/454; A61K 31/4545; A61K 45/06; C07D 405/14; C07D 417/14

(57) ABSTRACT

The present invention relates to compounds of formula (I):

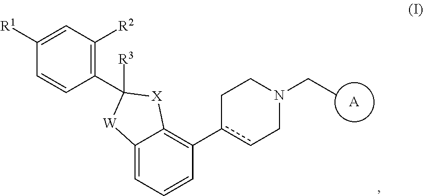

or pharmaceutically acceptable salts thereof, pharmaceutical compositions comprising these compounds, as well as methods and uses of these compounds in the treatment of diseases.

### TECHNICAL FIELD

The present invention relates to 1,3-benzodioxole derivatives, to their preparation, to pharmaceutical compositions comprising them and to their use in the treatment of conditions, diseases and disorders treatable by activating Glucagon-like Peptide 1 Receptor (GLP1R).

### BACKGROUND

Glucagon-like Peptide 1 receptor (GLP1R) belongs to family 1 of the G protein-coupled receptors (GPCRs) and is expressed in many tissues including pancreas, heart, gut and brain (Kieffer T. J. and Habener, J. F. Endocrin. Rev. 20:876-913 (1999); Drucker, D. J., Endocrinology 142:521-7 (2001); Hoist, J. J., Diabetes Metab. Res. Rev. 18:430-41 (2002); Regard J. B., Cell 135:561-71 (2008)). GLP1R natural agonist ligands are GLP-1 (7-36, 30 aa) and oxyntomodulin (OXM, 37 aa), both derived from pro-glucagon. Upon activation, GLP1Rs couple to G_{αs}-protein with subsequent activation of adenylate cyclase and intracellular increase of cAMP levels, thereby potentiating glucose-stimulated insulin secretion acting on the pancreatic beta-cells. Therefore, GLP1R is an attractive therapeutic target for lowering blood glucose in diabetic patients. Several GLP1R agonist peptides (e.g., liraglutide, albiglutide, exenatide, lixisenatide, dulaglutide, semaglutide) are being developed for the treatment for patients suffering from diabetes, NASH and/or obesity.

Obesity is a chronic disease that is highly prevalent in modern society and is associated with a number of co-morbidities including hypertension, hypercholesterolemia, and coronary heart disease. It is further highly correlated with type 2 diabetes mellitus (T2DM) and insulin resistance, the latter of which is generally accompanied by hyperinsulinemia or hyperglycemia, or both. In addition, T2DM is associated with a two- to four-fold increased risk of coronary artery disease. Currently, the most effective obesity treatment is bariatric surgery, which is, however, both costly and risky for patients. Pharmacological replacement of bariatric surgery is therefore an attractive alternative. That said, pharmacological intervention for the treatment of obesity has been shown to be less efficacious than bariatric surgery and also associated with side effects. Out of the marketed GLP1R agonist peptides, liraglutide has been approved as a once-daily treatment for obesity. Semaglutide currently is in a Phase 3 clinical trial as a once-weekly treatment for obesity. Presently, there is no approved pharmacotherapy for the remission of type 2 diabetes mellitus in adults who are unable to maintain normal glycemic control with anti-diabetics and/or insulin due to insulin resistance and no approved pharmacotherapy for heart failure with preserved ejection fraction (HFpEF). Alternatively, a GLP1R agonist may be an effective therapy for T2DM remission. A GLP1R receptor agonist may reduce the risk of cardiovascular death and hospitalization for patients with chronic HFpEF.

Metabolic disorders are therefore potentially treatable with small molecule agonists of GLP1R.

### SUMMARY

The present invention provides, inter alia, compounds of formula (I):

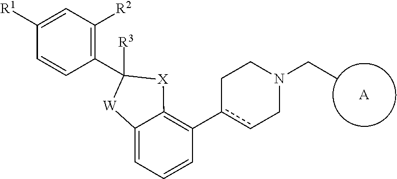

or pharmaceutically acceptable salts thereof, wherein constituent members are as defined herein.

The compounds of formula (I), and pharmaceutically acceptable salts thereof, as herein defined, are GLP1R agonists. Accordingly, these compounds may be useful in the treatment of metabolic diseases, disorders and conditions, such as obesity, type 2 diabetes mellitus, insulin resistance, hyperinsulinemia, glucose intolerance, hyperglycemia, one or more diabetic complications (including but not limited to chronic kidney disease), diabetic nephropathy, dyslipidemia and cardiovascular disease. The compounds may also be useful in the treatment of progressive liver disease and neuropathies.

Also provided herein are pharmaceutical compositions comprising a compound of formula (I), or a pharmaceutically acceptable salt thereof, and one or more pharmaceutically acceptable carriers.

Provided herein are methods of agonizing or activating Glucagon-like Peptide 1 Receptor (GLP1R), said methods comprising contacting the GLP1R with a compound of formula (I), or a pharmaceutically acceptable salt thereof.

Provided herein are methods of treating, preventing, or ameliorating a condition, disease, or disorder treatable by activating or agonizing GLP1R in a patient, said methods comprising administering to the patient a compound of formula (I), or a pharmaceutically acceptable salt thereof (in, e.g., a therapeutically effective amount).

Provided herein are methods of treating, preventing, or ameliorating a disease, disorder, or condition selected from metabolic and related disorders, including obesity and type 2 diabetes mellitus, cardiovascular disease such as heart failure (for example heart failure with preserved ejection fraction (HFpEF)), and non-alcoholic steatohepatitis (NASH) in a patient, said methods comprising administering to the patient a compound of formula (I), or a pharmaceutically acceptable salt thereof (in, e.g., a therapeutically effective amount).

Also provided herein is a compound of formula (I), or a pharmaceutically acceptable salt thereof, for use in any of the methods described herein.

Also provided herein is a use of a compound of formula (I), or a pharmaceutically acceptable salt thereof, for the preparation of a medicament for use in any of the methods described herein.

The compounds of the present invention may exhibit advantageous ADME (absorption, distribution, metabolism and excretion) properties, for example, in vivo exposure (in particular, when dosed orally), for example, as demonstrated by measurement of certain pharmacokinetic parameters such as maximum concentration in plasma (Cₘₐₓ) and/or total exposure (area under the curve (AUC)) values.

### DETAILED DESCRIPTION

Various aspects and embodiments of the invention are described herein. It will be recognized that certain features are specified in the context of separate embodiments for clarity (and/or brevity), however, such embodiments may be combined with other specified features (e.g., in any suitable sub-combination) to provide further embodiments of the present invention.

The definition of the substituents applies to compounds of any formulae provided herein, e.g., formulae (Ia), (II), (IIa), (III), (IIIa), (IIIb), (IV), (IVa), (IVb), (V), (Va) and (Vb), as appropriate. The definition of the substituents applies to the end-products as well as to the corresponding intermediates as appropriate.

In an aspect, provided herein is a compound of formula (I):

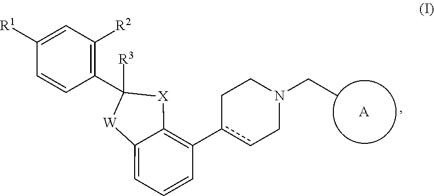

or a pharmaceutically acceptable salt thereof, wherein
[symbol] is a single or double bond;

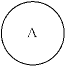

is

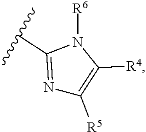

wherein
[symbol] indicates the point of attachment to the rest of the molecule;
W is O or CH₂;
X is O or CH₂;
R¹ and R² are each independently selected from H, C₁₋₃-alkyl, halo and CN;
R³ is selected from H and C₁₋₃-alkyl;
R⁴ is (a) selected from H, C₁₋₆-alkyl, C₂₋₆ alkenyl, C₂₋₆ alkynyl, C₃₋₆-cycloalkyl, 4-10 membered heterocycloalkyl, phenyl, C₃₋₆-cycloalkyl-C₁₋₃-alkyl-, (4-10 membered heterocycloalkyl)-C₁₋₃-alkyl-, phenyl-C₁₋₃-alkyl-, halo, CN, NO₂, OR^{4a}, SR^{4a}, C(O)R^{4b}, C(O)NR^{4c}R^{4d}, C(O)NR^{4c}(OR^{4a}), C(O)NR^{4c}(S(O)₂R^{4b}), C(O)NR^{4c}(S(O)₂NR^{4c}R^{4d}), NR^{4c}OR^{4a}, NR^{4c}R^{4d}, NR^{4c}(C(O)R^{4b}), NR^{4c}(C(O)OR^{4a}), N(OR^{4a})(C(O)R^{4b}), NR^{4c}(C(O)NR^{4c}R^{4d}), NR^{4c}(C(O)NR^{4c}(C(O)R^{4b})), NR^{4c}(S(O)₂R^{4b}), NR^{4c}(S(O)₂NR^{4c}R^{4d}), NR^{4c}(C(O)NR^{4c}(S(O)₂R^{4b})), OC(O)R^{4b}, OC(O)NR^{4c}R^{4d}, ONR^{4c}(C(O)R^{4b}), OS(O)₂R^{4b}, OP(O)(OR^{4e})(OR^{4f}), S(O)OR^{4a}, S(O)R^{4b}, S(O)₂R^{4b}, S(O)₂NR^{4c}R^{4d}, S(O)₂OR^{4a}, S(═NR^{4g})(O)R^{4b}, S(═NR^{4g})(O)NR^{4c}NR^{4d}, P(O)(OR^{4e})(OR^{4f}), and P(O)(OR^{4e})(R^{4f}), wherein the C₁₋₆-alkyl, C₂₋₆ alkenyl, C₂₋₆ alkynyl, C₃₋₆-cycloalkyl, 4-10 membered heterocycloalkyl, phenyl, C₃₋₆-cycloalkyl-C₁₋₃-alkyl-, (4-10 membered heterocycloalkyl)-C₁₋₃-alkyl-, and phenyl-C₁₋₃-alkyl- of R⁴ are each optionally substituted with 1, 2, or 3 groups independently selected from halo, CN, NO₂, OR^{4A}, SR^{4A}, C(O)R^{4B}, C(O)NR^{4C}R^{4D}, C(O)NR^{4C}(OR^{4A}), C(O)NR^{4C}(S(O)₂R^{4B}), C(O)NR^{4C}(S(O)₂NR^{4C}R^{4D}), NR^{4C}OR^{4A}, NR^{4C}R^{4D}, NR^{4C}(C(O)R^{4B}), NR^{4C}(C(O)OR^{4A}), N(OR^{4A})(C(O)R^{4B}), NR^{4C}(C(O)NR^{4C}R^{4D}), NR^{4C}(C(O)NR^{4C}(C(O)R^{4B})), NR^{4C}(S(O)₂R^{4B}), NR^{4C}(S(O)₂NR^{4C}R^{4D}), NR^{4C}(C(O)NR^{4C}(S(O)₂R^{4B})), OC(O)R^{4B}, OC(O)NR^{4C}R^{4D}, ONR^{4C}(C(O)R^{4B}), OS(O)₂R^{4B}, OP(O)(OR^{4E})(OR^{4F}), S(O)OR^{4A}, S(O)R^{4B}, S(O)₂R^{4B}, S(O)₂NR^{4C}R^{4D}, S(O)₂OR^{4A}, S(═NR^{4G})(O)R^{4B}, S(═NR^{4G})(O)NR^{4C}NR^{4D}, P(O)(OR^{4E})(OR^{4F}), and P(O)(OR^{4E})(R^{4F}), or
R⁴ is (b) 5-10 membered heteroaryl or (5-10 membered heteroaryl)-C₁₋₃-alkyl-, wherein the 5-10 membered heteroaryl and (5-10 membered heteroaryl)-C₁₋₃-alkyl- are each substituted with C(O)OR^{4A} or with a carboxylic acid isostere, and optionally with 1, 2, or 3 groups independently selected from halo, CN, NO₂, OR^{4A}, SR^{4A}, C₁₋₃-alkyl, C₁₋₃-alkyl substituted with 1, 2 or 3 halo, C(O)R^{4B}, C(O)NR^{4C}R^{4D}, C(O)NR^{4C}(OR^{4A}), C(O)NR^{4C}(S(O)₂R^{4B}), C(O)NR^{4C}(S(O)₂NR^{4C}R^{4D}), NR^{4C}OR^{4A}, NR^{4C}R^{4D}, NR^{4C}(C(O)R^{4B}), NR^{4C}(C(O)OR^{4A}), N(OR^{4A})(C(O)R^{4B}), NR^{4C}(C(O)NR^{4C}R^{4D}), NR^{4C}(C(O)NR^{4C}(C(O)R^{4B})), NR^{4C}(S(O)₂R^{4B}), NR^{4C}(S(O)₂NR^{4C}R^{4D}), NR^{4C}(C(O)NR^{4C}(S(O)₂R^{4B})), OC(O)R^{4B}, OC(O)NR^{4C}R^{4D}, ONR^{4C}(C(O)R^{4B}), OS(O)₂R^{4B}, OP(O)(OR^{4E})(OR^{4F}), S(O)OR^{4A}, S(O)R^{4B}, S(O)₂R^{4B}, S(O)₂NR^{4C}R^{4D}, S(O)₂OR^{4A}, S(═NR^{4G})(O)R^{4B}, S(═NR^{4G})(O)NR^{4C}NR^{4D}, P(O)(OR^{4E})(OR^{4F}), and P(O)(OR^{4E})(R^{4F});
R⁵ is (a) selected from H, C₁₋₆-alkyl, C₂₋₆ alkenyl, C₂₋₆ alkynyl, C₃₋₆-cycloalkyl, 4-10 membered heterocycloalkyl, phenyl, C₃₋₆-cycloalkyl-C₁₋₃-alkyl, (4-10 membered heterocycloalkyl)-C₁₋₃-alkyl-, phenyl-C₁₋₃-alkyl-, halo, CN, NO₂, OR^{5a}, SR^{5a}, C(O)R^{5b}, C(O)NR^{5c}R^{5d}, C(O)NR^{5c}(OR^{5a}), C(O)NR^{5c}(S(O)₂R^{5b}), C(O)NR^{5c}(S(O)₂NR^{5c}R^{5d}), NR^{5c}OR^{5a}, NR^{5c}R^{5d}, NR^{5c}(C(O)R^{5b}), NR^{5c}(C(O)OR^{5a}), N(OR^{5a})(C(O)R^{5b}), NR^{5c}(C(O)NR^{5c}R^{5d}), NR^{5c}(C(O)NR^{5c}(C(O)R^{5b})), NR^{5c}(S(O)₂R^{5b}), NR^{5c}(S(O)₂NR^{5c}R^{5d}), NR^{5c}(C(O)NR^{5c}(S(O)₂R^{5b})), OC(O)R^{5b}, OC(O)NR^{5c}R^{5d}, ONR^{5c}(C(O)R^{5b}), OS(O)₂R^{5b}, OP(O)(OR^{5e})(OR^{5f}), S(O)OR^{5a}, S(O)R^{5b}, S(O)₂R^{5b}, S(O)₂NR^{5c}R^{5d} S(O)₂OR^{5a}, S(═NR^{5g})(O)R^{5b}, S(═NR^{5g})(O)NR^{5c}NR^{5d}, P(O)(OR^{5e})(OR^{5f}), and P(O)(OR^{5e})(R^{5f}), wherein the C₁₋₆-alkyl, C₂₋₆ alkenyl, C₂₋₆ alkynyl, C₃₋₆-cycloalkyl, 4-10 membered heterocycloalkyl, phenyl, C₃₋₆-cycloalkyl-C₁₋₃-alkyl-, (4-10 membered heterocycloalkyl)-C₁₋₃-alkyl-, and phenyl-C₁₋₃-alkyl- of R⁵ are each optionally substituted with 1, 2, or 3 groups independently selected from halo, CN, NO₂, OR^{5A}, SR^{5A}, C(O)R^{5B}, C(O)NR^{5C}R^{5D}, C(O)NR^{5C}(OR^{5A}), C(O)NR^{5C}(S(O)₂R^{5B}), C(O)NR^{5C}(S(O)₂NR^{5C}R^{5D}), NR^{5C}OR^{5A}, NR^{5C}R^{5D}, NR^{5C}(C(O)R^{5B}), NR^{5C}(C(O)OR^{5A}), N(OR^{5A})(C(O)R^{5B}), NR^{5C}(C(O)NR^{5C}R^{5D}), NR^{5C}(C(O)NR^{5C}(C(O)R^{5B})), NR^{5C}(S(O)₂R^{5B}), NR^{5C}(S(O)₂NR^{5C}R^{5D}), NR^{5C}(C(O)NR^{5C}(S(O)₂R^{5B})), OC(O)R^{5B}, OC(O)NR^{5C}R^{5D}, ONR^{5C}(C(O)R^{5B}), OS(O)₂R^{5B}, OP(O)(OR^{5E})(OR^{5F}), S(O)OR^{5A}, S(O)R^{5B}, S(O)₂R^{5B}, S(O)₂NR^{5C}R^{5D}, S(O)₂OR^{5A}, S(═NR^{5G})(O)R^{5B}, S(═NR^{5G})(O)NR^{5C}NR^{5D}, P(O)(OR^{5E})(OR^{5F}), and P(O)(OR^{5E})(R^{5F}), or
R⁵ is (b) 5-10 membered heteroaryl or (5-10 membered heteroaryl)-C₁₋₃-alkyl-, wherein the 5-10 membered heteroaryl and (5-10 membered heteroaryl)-C₁₋₃-alkyl- are each substituted with C(O)OR^{5A} or with a carboxylic acid isostere, and optionally with 1, 2, or 3 groups independently selected from halo, CN, NO₂, OR^{5A}, SR^{5A}, C₁₋₃-alkyl, C₁₋₃-alkyl substituted with 1, 2 or 3 halo, C(O)R^{5B}, C(O)NR^{5C}R^{5D}, C(O)NR^{5C}(OR^{5A}), C(O)NR^{5C}(S(O)₂R^{5B}), C(O)NR^{5C}(S(O)₂NR^{5C}R^{5D}), NR^{5C}OR^{5A}, NR^{5C}R^{5D}, NR^{5C}(C(O)R^{5B}), NR^{5C}(C(O)OR^{5A}), N(OR^{5A})(C(O)R^{5B}), NR^{5C}(C(O)NR^{5C}R^{5D}), NR^{5C}(C(O)NR^{5C}(C(O)R^{5B})), NR^{5C}(S(O)₂R^{5B}), NR^{5C}(S(O)₂NR^{5C}R^{5D}), NR^{5C}(C(O)NR^{5C}(S(O)₂R^{5B})), OC(O)R^{5B}, OC(O)NR^{5C}R^{5D}, ONR^{5C}(C(O)R^{5B}), OS(O)₂R^{5B}, OP(O)(OR^{5E})(OR^{5F}), S(O)OR^{5A}, S(O)R^{5B}, S(O)₂R^{5B}, S(O)₂NR^{5C}R^{5D}, S(O)₂OR^{5A}, S(═NR^{5G})(O)R^{5B}, S(═NR^{5G})(O)NR^{5C}NR^{5D}, P(O)(OR^{5E})(OR^{5F}), and P(O)(OR^{5E})(R^{5F});
R⁶ is selected from (4-10 membered heterocycloalkyl)-C₁₋₃-alkyl- and (5-10 membered heteroaryl)-C₁₋₃-alkyl-, wherein the (4-10 membered heterocycloalkyl)-C₁₋₃-alkyl- and (5-10 membered heteroaryl)-C₁₋₃-alkyl- of R⁶ are each optionally substituted with 1, 2, or 3 groups independently selected from C₁₋₆-alkyl, —OH, and halo;
R^{4a}, R^{4b}, R^{4c}, and R^{4d} are each independently selected from H, C₁₋₆-alkyl, C₂₋₆ alkenyl, C₂₋₆ alkynyl, C₃₋₆-cycloalkyl, 4-10 membered heterocycloalkyl, 5-10 membered heteroaryl, and phenyl, wherein the C₁₋₆-alkyl, C₂₋₆ alkenyl, and C₂₋₆ alkynyl of R^{4a}, R^{4b}, R^{4c}, and R^{4d} are each optionally substituted with 1, 2, or 3 groups independently selected from —OH and halo; and the C₃₋₆-cycloalkyl, 4-10 membered heterocycloalkyl, 5-10 membered heteroaryl, and phenyl of R^{4a}, R^{4b}, R^{4c}, and R^{4d} are each optionally substituted with 1, 2, or 3 groups independently selected from C₁₋₆-alkyl, —OH, and halo;
R^{4e}, R^{4f}, and R^{4g} are each independently selected from H and C₁₋₆-alkyl;
R^{4A}, R^{4B}, R^{4C} and R^{4D} are each independently selected from H, C₁₋₆alkyl, C₂₋₆ alkenyl, C₂₋₆ alkynyl, C₃₋₆-cycloalkyl, 4-10 membered heterocycloalkyl, 4-10 membered heterocycloalkyl-C₁₋₃-alkyl-, 5-10 membered heteroaryl, and phenyl, wherein the C₁₋₆-alkyl, C₂₋₆ alkenyl, and C₂₋₆ alkynyl of R^{4A}, R^{4B}, R^{4C} and R^{4D} are each optionally substituted with 1, 2, or 3 groups independently selected from —OH, halo and —OC(O)—C₁₋₁₅-alkyl; and the C₃₋₆-cycloalkyl, 4-10 membered heterocycloalkyl, 4-10 membered heterocycloalkyl-C₁₋₃-alkyl-, 5-10 membered heteroaryl, and phenyl of R^{4A}, R^{4B}, R^{4C} and R^{4D} are each optionally substituted with 1, 2, or 3 groups independently selected from C₁₋₆-alkyl, —OH, and halo;
R^{4E}, R^{4F} and R^{4G} are each independently selected from H and C₁₋₆-alkyl;
R^{5a}, R^{5b}, R^{5c}, and R^{5d} are each independently selected from H, C₁₋₆-alkyl, C₂₋₆ alkenyl, C₂₋₆ alkynyl, C₃₋₆-cycloalkyl, 4-10 membered heterocycloalkyl, 5-10 membered heteroaryl, and phenyl, wherein the C₁₋₆-alkyl, C₂₋₆ alkenyl, and C₂₋₆ alkynyl of R⁵, R^{5b}, R^{5c}, and R^{5d} are each optionally substituted with 1, 2, or 3 groups independently selected from —OH and halo; and the C₃₋₆-cycloalkyl, 4-10 membered heterocycloalkyl, 5-10 membered heteroaryl, and phenyl of R^{5a}, R^{5b}, R^{5c}, and R^{5d} are each optionally substituted with 1, 2, or 3 groups independently selected from C₁₋₆-alkyl, —OH, and halo;
R^{5e}, R^{5f}, and R^{5g} are each independently selected from H and C₁₋₆-alkyl;
R^{5A}, R^{5B}, R^{5C} and R^{5D} are each independently selected from H, C₁₋₆-alkyl, C₂₋₆ alkenyl, C₂₋₆ alkynyl, C₃₋₆-cycloalkyl, 4-10 membered heterocycloalkyl, 4-10 membered heterocycloalkyl-C₁₋₃-alkyl-, 5-10 membered heteroaryl, and phenyl, wherein the C₁₋₆-alkyl, C₂₋₆ alkenyl, and C₂₋₆ alkynyl of R^{5A}, R^{5B}, R^{5C} and R^{5D} are each optionally substituted with 1, 2, or 3 groups independently selected from —OH, halo and —OC(O)—C₁₋₁₅-alkyl; and the C₃₋₆-cycloalkyl, 4-10 membered heterocycloalkyl, 4-10 membered heterocycloalkyl-C₁₋₃-alkyl-, 5-10 membered heteroaryl, and phenyl of R^{5A}, R^{5B}, R^{5C} and R^{5D} are each optionally substituted with 1, 2, or 3 groups independently selected from C₁₋₆-alkyl, —OH, and halo; and
R^{5E}, R^{5F} and R^{5G} are each independently selected from H and C₁₋₆-alkyl.

In a preferred embodiment of the invention, provided herein is a compound of formula (I):

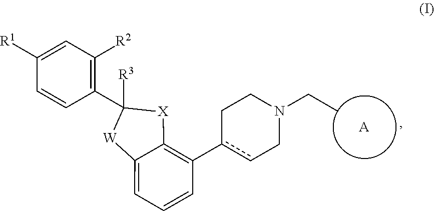

or a pharmaceutically acceptable salt thereof, wherein
[symbol] is a single or double bond;

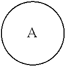

is

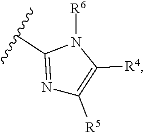

wherein
[symbol] indicates the point of attachment to the rest of the molecule;
W is O or CH₂;
X is O or CH₂;
R¹ and R² are each independently selected from H, C₁₋₃-alkyl, halo and CN;
R³ is selected from H and C₁₋₃-alkyl;
R⁴ and R⁵ are selected from:
(aa) R⁴ is selected from H, C₁₋₆-alkyl, C₂₋₆ alkenyl, C₂₋₆ alkynyl, C₃₋₆-cycloalkyl, 4-10 membered heterocycloalkyl, phenyl, C₃₋₆-cycloalkyl-C₁₋₃-alkyl-, (4-10 membered heterocycloalkyl)-C₁₋₃-alkyl-, phenyl-C₁₋₃-alkyl-, halo, CN, NO₂, OR^{4a}, SR^{4a}, C(O)R^{4b}, C(O)NR^{4c}R^{4d}, C(O)NR^{4c}(OR^{4a}), C(O)NR^{4c}(S(O)₂R^{4b}), C(O)NR^{4c}(S(O)₂NR^{4c}R^{4d}), NR^{4c}OR^{4a}, NR^{4c}R^{4d}, NR^{4c}(C(O)R^{4b}), NR^{4c}(C(O)OR^{4a}), N(OR^{4a})(C(O)R^{4b}), NR^{4c}(C(O)NR^{4c}R^{4d}), NR^{4c}(C(O)NR^{4c}(C(O)R^{4b})), NR^{4c}(S(O)₂R^{4b}), NR^{4c}(S(O)₂NR^{4c}R^{4d}), NR^{4c}(C(O)NR^{4c}(S(O)₂R^{4b})), OC(O)R^{4b}, OC(O)NR^{4c}R^{4d}, ONR^{4c}(C(O)R^{4b}), OS(O)₂R^{4b}, OP(O)(OR^{4e})(OR^{4f}), S(O)OR^{4a}, S(O)R^{4b}, S(O)₂R^{4b}, S(O)₂NR^{4c}R^{4d}, S(O)₂OR^{4a}, S(═NR^{4g})(O)R^{4b}, S(═NR^{4g})(O)NR^{4c}NR^{4d}, P(O)(OR^{4e})(OR^{4f}), and P(O)(OR^{4e})(R^{4f}), wherein the C₁₋₆-alkyl, C₂₋₆ alkenyl, C₂₋₆ alkynyl, C₃₋₆-cycloalkyl, 4-10 membered heterocycloalkyl, phenyl, C₃₋₆-cycloalkyl-C₁₋₃-alkyl-, (4-10 membered heterocycloalkyl)-C₁₋₃-alkyl-, and phenyl-C₁₋₃-alkyl- of R⁴ are each optionally substituted with 1, 2, or 3 groups independently seleCted from halo, CN, NO₂, OR^{4A}, SR^{4A}, C(O)R^{4B}, C(O)NR^{4C}R^{4D}, C(O)NR^{4C}(OR^{4A}), C(O)NR^{4C}(S(O)₂R^{4B}), C(O)NR^{4C}(S(O)₂NR^{4C}R^{4D}), NR^{4C}OR^{4A}, NR^{4C}R^{4D}, NR^{4C}(C(O)R^{4B}), NR^{4C}(C(O)OR^{4A}), N(OR^{4A})(C(O)R^{4B}), NR^{4C}(C(O)NR^{4C}R^{4D}), NR^{4C}(C(O)NR^{4C}(C(O)R^{4B})), NR^{4C}(S(O)₂R^{4B}), NR^{4C}(S(O)₂NR^{4C}R^{4D}), NR^{4C}(C(O)NR^{4C}(S(O)₂R^{4B})), OC(O)R^{4B}, OC(O)NR^{4C}R^{4D}, ONR^{4C}(C(O)R^{4B}), OS(O)₂R^{4B}, OP(O)(OR^{4E})(OR^{4F}), S(O)OR^{4A}, S(O)R^{4B}, S(O)₂R^{4B} S(O)₂NR^{4C}R^{4D}, S(O)₂OR^{4A}, S(═NR^{4G})(O)R^{4B}, S(═NR^{4G})(O)NR^{4C}NR^{4D}, P(O)(OR^{4E})(OR^{4F}), and P(O)(OR^{4E})(R^{4F}), and
R⁵ is 5-10 membered heteroaryl or (5-10 membered heteroaryl)-C₁₋₃-alkyl-, wherein the 5-10 membered heteroaryl and (5-10 membered heteroaryl)-C₁₋₃-alkyl- are each substituted with C(O)OR^{5A} or with a carboxylic acid isostere, and optionally with 1, 2, or 3 groups independently selected from halo, CN, NO₂, OR^{5A}, SR^{5A}, C₁₋₃-alkyl, C₁₋₃-alkyl substituted with 1, 2 or 3 halo, C(O)R^{5B}, C(O)NR^{5C}R^{5D}, C(O)NR^{5C}(OR^{5A}), C(O)NR^{5C}(S(O)₂R^{5B}), C(O)NR^{5C}(S(O)₂NR^{5C}R^{5D}), NR^{5C}OR^{5A}, NR^{5C}R^{5D}, NR^{5C}(C(O)R^{5B}), NR^{5C}(C(O)OR^{5A}), N(OR^{5A})(C(O)R^{5B}), NR^{5C}(C(O)NR^{5C}R^{5D}), NR^{5C}(C(O)NR^{5C}(C(O)R^{5B})), NR^{5C}(S(O)₂R^{5B}), NR^{5C}(S(O)₂NR^{5C}R^{5D}), NR^{5C}(C(O)NR^{5C}(S(O)₂R^{5B})), OC(O)R^{5B}, OC(O)NR^{5C}R^{5D}, ONR^{5C}(C(O)R^{5B}), OS(O)₂R^{5B}, OP(O)(OR^{5E})(OR^{5F}), S(O)OR^{5A}, S(O)R^{5B} S(O)₂R^{5B} S(O)₂NR^{5C}R^{5D}, S(O)₂OR^{5A}, S(═NR^{5G})(O)R^{5B}, S(═NR^{5G})(O)NR^{5C}NR^{5D}, P(O)(OR^{5E})(OR^{5F}), and P(O)(OR^{5E})(R^{5F}); or
(bb) R⁴ is 5-10 membered heteroaryl or (5-10 membered heteroaryl)-C₁₋₃-alkyl-, wherein the 5-10 membered heteroaryl and (5-10 membered heteroaryl)-C₁₋₃-alkyl- are each substituted with C(O)OR^{4A} or with a carboxylic acid isostere, and optionally with 1, 2, or 3 groups independently selected from halo, CN, NO₂, OR^{4A}, SR^{4A}, C₁₋₃-alkyl, C₁₋₃-alkyl substituted with 1, 2 or 3 halo, C(O)R^{4B}, C(O)NR^{4C}R^{4D}, C(O)NR^{4C}(OR^{4A}), C(O)NR^{4C}(S(O)₂R^{4B}), C(O)NR^{4C}(S(O)₂NR^{4C}R^{4D}), NR^{4C}OR^{4A}, NR^{4C}R^{4D}, NR^{4C}(C(O)R^{4B}), NR^{4C}(C(O)OR^{4A}), N(OR^{4A})(C(O)R^{4B}), NR^{4C}(C(O)NR^{4C}R^{4D}), NR^{4C}(C(O)NR^{4C}(C(O)R^{4B})), NR^{4C}(S(O)₂R^{4B}), NR^{4C}(S(O)₂NR^{4C}R^{4D}), NR^{4C}(C(O)NR^{4C}(S(O)₂R^{4B})), OC(O)R^{4B}, OC(O)NR^{4C}R^{4D}, ONR^{4C}(C(O)R^{4B}), OS(O)₂R^{4B}, OP(O)(OR^{4E})(OR^{4F}), S(O)OR^{4A}, S(O)R^{4B}, S(O)₂R^{4B}, S(O)₂NR^{4C}R^{4D}, S(O)₂OR^{4A}, S(═NR^{4G})(O)R^{4B}, S(═NR^{4G})(O)NR^{4C}NR^{4D}, P(O)(OR^{4E})(OR^{4F}), and P(O)(OR^{4E})(R^{4F}), and
R⁵ is selected from H, C₁₋₆-alkyl, C₂₋₆ alkenyl, C₂₋₆ alkynyl, C₃₋₆-cycloalkyl, 4-10 membered heterocycloalkyl, phenyl, C₃₋₆-cycloalkyl-C₁₋₃-alkyl, (4-10 membered heterocycloalkyl)-C₁₋₃-alkyl-, phenyl-C₁₋₃-alkyl-, halo, CN, NO₂, OR^{5a}, SR^{5a}, C(O)R^{5b}, C(O)NR^{5c}R^{5d}, C(O)NR^{5c}(OR^{5a}), C(O)NR^{5c} (S(O)₂R^{5b}), C(O)NR^{5c}(S(O)₂NR^{5c}R^{5d}), NR^{5c} OR^{5a}, NR^{5c}R^{5d}, NR^{5c}(C(O)R^{5b}), NR^{5c}(C(O)OR^{5a}), N(OR^{5a})(C(O)R^{5b}), NR^{5c}(C(O)NR^{5c}R^{5d}), NR^{5c}(C(O)NR^{5c}(C(O)R^{5b})), NR^{5c}(S(O)₂R^{5b}), NR^{5c}(S(O)₂NR^{5c}R^{5d}), NR^{5c}(C(O)NR^{5c}(S(O)₂R^{5b})), OC(O)R^{5b}, OC(O)NR^{5c}R^{5d}, ONR^{5c}(C(O)R^{5b}), OS(O)₂R^{5b}, OP(O)(OR^{5e})(OR^{5f}), S(O)OR^{5a}, S(O)R^{5b}, S(O)₂R^{5b}, S(O)₂NR^{5c}R^{5d}, S(O)₂OR^{5a}, S(═NR^{5g})(O)R^{5b}, S(═NR^{5g})(O)NR^{5c}NR^{5d}, P(O)(OR^{5e})(OR^{5f}), and P(O)(OR^{5e})(R^{5f}), wherein the C₁₋₆-alkyl, C₂₋₆ alkenyl, C₂₋₆ alkynyl, C₃₋₆-cycloalkyl, 4-10 membered heterocycloalkyl, phenyl, C₃₋₆-cycloalkyl-C₁₋₃-alkyl-, (4-10 membered heterocycloalkyl)-C₁₋₃-alkyl-, and phenyl-C₁₋₃-alkyl- of R⁵ are each optionally substituted with 1, 2, or 3 groups independently selected from halo, CN, NO₂, OR^{5A}, SR^{5A}, C(O)R^{5B}, C(O)NR^{5C}R^{5D}, C(O)NR^{5C}(OR^{5A}), C(O)NR^{5C}(S(O)₂R^{5B}), C(O)NR^{5C}(S(O)₂NR^{5C}R^{5D}), NR^{5C}OR^{5A}, NR^{5C}R^{5D}, NR^{5C}(C(O)R^{5B}), NR^{5C}(C(O)OR^{5A}), N(OR^{5A})(C(O)R^{5B}), NR^{5C}(C(O)NR^{5C}R^{5D}), NR^{5C}(C(O)NR^{5C}(C(O)R^{5B})), NR^{5C}(S(O)₂R^{5B}), NR^{5C}(S(O)₂NR^{5C}R^{5D}), NR^{5C}(C(O)NR^{5C}(S(O)₂R^{5B})), OC(O)R^{5B}, OC(O)NR^{5C}R^{5D}, ONR^{5C}(C(O)R^{5B}), OS(O)₂R^{5B}, OP(O)(OR^{5E})(OR^{5F}), S(O)OR^{5A}, S(O)R^{5B}, S(O)₂R^{5B}, S(O)₂NR^{5C}R^{5D}, S(O)₂OR^{5A}, S(═NR^{5G})(O)R^{5B}, S(═NR^{5G})(O)NR^{5C}NR^{5D}, P(O)(OR^{5E})(OR^{5F}), and P(O)(OR^{5E})(R^{5F});
R⁶ is selected from (4-10 membered heterocycloalkyl)-C₁₋₃-alkyl- and (5-10 membered heteroaryl)-C₁₋₃-alkyl-, wherein the (4-10 membered heterocycloalkyl)-C₁₋₃-alkyl- and (5-10 membered heteroaryl)-C₁₋₃-alkyl- of R⁶ are each optionally substituted with 1, 2, or 3 groups independently selected from C₁₋₆-alkyl, —OH, and halo;
R^{4a}, R^{4b}, R^{4c}, and R^{4d} are each independently selected from H, C₁₋₆-alkyl, C₂₋₆ alkenyl, C₂₋₆ alkynyl, C₃₋₆-cycloalkyl, 4-10 membered heterocycloalkyl, 5-10 membered heteroaryl, and phenyl, wherein the C₁₋₆-alkyl, C₂₋₆ alkenyl, and C₂₋₆ alkynyl of R^{4a}, R^{4b}, R^{4c}, and R^{4d} are each optionally substituted with 1, 2, or 3 groups independently selected from —OH and halo; and the C₃₋₆-cycloalkyl, 4-10 membered heterocycloalkyl, 5-10 membered heteroaryl, and phenyl of R^{4a}, R^{4b}, R^{4c}, and R^{4d} are each optionally substituted with 1, 2, or 3 groups independently selected from C₁₋₆-alkyl, —OH, and halo;
R⁴, R^{4f}, and R^{4g} are each independently selected from H and C₁₋₆-alkyl;
R^{4A}, R^{4B}, R^{4C} and R^{4D} are each independently selected from H, C₁₋₆alkyl, C₂₋₆ alkenyl, C₂₋₆ alkynyl, C₃₋₆-cycloalkyl, 4-10 membered heterocycloalkyl, 4-10 membered heterocycloalkyl-C₁₋₃-alkyl-, 5-10 membered heteroaryl, and phenyl, wherein the C₁₋₆-alkyl, C₂₋₆ alkenyl, and C₂₋₆ alkynyl of R^{4A}, R^{4B}, R^{4C} and R^{4D} are each optionally substituted with 1, 2, or 3 groups independently selected from —OH, halo and —OC(O)—C₁₋₁₅-alkyl; and the C₃₋₆-cycloalkyl, 4-10 membered heterocycloalkyl, 4-10 membered heterocycloalkyl-C₁₋₃-alkyl-, 5-10 membered heteroaryl, and phenyl of R^{4A}, R^{4B}, R^{4C} and R^{4D} are each optionally substituted with 1, 2, or 3 groups independently selected from C₁₋₆-alkyl, —OH, and halo;
R^{4E}, R^{4F} and R^{4G} are each independently selected from H and C₁₋₆-alkyl;
R^{5a}, R^{5b}, R^{5c}, and R^{5d} are each independently selected from H, C₁₋₆-alkyl, C₂₋₆ alkenyl, C₂₋₆ alkynyl, C₃₋₆-cycloalkyl, 4-10 membered heterocycloalkyl, 5-10 membered heteroaryl, and phenyl, wherein the C₁₋₆-alkyl, C₂₋₆ alkenyl, and C₂₋₆ alkynyl of R⁵, R^{5b}, R^{5c}, and R^{5d} are each optionally substituted with 1, 2, or 3 groups independently selected from —OH and halo; and the C₃₋₆-cycloalkyl, 4-10 membered heterocycloalkyl, 5-10 membered heteroaryl, and phenyl of R^{5a}, R^{5b}, R^{5c}, and R^{5d} are each optionally substituted with 1, 2, or 3 groups independently selected from C₁₋₆-alkyl, —OH, and halo;
R^{5e}, R^{5f}, and R^{5g} are each independently selected from H and C₁₋₆-alkyl;
R^{5A}, R^{5B}, R^{5C} and R^{5D} are each independently selected from H, C₁₋₆-alkyl, C₂₋₆ alkenyl, C₂₋₆ alkynyl, C₃₋₆-cycloalkyl, 4-10 membered heterocycloalkyl, 4-10 membered heterocycloalkyl-C₁₋₃-alkyl-, 5-10 membered heteroaryl, and phenyl, wherein the C₁₋₆-alkyl, C₂₋₆alkenyl, and C₂₋₆ alkynyl of R^{5A}, R^{5B}, R^{5C} and R^{5D} are each optionally substituted with 1, 2, or 3 groups independently selected from —OH, halo and —OC(O)—C₁₋₁₅-alkyl; and the C₃₋₆-cycloalkyl, 4-10 membered heterocycloalkyl, 4-10 membered heterocycloalkyl-C₁₋₃-alkyl-, 5-10 membered heteroaryl, and phenyl of R^{5A}, R^{5B}, R^{5C} and R^{5D} are each optionally substituted with 1, 2, or 3 groups independently selected from C₁₋₆-alkyl, —OH, and halo; and
R^{5E}, R^{5F} and R^{5G} are each independently selected from H and C₁₋₆-alkyl.

In an embodiment of the compound of formula (I), or a pharmaceutically acceptable salt thereof, R¹ is halo.

In an embodiment of the compound of formula (I), or a pharmaceutically acceptable salt thereof, R¹ is CN.

In an embodiment of the compound of formula (I), or a pharmaceutically acceptable salt thereof, R² is halo.

In an embodiment of the compound of formula (I), or a pharmaceutically acceptable salt thereof, R¹ is chloro, fluoro or CN; and R² is chloro or fluoro.

In an embodiment of the compound of formula (I), or a pharmaceutically acceptable salt thereof, R¹ is chloro; and R² is fluoro.

In an embodiment of the compound of formula (I), or a pharmaceutically acceptable salt thereof, R¹ is CN; and R² is chloro or fluoro.

In an embodiment of the compound of formula (I), or a pharmaceutically acceptable salt thereof, R¹ is CN; and R² is fluoro.

In an embodiment of the compound of formula (I), or a pharmaceutically acceptable salt thereof, R³ is H or —CH₃.

In an embodiment of the compound of formula (I), or a pharmaceutically acceptable salt thereof, R³ is —CH₃.

In an embodiment of the compound of formula (I), or a pharmaceutically acceptable salt thereof, R⁴ is (a) selected from C₁₋₆-alkyl, C₂₋₆ alkenyl, C₂₋₆ alkynyl, C₃₋₆-cycloalkyl, 4-10 membered heterocycloalkyl, phenyl, C₃₋₆-cycloalkyl-C₁₋₃-alkyl-, (4-10 membered heterocycloalkyl)-C₁₋₃-alkyl-, phenyl-C₁₋₃-alkyl-, halo, CN, NO₂, OR^{4a}, SR^{4a}, C(O)R^{4b}, C(O)NR^{4c}R^{4d}, C(O)NR^{4c}(OR^{4a}), C(O)NR^{4c}(S(O)₂R^{4b}), C(O)NR^{4c}(S(O)₂NR^{4c}R^{4d}), NR^{4c}OR^{4a}, NR^{4c}R^{4d}, NR^{4c}(C(O)R^{4b}), NR^{4c}(C(O)OR^{4a}), N(OR^{4a})(C(O)R^{4b}), NR^{4c}(C(O)NR^{4c}R^{4d}), NR^{4c}(C(O)NR^{4c}(C(O)R^{4b})), NR^{4c}(S(O)₂R^{4b}), NR^{4c}(S(O)₂NR^{4c}R^{4d}), NR^{4c}(C(O)NR^{4c}(S(O)₂R^{4b})), OC(O)R^{4b}, OC(O)NR^{4c}R^{4d}, ONR^{4c}(C(O)R^{4b}), OS(O)₂R^{4b}, OP(O)(OR^{4e})(OR^{4f}), S(O)OR^{4a}, S(O)R^{4b}, S(O)₂R^{4b}, S(O)₂NR^{4c}R^{4d}, S(O)₂OR^{4a}, S(═NR^{4g})(O)R^{4b}, S(═NR^{4g})(O)NR^{4c}NR^{4d}, P(O)(OR^{4e})(OR^{4f}), and P(O)(OR^{4e})(R⁴), wherein the C₁₋₆-alkyl, C₂₋₆ alkenyl, C₂₋₆ alkynyl, C₃₋₆-cycloalkyl, 4-10 membered heterocycloalkyl, phenyl, C₃₋₆-cycloalkyl-C₁₋₃-alkyl-, (4-10 membered heterocycloalkyl)-C₁₋₃-alkyl-, and phenyl-C₁₋₃-alkyl- of R⁴ are each optionally substituted with 1, 2, or 3 groups independently selected from halo, CN, NO₂, OR^{4A}, SR^{4A}, C(O)R^{4B}, C(O)NR^{4C}R^{4D}, C(O)NR^{4C}(OR^{4A}), C(O)NR^{4C}(S(O)₂R^{4B}), C(O)NR^{4C}(S(O)₂NR^{4C}R^{4D}), NR^{4C}OR^{4A}, NR^{4C}R^{4D}, NR^{4C}(C(O)R^{4B}), NR^{4C}(C(O)OR^{4A}), N(OR^{4A})(C(O)R^{4B}), NR^{4C}(C(O)NR^{4C}R^{4D}), NR^{4C}(C(O)NR^{4C}(C(O)R^{4B})), NR^{4C}(S(O)₂R^{4B}), NR^{4C}(S(O)₂NR^{4C}R^{4D}), NR^{4C}(C(O)NR^{4C}(S(O)₂R^{4B})), OC(O)R^{4B}, OC(O)NR^{4C}R^{4D}, ONR^{4C}(C(O)R^{4B}), OS(O)₂R^{4B}, OP(O)(OR^{4E})(OR^{4F}), S(O)OR^{4A}, S(O)R^{4B} S(O)₂R^{4B} S(O)₂NR^{4C}R^{4D}, S(O)₂OR^{4A}, S(═NR^{4G})(O)R^{4B}, S(═NR^{4G})(O)NR^{4C}NR^{4D}, P(O)(OR^{4E})(OR^{4F}), and P(O)(OR^{4E})(R^{4F}) or R⁴ is (b) 5-10 membered heteroaryl or (5-10 membered heteroaryl)-C₁₋₃-alkyl-, wherein the 5-10 membered heteroaryl and (5-10 membered heteroaryl)-C₁₋₃-alkyl- are each substituted with C(O)OR^{4A} or with a carboxylic acid isostere, and optionally with 1, 2, or 3 groups independently selected from halo, CN, NO₂, OR^{4A}, SR^{4A}, C₁₋₃-alkyl, C₁₋₃-alkyl substituted with 1, 2 or 3 halo, C(O)R^{4B}, C(O)NR^{4C}R^{4D}, C(O)NR^{4C}(OR^{4A}), C(O)NR^{4C}(S(O)₂R^{4B}), C(O)NR^{4C}(S(O)₂NR^{4C}R^{4D}), NR^{4C}OR^{4A}, NR^{4C}R^{4D}, NR^{4C}(C(O)R^{4B}), NR^{4C}(C(O)OR^{4A}), N(OR^{4A})(C(O)R^{4B}), NR^{4C}(C(O)NR^{4C}R^{4D}), NR^{4C}(C(O)NR^{4C}(C(O)R^{4B})), NR^{4C}(S(O)₂R^{4B}), NR^{4C}(S(O)₂NR^{4C}R^{4D}), NR^{4C}(C(O)NR^{4C}(S(O)₂R^{4B})), OC(O)R^{4B}, OC(O)NR^{4C}R^{4D}, ONR^{4C}(C(O)R^{4B}) OS(O)₂R^{4B} OP(O)(OR^{4E})(OR^{4F}) S(O)OR^{4A} S(O)R^{4B} S(O)₂R^{4B}, S(O)₂NR^{4C}R^{4D}, S(O)₂OR^{4A}, S(═NR^{4G})(O)R^{4B}, S(═NR^{4G})(O)NR^{4C}NR^{4D}, P(O)(OR^{4E})(OR^{4F}), and P(O)(OR^{4E})(R^{4F}),
wherein R^{4a}, R^{4b}, R^{4c}, R^{4d}, R^{4e}, R^{4f}, R^{4g}, R^{4A}, R^{4B}, R^{4C}, R^{4D}, R^{4E}, R^{4F} and R^{4G} are as defined herein.

In an embodiment of the compound of formula (I), or a pharmaceutically acceptable salt thereof, R⁴ is (a) selected from C₁₋₃-alkyl, C₂₋₄ alkenyl, C₂₋₄ alkynyl, C₃₋₆-cycloalkyl, 4-10 membered heterocycloalkyl, phenyl, and halo, wherein the C₁₋₃-alkyl, C₂₋₄ alkenyl, C₂₋₄ alkynyl, C₃₋₆-cycloalkyl, 4-10 membered heterocycloalkyl, and phenyl of R⁴ are each optionally substituted with 1, 2, or 3 groups independently selected from halo and C₁₋₃-alkyl, or R⁴ is (b) 5-membered heteroaryl or (5-10 membered heteroaryl)-C₁₋₃-alkyl-, wherein the 5-10 membered heteroaryl and (5-10 membered heteroaryl)-C₁₋₃-alkyl- are each substituted with C(O)OR^{4A} or with a carboxylic acid isostere, and optionally with 1, 2, or 3 groups independently selected from halo, C₁₋₃-alkyl and C₁₋₃-alkyl substituted with 1, 2 or 3 halo; wherein R^{4A} is selected from H and C₁₋₃-alkyl.

In an embodiment of the compound of formula (I), or a pharmaceutically acceptable salt thereof, R⁴ is C₁₋₃-alkyl or C₁₋₃-alkyl substituted with 1, 2 or 3 halo, for example methyl, ethyl or trifluoromethyl.

In an embodiment of the compound of formula (I), or a pharmaceutically acceptable salt thereof, R⁴ is 5-10 membered heteroaryl or (5-10 membered heteroaryl)-C₁₋₃-alkyl-, wherein the 5-10 membered heteroaryl and (5-10 membered heteroaryl)-C₁₋₃-alkyl- are each substituted with C(O)OR^{4A} or with a carboxylic acid isostere, and optionally with 1, 2 or 3 groups independently selected from halo, CN, NO₂, OR^{4A}, SR^{4A}, C₁₋₃-alkyl, C₁₋₃-alkyl substituted with 1, 2 or 3 halo, C(O)R^{4B}, C(O)NR^{4C}R^{4D}, C(O)NR^{4C}(OR^{4A}), C(O)NR^{4C}(S(O)₂R^{4B}), C(O)NR^{4C}(S(O)₂NR^{4C}R^{4D}), NR^{4C}OR^{4A}, NR^{4C}R^{4D}, NR^{4C}(C(O)R^{4B}), NR^{4C}(C(O)OR^{4A}), N(OR^{4A})(C(O)R^{4B}), NR^{4C}(C(O)NR^{4C}R^{4D}), NR^{4C}(C(O)NR^{4C}(C(O)R^{4B})), NR^{4C}(S(O)₂R^{4B}), NR^{4C}(S(O)₂NR^{4C}R^{4D}), NR^{4C}(C(O)NR^{4C}(S(O)₂R^{4B})), OC(O)R^{4B}, OC(O)NR^{4C}R^{4D}, ONR^{4C}(C(O)R^{4B}), OS(O)₂R^{4B}, OP(O)(OR^{4E})(OR^{4F}), S(O)OR^{4A}, S(O)R^{4B} S(O)₂R^{4B} S(O)₂NR^{4C}R^{4D}, S(O)₂OR^{4A}, S(═NR^{4G})(O)R^{4B}, S(═NR^{4G})(O)NR^{4C}NR^{4D}, P(O)(OR^{4E})(OR^{4F}), and P(O)(OR^{4E})(R^{4F}) wherein R^{4A}, R^{4B}, R^{4C}, R^{4D}, R^{4E}, R^{4F} and R^{4G} are as defined herein.

In an embodiment of the compound of formula (I), or a pharmaceutically acceptable salt thereof, R⁴ is 5-10 membered heteroaryl or (5-10 membered heteroaryl)-C₁₋₃-alkyl-, wherein the 5-10 membered heteroaryl and (5-10 membered heteroaryl)-C₁₋₃-alkyl- are each substituted with C(O)OR^{4A} or with a carboxylic acid isostere, and optionally with 1, 2, or 3 groups independently selected from halo, C₁₋₃-alkyl and C₁₋₃-alkyl substituted with 1, 2 or 3 halo; wherein R^{4A} is selected from H and C₁₋₃-alkyl.

In an embodiment of the compound of formula (I), or a pharmaceutically acceptable salt thereof, R⁴ has the structure of Formula H1 or H2:

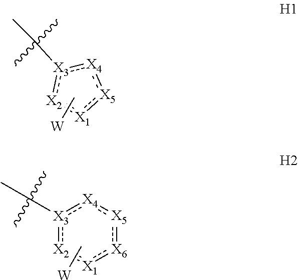

wherein:
X₁ is C or N, and each of X₂, X₃, X₄, X₅, and X₆ is independently C═O, CR^{4h}, NR⁴ⁱ, O, or S;
W is C(O)OR^{4A} or a carboxylic acid isostere;
each [symbol] is a single or a double bond;
each R^{4h} is independently selected from H, C₁₋₆-alkyl, C₂₋₆ alkenyl, C₂₋₆ alkynyl, C₃₋₆-cycloalkyl, 4-10 membered heterocycloalkyl, 5-10 membered heteroaryl, phenyl, C₃₋₆-cycloalkyl-C₁₋₃-alkyl-, (4-10 membered heterocycloalkyl)-C₁₋₃-alkyl-, (5-10 membered heteroaryl)-C₁₋₃-alkyl-, phenyl-C₁₋₃-alkyl-, halo, CN, NO₂, OR^{4a}, SR^{4a}, C(O)OR^{4a}, C(O)R^{4b}, C(O)NR^{4c}R^{4d}, C(O)NR^{4c}(OR^{4a}), C(O)NR^{4c}(S(O)₂R^{4b}), C(O)NR^{4c}(S(O)₂NR^{4c}R^{4d}), NR^{4c}OR^{4a}, NR^{4c}R^{4d}, NR^{4c}(C(O)R^{4b}), NR^{4c}(C(O)OR^{4a}), N(OR^{4a})(C(O)R^{4b}), NR^{4c}(C(O)NR^{4c}R^{4d}), NR^{4c}(C(O)NR^{4c}(C(O)R^{4b})), NR^{4c}(S(O)₂R^{4b}), NR^{4c}(S(O)₂NR^{4c}R^{4d}), NR^{4c}(C(O)NR^{4c}(S(O)₂R^{4b})), OC(O)R^{4b}, OC(O)NR^{4c}R^{4d}, ONR^{4c}(C(O)R^{4b}), OS(O)₂R^{4b}, OP(O)(OR^{4e})(OR^{4f}), S(O)OR^{4a}, S(O)R^{4b}, S(O)₂R^{4b}, S(O)₂NR^{4c}R^{4d}, S(O)₂OR^{4a}, S(═NR^{4g})(O)R^{4b}, S(═NR^{4g})(O)NR^{4c}NR^{4d}, P(O)(OR^{4e})(OR^{4f}), and P(O)(OR^{4e})(R^{4f}), wherein the C₁₋₆-alkyl, C₂₋₆ alkenyl, C₂₋₆ alkynyl, C₃₋₆-cycloalkyl, 4-10 membered heterocycloalkyl, 5-10 membered heteroaryl, phenyl, C₃₋₆-cycloalkyl-C₁₋₃-alkyl-, (4-10 membered heterocycloalkyl)-C₁₋₃-alkyl-, (5-10 membered heteroaryl)-C₁₋₃-alkyl-, and phenyl-C₁₋₃-alkyl- are each optionally substituted with 1, 2, or 3 groups independently selected from halo, CN, NO₂, OR^{4A}, SR^{4A}, C₁₋₃-alkyl, C₁₋₃-alkyl substituted with 1, 2 or 3 halo, C(O)OR^{4A}, C(O)R^{4B}, C(O)NR^{4C}R^{4D}, C(O)NR^{4C}(OR^{4A}), C(O)NR^{4C}(S(O)₂R^{4B}), C(O)NR^{4C}(S(O)₂NR^{4C}R^{4D}), NR^{4C}OR^{4A}, NR^{4C}R^{4D}, NR^{4C}(C(O)R^{4B}), NR^{4C}(C(O)OR^{4A}), N(OR^{4A})(C(O)R^{4B}), NR^{4C}(C(O)NR^{4C}R^{4D}), NR^{4C}(C(O)NR^{4C}(C(O)R^{4B})), NR^{4C}(S(O)₂R^{4B}), NR^{4C}(S(O)₂NR^{4C}R^{4D}), NR^{4C}(C(O)NR^{4C}(S(O)₂R^{4B})), OC(O)R^{4B}, OC(O)NR^{4C}R^{4D}, ONR^{4C}(C(O)R^{4B}), OS(O)₂R^{4B}, OP(O)(OR^{4E})(OR^{4F}), S(O)OR^{4A}, S(O)R^{4B}, S(O)₂R^{4B}, S(O)₂NR^{4C}R^{4D}, S(O)₂OR^{4A}, S(═NR^{4G})(O)R^{4B}, S(═NR^{4G})(O)NR^{4C}NR^{4D}, P(O)(OR^{4E})(OR^{4F}), and P(O)(OR^{4E})(R^{4F});
each R⁴ⁱ is independently selected from H, C₁₋₆-alkyl, C₂₋₆ alkenyl, C₂₋₆ alkynyl, C₃₋₆-cycloalkyl, 4-10 membered heterocycloalkyl, 5-10 membered heteroaryl, phenyl, C₃₋₆-cycloalkyl-C₁₋₃-alkyl-, (4-10 membered heterocycloalkyl)-C₁₋₃-alkyl-, (5-10 membered heteroaryl)-C₁₋₃-alkyl-, phenyl-C₁₋₃-alkyl-, CN, C(O)OR^{4a}, C(O)R^{4b}, C(O)NR^{4c}R^{4d}, C(O)NR^{4c}(OR^{4a}), C(O)NR^{4c}(S(O)₂R^{4b}), and C(O)NR^{4c}(S(O)₂NR^{4c}R^{4d}) wherein the C₁₋₆-alkyl, C₂₋₆ alkenyl, C₂₋₆ alkynyl, C₃₋₆-cycloalkyl, 4-10 membered heterocycloalkyl, 5-10 membered heteroaryl, phenyl, C₃₋₆-cycloalkyl-C₁₋₃-alkyl-, (4-10 membered heterocycloalkyl)-C₁₋₃-alkyl-, (5-10 membered heteroaryl)-C₁₋₃-alkyl-, and phenyl-C₁₋₃-alkyl- are each optionally substituted with 1, 2, or 3 groups independently selected from halo, CN, NO₂, OR^{4A}, SR^{4A}, C(O)OR^{4A}, C(O)R^{4B}, C(O)NR^{4C}R^{4D}, C(O)NR^{4C}(OR^{4A}), C(O)NR^{4C}(S(O)₂R^{4B}), C(O)NR^{4C}(S(O)₂NR^{4C}R^{4D}), NR^{4C}OR^{4A}, NR^{4C}R^{4D}, NR^{4C}(C(O)R^{4B}), NR^{4C}(C(O)OR^{4A}), N(OR^{4A})(C(O)R^{4B}), NR^{4C}(C(O)NR^{4C}R^{4D}), NR^{4C}(C(O)NR^{4C}(C(O)R^{4B})), NR^{4C}(S(O)₂R^{4B}), NR^{4C}(S(O)₂NR^{4C}R^{4D}), NR^{4C}(C(O)NR^{4C}(S(O)₂R^{4B})), OC(O)R^{4B}, OC(O)NR^{4C}R^{4D}, ONR^{4C}(C(O)R^{4B}), OS(O)₂R^{4B}, OP(O)(OR^{4E})(OR^{4F}), S(O)OR^{4A}, S(O)R^{4B}, S(O)₂R^{4B}, S(O)₂NR^{4C}R^{4D}, S(O)₂OR^{4A}, S(═NR^{4G})(O)R^{4B}, S(═NR^{4G})(O)NR^{4C}NR^{4D}, P(O)(OR^{4E})(OR^{4F}), and P(O)(OR^{4E})(R^{4F}); or
R^{4h} and R⁴ⁱ can be taken together to form a C₃₋₆-cycloalkyl, phenyl, 4-10 membered heterocycloalkyl, or 5-10 membered heteroaryl which may be further substituted with groups independently selected from C₁₋₆-alkyl, —OH, and halo,
wherein R^{4a}, R^{4b}, R^{4c}, R^{4d}, R^{4e}, R^{4f}, R^{4g}, R^{4A}, R^{4B}, R^{4C}, R^{4D}, R^{4E}, R^{4F} and R^{4G} are as defined herein.

In an embodiment of the compound of formula (I), or a pharmaceutically acceptable salt thereof, R⁴ has the structure of Formula H1a or H2a:

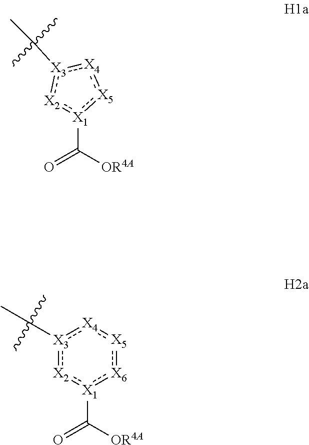

wherein the ring is aromatic; wherein
X₁ is C, and each of X₂, X₃, X₄, X₅, and X₆ is independently C═O, CR^{4h}, NR⁴ⁱ, O, or S, wherein at least one of X₂-X₆ is N, O, or S;
R^{4A} is selected from H and C₁₋₃-alkyl; and
if present, R^{4h} is halo, C₁₋₃-alkyl or C₁₋₃-alkyl substituted with 1, 2 or 3 halo, and R⁴ⁱ is C₁₋₃-alkyl.

In an embodiment of the compound of formula (I), or a pharmaceutically acceptable salt thereof, R⁴ is selected from the group consisting of:

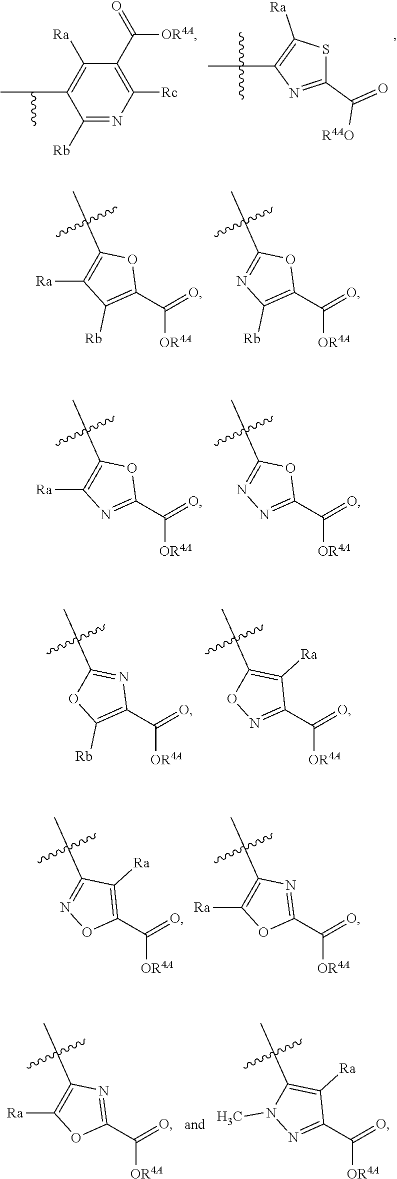

wherein Ra, Rb and Rc are independently selected from H, C₁₋₃-alkyl and C₁₋₃-alkyl substituted with 1, 2 or 3 halo, and R^{4A} is selected from H and C₁₋₃-alkyl.

In an embodiment of the compound of formula (I), or a pharmaceutically acceptable salt thereof, R⁵ is (a) selected from C₁₋₆-alkyl, C₂₋₆ alkenyl, C₂₋₆ alkynyl, C₃₋₆-cycloalkyl, 4-10 membered heterocycloalkyl, phenyl, C₃₋₆-cycloalkyl-C₁₋₃-alkyl, (4-10 membered heterocycloalkyl)-C₁₋₃-alkyl-, phenyl-C₁₋₃-alkyl-, halo, CN, NO₂, OR^{5a}, SR^{5a}, C(O)R^{5b}, C(O)NR^{5c}R^{5d}, C(O)NR^{5c}(OR^{5a}), C(O)NR^{5c}(S(O)₂R^{5b}), C(O)NR^{5c}(S(O)₂NR^{5c}R^{5d}), NR^{5c}OR^{5a}, NR^{5c}R^{5d}, NR^{5c}(C(O)R^{5b}), NR^{5c}(C(O)OR^{5a}), N(OR^{5a})(C(O)R^{5b}), NR^{5c}(C(O)NR^{5c}R^{5d}), NR^{5c}(C(O)NR^{5c}(C(O)R^{5b})), NR^{5c}(S(O)₂R^{5b}), NR^{5c}(S(O)₂NR^{5c}R^{5d}), NR^{5c}(C(O)NR^{5c}(S(O)₂R^{5b})), OC(O)R^{5b}, OC(O)NR^{5c}R^{5d}, ONR^{5c}(C(O)R^{5b}), OS(O)₂R^{5b}, OP(O)(OR^{5e})(OR^{5f}), S(O)OR^{5a}, S(O)R^{5b}, S(O)₂R^{5b}, S(O)₂NR^{5c}R^{5d}, S(O)₂OR^{5a}, S(═NR^{5g})(O)R^{5b}, S(═NR^{5g})(O)NR^{5c}NR^{5d}, P(O)(OR^{5e})(OR⁵), and P(O)(OR^{5e})(R⁵), wherein the C₁₋₆-alkyl, C₂₋₆ alkenyl, C₂₋₆ alkynyl, C₃₋₆-cycloalkyl, 4-10 membered heterocycloalkyl, phenyl, C₃₋₆-cycloalkyl-C₁₋₃-alkyl-, (4-10 membered heterocycloalkyl)-C₁₋₃-alkyl-, and phenyl-C₁₋₃-alkyl- of R⁵ are each optionally substituted with 1, 2, or 3 groups independently selected from halo, CN, NO₂, OR^{5A}, SR^{5A}, C(O)R^{5B}, C(O)NR^{5C}R^{5D}, C(O)NR^{5C}(OR^{5A}), C(O)NR^{5C}(S(O)₂R^{5B}), C(O)NR^{5C}(S(O)₂NR^{5C}R^{5D}), NR^{5C}OR^{5A}, NR^{5C}R^{5D}, NR^{5C}(C(O)R^{5B}), NR^{5C}(C(O)OR^{5A}), N(OR^{5A})(C(O)R^{5B}), NR^{5C}(C(O)NR^{5C}R^{5D}), NR^{5C}(C(O)NR^{5C}(C(O)R^{5B})), NR^{5C}(S(O)₂R^{5B}), NR^{5C}(S(O)₂NR^{5C}R^{5D}), NR^{5C}(C(O)NR^{5C}(S(O)₂R^{5B})), OC(O)R^{5B}, OC(O)NR^{5C}R^{5D}, ONR^{5C}(C(O)R^{5B}), OS(O)₂R^{5B}, OP(O)(OR^{5E})(OR^{5F}), S(O)OR^{5A}, S(O)R^{5B}, S(O)₂R^{5B} S(O)₂NR^{5C}R^{5D}, S(O)₂OR^{5A}, S(═NR^{5G})(O)R^{5B}, S(═NR^{5G})(O)NR^{5C}NR^{5D}, P(O)(OR^{5E})(OR^{5F}), and P(O)(OR^{5E})(R^{5F}) or R⁵ is (b) 5-10 membered heteroaryl or (5-10 membered heteroaryl)-C₁₋₃-alkyl-, wherein the 5-10 membered heteroaryl and (5-10 membered heteroaryl)-C₁₋₃-alkyl- are each substituted with C(O)OR^{5A} or with a carboxylic acid isostere, and optionally with 1, 2, or 3 groups independently selected from halo, CN, NO₂, OR^{5A}, SR^{5A}, C₁₋₃-alkyl, C₁₋₃-alkyl substituted with 1, 2 or 3 halo, C(O)R^{5B}, C(O)NR^{5C}R^{5D}, C(O)NR^{5C}(OR^{5A}), C(O)NR^{5C}(S(O)₂R^{5B}), C(O)NR^{5C}(S(O)₂NR^{5C}R^{5D}), NR^{5C}OR^{5A}, NR^{5C}R^{5D}, NR^{5C}(C(O)R^{5B}), NR^{5C}(C(O)OR^{5A}), N(OR^{5A})(C(O)R^{5B}), NR^{5C}(C(O)NR^{5C}R^{5D}), NR^{5C}(C(O)NR^{5C}(C(O)R^{5B})), NR^{5C}(S(O)₂R^{5B}), NR^{5C}(S(O)₂NR^{5C}R^{5D}), NR^{5C}(C(O)NR^{5C}(S(O)₂R^{5B})), OC(O)R^{5B}, OC(O)NR^{5C}R^{5D}, ONR^{5C}(C(O)R^{5B}), OS(O)₂R^{5B}, OP(O)(OR^{5E})(OR^{5F}), S(O)OR^{5A}, S(O)R^{5B}, S(O)₂R^{5B}, S(O)₂NR^{5C}R^{5D}, S(O)₂OR^{5A}, S(═NR^{5G})(O)R^{5B}, S(═NR^{5G})(O)NR^{5C}NR^{5D}, P(O)(OR^{5E})(OR^{5F}), and P(O)(OR^{5E})(R^{5F}),
wherein R^{5a}, R^{5b}, R^{5c}, R^{5d}, R^{5e}, R^{5f}, R^{5g}, R^{5A}, R^{5B}, R^{5C}, R^{5D}, R^{5E}, R^{5F} and R^{5G} are as defined herein.

In an embodiment of the compound of formula (I), or a pharmaceutically acceptable salt thereof, R⁵ is (a) selected from C₁₋₃-alkyl, C₂₋₄ alkenyl, C₂₋₄ alkynyl, C₃₋₆-cycloalkyl, 4-10 membered heterocycloalkyl, phenyl, and halo, wherein the C₁₋₃-alkyl, C₂₋₄ alkenyl, C₂₋₄ alkynyl, C₃₋₆-cycloalkyl, 4-10 membered heterocycloalkyl, and phenyl are each optionally substituted with 1, 2, or 3 groups independently selected from halo and C₁₋₃-alkyl, or R⁵ is (b) 5-10 membered heteroaryl or (5-10 membered heteroaryl)-C₁₋₃-alkyl-, wherein the 5-10 membered heteroaryl and (5-10 membered heteroaryl)-C₁₋₃-alkyl- are each substituted with C(O)OR^{5A} or with a carboxylic acid isostere, and optionally with 1, 2, or 3 groups independently selected from halo, C₁₋₃-alkyl and C₁₋₃-alkyl substituted with 1, 2 or 3 halo; wherein R^{5A} is selected from H and C₁₋₃-alkyl.

In an embodiment of the compound of formula (I), or a pharmaceutically acceptable salt thereof, R⁵ is selected from halo, C₁₋₃-alkyl, and phenyl, wherein the C₁₋₃-alkyl and phenyl are each optionally substituted with 1, 2, or 3 halo.

In an embodiment of the compound of formula (I), or a pharmaceutically acceptable salt thereof, R⁵ is selected from C₁₋₃-alkyl, C₂₋₄ alkenyl, and C₃₋₆-cycloalkyl, wherein the C₁₋₃-alkyl, C₂₋₄ alkenyl, and C₃₋₆-cycloalkyl of R⁵ are each optionally substituted with 1, 2, or 3 halo.

In an embodiment of the compound of formula (I), or a pharmaceutically acceptable salt thereof, R⁵ is selected from —CH₃, —CF₃, F, —CH₂CH₃, and

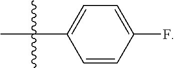 [SMILES: *c1ccc(F)cc1]

In an embodiment of the compound of formula (I), or a pharmaceutically acceptable salt thereof, R⁵ is C₁₋₃-alkyl or C₁₋₃-alkyl substituted with 1, 2 or 3 halo, for example methyl, ethyl or trifluoromethyl.

In an embodiment of the compound of formula (I), or a pharmaceutically acceptable salt thereof, R⁵ is 5-10 membered heteroaryl or (5-10 membered heteroaryl)-C₁₋₃-alkyl-, wherein the R⁵ is 5-10 membered heteroaryl and (5-10 membered heteroaryl)-C₁₋₃-alkyl- are each substituted with C(O)OR^{5A} or with a carboxylic acid isostere, optionally with 1, 2, or 3 groups independently selected from halo, CN, NO₂, OR^{5A}, SR^{5A}, C₁₋₃-alkyl, C₁₋₃-alkyl substituted with 1, 2 or 3 halo, C(O)R^{5B}, C(O)NR^{5C}R^{5D}, C(O)NR^{5C}(OR^{5A}), C(O)NR^{5C}(S(O)₂R^{5B}), C(O)NR^{5C}(S(O)₂NR^{5C}R^{5D}), NR^{5C}OR^{5A}, NR^{5C}R^{5D}, NR^{5C}(C(O)R^{5B}), NR^{5C}(C(O)OR^{5A}), N(OR^{5A})(C(O)R^{5B}), NR^{5C}(C(O)NR^{5C}R^{5D}), NR^{5C}(C(O)NR^{5C}(C(O)R^{5B})), NR^{5C}(S(O)₂R^{5B}), NR^{5C}(S(O)₂NR^{5C}R^{5D}), NR^{5C}(C(O)NR^{5C}(S(O)₂R^{5B})), OC(O)R^{5B}, OC(O)NR^{5C}R^{5D}, ONR^{5C}(C(O)R^{5B}), OS(O)₂R^{5B}, OP(O)(OR^{5E})(OR^{5F}), S(O)OR^{5A}, S(O)R^{5B}, S(O)₂R^{5B}, S(O)₂NR^{5C}R^{5D}, S(O)₂OR^{5A}, S(═NR^{5G})(O)R^{5B}, S(═NR^{5G})(O)NR^{5C}NR^{5D}, P(O)(OR^{5E})(OR^{5F}), and P(O)(OR^{5E})(R^{5F}) wherein R^{5A}, R^{5B}, R^{5C}, R^{5D}, R^{5E}, R^{5F} and R^{5G} are as defined herein.

In an embodiment of the compound of formula (I), or a pharmaceutically acceptable salt thereof, R⁵ has the structure of Formula H11 or H12:

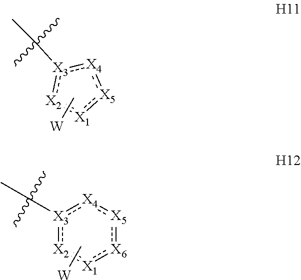

wherein:
X₁ is C or N, and each of X₂, X₃, X₄, X₅, and X₆ is independently C═O, CR^{5h}, NR⁵ⁱ, O, or S;
W is C(O)OR^{5A} or a carboxylic acid isostere;
each [symbol] is a single or a double bond;
each R^{5h} is independently selected from H, C₁₋₆-alkyl, C₂₋₆ alkenyl, C₂₋₆ alkynyl, C₃₋₆-cycloalkyl, 4-10 membered heterocycloalkyl, 5-10 membered heteroaryl, phenyl, C₃₋₆-cycloalkyl-C₁₋₃-alkyl, (4-10 membered heterocycloalkyl)-C₁₋₃-alkyl-, (5-10 membered heteroaryl)-C₁₋₃-alkyl-, phenyl-C₁₋₃-alkyl-, halo, CN, NO₂, OR^{5a}, SR^{5a}, C(O)OR^{5a}, C(O)R^{5b}, C(O)NR^{5c}R^{5d}, C(O)NR^{5c}(OR^{5a}), C(O)NR^{5c}(S(O)₂R^{5b}), C(O)NR^{5c}(S(O)₂NR^{5c}R^{5d}), NR^{5c}OR^{5a}, NR^{5c}R^{5d}, NR^{5c}(C(O)R^{5b}), NR^{5c}(C(O)OR^{5a}), N(OR^{5a})(C(O)R^{5b}), NR^{5c}(C(O)NR^{5c}R^{5d}), NR^{5c}(C(O)NR^{5c}(C(O)R^{5b})), NR^{5c}(S(O)₂R^{5b}), NR^{5c}(S(O)₂NR^{5c}R^{5d}), NR^{5c}(C(O)NR^{5c}(S(O)₂R^{5b})), OC(O)R^{5b}, OC(O)NR^{5c}R^{5d}, ONR^{5c}(C(O)R^{5b}), OS(O)₂R^{5b}, OP(O)(OR^{5e})(OR^{5g}, S(O)OR^{5a}, S(O)R^{5b}, S(O)₂R^{5b}, S(O)₂NR^{5c}R^{5d}, S(O)₂OR^{5a}, S(═NR^{5g})(O)R^{5b}, S(═NR^{5g})(O)NR^{5c}NR^{5d}, P(O)(OR^{5e})(OR⁵), and P(O)(OR^{5e})(R⁵), wherein the C₁₋₆-alkyl, C₂₋₆ alkenyl, C₂₋₆ alkynyl, C₃₋₆-cycloalkyl, 4-10 membered heterocycloalkyl, 5-10 membered heteroaryl, phenyl, C₃₋₆-cycloalkyl-C₁₋₃-alkyl-, (4-10 membered heterocycloalkyl)-C₁₋₃-alkyl-, (5-10 membered heteroaryl)-C₁₋₃-alkyl-, and phenyl-C₁₋₃-alkyl- of are each optionally substituted with 1, 2, or 3 groups independently selected from halo, CN, NO₂, OR^{5A}, SR^{5A}, C₁₋₃-alkyl, C₁₋₃-alkyl substituted with 1, 2 or 3 halo, C(O)OR^{5A}, C(O)R^{5B}, C(O)NR^{5C}R^{5D}, C(O)NR^{5C}(OR^{5A}), C(O)NR^{5C}(S(O)₂R^{5B}), C(O)NR^{5C}(S(O)₂NR^{5C}R^{5D}), NR^{5C}OR^{5A}, NR^{5C}R^{5D}, NR^{5C}(C(O)R^{5B}), NR^{5C}(C(O)OR^{5A}), N(OR^{5A})(C(O)R^{5B}), NR^{5C}(C(O)NR^{5C}R^{5D}), NR^{5C}(C(O)NR^{5C}(C(O)R^{5B})), NR^{5C}(S(O)₂R^{5B}), NR^{5C}(S(O)₂NR^{5C}R^{5D}), NR^{5C}(C(O)NR^{5C}(S(O)₂R^{5B})), OC(O)R^{5B}, OC(O)NR^{5C}R^{5D}, ONR^{5C}(C(O)R^{5B}), OS(O)₂R^{5B}, OP(O)(OR^{5E})(OR^{5F}) S(O)OR^{5A} S(O)R^{5B} S(O)₂R^{5B} S(O)₂NR^{5C}R^{5D}, S(O)₂OR^{5A}, S(═NR^{5G})(O)R^{5B}, S(═NR^{5G})(O)NR^{5C}NR^{5D}, P(O)(OR^{5E})(OR^{5F}), and P(O)(OR^{5E})(R^{5F});
each R⁵ⁱ is independently selected from H, C₁₋₆-alkyl, C₂₋₆ alkenyl, C₂₋₆ alkynyl, C₃₋₆-cycloalkyl, 4-10 membered heterocycloalkyl, 5-10 membered heteroaryl, phenyl, C₃₋₆-cycloalkyl-C₁₋₃-alkyl, (4-10 membered heterocycloalkyl)-C₁₋₃-alkyl-, (5-10 membered heteroaryl)-C₁₋₃-alkyl-, phenyl-C₁₋₃-alkyl-, CN, C(O)OR^{5a}, C(O)R^{5b}, C(O)NR^{5c}R^{5d}, C(O)NR^{5c}(OR^{5a}), C(O)NR^{5c}(S(O)₂R^{5b}), C(O)NR^{5c}(S(O)₂NR^{5c}R^{5d}) wherein the C₁₋₆-alkyl, C₂₋₆ alkenyl, C₂₋₆ alkynyl, C₃₋₆-cycloalkyl, 4-10 membered heterocycloalkyl, 5-10 membered heteroaryl, phenyl, C₃₋₆-cycloalkyl-C₁₋₃-alkyl-, (4-10 membered heterocycloalkyl)-C₁₋₃-alkyl-, (5-10 membered heteroaryl)-C₁₋₃-alkyl-, and phenyl-C₁₋₃-alkyl- are each optionally substituted with 1, 2, or 3 groups independently selected from halo, CN, NO₂, OR^{5A}, SR^{5A}, C(O)OR^{5A}, C(O)R^{5B}, C(O)NR^{5C}R^{5D}, C(O)NR^{5C}(OR^{5A}), C(O)NR^{5C}(S(O)₂R^{5B}), C(O)NR^{5C}(S(O)₂NR^{5C}R^{5D}), NR^{5C}OR^{5A}, NR^{5C}R^{5D}, NR^{5C}(C(O)R^{5B}), NR^{5C}(C(O)OR^{5A}), N(OR^{5A})(C(O)R^{5B}), NR^{5C}(C(O)NR^{5C}R^{5D}), NR^{5C}(C(O)NR^{5C}(C(O)R^{5B})), NR^{5C}(S(O)₂R^{5B}), NR^{5C}(S(O)₂NR^{5C}R^{5D}), NR^{5C}(C(O)NR^{5C}(S(O)₂R^{5B})), OC(O)R^{5B}, OC(O)NR^{5C}R^{5D}, ONR^{5C}(C(O)R^{5B}), OS(O)₂R^{5B}, OP(O)(OR^{5E})(OR^{5F}), S(O)OR^{5A}, S(O)R^{5B}, S(O)₂R^{5B}, S(O)₂NR^{5C}R^{5D}, S(O)₂OR^{5A}, S(═NR^{5G})(O)R^{5B}, S(═NR^{5G})(O)NR^{5C}NR^{5D}, P(O)(OR^{5E})(OR^{5F}), and P(O)(OR^{5E})(R^{5F}); or R^{5h} and R⁵ⁱ Can be taken together to form a C₃₋₆-cycloalkyl, phenyl, 4-10 membered heterocycloalkyl, or 5-10 membered heteroaryl which may be further substituted with groups independently selected from C₁₋₆-alkyl, —OH, and halo,
wherein R^{5a}, R^{5b}, R^{5c}, R^{5d}, R^{5e}, R^{5f} and R^{5g}, R^{5A}, R^{5B}, R^{5C}, R^{5D}, R^{5E}, R^{5F} and R^{5G} are as defined herein.

In an embodiment of the compound of formula (I), or a pharmaceutically acceptable salt thereof, R⁵ has the structure of Formula H11a or H12a:

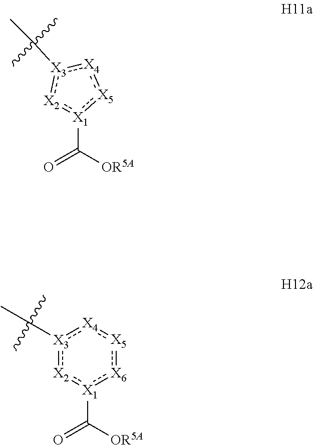

wherein the ring is aromatic; wherein
X₁ is C, and each of X₂, X₃, X₄, X₅, and X₆ is independently C═O, CR^{5h}, NR⁵ⁱ, O, or S, wherein at least one of X₂—X₆ is N, O, or S;
R^{5A} is selected from H and C₁₋₃-alkyl; and
if present, R^{5h} is halo, C₁₋₃-alkyl or C₁₋₃-alkyl substituted with 1, 2 or 3 halo, and R⁵ⁱ is C₁₋₃-alkyl.

In an embodiment of the compound of formula (I), or a pharmaceutically acceptable salt thereof, R⁵ has the structure of Formula H11a, for example wherein R⁵ is selected from the group consisting of:

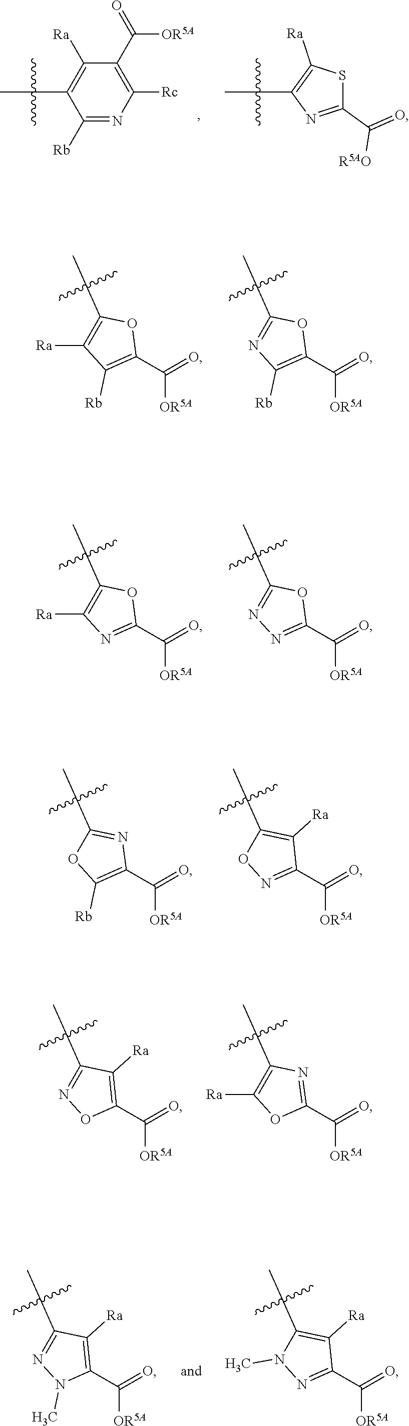

wherein Ra, Rb and Rc are independently selected from H, C₁₋₃-alkyl and C₁₋₃-alkyl substituted with 1, 2 or 3 halo, and R^{5A} is selected from H and C₁₋₃-alkyl.

In an embodiment of the compound of formula (I), or a pharmaceutically acceptable salt thereof, R⁶ is selected from (4-6 membered heterocycloalkyl)-CH₂— and (5-6 membered heteroaryl)-CH₂—, wherein the (4-6 membered heterocycloalkyl)-CH₂— and (5-6 membered heteroaryl)-CH₂— of R⁶ are each optionally substituted with 1, 2, or 3 groups independently selected from C₁₋₆-alkyl, —OH, and halo.

In an embodiment of the compound of formula (I), or a pharmaceutically acceptable salt thereof, R⁶ is selected from the group consisting of:

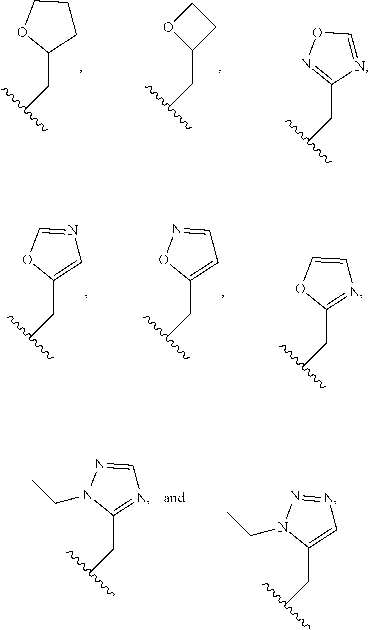

wherein [symbol] indicates the point of attachment to the rest of the molecule.

In an embodiment of the compound of formula (I), or a pharmaceutically acceptable salt thereof, R⁶ is of formula:

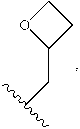 [SMILES: *CC1CCO1]

wherein [symbol] indicates the point of attachment to the rest of the molecule.

In an embodiment of the compound of formula (I), or a pharmaceutically acceptable salt thereof, R⁶ is of formula:

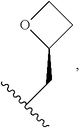 [SMILES: *C[C@@H]1CCO1]

wherein [symbol] indicates the point of attachment to the rest of the molecule.

In an embodiment of the compound of formula (I), or a pharmaceutically acceptable salt thereof, X is O.

In an embodiment of the compound of formula (I), or a pharmaceutically acceptable salt thereof, W is O.

In an embodiment of the compound of formula (I), or a pharmaceutically acceptable salt thereof, [symbol] is a single bond.

In an embodiment, the compound of formula (I), or a pharmaceutically acceptable salt thereof, is a compound of formula (Ia):

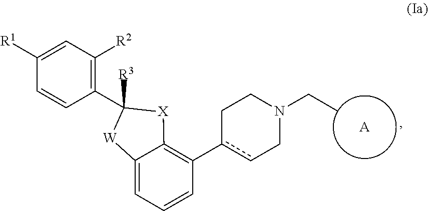

wherein A, W, X, R¹, R² and R³ are as defined herein, or a pharmaceutically acceptable salt thereof.

In an embodiment, the compound of formula (I), or a pharmaceutically acceptable salt thereof, is a compound of formula (II):

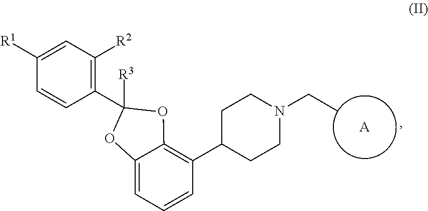

wherein A, R¹, R² and R³ are as defined herein, or a pharmaceutically acceptable salt thereof.

In an embodiment, the compound of formula (I), or a pharmaceutically acceptable salt thereof, is a compound of formula (IIa):

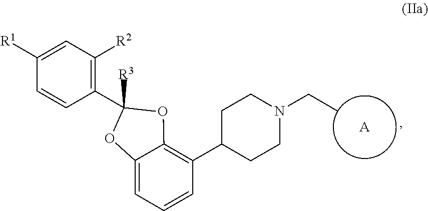

wherein A, R¹, R² and R³ are as defined herein, or a pharmaceutically acceptable salt thereof.

In an embodiment, the compound of formula (I), or a pharmaceutically acceptable salt thereof, is a compound of formula (III):

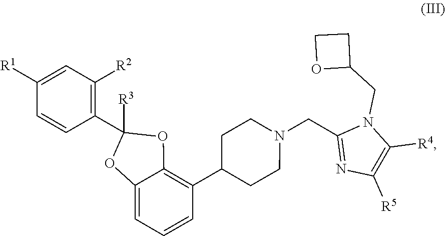

wherein R¹, R², R³, R⁴ and R⁵ are as defined herein, or a pharmaceutically acceptable salt thereof.

In an embodiment, the compound of formula (I), or a pharmaceutically acceptable salt thereof, is a compound of formula (IIIa)

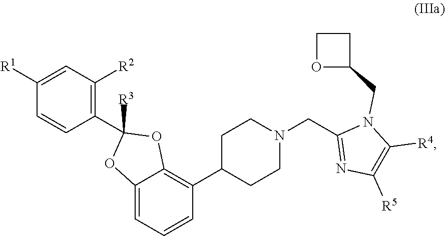

wherein R¹, R², R³, R⁴ and R⁵ are as defined herein, or a pharmaceutically acceptable salt thereof.

In an embodiment, the compound of formula (I), or a pharmaceutically acceptable salt thereof, is a compound of formula (IIIb):

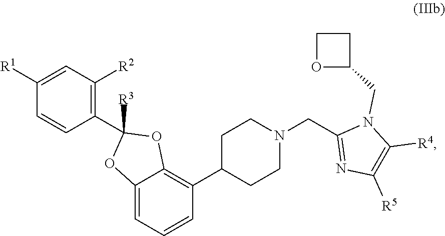

wherein R¹, R², R³, R⁴ and R⁵ are as defined herein, or a pharmaceutically acceptable salt thereof.

In an embodiment, the compound of formula (I), or a pharmaceutically acceptable salt thereof, is a compound of formula (IV):

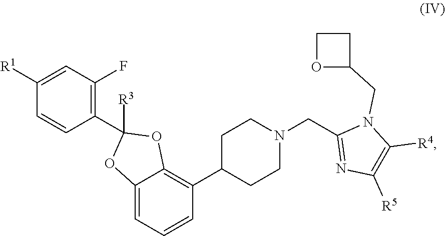

wherein R¹ is chloro or CN, and R³, R⁴ and R⁵ are as defined herein, or a pharmaceutically acceptable salt thereof.

In an embodiment, the compound of formula (I), or a pharmaceutically acceptable salt thereof, is a compound of formula (IVa):

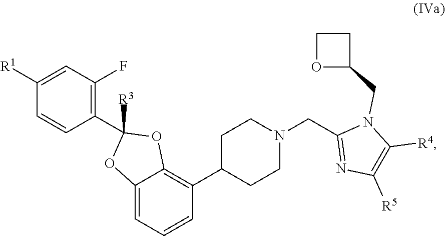

wherein R¹ is chloro or CN, and R³, R⁴ and R⁵ are as defined herein, or a pharmaceutically acceptable salt thereof.

In an embodiment, the compound of formula (I), or a pharmaceutically acceptable salt thereof, is a compound of formula (IVb):

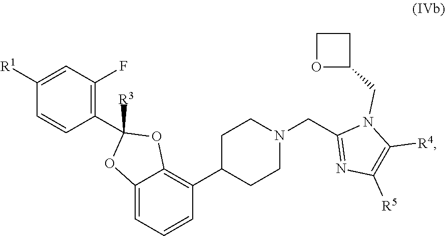

wherein R¹ is chloro or CN, and R³, R⁴ and R⁵ are as defined herein, or a pharmaceutically acceptable salt thereof.

In an embodiment, the compound of formula (I), or a pharmaceutically acceptable salt thereof, is a compound of formula (V):

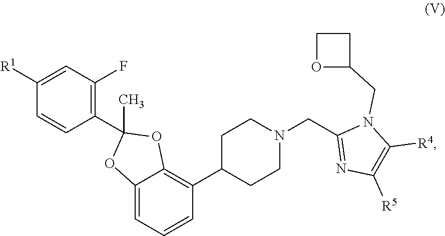

wherein R¹ is chloro or CN, and R⁴ and R⁵ are as defined herein, or a pharmaceutically acceptable salt thereof.

In an embodiment, the compound of formula (I), or a pharmaceutically acceptable salt thereof, is a compound of formula (Va):

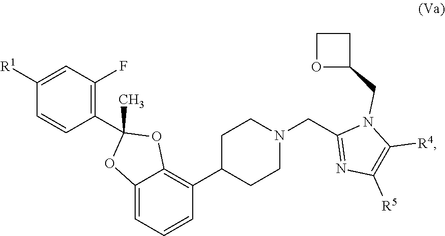

wherein R¹ is chloro or CN, and R⁴ and R⁵ are as defined herein, or a pharmaceutically acceptable salt thereof.

In an embodiment, the compound of formula (I), or a pharmaceutically acceptable salt thereof, is a compound of formula (Vb):

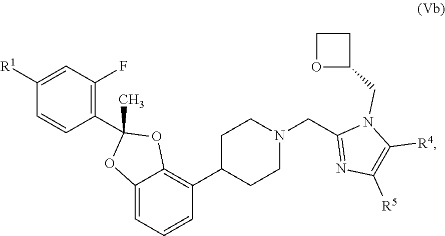

wherein R¹ is chloro or CN, and R⁴ and R⁵ are as defined herein, or a pharmaceutically acceptable salt thereof.

In an embodiment of any of the compounds of formula (Ia), (II), (IIa), (III), (IIIa), (IIIb), (IV), (IVa), (IVb), (V), (Va) and (Vb), or a pharmaceutically acceptable salt thereof, R⁴ is as defined herein.

In an embodiment of any of the compounds of formula (Ia), (II), (IIa), (III), (IIIa), (IIIb), (IV), (IVa), (IVb), (V), (Va) and (Vb), or a pharmaceutically acceptable salt thereof, R⁵ is as defined herein.

In an embodiment of any of the compounds of formula (Ia), (II), (IIa), (III), (IIIa), (IIIb), (IV), (IVa), (IVb), (V), (Va) and (Vb), or a pharmaceutically acceptable salt thereof,
R⁴ is 5-10 membered heteroaryl or (5-10 membered heteroaryl)-C₁₋₃-alkyl-, wherein the 5-10 membered heteroaryl and (5-10 membered heteroaryl)-C₁₋₃-alkyl- are each substituted with C(O)OR^{4A} or with a carboxylic acid isostere, and optionally with 1, 2, or 3 groups independently selected from halo, C₁₋₃-alkyl and C₁₋₃-alkyl substituted with 1, 2 or 3 halo; wherein R^{4A} is selected from H and C₁₋₃-alkyl; and
R⁵ is C₁₋₃-alkyl or C₁₋₃-alkyl substituted with 1, 2 or 3 halo, for example methyl, ethyl or trifluoromethyl.

In an embodiment of any of the compounds of formula (Ia), (II), (IIa), (III), (IIIa), (IIIb), (IV), (IVa), (IVb), (V), (Va) and (Vb), or a pharmaceutically acceptable salt thereof,
R⁴ has the structure of Formula H1 or H2 as defined herein; and
R⁵ is C₁₋₃-alkyl or C₁₋₃-alkyl substituted with 1, 2 or 3 halo, for example methyl, ethyl or trifluoromethyl.

In an embodiment of any of the compounds of formula (Ia), (II), (IIa), (III), (IIIa), (IIIb), (IV), (IVa), (IVb), (V), (Va) and (Vb), or a pharmaceutically acceptable salt thereof,
R⁴ has the structure of Formula H1a or H2a as defined herein; and
R⁵ is C₁₋₃-alkyl or C₁₋₃-alkyl substituted with 1, 2 or 3 halo, for example methyl, ethyl or trifluoromethyl.

In an embodiment of any of the compounds of formula (Ia), (II), (IIa), (III), (IIIa), (IIIb), (IV), (IVa), (IVb), (V), (Va) and (Vb), or a pharmaceutically acceptable salt thereof,
R⁴ is selected from the group consisting of:

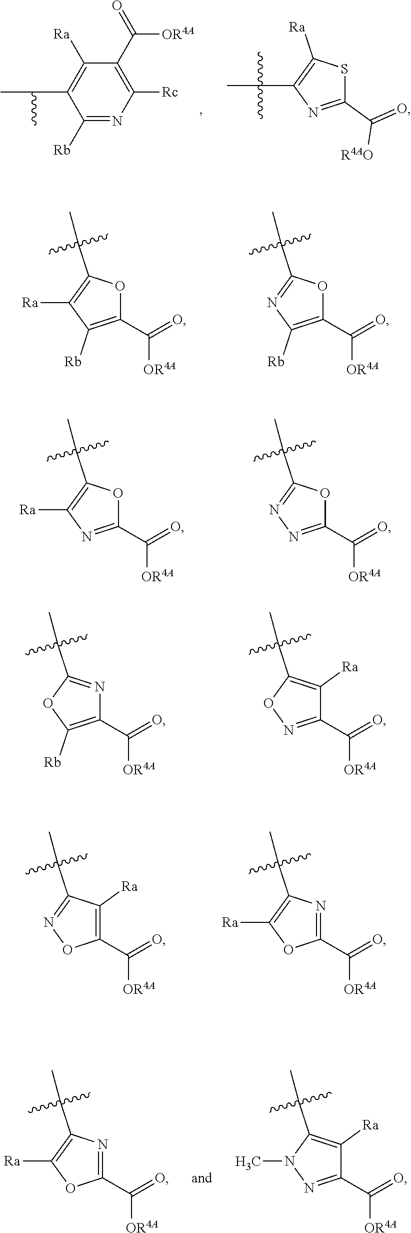

wherein Ra, Rb and Rc are independently selected from H, C₁₋₃-alkyl and C₁₋₃-alkyl substituted with 1, 2 or 3 halo, and R^{4A} is selected from H and C₁₋₃-alkyl, and
R⁵ is C₁₋₃-alkyl or C₁₋₃-alkyl substituted with 1, 2 or 3 halo, for example methyl, ethyl or trifluoromethyl.

In an embodiment of any of the compounds of formula (Ia), (II), (IIa), (III), (IIIa), (IIIb), (IV), (IVa), (IVb), (V), (Va) and (Vb), or a pharmaceutically acceptable salt thereof,
R⁴ is C₁₋₃-alkyl or C₁₋₃-alkyl substituted with 1, 2 or 3 halo, for example methyl, ethyl or trifluoromethyl, and
R⁵ is 5-10 membered heteroaryl or (5-10 membered heteroaryl)-C₁₋₃-alkyl-, wherein the 5-10 membered heteroaryl and (5-10 membered heteroaryl)-C₁₋₃-alkyl- are each substituted with C(O)OR^{5A} or with a carboxylic acid isostere, and optionally with 1, 2, or 3 groups independently selected from halo, C₁₋₃-alkyl and C₁₋₃-alkyl substituted with 1, 2 or 3 halo; wherein R^{5A} is selected from H and C₁₋₃-alkyl.

In an embodiment of any of the compounds of formula (Ia), (II), (IIa), (III), (IIIa), (IIIb), (IV), (IVa), (IVb), (V), (Va) and (Vb), or a pharmaceutically acceptable salt thereof,
R⁴ is C₁₋₃-alkyl or C₁₋₃-alkyl substituted with 1, 2 or 3 halo, for example methyl, ethyl or trifluoromethyl, and
R⁵ has the structure of Formula H11 or H12 as defined herein.

In an embodiment of any of the compounds of formula (Ia), (II), (IIa), (III), (IIIa), (IIIb), (IV), (IVa), (IVb), (V), (Va) and (Vb), or a pharmaceutically acceptable salt thereof,
R⁴ is C₁₋₃-alkyl or C₁₋₃-alkyl substituted with 1, 2 or 3 halo, for example methyl, ethyl or trifluoromethyl, and
R⁵ has the structure of Formula H11a or H12a as defined herein.
   In an embodiment of any of the compounds of formula (Ia), (II), (IIa), (III), (IIIa), (IIIb), (IV), (IVa), (IVb), (V), (Va) and (Vb), or a pharmaceutically acceptable salt thereof,
R⁴ is C₁₋₃-alkyl or C₁₋₃-alkyl substituted with 1, 2 or 3 halo, for example methyl, ethyl or trifluoromethyl, and
R⁵ is selected from the group consisting of:

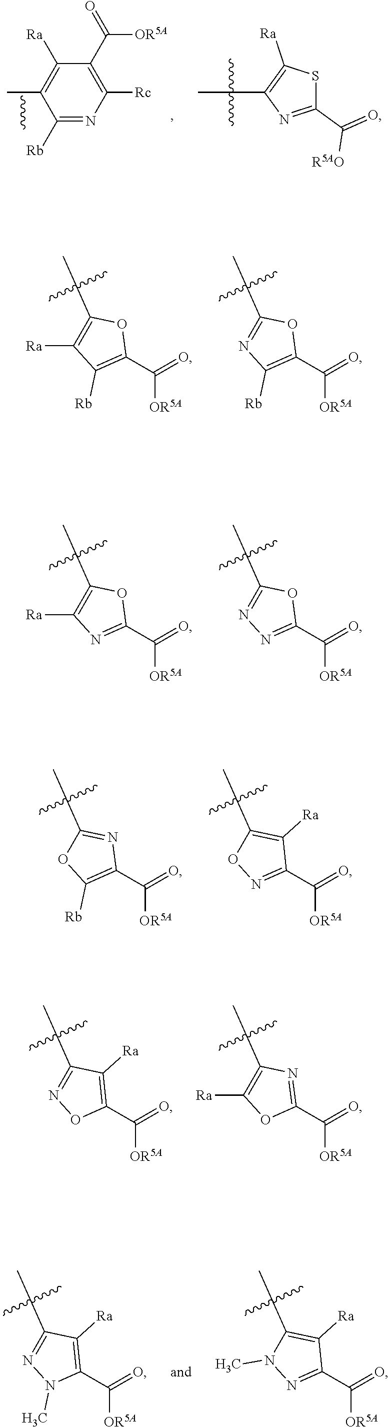

wherein Ra, Rb and Rc are independently selected from H, C₁₋₃-alkyl and C₁₋₃-alkyl substituted with 1, 2 or 3 halo, and R^{5A} is selected from H and C₁₋₃-alkyl.

In an embodiment, the compound of formula (I) is selected from the group consisting of:

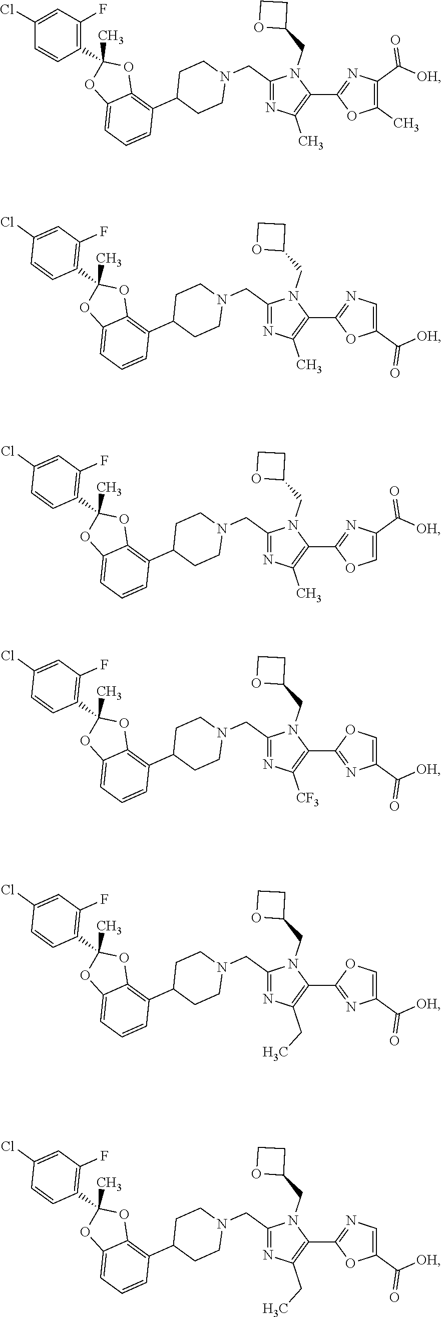

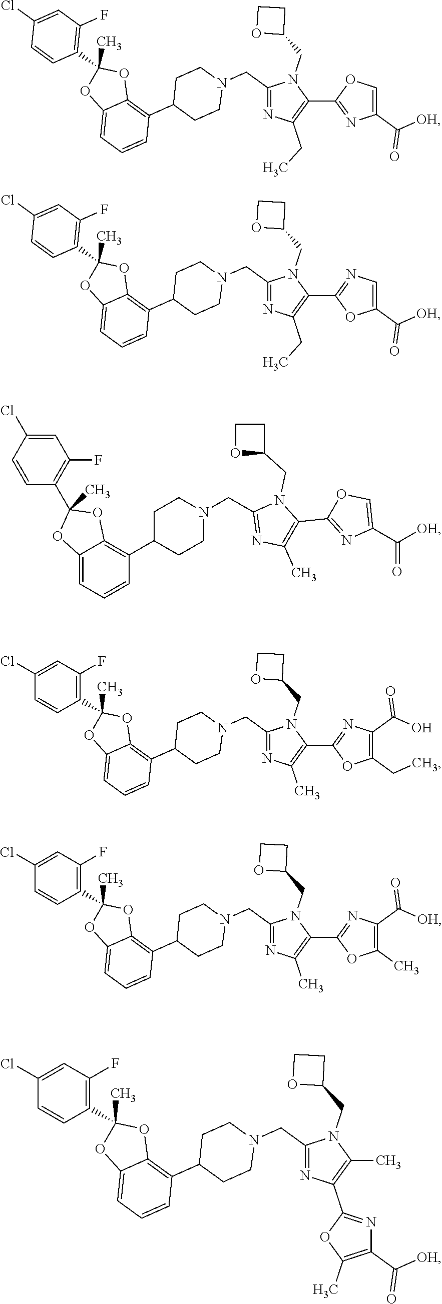

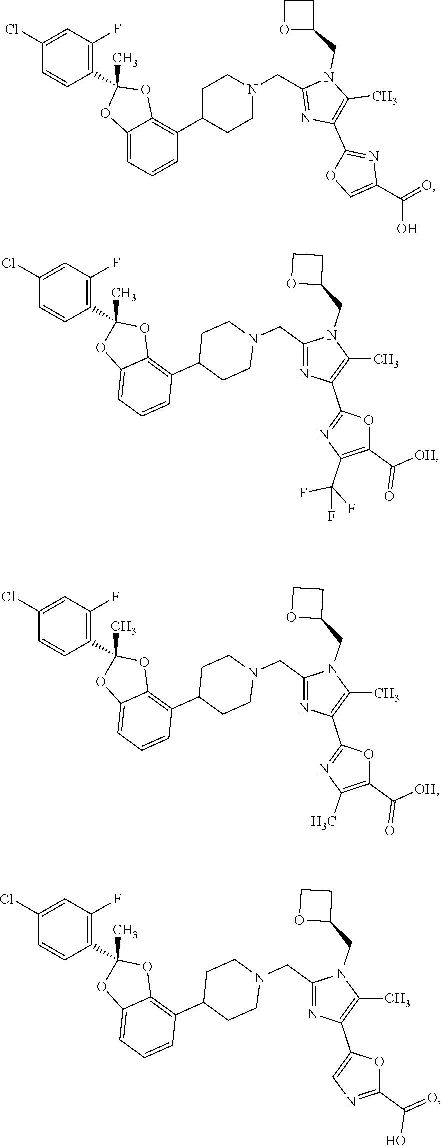 [SMILES: Cc1c(-c2nc(C(=O)O)co2)nc(CN2CCC(c3cccc4c3O[C@@](C)(c3ccc(Cl)cc3F)O4)CC2)n1C[C@@H]1CCO1 ; Cc1c(-c2nc(C(F)(F)F)c(C(=O)O)o2)nc(CN2CCC(c3cccc4c3O[C@@](C)(c3ccc(Cl)cc3F)O4)CC2)n1C[C@@H]1CCO1 ; Cc1nc(-c2nc(CN3CCC(c4cccc5c4O[C@@](C)(c4ccc(Cl)cc4F)O5)CC3)n(C[C@@H]3CCO3)c2C)oc1C(=O)O ; Cc1c(-c2cnc(C(=O)O)o2)nc(CN2CCC(c3cccc4c3O[C@@](C)(c3ccc(Cl)cc3F)O4)CC2)n1C[C@@H]1CCO1]

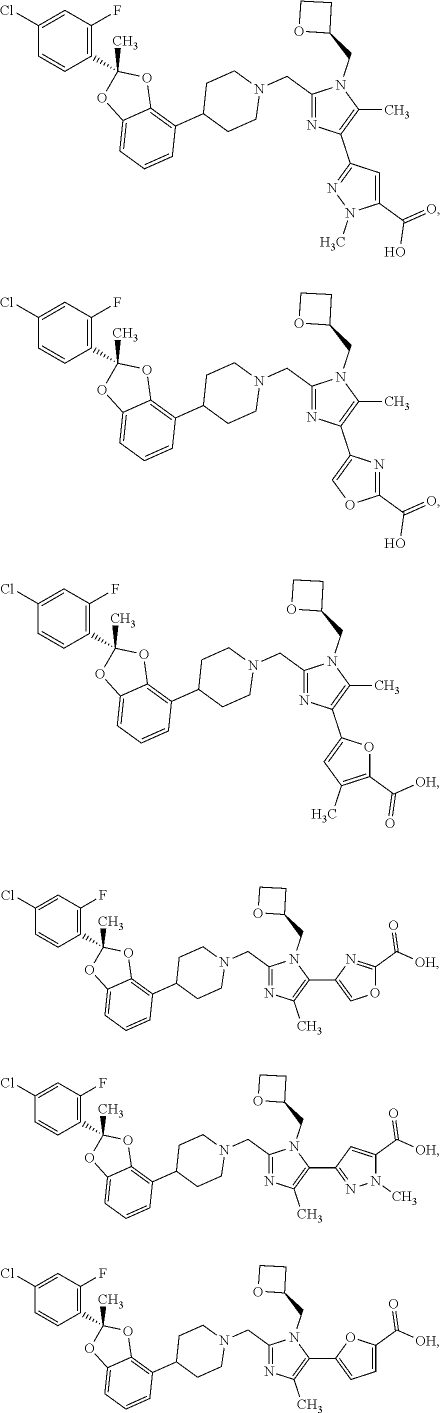

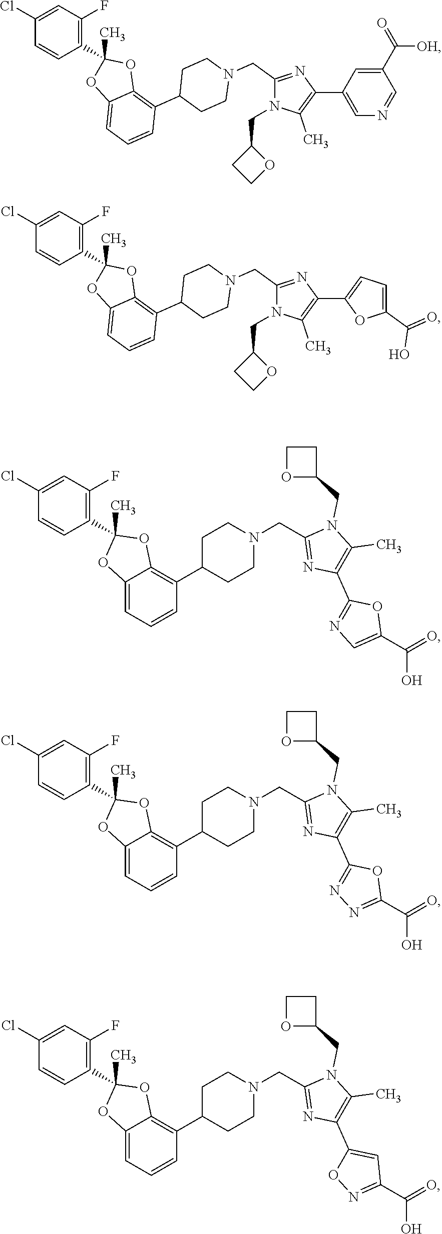 [SMILES: Cc1c(-c2cncc(C(=O)O)c2)nc(CN2CCC(c3cccc4c3O[C@@](C)(c3ccc(Cl)cc3F)O4)CC2)n1C[C@@H]1CCO1 ; Cc1c(-c2ccc(C(=O)O)o2)nc(CN2CCC(c3cccc4c3O[C@@](C)(c3ccc(Cl)cc3F)O4)CC2)n1C[C@@H]1CCO1 ; Cc1c(-c2ncc(C(=O)O)o2)nc(CN2CCC(c3cccc4c3O[C@@](C)(c3ccc(Cl)cc3F)O4)CC2)n1C[C@@H]1CCO1 ; Cc1c(-c2nnc(C(=O)O)o2)nc(CN2CCC(c3cccc4c3O[C@@](C)(c3ccc(Cl)cc3F)O4)CC2)n1C[C@@H]1CCO1 ; Cc1c(-c2cc(C(=O)O)no2)nc(CN2CCC(c3cccc4c3O[C@@](C)(c3ccc(Cl)cc3F)O4)CC2)n1C[C@@H]1CCO1]

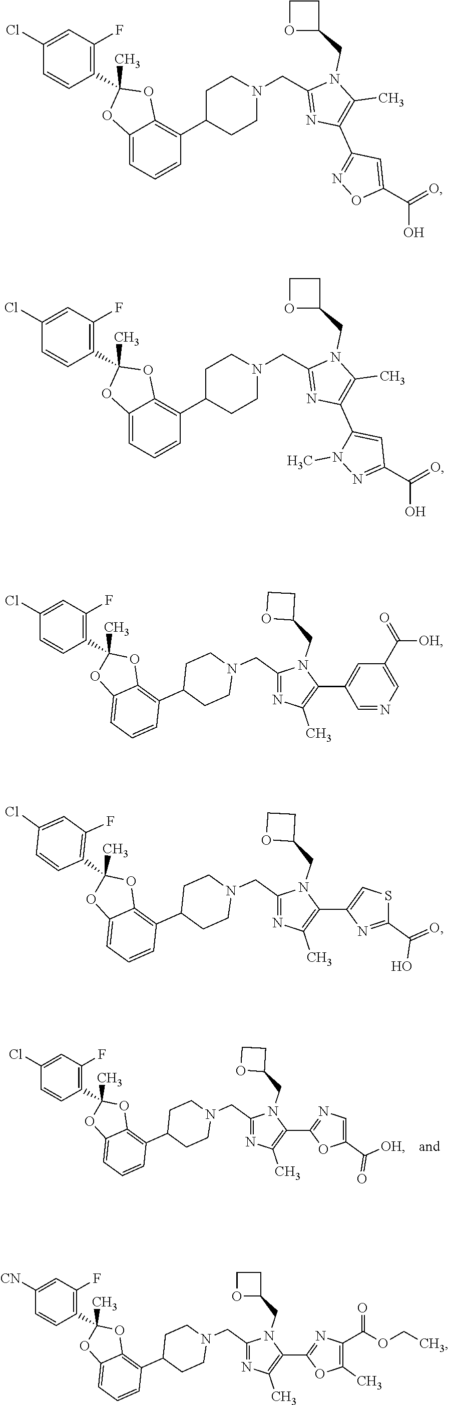

or a pharmaceutically acceptable salt thereof.

In an embodiment, the compound of formula (I) is selected from the group consisting of:
2-(2-((4-((S)-2-(4-chloro-2-fluorophenyl)-2-methylbenzo[d][1,3]dioxol-4-yl)piperidin-1-yl)methyl)-4-methyl-1-(((S)-oxetan-2-yl)methyl)-1H-imidazol-5-yl)-5-methyloxazole-4-carboxylic acid,
2-(2-((4-((S)-2-(4-chloro-2-fluorophenyl)-2-methylbenzo[d][1,3]dioxol-4-yl)piperidin-1-yl)methyl)-4-methyl-1-(((R)-oxetan-2-yl)methyl)-1H-imidazol-5-yl)oxazole-5-carboxylic acid,
2-(2-((4-((S)-2-(4-chloro-2-fluorophenyl)-2-methylbenzo[d][1,3]dioxol-4-yl)piperidin-1-yl)methyl)-4-methyl-1-(((R)-oxetan-2-yl)methyl)-1H-imidazol-5-yl)oxazole-4-carboxylic acid,
2-(2-((4-((S)-2-(4-chloro-2-fluorophenyl)-2-methylbenzo[d][1,3]dioxol-4-yl)piperidin-1-yl)methyl)-1-(((S)-oxetan-2-yl)methyl)-4-(trifluoromethyl)-1H-imidazol-5-yl)oxazole-4-carboxylic acid,
2-(2-((4-((S)-2-(4-chloro-2-fluorophenyl)-2-methylbenzo[d][1,3]dioxol-4-yl)piperidin-1-yl)methyl)-4-ethyl-1-(((S)-oxetan-2-yl)methyl)-1H-imidazol-5-yl)oxazole-4-carboxylic acid,
2-(2-((4-((S)-2-(4-chloro-2-fluorophenyl)-2-methylbenzo[d][1,3]dioxol-4-yl)piperidin-1-yl)methyl)-4-ethyl-1-(((S)-oxetan-2-yl)methyl)-1H-imidazol-5-yl)oxazole-5-carboxylic acid,
2-(2-((4-((S)-2-(4-chloro-2-fluorophenyl)-2-methylbenzo[d][1,3]dioxol-4-yl)piperidin-1-yl)methyl)-4-ethyl-1-(((R)-oxetan-2-yl)methyl)-1H-imidazol-5-yl)oxazole-4-carboxylic acid,
2-(2-((4-((S)-2-(4-chloro-2-fluorophenyl)-2-methylbenzo[d][1,3]dioxol-4-yl)piperidin-1-yl)methyl)-4-ethyl-1-(((R)-oxetan-2-yl)methyl)-1H-imidazol-5-yl)oxazole-5-carboxylic acid,
2-(2-((4-((S)-2-(4-chloro-2-fluorophenyl)-2-methylbenzo[d][1,3]dioxol-4-yl)piperidin-1-yl)methyl)-4-methyl-1-(((S)-oxetan-2-yl)methyl)-1H-imidazol-5-yl)oxazole-4-carboxylic acid,
2-(2-((4-((S)-2-(4-chloro-2-fluorophenyl)-2-methylbenzo[d][1,3]dioxol-4-yl)piperidin-1-yl)methyl)-4-methyl-1-(((S)-oxetan-2-yl)methyl)-1H-imidazol-5-yl)-5-ethyloxazole-4-carboxylic acid,
2-(2-((4-((S)-2-(4-cyano-2-fluorophenyl)-2-methylbenzo[d][1,3]dioxol-4-yl)piperidin-1-yl)methyl)-4-methyl-1-(((S)-oxetan-2-yl)methyl)-1H-imidazol-5-yl)-5-methyloxazole-4-carboxylic acid,
2-(2-((4-((S)-2-(4-chloro-2-fluorophenyl)-2-methylbenzo[d][1,3]dioxol-4-yl) piperidin-1-yl)methyl)-5-methyl-1-(((S)-oxetan-2-yl)methyl)-1H-imidazol-4-yl)-5-methyloxazole-4-carboxylic acid,
2-(2-((4-((S)-2-(4-chloro-2-fluorophenyl)-2-methylbenzo[d][1,3]dioxol-4-yl) piperidin-1-yl)methyl)-5-methyl-1-(((S)-oxetan-2-yl)methyl)-1H-imidazol-4-yl)oxazole-4-carboxylic acid,
2-(2-((4-((S)-2-(4-chloro-2-fluorophenyl)-2-methylbenzo[d][1,3]dioxol-4-yl) piperidin-1-yl)methyl)-5-methyl-1-(((S)-oxetan-2-yl)methyl)-1H-imidazol-4-yl)-4-(trifluoromethyl)oxazole-5-carboxylic acid,
2-(2-((4-((S)-2-(4-chloro-2-fluorophenyl)-2-methylbenzo[d][1,3]dioxol-4-yl)piperidin-1-yl)methyl)-5-methyl-1-(((S)-oxetan-2-yl)methyl)-1H-imidazol-4-yl)-4-methyloxazole-5-carboxylic acid,
5-(2-((4-((S)-2-(4-chloro-2-fluorophenyl)-2-methylbenzo[d][1,3]dioxol-4-yl)piperidin-1-yl)methyl)-5-methyl-1-(((S)-oxetan-2-yl)methyl)-1H-imidazol-4-yl)oxazole-2-carboxylic acid,
3-(2-((4-((S)-2-(4-chloro-2-fluorophenyl)-2-methylbenzo[d][1,3]dioxol-4-yl)piperidin-1-yl)methyl)-5-methyl-1-(((S)-oxetan-2-yl)methyl)-1H-imidazol-4-yl)-1-methyl-1H-pyrazole-5-carboxylic acid,
4-(2-((4-((S)-2-(4-chloro-2-fluorophenyl)-2-methylbenzo[d][1,3]dioxol-4-yl)piperidin-1-yl)methyl)-5-methyl-1-(((S)-oxetan-2-yl)methyl)-1H-imidazol-4-yl)oxazole-2-carboxylic acid,
5-(2-((4-((S)-2-(4-chloro-2-fluorophenyl)-2-methylbenzo[d][1,3]dioxol-4-yl)piperidin-1-yl)methyl)-5-methyl-1-(((S)-oxetan-2-yl)methyl)-1H-imidazol-4-yl)-3-methylfuran-2-carboxylic acid,
4-(2-((4-((S)-2-(4-chloro-2-fluorophenyl)-2-methylbenzo[d][1,3]dioxol-4-yl)piperidin-1-yl)methyl)-4-methyl-1-(((S)-oxetan-2-yl)methyl)-1H-imidazol-5-yl)oxazole-2-carboxylic acid,
3-(2-((4-((S)-2-(4-chloro-2-fluorophenyl)-2-methylbenzo[d][1,3]dioxol-4-yl)piperidin-1-yl)methyl)-4-methyl-1-(((S)-oxetan-2-yl)methyl)-1H-imidazol-5-yl)-1-methyl-1H-pyrazole-5-carboxylic acid,
5-(2-((4-((S)-2-(4-chloro-2-fluorophenyl)-2-methylbenzo[d][1,3]dioxol-4-yl)piperidin-1-yl)methyl)-4-methyl-1-(((S)-oxetan-2-yl)methyl)-1H-imidazol-5-yl)furan-2-carboxylic acid,
5-(2-((4-((S)-2-(4-chloro-2-fluorophenyl)-2-methylbenzo[d][1,3]dioxol-4-yl)piperidin-1-yl)methyl)-5-methyl-1-(((S)-oxetan-2-yl)methyl)-1H-imidazol-4-yl)nicotinic acid,
5-(2-((4-((S)-2-(4-chloro-2-fluorophenyl)-2-methylbenzo[d][1,3]dioxol-4-yl)piperidin-1-yl)methyl)-5-methyl-1-(((S)-oxetan-2-yl)methyl)-1H-imidazol-4-yl)furan-2-carboxylic acid,
2-(2-((4-((S)-2-(4-chloro-2-fluorophenyl)-2-methylbenzo[d][1,3]dioxol-4-yl)piperidin-1-yl)methyl)-5-methyl-1-(((S)-oxetan-2-yl)methyl)-1H-imidazol-4-yl)oxazole-5-carboxylic acid,
5-(2-((4-((S)-2-(4-chloro-2-fluorophenyl)-2-methylbenzo[d][1,3]dioxol-4-yl) piperidin-1-yl)methyl)-5-methyl-1-(((S)-oxetan-2-yl)methyl)-1H-imidazol-4-yl)-1,3,4-oxadiazole-2-carboxylic acid,
5-(2-((4-((S)-2-(4-chloro-2-fluorophenyl)-2-methylbenzo[d][1,3]dioxol-4-yl) piperidin-1-yl)methyl)-5-methyl-1-(((S)-oxetan-2-yl)methyl)-1H-imidazol-4-yl)isoxazole-3-carboxylic acid,
3-(2-((4-((S)-2-(4-chloro-2-fluorophenyl)-2-methylbenzo[d][1,3]dioxol-4-yl) piperidin-1-yl)methyl)-5-methyl-1-(((S)-oxetan-2-yl)methyl)-1H-imidazol-4-yl)isoxazole-5-carboxylic acid,
5-(2-((4-((S)-2-(4-chloro-2-fluorophenyl)-2-methylbenzo[d][1,3]dioxol-4-yl) piperidin-1-yl)methyl)-5-methyl-1-(((S)-oxetan-2-yl)methyl)-1H-imidazol-4-yl)-1-methyl-1H-pyrazole-3-carboxylic acid,
5-(2-((4-((S)-2-(4-chloro-2-fluorophenyl)-2-methylbenzo[d][1,3]dioxol-4-yl)piperidin-1-yl)methyl)-4-methyl-1-(((S)-oxetan-2-yl)methyl)-1H-imidazol-5-yl)nicotinic acid,
4-(2-((4-((S)-2-(4-chloro-2-fluorophenyl)-2-methylbenzo[d][1,3]dioxol-4-yl)piperidin-1-yl)methyl)-4-methyl-1-(((S)-oxetan-2-yl)methyl)-1H-imidazol-5-yl)thiazole-2-carboxylic acid,
2-(2-((4-((S)-2-(4-chloro-2-fluorophenyl)-2-methylbenzo[d][1,3]dioxol-4-yl)piperidin-1-yl)methyl)-4-methyl-1-(((S)-oxetan-2-yl)methyl)-1H-imidazol-5-yl)oxazole-5-carboxylic acid, and
ethyl 2-(2-((4-((S)-2-(4-cyano-2-fluorophenyl)-2-methylbenzo[d][1,3]dioxol-4-yl)piperidin-1-yl)methyl)-4-methyl-1-(((S)-oxetan-2-yl)methyl)-1H-imidazol-5-yl)-5-methyloxazole-4-carboxylate,
or a pharmaceutically acceptable salt thereof.

In an embodiment, the compound of formula (I), or a pharmaceutically acceptable salt thereof, is selected from Table A:

TABLE A

| # | Compound | Name |
|---|---|---|
| C-1 | 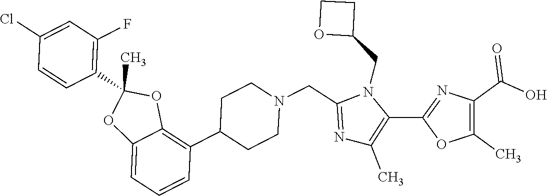 | 2-(2-((4-((S)-2-(4-chloro-2- fluorophenyl)-2- methylbenzo[d][1,3]dioxol-4- yl)piperidin-1-yl)methyl)-4- methyl-1-(((S)-oxetan-2- yl)methyl)-1H-imidazol-5-yl)-5- methyloxazole-4-carboxylic acid |
| C-2 | 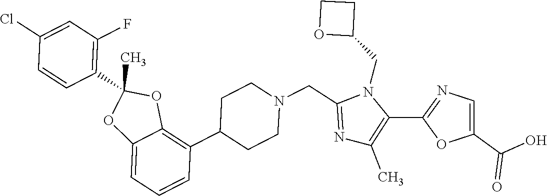 | 2-(2-((4-((S)-2-(4-chloro-2- fluorophenyl)-2- methylbenzo[d][1,3]dioxol-4- yl)piperidin-1-yl)methyl)-4- methyl-1-(((R)-oxetan-2- yl)methyl)-1H-imidazol-5- yl)oxazole-5-carboxylic acid |
| C-3 | 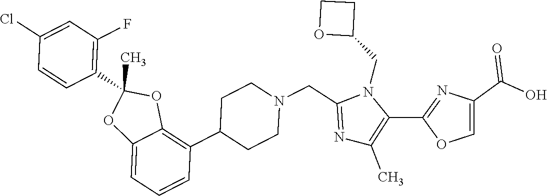 | 2-(2-((4-((S)-2-(4-chloro-2- fluorophenyl)-2- methylbenzo[d][1,3]dioxol-4- yl)piperidin-1-yl)methyl)-4- methyl-1-(((R)-oxetan-2- yl)methyl)-1H-imidazol-5- yl)oxazole-4-carboxylic acid |
| C-4 | 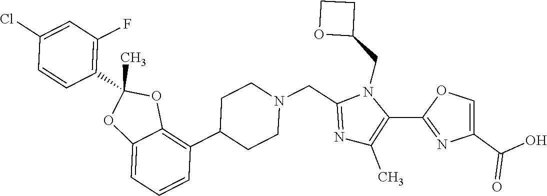 | 2-(2-((4-((S)-2-(4-chloro-2- fluorophenyl)-2- methylbenzo[d][1,3]dioxol-4- yl)piperidin-1-yl)methyl)-1-(((S)- oxetan-2-yl)methyl)-4- (trifluoromethyl)-1H-imidazol-5- yl)oxazole-4-carboxylic acid |
| C-5 | 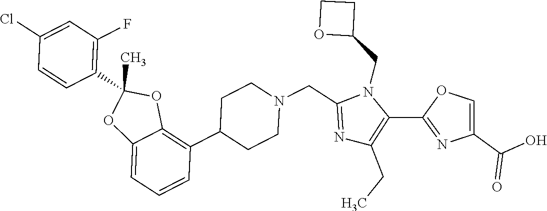 | 2-(2-((4-((S)-2-(4-chloro-2- fluorophenyl)-2- methylbenzo[d][1,3]dioxol-4- yl)piperidin-1-yl)methyl)-4-ethyl- 1-(((S)-oxetan-2-yl)methyl)-1H- imidazol-5-yl)oxazole-4- carboxylic acid |
| C-6 | 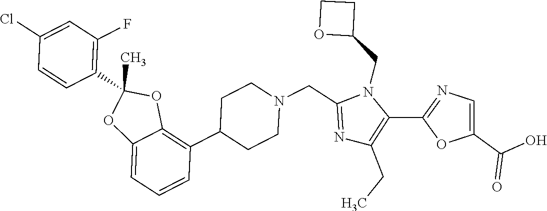 | 2-(2-((4-((S)-2-(4-chloro-2- fluorophenyl)-2- methylbenzo[d][1,3]dioxol-4- yl)piperidin-1-yl)methyl)-4-ethyl- 1-(((S)-oxetan-2-yl)methyl)-1H- imidazol-5-yl)oxazole-5- carboxylic acid |
| C-7 | 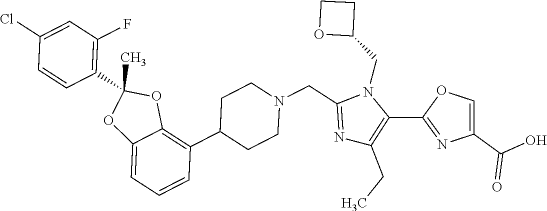 | 2-(2-((4-((S)-2-(4-chloro-2- fluorophenyl)-2- methylbenzo[d][1,3]dioxol-4- yl)piperidin-1-yl)methyl)-4-ethyl- 1-(((R)-oxetan-2-yl)methyl)-1H- imidazol-5-yl)oxazole-4- carboxylic acid |
| C-8 | 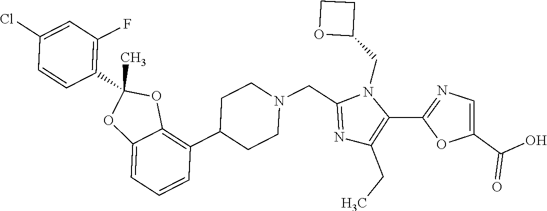 | 2-(2-((4-((S)-2-(4-chloro-2- fluorophenyl)-2- methylbenzo[d][1,3]dioxol-4- yl)piperidin-1-yl)methyl)-4-ethyl- 1-(((R)-oxetan-2-yl)methyl)-1H- imidazol-5-yl)oxazole-5- carboxylic acid |
| C-9 | 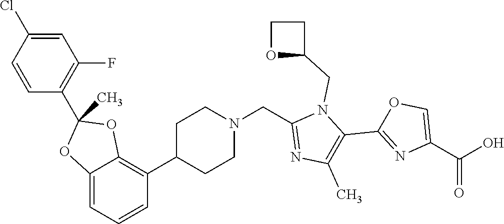 [SMILES: Cc1nc(CN2CCC(c3cccc4c3O[C@@](C)(c3ccc(Cl)cc3F)O4)CC2)n(C[C@@H]2CCO2)c1-c1nc(C(=O)O)co1] | 2-(2-((4-((S)-2-(4-chloro-2- fluorophenyl)-2- methylbenzo[d][1,3]dioxol-4- yl)piperidin-1-yl)methyl)-4- methyl-1-(((S)-oxetan-2- yl)methyl)-1H-imidazol-5- yl)oxazole-4-carboxylic acid |
| C-10 | 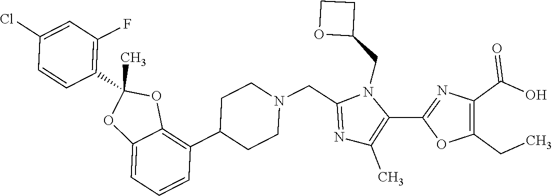 | 2-(2-((4-((S)-2-(4-chloro-2- fluorophenyl)-2- methylbenzo[d][1,3]dioxol-4- yl)piperidin-1-yl)methyl)-4- methyl-1-(((S)-oxetan-2- yl)methyl)-1H-imidazol-5-yl)-5- ethyloxazole-4-carboxylic acid |
| C-11 | 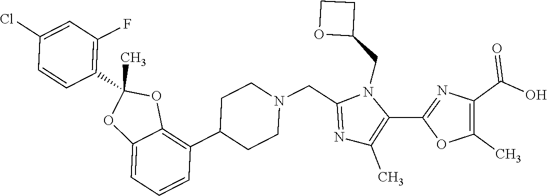 | 2-(2-((4-((S)-2-(4-cyano-2- fluorophenyl)-2- methylbenzo[d][1,3]dioxol-4- yl)piperidin-1-yl)methyl)-4- methyl-1-(((S)-oxetan-2- yl)methyl)-1H-imidazol-5-yl)-5- methyloxazole-4-carboxylic acid |
| C-12 | 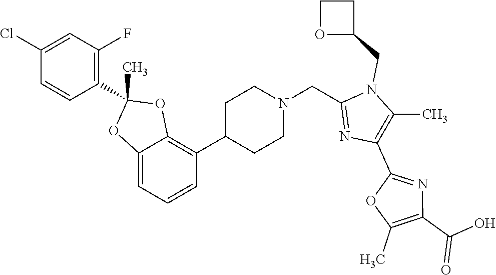 | 2-(2-((4-((S)-2-(4-chloro-2- fluorophenyl)-2- methylbenzo[d][1,3]dioxol-4- yl)piperidin-1-yl)methyl)-5- methyl-1-(((S)-oxetan-2- yl)methyl)-1H-imidazol-4-yl)-5- methyloxazole-4-carboxylic acid |
| C-13 | 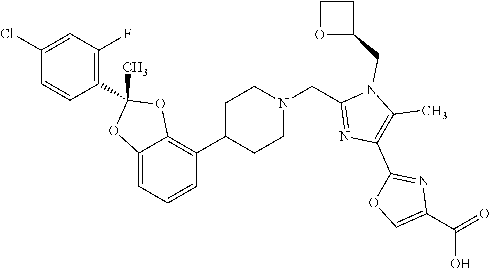 | 2-(2-((4-((S)-2-(4-chloro-2- fluorophenyl)-2- methylbenzo[d][1,3]dioxol-4- yl)piperidin-1-yl)methyl)-5- methyl-1-(((S)-oxetan-2- yl)methyl)-1H-imidazol-4- yl)oxazole-4-carboxylic acid |
| C-14 | 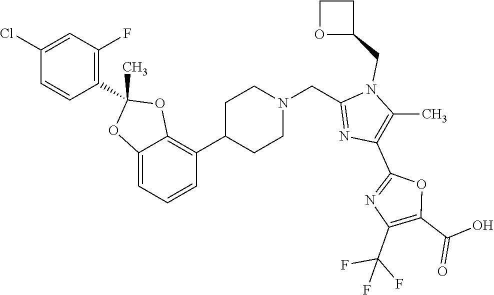 | 2-(2-((4-((S)-2-(4-chloro-2- fluorophenyl)-2- methylbenzo[d][1,3]dioxol-4- yl)piperidin-1-yl)methyl)-5- methyl-1-(((S)-oxetan-2- yl)methyl)-1H-imidazol-4-yl)-4- (trifluoromethyl)oxazole-5- carboxylic acid |
| C-15 | 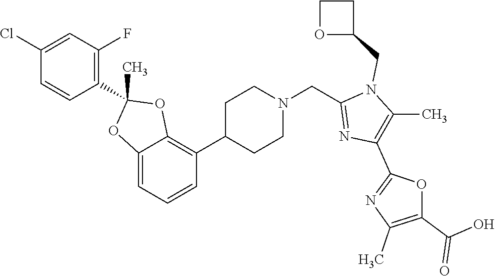 | 2-(2-((4-((S)-2-(4-chloro-2- fluorophenyl)-2- methylbenzo[d][1,3]dioxol-4- yl)piperidin-1-yl)methyl)-5- methyl-1-(((S)-oxetan-2- yl)methyl)-1H-imidazol-4-yl)-4- methyloxazole-5-carboxylic acid |
| C-16 | 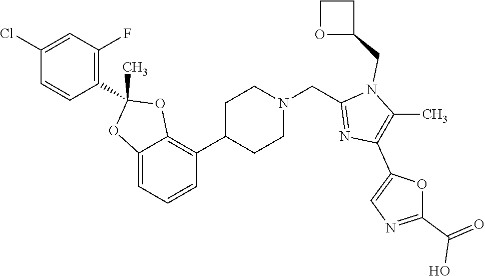 | 5-(2-((4-((S)-2-(4-chloro-2- fluorophenyl)-2- methylbenzo[d][1,3]dioxol-4- yl)piperidin-1-yl)methyl)-5- methyl-1-(((S)-oxetan-2- yl)methyl)-1H-imidazol-4- yl)oxazole-2-carboxylic acid |
| C-17 | 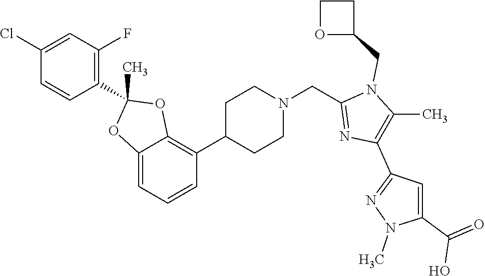 | 3-(2-((4-((S)-2-(4-chloro-2- fluorophenyl)-2- methylbenzo[d][1,3]dioxol-4- yl)piperidin-1-yl)methyl)-5- methyl-1-(((S)-oxetan-2- yl)methyl)-1H-imidazol-4-yl)-1- methyl-1H-pyrazole-5-carboxylic acid |
| C-18 | 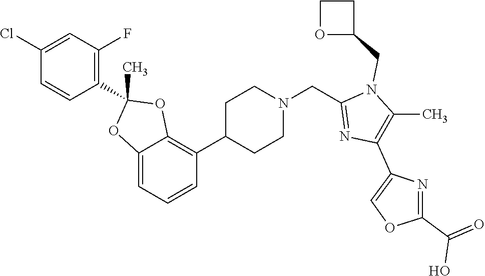 | 4-(2-((4-((S)-2-(4-chloro-2- fluorophenyl)-2- methylbenzo[d][1,3]dioxol-4- yl)piperidin-1-yl)methyl)-5- methyl-1-(((S)-oxetan-2- yl)methyl)-1H-imidazol-4- yl)oxazole-2-carboxylic acid |
| C-19 | 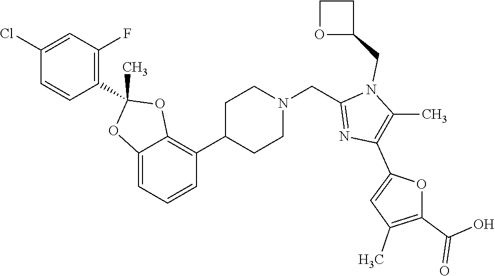 | 5-(2-((4-((S)-2-(4-chloro-2- fluorophenyl)-2- methylbenzo[d][1,3]dioxol-4- yl)piperidin-1-yl)methyl)-5- methyl-1-(((S)-oxetan-2- yl)methyl)-1H-imidazol-4-yl)-3- methylfuran-2-carboxylic acid |
| C-20 | 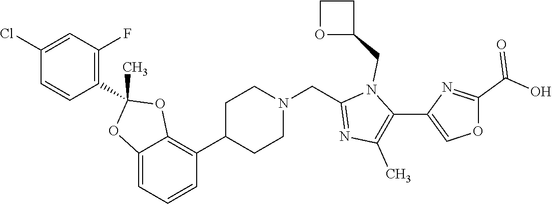 | 4-(2-((4-((S)-2-(4-chloro-2- fluorophenyl)-2- methylbenzo[d][1,3]dioxol-4- yl)piperidin-1-yl)methyl)-4- methyl-1-(((S)-oxetan-2- yl)methyl)-1H-imidazol-5- yl)oxazole-2-carboxylic acid |
| C-21 | 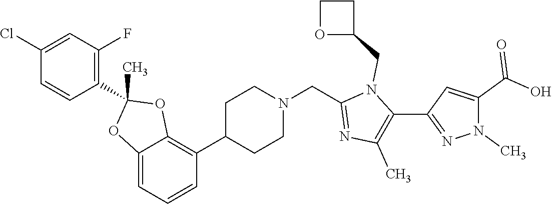 | 3-(2-((4-((S)-2-(4-chloro-2- fluorophenyl)-2- methylbenzo[d][1,3]dioxol-4- yl)piperidin-1-yl)methyl)-4- methyl-1-(((S)-oxetan-2- yl)methyl)-1H-imidazol-5-yl)-1- methyl-1H-pyrazole-5-carboxylic acid |
| C-22 | 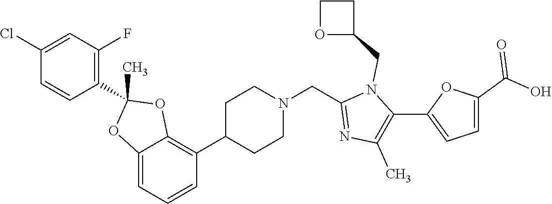 | 5-(2-((4-((S)-2-(4-chloro-2- fluorophenyl)-2- methylbenzo[d][1,3]dioxol-4- yl)piperidin-1-yl)methyl)-4- methyl-1-(((S)-oxetan-2- yl)methyl)-1H-imidazol-5- yl) furan-2-carboxylic acid |
| C-23 | 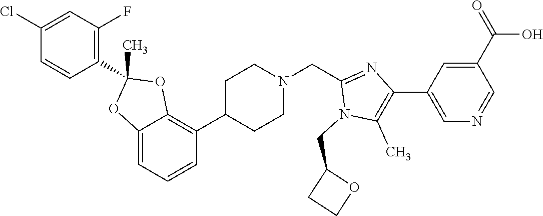 | 5-(2-((4-((S)-2-(4-chloro-2- fluorophenyl)-2- methylbenzo[d][1,3]dioxol-4- yl)piperidin-1-yl)methyl)-5- methyl-1-(((S)-oxetan-2- yl)methyl)-1H-imidazol-4- yl)nicotinic acid |
| C-24 | 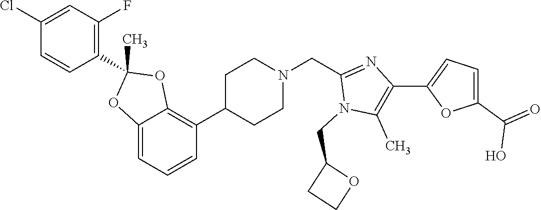 | 5-(2-((4-((S)-2-(4-chloro-2- fluorophenyl)-2- methylbenzo[d][1,3]dioxol-4- yl)piperidin-1-yl)methyl)-5- methyl-1-(((S)-oxetan-2- yl)methyl)-1H-imidazol-4- yl)furan-2-carboxylic acid |
| C-25 | 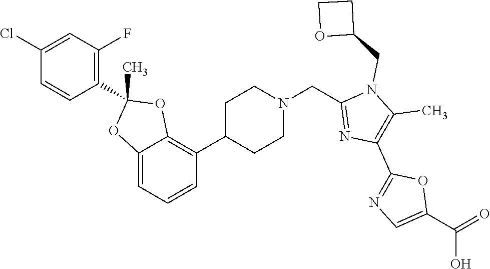 | 2-(2-((4-((S)-2-(4-chloro-2- fluorophenyl)-2- methylbenzo[d][1,3]dioxol-4- yl)piperidin-1-yl)methyl)-5- methyl-1-(((S)-oxetan-2- yl)methyl)-1H-imidazol-4- yl)oxazole-5-carboxylic acid |
| C-26 | 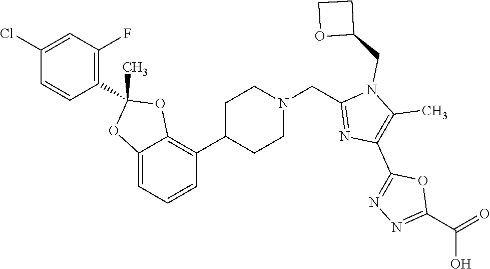 | 5-(2-((4-((S)-2-(4-chloro-2- fluorophenyl)-2- methylbenzo[d][1,3]dioxol-4- yl)piperidin-1-yl)methyl)-5- methyl-1-(((S)-oxetan-2- yl)methyl)-1H-imidazol-4-yl)- 1,3,4-oxadiazole-2-carboxylic acid |
| C-27 | 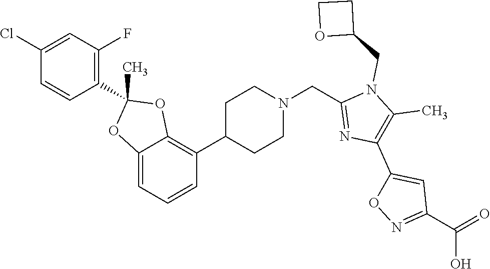 | 5-(2-((4-((S)-2-(4-chloro-2- fluorophenyl)-2- methylbenzo[d][1,3]dioxol-4- yl)piperidin-1-yl)methyl)-5- methyl-1-(((S)-oxetan-2- yl)methyl)-1H-imidazol-4- yl)isoxazole-3-carboxylic acid |
| C-28 | 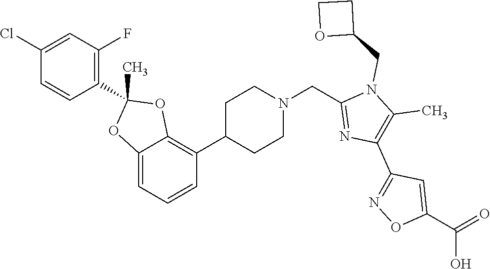 | 3-(2-((4-((S)-2-(4-chloro-2- fluorophenyl)-2- methylbenzo[d][1,3]dioxol-4- yl)piperidin-1-yl)methyl)-5- methyl-1-(((S)-oxetan-2- yl)methyl)-1H-imidazol-4- yl)isoxazole-5-carboxylic acid |
| C-29 | 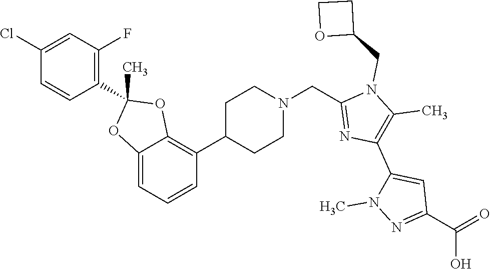 | 5-(2-((4-((S)-2-(4-chloro-2- fluorophenyl)-2- methylbenzo[d][1,3]dioxol-4- yl)piperidin-1-yl)methyl)-5- methyl-1-(((S)-oxetan-2- yl)methyl)-1H-imidazol-4-yl)-1- methyl-1H-pyrazole-3-carboxylic acid |
| C-30 | 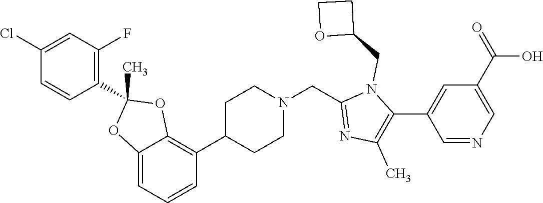 | 5-(2-((4-((S)-2-(4-chloro-2- fluorophenyl)-2- methylbenzo[d][1,3]dioxol-4- yl)piperidin-1-yl)methyl)-4- methyl-1-(((S)-oxetan-2- yl)methyl)-1H-imidazol-5- yl)nicotinic acid |
| C-31 | 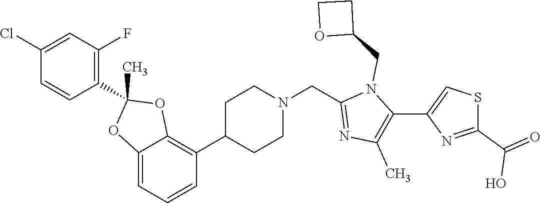 | 4-(2-((4-((S)-2-(4-chloro-2- fluorophenyl)-2- methylbenzo[d][1,3]dioxol-4- yl)piperidin-1-yl)methyl)-4- methyl-1-(((S)-oxetan-2- yl)methyl)-1H-imidazol-5- yl)thiazole-2-carboxylic acid |
| C-32 | 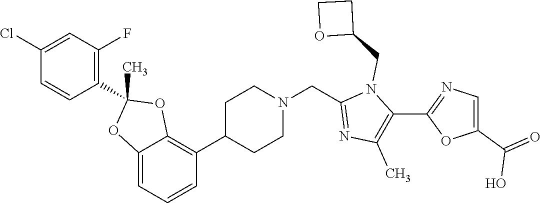 | 2-(2-((4-((S)-2-(4-chloro-2- fluorophenyl)-2- methylbenzo[d][1,3]dioxol-4- yl)piperidin-1-yl)methyl)-4- methyl-1-(((S)-oxetan-2- yl)methyl)-1H-imidazol-5- yl)oxazole-5-carboxylic acid |
| C-33 | 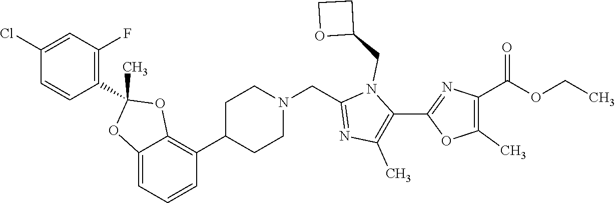 | ethyl 2-(2-((4-((S)-2-(4-cyano-2- fluorophenyl)-2- methylbenzo[d][1,3]dioxol-4- yl)piperidin-1-yl)methyl)-4- methyl-1-(((S)-oxetan-2- yl)methyl)-1H-imidazol-5-yl)-5- methyloxazole-4-carboxylate |

In an embodiment of any of the formulae provided herein, or a pharmaceutically acceptable salt thereof, one or both of optionally substituted variables R⁴ and R⁵ comprise a carboxylic acid, a carboxylic acid derivative (e.g., carboxylic acid ester derivative such as carboxylic acid methyl ester or carboxylic acid ethyl ester) or a carboxylic acid isostere (see, e.g., J. Med. Chem. 2016, 59, 3183-3203). In a preferred embodiment, R⁴ and R⁵ are not the same. For example, in a preferred embodiment, only one of R⁴ and R⁵ comprises a carboxylic acid, a carboxylic acid derivative (e.g., carboxylic acid ester derivative such as carboxylic acid methyl ester or carboxylic acid ethyl ester) or a carboxylic acid isostere.

A non-limiting list of example non-cyclic carboxylic acid isosteres is as follows:

| Name | Sample structure | Sample Formula: |
|---|---|---|
| carboxylic acid | 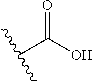 [SMILES: *C(=O)O] | COOH |
| hydroxamic acids | 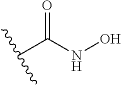 [SMILES: *C(=O)NO] | C(O)NH(OH) |
| | 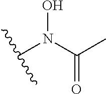 [SMILES: *N(O)C(C)=O] | N(OH)(C(O)CH₃) |
| hydroxamic esters | 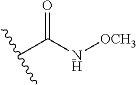 [SMILES: *C(=O)NOC] | C(O)NH(OCH₃) |
| | 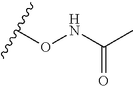 [SMILES: *ONC(C)=O] | ONH(C(O)CH₃) |
| phosphonic acid | 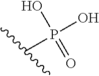 [SMILES: *P(=O)(O)O] | P(O)(OH)₂ |
| phosphinic acid | 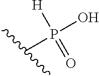 [SMILES: *[PH](=O)O] | P(O)(OH)(H) |
| sulfonic acid | 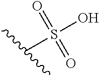 [SMILES: *S(=O)(=O)O] | S(O)₂OH |
| sulfinic acid | 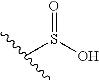 [SMILES: *S(=O)O] | S(O)OH |
| sulfonamides | 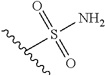 [SMILES: *S(N)(=O)=O] | S(O)₂NH₂ |
| | 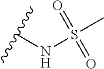 [SMILES: *NS(C)(=O)=O] | NH(S(O)₂CH₃) |
| acyl-sulfonamides | 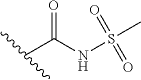 [SMILES: *C(=O)NS(C)(=O)=O] | C(O)NH(S(O)₂CH₃) |
| | 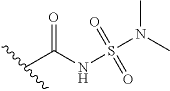 [SMILES: *C(=O)NS(=O)(=O)N(C)C] | C(O)NH(S(O)₂N(CH₃)₂) |
| sulfonylurea | 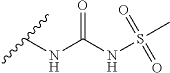 [SMILES: *NC(=O)NS(C)(=O)=O] | NH(C(O)NH(S(O)₂CH₃)) |
| acylurea | 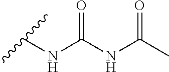 [SMILES: *NC(=O)NC(C)=O] | NH(C(O)NH(C(O)CH₃)) |
| iminosulfanone | 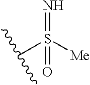 [SMILES: *S(C)(=N)=O] | S(═NH)(O)CH₃ |
| sulfonimidamide | 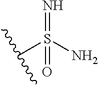 [SMILES: *S(=N)(N)=O] | S(═NH)(O)NH₂ |

A non-limiting list of cyclic carboxylic acid isosteres is as follows:

| Name | Sample structure |
|---|---|
| tetrazole | 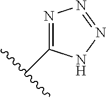 [SMILES: *c1nnn[nH]1] |
| thiazolidine dione | 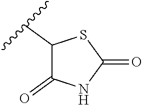 [SMILES: *C1SC(=O)NC1=O] |
| oxazolidine dione | 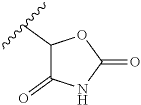 [SMILES: *C1OC(=O)NC1=O] |
| oxadiazol-5(4H)-one | 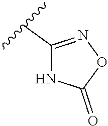 [SMILES: *c1noc(=O)[nH]1] |
| thiadiazol-5(4H)-one | 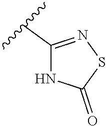 [SMILES: *c1nsc(=O)[nH]1] |
| oxathiadiazole-2-oxide | 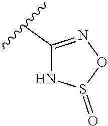 [SMILES: *C1=NOS(=O)N1] |
| oxadiazol-5(4H)-thione | 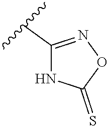 [SMILES: *c1noc(=S)[nH]1] |
| isoxazole | 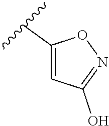 [SMILES: *c1cc(O)no1] |
| tetramic acid | 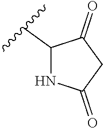 [SMILES: *C1NC(=O)CC1=O] |
| cyclopentane 1,3-diones | 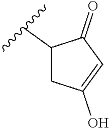 [SMILES: *C1CC(O)=CC1=O] |
| | 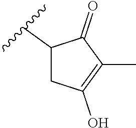 [SMILES: *C1CC(O)=C(C)C1=O] |
| | 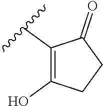 [SMILES: *C1=C(O)CCC1=O] |
| cyclopentane 1,2-diones | 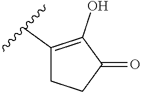 [SMILES: *C1=C(O)C(=O)CC1] |
| squaric acid derivatives | 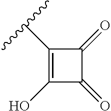 [SMILES: *c1c(O)c(=O)c1=O] |
| | 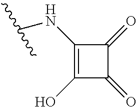 [SMILES: *Nc1c(O)c(=O)c1=O] |
| substituted phenols | 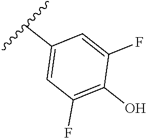 [SMILES: *c1cc(F)c(O)c(F)c1] |
| | 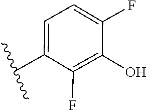 [SMILES: *c1ccc(F)c(O)c1F] |
| | 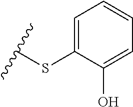 [SMILES: *Sc1ccccc1O] |
| | 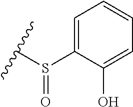 [SMILES: *S(=O)c1ccccc1O] |
| | 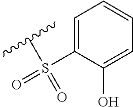 [SMILES: *S(=O)(=O)c1ccccc1O] |

Unless specified otherwise, the terms “compounds of the present invention”, “compound of the present invention”, “compound of the invention”, or “compounds of the invention” refer to a compound of formula (I), subformulae thereof (e.g., formulae (Ia), (II), (IIa), (III), (IIIa), (IIIb), (IV), (IVa), (IVb), (V), (Va) and (Vb)) and exemplified compounds, and salts thereof, as well as all stereoisomers (including diastereoisomers and enantiomers), rotamers, tautomers and isotopically labeled compounds (including deuterium substitutions), as well as inherently formed moieties.

Depending on the choice of the starting materials and procedures, the compounds can be present in the form of one of the possible stereoisomers or as mixtures thereof, for example as pure optical isomers, or as stereoisomer mixtures, such as racemates and diastereoisomer mixtures, depending on the number of asymmetric carbon atoms. The present invention is meant to include all such possible stereoisomers, including racemic mixtures, diasteriomeric mixtures and optically pure forms. Optically active (R)- and (S)-stereoisomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration. All tautomeric forms are also intended to be included.

The phrase “optionally substituted” means unsubstituted or substituted. The substituents are independently selected, and substitution may be at any chemically accessible position. The term “substituted” means that a hydrogen atom is removed and replaced by a substituent. A single divalent substituent, e.g., oxo, can replace two hydrogen atoms. It is to be understood that substitution at a given atom is limited by valency.

As used herein, “Cₙ₋ₘ alkyl”, employed alone or in combination with other terms, refers to a saturated hydrocarbon group that may be straight-chain or branched, having n to m carbons.

In some embodiments, the alkyl group contains from 1 to 6 carbon atoms, from 1 to 4 carbon atoms, from 1 to 3 carbon atoms, or from 1 to 2 carbon atoms.

As used herein, “Cₙ₋ₘ alkenyl” refers to an alkyl group having one or more double carbon-carbon bonds and having n to m carbons. In some embodiments, the alkenyl group contains from 2 to 6 carbon atoms, from 2 to 4 carbon atoms, or from 2 to 3 carbon atoms.

As used herein, “Cₙ₋ₘ alkynyl” refers to an alkyl group having one or more triple carbon-carbon bonds and having n to m carbons. In some embodiments, the alkynyl group contains from 2 to 6 carbon atoms, from 2 to 4 carbon atoms, or from 2 to 3 carbon atoms.

As used herein, “aryl” refers to an aromatic hydrocarbon group, which may be monocyclic or polycyclic (e.g., having 2, 3, or 4 fused rings). The term Cn-rₙ aryl refers to an aryl group having from n to m ring carbon atoms. In some embodiments, the aryl group has from 5 to 10 carbon atoms. In some embodiments, the aryl group is phenyl.

As used herein, “cycloalkyl” refers to non-aromatic cyclic hydrocarbons including cyclized alkyl and alkenyl groups. Cycloalkyl groups can include mono- or polycyclic (e.g., having two fused rings) groups, spirocycles, and bridged rings. Ring-forming carbon atoms of a cycloalkyl can be optionally substituted by oxo or sulfido (e.g., C(O) or C(S)). In some embodiments, the cycloalkyl groups have 3, 4, 5, 6, 7, 8, 9, or 10 ring-forming carbon atoms (i.e., C₃₋₁₀ cycloalkyl). In some embodiments, the cycloalkyl groups have 3, 4, 5, or 6 ring-forming carbon atoms (i.e., C₃₋₆ cycloalkyl).

As used herein, “heteroaryl” refers to a monocyclic or polycyclic (e.g., having 2 fused rings) aromatic heterocycle having at least one heteroatom ring member selected from N, O, and S. In some embodiments, a ring-forming N in a heteroaryl group can be an N-oxide. In some embodiments, the heteroaryl is a 5-10 membered monocylic or bicyclic heteroaryl having 1, 2, 3, or 4 heteroatom ring members independently selected from N, O, and S. In some embodiments, the heteroaryl is a 5-10 membered monocylic or bicyclic heteroaryl having 1, 2, 3, or 4 heteroatom ring members independently selected from N, O, and S.

As used herein, “heterocycloalkyl” refers to monocyclic or polycyclic heterocycles having at least one non-aromatic ring (saturated or partially unsaturated ring), wherein one or more of the ring-forming carbon atoms of the heterocycloalkyl is replaced by a heteroatom selected from N, O, S, and B, and wherein a ring-forming carbon or heteroatom of a heterocycloalkyl group can be optionally substituted by one or more oxo or sulfido (e.g., C(O), S(O), C(S), S(O)₂), etc.). In some embodiments, the heterocycloalkyl group contains 4 to 10 ring-forming atoms (i.e., 4-10 membered), wherein 1 to 4 atoms are heteroatoms independently selected from N, O, and S.

As used herein, “Cₒ₋ₚ-cycloalkyl-Cₙ₋ₘ-alkyl” refers to a group of formula cycloalkyl-alkylene, wherein the cycloalkyl has o to p carbon atoms and the alkylene linking group has n to m carbon atoms.

As used herein, “heterocycloalkyl-Cₙ₋ₘ-alkyl” refers to a group of formula heterocycloalkyl-alkylene, wherein the alkylene linking group has n to m carbon atoms.

As used herein, “heteroaryl-Cₙ₋ₘ-alkyl” refers to a group of formula heteroaryl-alkylene, wherein the alkylene linking group has n to m carbon atoms.

As used herein, “aryl-Cₙ₋ₘ-alkyl” (e.g., “phenyl-Cₙ₋ₘ-alkyl”) refers to a group of formula aryl-alkylene, wherein the alkylene linking group has n to m carbon atoms.

As used herein, the terms “salt” or “salts” refers to an acid addition or base addition salt of a compound provided herein. “Salts” include in particular “pharmaceutically acceptable salts”. The term “pharmaceutically acceptable salts” refers to salts that retain the biological effectiveness and properties of the compounds provided herein and, which typically are not biologically or otherwise undesirable. In many cases, the compounds provided herein are capable of forming acid and/or base salts by virtue of the presence of basic nitrogen atoms, for example as found in amino and pyridine groups or other groups similar thereto and/or acidic protons, for example as found in carboxylic acid or other groups similar thereto.

Pharmaceutically acceptable acid addition salts can be formed with inorganic acids and organic acids.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

In another aspect, compounds provided herein are in sodium, potassium, ammonium, calcium, magnesium, iron, silver, zinc, copper, isopropylamine, benzathine, cholinate, diethanolamine, diethylamine, lysine, meglumine, piperazine or tromethamine salt form.

In another aspect, compounds provided herein are in acetate, ascorbate, adipate, aspartate, benzoate, besylate, bromide/hydrobromide, bicarbonate/carbonate, bisulfate/sulfate, camphorsulfonate, caprate, chloride/hydrochloride, chlortheophyllonate, citrate, ethandisulfonate, fumarate, gluceptate, gluconate, glucuronate, glutamate, glutarate, glycolate, hippurate, hydroiodide/iodide, isethionate, lactate, lactobionate, laurylsulfate, malate, maleate, malonate, mandelate, mesylate, methylsulfate, mucate, naphthoate, napsylate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfosalicylate, sulfate, tartrate, tosylate trifenatate, trifluoroacetate or xinafoate salt form.

Any formula given herein is also intended to represent unlabeled forms as well as isotopically labeled forms of the compounds. Isotopically labeled compounds have structures depicted by the formulae given herein except that one or more atoms are replaced by an atom having a selected atomic mass or mass number. Isotopes that can be incorporated into compounds of the invention include, for example, isotopes of hydrogen.

Further, incorporation of certain isotopes, particularly deuterium (i.e., ²H or D) may afford certain therapeutic advantages resulting from greater metabolic stability, for example increased in vivo half-life or reduced dosage requirements or an improvement in therapeutic index or tolerability. It is understood that deuterium in this context is regarded as a substituent of a compound of the present invention. The concentration of deuterium, may be defined by the isotopic enrichment factor. The term “isotopic enrichment factor” as used herein means the ratio between the isotopic abundance and the natural abundance of a specified isotope. If a substituent in a compound of this invention is denoted as being deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). It should be understood that the term “isotopic enrichment factor” can be applied to any isotope in the same manner as described for deuterium.

Other examples of isotopes that can be incorporated into compounds of the invention include isotopes of hydrogen, carbon, nitrogen, oxygen, fluorine and sulfur, such as ³H, ¹¹C, ¹³C, ¹⁴C, ¹⁵N, ¹⁸F, ³⁵S respectively. Accordingly, it should be understood that the invention includes compounds that incorporate one or more of any of the aforementioned isotopes, including for example, radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. Isotopically-labeled compounds of the present invention can generally be prepared by conventional techniques known to those skilled in the art or by processes analogous to those described in the accompanying Examples and Preparations using an appropriate isotopically-labeled reagent in place of the non-labeled reagent previously employed.

As used herein, the term “pharmaceutical composition” refers to a compound of the invention, or a pharmaceutically acceptable salt thereof, together with at least one pharmaceutically acceptable carrier, in a form suitable for oral or parenteral administration.

As used herein, the term “pharmaceutically acceptable carrier” refers to a substance useful in the preparation or use of a pharmaceutical composition and includes, for example, suitable diluents, solvents, dispersion media, surfactants, antioxidants, preservatives, isotonic agents, buffering agents, emulsifiers, absorption delaying agents, salts, drug stabilizers, binders, excipients, disintegration agents, lubricants, wetting agents, sweetening agents, flavoring agents, dyes, and combinations thereof, as would be known to those skilled in the art (see, for example, Remington The Science and Practice of Pharmacy, 22^{nd} Ed. Pharmaceutical Press, 2013, pp. 1049-1070).

The term “a therapeutically effective amount” of a compound of the present invention refers to an amount of a compound of the present invention that will elicit the biological or medical response of a subject, for example, agonize GLP1R activity, ameliorate symptoms, alleviate conditions, slow or delay the progression of a disease, disorder or condition, or prevent a disease, disorder or condition. In one embodiment, the term “a therapeutically effective amount” refers to the amount of a compound of the present invention that, when administered to a subject, is effective to at least partially alleviate, prevent and/or ameliorate a condition, or a disorder or a disease responsive to increasing or agonizing the activity of GLP1R. In another embodiment, the term “a therapeutically effective amount” refers to the amount of a compound of the present invention that, when administered to a subject, cell, or a tissue, or a non-cellular biological material, or a medium, is effective to partially or fully agonize the activity of GLP1R. In another embodiment, the term “a therapeutically effective amount” refers to the amount of a compound of the present invention that, when administered to a subject, is effective to cause an observable level of one or more desired biological or medicinal responses, for example selected from: lowering glucose levels (or improving glucose homeostasis), increasing insulin sensitivity, lowering triglyceride or cholesterol levels, reducing body weight, reducing food intake and reducing body fat mass (such as peripheral fat and/or visceral fat).

As used herein, the term “subject” refers to primates (e.g., humans, male or female), dogs, rabbits, guinea pigs, pigs, rats and mice. In certain embodiments, the subject is a primate. In yet other embodiments, the subject is a human.

As used herein, the terms “agonize”, “agonism” and “agonizing” refer to an increase of signaling and/or activity of GLP1R, for example, as measured by an increase in intracellular cyclic adenosine mono-phosphate (cAMP).

As used herein, the term “treat”, “treating” or “treatment” of any disease, disorder or condition refers to alleviating or ameliorating the disease, disorder or condition (i.e., slowing or arresting the development or progression of the disease, disorder or condition, or at least one of the clinical symptoms thereof); or alleviating or ameliorating at least one physical parameter or biomarker associated with the disease, disorder or condition, including those which may not be discernible to the patient.

As used herein, the term “prevent”, “preventing” or “prevention” of any disease, disorder or condition refers to the prophylactic treatment of the disease, disorder or condition; or delaying the onset or progression of the disease, disorder or condition.

As used herein, a subject is “in need of” a treatment if such subject would benefit biologically, medically or in quality of life from such treatment.

As used herein, the term “a”, “an”, “the” and similar terms used in the context of the present invention (especially in the context of the claims) are to be construed to cover both the singular and plural unless otherwise indicated herein or clearly contradicted by the context.

All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed.

Any asymmetric atom (e.g., carbon or the like) of the compound(s) of the present invention can be present in racemic or enantiomerically enriched, for example the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration.

Accordingly, as used herein a compound of the present invention may be in the form of one of the possible stereoisomers, rotamers, atropisomers, tautomers or mixtures thereof, for example, as substantially pure diastereomers, optical isomers (antipodes), racemates or mixtures thereof.

Any resulting mixtures of stereoisomers can be separated on the basis of the physicochemical differences of the constituents, into the pure or substantially pure geometric or optical isomers, diastereomers, racemates, for example, by chromatography and/or fractional crystallization.

Any resulting racemates of compounds of the present invention or of intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic compounds of the present invention or racemic intermediates can also be resolved by chiral chromatography, e.g., high pressure liquid chromatography (HPLC) using a chiral adsorbent.

The compounds provided herein (e.g., compounds of formulae (Ia), (II), (IIa), (III), (IIIa), (IIIb), (IV), (IVa), (IVb), (V), (Va) and (Vb), and pharmaceutically acceptable salts thereof) can be prepared in a number of ways well known to those skilled in the art of organic synthesis. By way of example, compounds provided herein can be synthesized using the methods described in the Examples, together with synthetic methods known in the art of synthetic organic chemistry, or variations thereon as appreciated by those skilled in the art.

It is well understood that protecting groups for sensitive or reactive groups are employed where necessary in accordance with general principles of chemistry. Protecting groups are manipulated according to standard methods of organic synthesis as described for example in Protective Groups in Organic Synthesis, 3rd edition, John Wiley & Sons: New York, 1999 or Protecting Groups, 3rd edition, Thieme, Stuttgart, 2004. Protective groups are removed at a convenient stage of the compound synthesis using methods that are readily apparent to those skilled in the art.

Those skilled in the art will recognize if a stereocenter exists in the compounds disclosed herein. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-Interscience, 1994).

The compounds of formula (I) according to the invention, as well as compound of any of formula (Ia), (II), (IIa), (III), (IIIa), (IIIb), (IV), (IVa), (IVb), (V), (Va) and (Vb), and pharmaceutically acceptable salts thereof, can be prepared in a number of ways well known to those skilled in the art of organic synthesis. By way of example, compounds provided herein can be synthesized using the methods described in any of general synthetic Schemes A and B or in the Examples, together with synthetic methods known in the art of synthetic organic chemistry, or variations thereon as appreciated by those skilled in the art.

As set forth in Scheme A, intermediate II.A, wherein R¹, R² and R³ are as defined herein, is reacted with an intermediate III.A, wherein Y is Br or I, and R⁴ is as defined herein, to afford an intermediate I.A. The intermediate I.A, wherein Y is Br or I, and R¹, R², R³ and R⁴ are as defined herein, is then reacted with a carboxylic acid ester derivative B1 in the presence of a Pd catalyst (e.g., Xphos Pd G2) and a base in a solvent (e.g., toluene) to afford a compound of formula (I). In the carboxylic acid ester derivative B1 of this embodiment, R is C₁₋₃-alkyl and H^{A} is 5-10 membered heteroaryl or (5-10 membered heteroaryl)-C₁₋₃-alkyl-, wherein the 5-10 membered heteroaryl and (5-10 membered heteroaryl)-C₁₋₃-alkyl- are each optionally substituted with 1, 2, or 3 groups independently selected from halo, CN, NO₂, OR^{5A}, SR^{5A}, C₁₋₃-alkyl, C₁₋₃-alkyl substituted with 1, 2 or 3 halo, C(O)R^{5B}, C(O)NR^{5C}R^{5D}, C(O)NR^{5C}(OR^{5A}), C(O)NR^{5C}(S(O)₂R^{5B}), C(O)NR^{5C}(S(O)₂NR^{5C}R^{5D}), NR^{5C}OR^{5A}, NR^{5C}R^{5D}, NR^{5C}(C(O)R^{5B}), NR^{5C}(C(O)OR^{5A}), N(OR^{5A})(C(O)R^{5B}), NR^{5C}(C(O)NR^{5C}R^{5D}), NR^{5C}(C(O)NR^{5C}(C(O)R^{5B})), NR^{5C}(S(O)₂R^{5B}), NR^{5C}(S(O)₂NR^{5C}R^{5D}), NR^{5C}(C(O)NR^{5C}(S(O)₂R^{5B})), OC(O)R^{5B}, OC(O)NR^{5C}R^{5D}, ONR^{5C}(C(O)R^{5B}), OS(O)₂R^{5B}, OP(O)(OR^{5E})(OR^{5F}), S(O)OR^{5A}, S(O)R^{5B}, S(O)₂R^{5B}, S(O)₂NR^{5C}R^{5D}, S(O)₂OR^{5A}, S(═NR^{5G})(O)R^{5B}, S(═NR^{5G})(O)NR^{5C}NR^{5D}, P(O)(OR^{5E})(OR^{5F}), and P(O)(OR^{5E})(R^{5F}) wherein R^{5A}, R^{5B}, R^{5C}, R^{5D}, R^{5E}, R^{5F} and R^{5G} are as defined herein.

In another embodiment, intermediate I.A, wherein Y is Br or I, and R¹, R², R³ and R⁴ are as defined herein, is reacted with a carboxylic acid ester derivative B1, wherein R is C₁₋₃-alkyl and H^{A} is 5-10 membered heteroaryl or (5-10 membered heteroaryl)-C₁₋₃-alkyl-, wherein the 5-membered heteroaryl and (5-10 membered heteroaryl)-C₁₋₃-alkyl- are each substituted with boronic acid or a dioxaborolan protecting group (e.g., 4,4,5,5-tetramethyl-1,3,2-dioxaborolan-2-yl), and optionally with 1, 2, or 3 groups independently selected from halo, CN, NO₂, OR^{5A}, SR^{5A}, C₁₋₃-alkyl, C₁₋₃-alkyl substituted with 1, 2 or 3 halo, C(O)R^{5B}, C(O)NR^{5C}R^{5D}, C(O)NR^{5C}(OR^{5A}), C(O)NR^{5C}(S(O)₂R^{5B}), C(O)NR^{5C}(S(O)₂NR^{5C}R^{5D}), NR^{5C}OR^{5A}, NR^{5C}R^{5D}, NR^{5C}(C(O)R^{5B}), NR^{5C}(C(O)OR^{5A}), N(OR^{5A})(C(O)R^{5B}), NR^{5C}(C(O)NR^{5C}R^{5D}), NR^{5C}(C(O)NR^{5C}(C(O)R^{5B})), NR^{5C}(S(O)₂R^{5B}), NR^{5C}(S(O)₂NR^{5C}R^{5D}), NR^{5C}(C(O)NR^{5C}(S(O)₂R^{5B})), OC(O)R^{5B}, OC(O)NR^{5C}R^{5D}, ONR^{5C}(C(O)R^{5B}), OS(O)₂R^{5B}, OP(O)(OR^{5E})(OR^{5F}), S(O)OR^{5A}, S(O)R^{5B}, S(O)₂R^{5B}, S(O)₂NR^{5C}R^{5D}, S(O)₂OR^{5A}, S(═NR^{5G})(O)R^{5B}, S(═NR^{5G})(O)NR^{5C}NR^{5D}, P(O)(OR^{5E})(OR^{5F}), and P(O)(OR^{5E})(R^{5F}) wherein R^{5A}, R^{5B}, R^{5C}, R^{5D}, R^{5E}, R^{5F} and R^{5G} are as defined herein.

In one embodiment, the compound of formula (I) so obtained is a carboxylic acid ester derivative, wherein R¹, R², R³ and R⁴ are as defined herein, and H^{A′} is 5-10 membered heteroaryl or (5-10 membered heteroaryl)-C₁₋₃-alkylene-, wherein the 5-10 membered heteroaryl and (5-10 membered heteroaryl)-C₁₋₃-alkylene- are each optionally substituted with 1, 2, or 3 groups independently selected from halo, CN, NO₂, OR^{5A}, SR^{5A}, C₁₋₃-alkyl, C₁₋₃-alkyl substituted with 1, 2 or 3 halo, C(O)R^{5B}, C(O)NR^{5C}R^{5D}, C(O)NR^{5C}(OR^{5A}), C(O)NR^{5C}(S(O)₂R^{5B}), C(O)NR^{5C}(S(O)₂NR^{5C}R^{5D}), NR^{5C}OR^{5A}, NR^{5C}R^{5D}, NR^{5C}(C(O)R^{5B}), NR^{5C}(C(O)OR^{5A}), N(OR^{5A})(C(O)R^{5B}), NR^{5C}(C(O)NR^{5C}R^{5D}), NR^{5C}(C(O)NR^{5C}(C(O)R^{5B})), NR^{5C}(S(O)₂R^{5B}), NR^{5C}(S(O)₂NR^{5C}R^{5D}), NR^{5C}(C(O)NR^{5C}(S(O)₂R^{5B})), OC(O)R^{5B}, OC(O)NR^{5C}R^{5D}, ONR^{5C}(C(O)R^{5B}), OS(O)₂R^{5B}, OP(O)(OR^{5E})(OR^{5F}), S(O)OR^{5A}, S(O)R^{5B}, S(O)₂R^{5B}, S(O)₂NR^{5C}R^{5D}, S(O)₂OR^{5A}, S(═NR^{5G})(O)R^{5B}, S(═NR^{5G})(O)NR^{5C}NR^{5D}, P(O)(OR^{5E})(OR^{5F}), and P(O)(OR^{5E})(R^{5F}) wherein R^{5A}, R^{5B}, R^{5C}, R^{5D}, R^{5E}, R^{5F} and R^{5G} are as defined herein, wherein said carboxylic acid ester derivative may be converted into further compounds of formula (I) by hydrolysis of the carboxylic acid ester moiety into a carboxylic acid moiety in the presence of one or more hydroxide sources (e.g., MeOH and/or LiOH) in water.

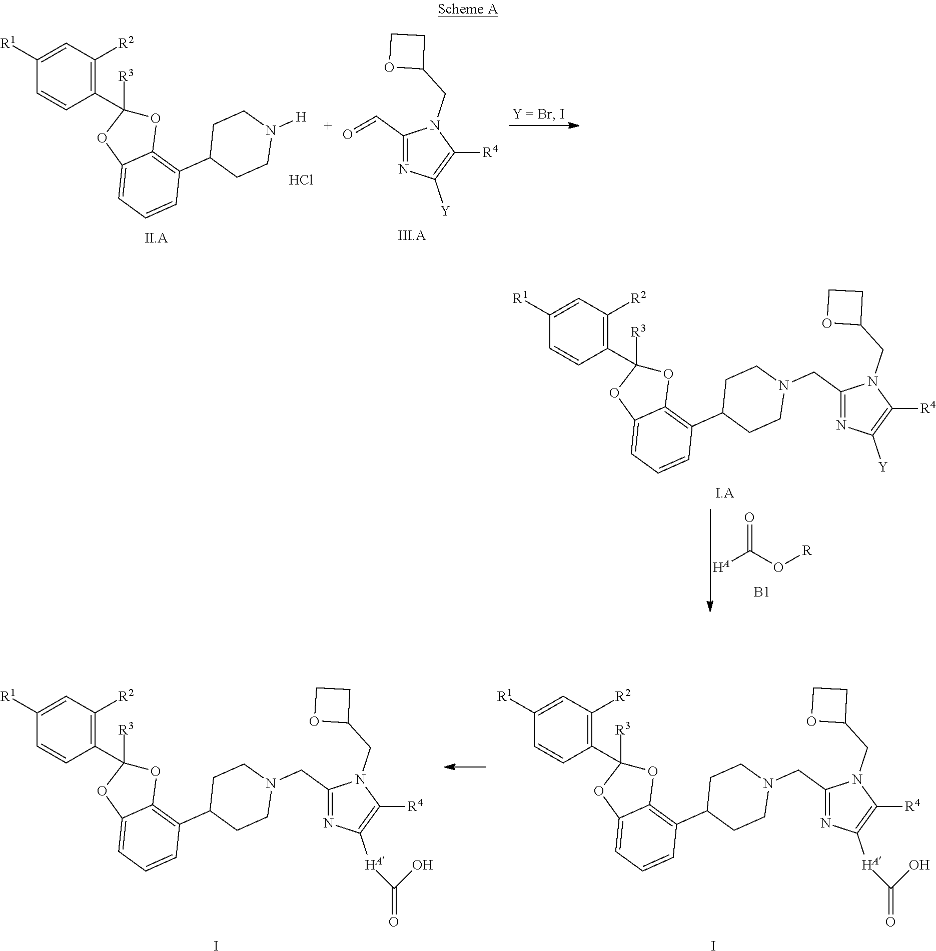

As set forth in Scheme B, intermediate II.A, wherein R¹, R² and R³ are as defined herein, is reacted with an intermediate III.B, wherein Y is Br or I, and R⁵ is as defined herein, to afford an intermediate I.B. The intermediate I.B, wherein R¹, R², R³ and R⁵ are as defined herein and Y is Br or I, is then reacted with a carboxylic acid ester derivative B2 in the presence of a Pd catalyst (e.g., Xphos Pd G2) and a base in a solvent (e.g., toluene) to afford a compound of formula (I). In the carboxylic acid ester derivative B2 of this embodiment, R is C₁₋₃-alkyl and H^{B} is 5-10 membered heteroaryl or (5-10 membered heteroaryl)-C₁₋₃-alkyl-, wherein the 5-10 membered heteroaryl and (5-10 membered heteroaryl)-C₁₋₃-alkyl- are each optionally substituted with 1, 2, or 3 groups independently selected from halo, CN, NO₂, OR^{4A}, SR^{4A}, C₁₋₃-alkyl, C₁₋₃-alkyl substituted with 1, 2 or 3 halo, C(O)R^{4B}, C(O)NR^{4C}R^{4D}, C(O)NR^{4C}(OR^{4A}), C(O)NR^{4C}(S(O)₂R^{4B}), C(O)NR^{4C}(S(O)₂NR^{4C}R^{4D}), NR^{4C}OR^{4A}, NR^{4C}R^{4D}, NR^{4C}(C(O)R^{4B}), NR^{4C}(C(O)OR^{4A}), N(OR^{4A})(C(O)R^{4B}), NR^{4C}(C(O)NR^{4C}R^{4D}), NR^{4C}(C(O)NR^{4C}(C(O)R^{4B})), NR^{4C}(S(O)₂R^{4B}), NR^{4C}(S(O)₂NR^{4C}R^{4D}), NR^{4C}(C(O)NR^{4C}(S(O)₂R^{4B})), OC(O)R^{4B}, OC(O)NR^{4C}R^{4D}, ONR^{4C}(C(O)R^{4B}), OS(O)₂R^{4B}, OP(O)(OR^{4E})(OR^{4F}), S(O)OR^{4A}, S(O)R^{4B}, S(O)₂R^{4B}, S(O)₂NR^{4C}R^{4D}, S(O)₂OR^{4A}, S(═NR^{4G})(O)R^{4B}, S(═NR^{4G})(O)NR^{4C}NR^{4D}, P(O)(OR^{4E})(OR^{4F}), and P(O)(OR^{4E})(R^{4F}), wherein R^{4A}, R^{4B}, R^{4C}, R^{4D}, R^{4E}, R^{4F} and R^{4G} are as defined herein.

In another embodiment, intermediate I.B, wherein R¹, R², R³ and R⁵ are as defined herein and Y is Br or I, is reacted with a carboxylic acid ester derivative B2, wherein R is C₁₋₃-alkyl and H^{B} is 5-10 membered heteroaryl or (5-10 membered heteroaryl)-C₁₋₃-alkyl-, wherein the 5-10 membered heteroaryl and (5-10 membered heteroaryl)-C₁₋₃-alkyl- are each substituted with boronic acid or a dioxaborolan protecting group (e.g., 4,4,5,5-tetramethyl-1,3,2-dioxaborolan-2-yl), and optionally with 1, 2, or 3 groups independently selected from halo, CN, NO₂, OR^{4A}, SR^{4A}, C₁₋₃-alkyl, C₁₋₃-alkyl substituted with 1, 2 or 3 halo, C(O)R^{4B}, C(O)NR^{4C}R^{4D}, C(O)NR^{4C}(OR^{4A}), C(O)NR^{4C}(S(O)₂R^{4B}), C(O)NR^{4C}(S(O)₂NR^{4C}R^{4D}), NR^{4C}OR^{4A}, NR^{4C}R^{4D}, NR^{4C}(C(O)R^{4B}), NR^{4C}(C(O)OR^{4A}), N(OR^{4A})(C(O)R^{4B}), NR^{4C}(C(O)NR^{4C}R^{4D}), NR^{4C}(C(O)NR^{4C}(C(O)R^{4B})), NR^{4C}(S(O)₂R^{4B}), NR^{4C}(S(O)₂NR^{4C}R^{4D}), NR^{4C}(C(O)NR^{4C}(S(O)₂R^{4B})), OC(O)R^{4B}, OC(O)NR^{4C}R^{4D}, ONR^{4C}(C(O)R^{4B}), OS(O)₂R^{4B}, OP(O)(OR^{4E})(OR^{4F}), S(O)OR^{4A}, S(O)R^{4B} S(O)₂R^{4B} S(O)₂NR^{4C}R^{4D}, S(O)₂OR^{4A}, S(═NR^{4G})(O)R^{4B}, S(═NR^{4G})(O)NR^{4C}NR^{4D}, P(O)(OR^{4E})(OR^{4F}), and P(O)(OR^{4E})(R^{4F}), wherein R^{4A}, R^{4B}, R^{4C}, R^{4D}, R^{4E}, R^{4F} and R^{4G} are as defined herein.

In one embodiment, the compound of formula (I) so obtained is a carboxylic acid ester derivative, wherein R¹, R², R³ and R⁵ are as defined herein, and H^{B′} is 5-10 membered heteroaryl or (5-10 membered heteroaryl)-C₁₋₃-alkylene-, wherein the 5-10 membered heteroaryl and (5-10 membered heteroaryl)-C₁₋₃-alkylene- are each optionally substituted with 1, 2, or 3 groups independently selected from halo, CN, NO₂, OR^{4A}, SR^{4A}, C₁₋₃-alkyl, C₁₋₃-alkyl substituted with 1, 2 or 3 halo, C(O)R^{4B}, C(O)NR^{4C}R^{4D}, C(O)NR^{4C}(OR^{4A}), C(O)NR^{4C}(S(O)₂R^{4B}), C(O)NR^{4C}(S(O)₂NR^{4C}R^{4D}), NR^{4C}OR^{4A}, NR^{4C}R^{4D}, NR^{4C}(C(O)R^{4B}), NR^{4C}(C(O)OR^{4A}), N(OR^{4A})(C(O)R^{4B}), NR^{4C}(C(O)NR^{4C}R^{4D}), NR^{4C}(C(O)NR^{4C}(C(O)R^{4B})), NR^{4C}(S(O)₂R^{4B}), NR^{4C}(S(O)₂NR^{4C}R^{4D}), NR^{4C}(C(O)NR^{4C}(S(O)₂R^{4B})), OC(O)R^{4B}, OC(O)NR^{4C}R^{4D}, ONR^{4C}(C(O)R^{4B}), OS(O)₂R^{4B}, OP(O)(OR^{4E})(OR^{4F}), S(O)OR^{4A}, S(O)R^{4B}, S(O)₂R^{4B}, S(O)₂NR^{4C}R^{4D}, S(O)₂OR^{4A}, S(═NR^{4G})(O)R^{4B}, S(═NR^{4G})(O)NR^{4C}NR^{4D}, P(O)(OR^{4E})(OR^{4F}), and P(O)(OR^{4E})(R^{4F}) wherein R^{4A}, R^{4B}, R^{4C}, R^{4D}, R^{4E}, R^{4F} and R^{4G} are as defined herein, wherein said carboxylic acid ester derivative may be converted into further compounds of formula (I) by hydrolysis of the carboxylic acid ester moiety into a carboxylic acid moiety in the presence of one or more hydroxide sources (e.g., MeOH and/or LiOH) in water.

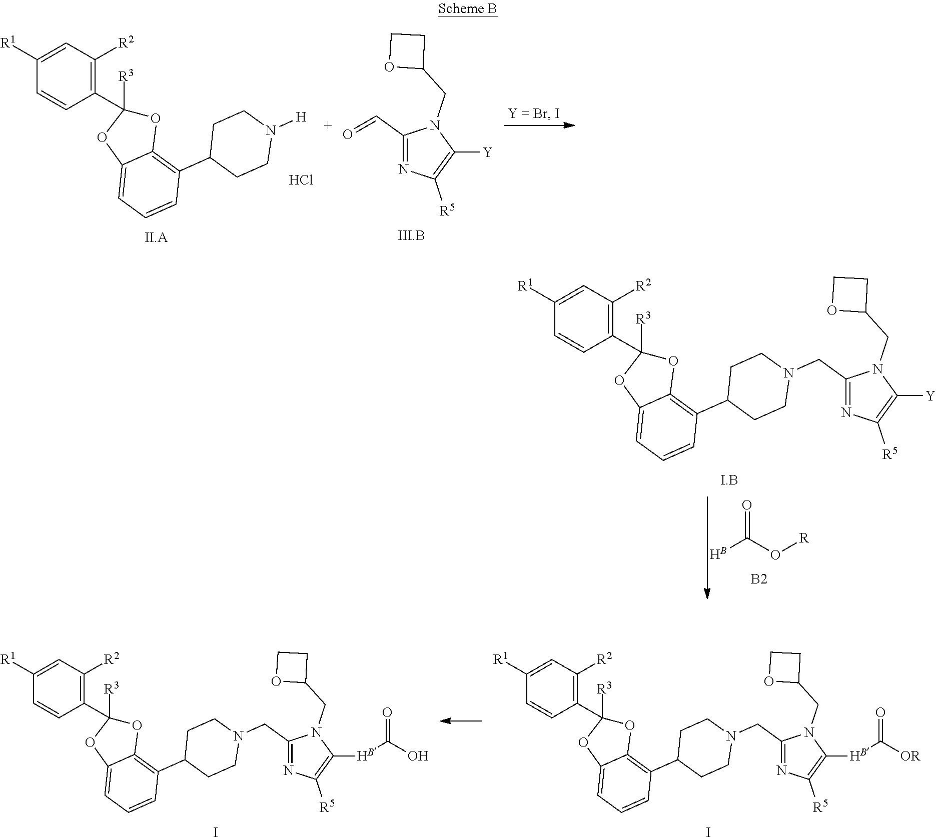

Intermediate II.A is either commercially available or can be produced by organic synthesis methods known to one of ordinary skill in the art. For example, intermediate II.A can be prepared as described below in Scheme C.

As shown in Scheme C, intermediate II.1, wherein R¹, R² and R³ are as defined herein, is reacted with 3-bromobenzene-1,2-diol in the presence of p-TsOH in a solvent (e.g., toluene) to afford intermediate II.2. Intermediate II.2, wherein R¹, R² and R³ are as defined herein, is then reacted with tert-butyl 4-(4,4,5,5-tetramethyl-1,3,2-dioxaborolan-2-yl)-3,6-dihydropyridine-1(2H)-carboxylate in the presence of a Pd catalyst and a base (e.g., Na₂CO₃) in a solvent (e.g., dioxane) to afford intermediate II.3. Intermediate II.3, wherein R¹, R² and R³ are as defined herein, is converted under H₂ in the presence of a catalyst (e.g., ((C₆H₅)₃P)₃RhCl) into intermediate II.4. Intermediate II.4, wherein R¹, R² and R³ are as defined herein, is then purified by SFC into intermediates II.5a and II.5b. Intermediates II.5a and II.5b, wherein R¹, R² and R³ are as defined herein, are converted into intermediates II.Aa and II.Ab, respectively, wherein R¹, R² and R³ are as defined herein, in the presence of HCl in a solvent (e.g., dioxane).

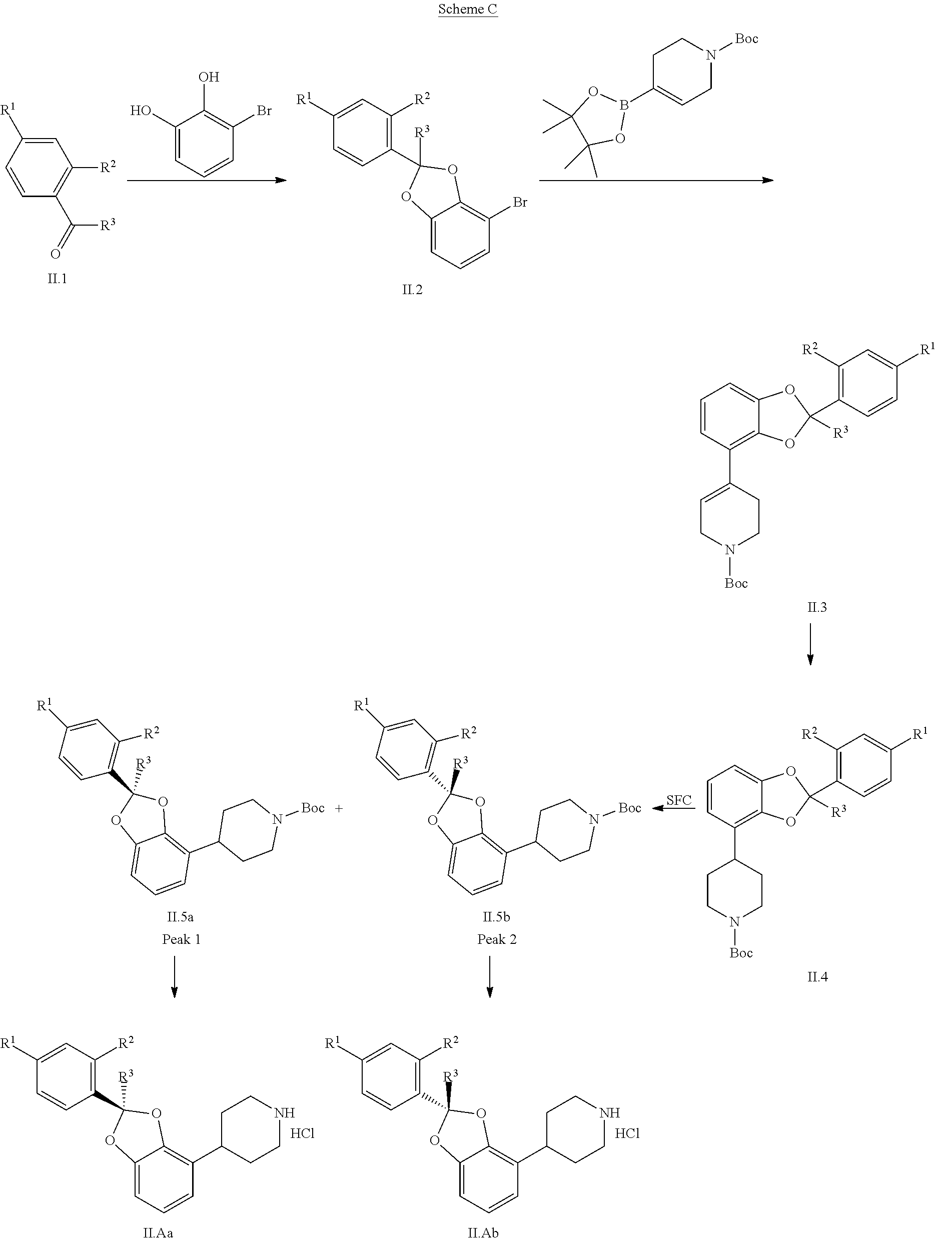

Intermediates III.A and III.B are either commercially available or can be produced by organic synthesis methods known to one of ordinary skill in the art. For example, intermediates III.A and III.B can be prepared as described below in Scheme D.

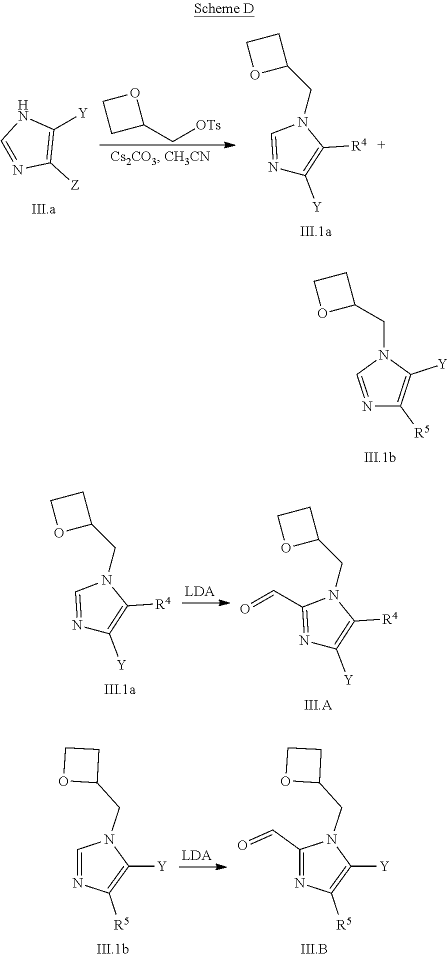

As shown in scheme D, intermediate III.a, wherein Z is R⁴ or R⁵ as defined herein and Y is Br or I, is reacted with oxetan-2-ylmethyl 4-methylbenzenesulfonate in the presence of cesium carbonate in a solvent (e.g., acetonitrile). The residue is purified via chromatography to separate the isomers to afford intermediates III.1a and III.1b, wherein R⁴ or R⁵ is as defined herein and Y is Br or I. Intermediates III.1a and III.1b, wherein R⁴ or R⁵ is as defined herein and Y is Br or I, are independently reacted with LDA in a solvent (e.g., THF/heptane/ethylbenzene) to afford intermediates III.A and III.B, respectively.

In another aspect, provided herein is a pharmaceutical composition comprising a compound of formula (I), or a pharmaceutically acceptable salt thereof, and a pharmaceutically acceptable carrier.

In another aspect, provided herein is a pharmaceutical composition comprising a therapeutically effective amount of a compound of formula (I), or a pharmaceutically acceptable salt thereof, and a pharmaceutically acceptable carrier.

In a further embodiment, the composition comprises at least two pharmaceutically acceptable carriers, such as those described herein. The pharmaceutical composition can be formulated for particular routes of administration such as oral administration, parenteral administration (e.g., by injection, infusion, transdermal or topical administration), and rectal administration. Topical administration may also pertain to inhalation or intranasal application. The pharmaceutical compositions of the present invention can be made up in a solid form (including, without limitation, capsules, tablets, pills, granules, powders or suppositories), or in a liquid form (including, without limitation, solutions, suspensions or emulsions). Tablets may be either film coated or enteric coated according to methods known in the art. Typically, the pharmaceutical compositions are tablets or gelatin capsules comprising the active ingredient together with one or more of:
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and
e) absorbents, colorants, flavors and sweeteners.

The compounds of the present invention (e.g., compounds of formulae (Ia), (II), (IIa), (III), (IIIa), (IIIb), (IV), (IVa), (IVb), (V), (Va) and (Vb), and pharmaceutically acceptable salts thereof) in free form or in pharmaceutically acceptable salt form, exhibit valuable pharmacological properties, for example as agonists of GLP1R, e.g., as indicated in in vitro and in vivo tests as provided in the next sections, and are therefore indicated for therapy or for use as research chemicals, e.g., as tool compounds.

The compounds of the present invention (e.g., compounds of formulae (Ia), (II), (IIa), (III), (IIIa), (IIIb), (IV), (IVa), (IVb), (V), (Va) and (Vb), and pharmaceutically acceptable salts thereof) may be useful in the treatment of metabolic and related diseases, disorders and conditions. In particular, the compounds of the present invention may be useful in the treatment of metabolic and related diseases, disorders and conditions selected from: obesity, type 2 diabetes mellitus, insulin resistance, hyperinsulinemia, glucose intolerance, hyperglycemia, one or more diabetic complications (including but not limited to chronic kidney disease), diabetic nephropathy, dyslipidemia, metabolic syndrome, progressive liver disease, cardiovascular diseases and neuropathy (in particular, peripheral neuropathy, e.g., associated with diabetes).

The progressive liver disease may be, for example, non-alcoholic fatty liver disease (NAFLD), or, for example, non-alcoholic steatohepatitis (NASH).

The cardiovascular disease may be selected, for example, from: hypertension, atherosclerosis, peripheral arterial disease, stroke, cardiomyopathy, atrial fibrillation, heart failure (for example heart failure with reduced ejection fraction (HFrEF), heart failure with mid-range ejection fraction (HFmrEF)) and heart failure with preserved ejection fraction (HFpEF), coronary heart disease and arrhythmias (for example atrial arrhythmias and ventricular arrhythmias)).

The compounds of the present invention may be useful in the treatment of several diseases, disorders or conditions co-occurring in a subject (termed ‘co-morbidities’).

Co-morbidities, for example, may be those in subjects which are type 2 diabetic and are additionally obese and/or additionally exhibit heart failure and/or NASH. For example, an obese subject may also exhibit type 2 diabetes and/or exhibit cardiovascular disease (for example heart failure). Such subject may also exhibit a progressive liver disease (for example NASH). For example, an obese subject may also exhibit type 2 diabetes and/or exhibit cardiovascular disease (for example heart failure) and/or exhibit a progressive liver disease (for example NASH). The subject may also have high blood pressure and/or high blood cholesterol level. The subject may also suffer from peripheral neuropathy.

Therefore, a compound provided herein may be useful in the treatment of a disease, disorder or condition selected from: obesity, type 2 diabetes mellitus, insulin resistance, hyperinsulinemia, glucose intolerance, hyperglycemia, one or more diabetic complications (including but not limited to chronic kidney disease), diabetic nephropathy, dyslipidemia, non-alcoholic fatty liver disease (NAFLD), non-alcoholic steatohepatitis (NASH), hypertension, atherosclerosis, peripheral arterial disease, stroke, cardiomyopathy, atrial fibrillation, heart failure (in particular, heart failure with reduced ejection fraction (HFrEF), heart failure with mid-range ejection fraction (HFmrEF) and heart failure with preserved ejection fraction (HFpEF)), coronary heart disease, arrhythmias (in particular, atrial arrhythmias and ventricular arrhythmias) and neuropathy (in particular, peripheral neuropathy).

In an embodiment, the disease, disorder or condition is selected from obesity, type 2 diabetes, atherosclerosis, heart failure (in particular, HFpEF) and NASH.

In an embodiment, the disease, disorder or condition is selected from obesity, type 2 diabetes, atherosclerosis and heart failure (in particular, HFpEF).

Thus, as a further aspect, provided herein is the use of a compound of formula (I) or a pharmaceutically acceptable salt thereof (including, e.g., a compound of any of formula (Ia), (II), (IIa), (III), (IIIa), (IIIb), (IV), (IVa), (IVb), (V), (Va) and (Vb), or a pharmaceutically acceptable salt thereof), in therapy. In a further embodiment, the therapy is treatment of a disease, disorder or condition which may be treated by agonism of GLP1R. In another embodiment, the therapy is treatment of a disease, disorder or condition selected from the afore-mentioned lists, suitably obesity, type 2 diabetes, atherosclerosis and heart failure (in particular, heart failure with preserved ejection fraction), including where these present as co-morbidities, for example, in a subject with type 2 diabetes who is also obese, and/or a subject with type 2 diabetes who also has heart failure and/or a subject with type 2 diabetes who also has NASH.

Thus, as a further aspect, provided herein is a compound of formula (I) or a pharmaceutically acceptable salt thereof (including, e.g., a compound of any of formula (Ia), (II), (IIa), (III), (IIIa), (IIIb), (IV), (IVa), (IVb), (V), (Va) and (Vb), or a pharmaceutically acceptable salt thereof), for use in therapy. In a further embodiment, the therapy is treatment of a disease, disorder or condition which may be treated by agonism of GLP1R. In another embodiment, the therapy is treatment of a disease, disorder or condition selected from the afore-mentioned lists, suitably obesity, type 2 diabetes, atherosclerosis and heart failure (in particular, heart failure with preserved ejection fraction), including where these present as co-morbidities, for example, in a subject with type 2 diabetes who is also obese, and/or a subject with type 2 diabetes who also has heart failure and/or a subject with type 2 diabetes who also has NASH.

In another aspect, the provided herein is a method of treating a disease, disorder or condition which is treatable by agonism of GLP1R comprising administration of a therapeutically acceptable amount of a compound of formula (I) or a pharmaceutically acceptable salt thereof (including, e.g., a compound of any of formula (Ia), (II), (IIa), (III), (IIIa), (IIIb), (IV), (Iva), (Ivb), (V), (Va) and (Vb), or a pharmaceutically acceptable salt thereof). In another embodiment, the invention provides a method of treating a disease, disorder or condition in a subject in need thereof, the method comprising administering to the subject a therapeutically effective amount of a compound of formula (I), or a pharmaceutically acceptable salt thereof, wherein the disease, disorder or condition is selected from the afore-mentioned lists, suitably obesity, type 2 diabetes, atherosclerosis and heart failure (in particular, heart failure with preserved ejection fraction), including where these present as co-morbidities, for example, in a subject with type 2 diabetes who is also obese, and/or a subject with type 2 diabetes who also has heart failure and/or a subject with type 2 diabetes who also has NASH.

In a further aspect, the provided herein is a use of a compound of formula (I) or a pharmaceutically acceptable salt thereof (including, e.g., a compound of any of formula (Ia), (II), (IIa), (III), (IIIa), (IIIb), (IV), (IVa), (IVb), (V), (Va) and (Vb), or a pharmaceutically acceptable salt thereof), for the manufacture of a medicament. In a further embodiment, the medicament is for treatment of a disease which may be treated by agonism of GLP1R. In another embodiment, the disease is selected from the afore-mentioned lists, suitably obesity, type 2 diabetes, atherosclerosis and heart failure (in particular, heart failure with preserved ejection fraction), including where these present as co-morbidities, for example, in a subject with type 2 diabetes who is also obese, and/or a subject with type 2 diabetes who also has heart failure and/or a subject with type 2 diabetes who also has NASH.

The term “metabolic disorders or diseases” refers to an associated cluster of traits that includes, but is not limited to, glucose intolerance, insulin resistance, hyperinsulinemia, obesity, excess visceral adiposity, hypertension, dyslipidemia characterized by high triglycerides, low high-density lipoprotein (HDL)-cholesterol, and high low-density lipoprotein (LDL) cholesterol. Subjects having metabolic disease or disorder are at risk of developing of type 2 diabetes mellitus and, for example, atherosclerosis.

The term “type 2 diabetes mellitus” is a condition characterized by persistently high glucose levels both in the fasted and fed state which results from a combination of impaired glucose utilization and excess glucose production. This may result from either inadequate production of insulin from the pancreas or peripheral insulin resistance.

The term “insulin resistance” as used herein refers to a condition where a normal quantity of insulin cannot induce the expected physiological response and cannot activate downstream pathways. In many examples insulin beyond the physiologic range either endogenously produced or exogenously administered, is sufficient to induce a complete or partial biologic response to induce the expected physiological response.

The term “hyperinsulinemia” refers to a condition where excess insulin can be detected in the blood.

The term “glucose intolerance” encompasses any disorder characterized by a clinical symptom or a combination of clinical symptoms that is associated with an elevated level of basal or post-prandial glucose and/or an elevated level of insulin or abnormal glucose stimulated insulin release or HOMA-IR (homeostatic model assessment of insulin resistance) in a subject relative to a healthy individual. Elevated levels of glucose and/or insulin may be manifested in the following diseases, disorders and conditions: obesity, metabolic syndrome, impaired glucose tolerance, type II diabetes, gestational diabetes, type I diabetes, insulin resistance, hyperinsulinemia, lipodystrophy, lipoatrophy and various MODY (maturity onset diabetes of the young) mutations. The GLP1R agonists provided herein, and compositions thereof, can be used, for example, to achieve and/or maintain glucose homeostasis, e.g., to reduce glucose level in the bloodstream and/or to reduce insulin level to a range found in a healthy subject.

The term “hyperglycemia”, as used herein, refers to a condition in which an elevated amount of glucose circulates in the blood plasma of a subject relative to a healthy individual. Hyperglycemia can be diagnosed using methods known in the art, including measurement of fasting blood glucose levels as described herein.

The term “diabetic complications” are problems caused by persistently high blood glucose levels that damage other organs including kidneys, peripheral limbs, and eyes (e.g., retinopathies) or induce vascular disease and neuropathy. Impaired vascular function contributes to erectile dysfunction and can lead to increased risk of skin infections. Diabetes also increases the risk for heart disease and bone and joint disorders. Other long-term complications of diabetes include excess risk of cancer including hepatocellular carcinoma, endometrial cancer, breast cancer, and pancreatic cancer.

The term “diabetic nephropathy” is a condition resulting from diabetes and caused by damage to blood vessels and other cells in the kidney that reduces kidney function

The term “obesity” in human adults refers to a Body Mass Index (BMI) of 30 or greater (Centers for Disease Control and Prevention). Such subject may also be referred to as obese. This is referred to as Class I obesity. Class II obesity includes individuals with a BMI of 35-39.9 and Class III obesity refers to individuals with a BMI of greater than 40. Body mass index (BMI) is a measure of body fat based on height and weight. The formula for calculation is BMI=weight in kilograms/height in meters².

In an embodiment the human subject suffering from obesity has a BMI of ≥30 or ≥35 or a BMI in the range≥35 to <40 or ≥30 to <40. The amount<40 can, for example, be 39.9. In some embodiments the obesity is severe obesity or morbid obesity, wherein the human subject has a BMI of ≥40.

The term “dyslipidemia” refers to complex disorders of lipoprotein metabolism, including lipoprotein overproduction or abnormal metabolism. Dyslipidemias may be manifested by elevation of the total cholesterol, low-density lipoprotein (LDL) cholesterol and triglyceride concentrations, and a decrease in high-density lipoprotein (HDL) cholesterol concentration in the blood.

The term “atherosclerosis” refers to vascular disease characterized by irregularly distributed lipid deposits in the intima of large and medium-sized arteries, sometimes causing narrowing of arterial lumens and proceeding eventually to fibrosis and calcification. Lesions are usually focal and progress slowly and intermittently. Limitation of blood flow accounts for most clinical manifestations, which vary with the distribution and severity of lesions.

The term “progressive liver disease” refers to the progression from a benign state of hepatosteatosis evidenced by fibrosis and cirrhosis, which predispose to hepatocellular carcinoma. The progression of obesity associated non-alcoholic fatty liver (NAFL) to NASH, fibrosis and cirrhosis is well documented.

The term “non-alcoholic fatty liver disease (FLD)”, also known as NAFLD is a condition wherein excess lipid accumulates in hepatocytes, which can result from either excess de novo lipogenesis in the liver or abnormal clearance and oxidation of fatty acids. NAFLD is excluded from other causes of liver disease including alcoholic liver disease and viral liver disease. NAFLD includes three histologic entities that reflect progression of the disease: fatty liver, hepatosteatosis and fibrosis or cirrhosis. The most common cause of NAFLD is obesity, although NAFLD can also be seen in lean individuals. Accumulation of fat may progress inflammation accompanied by infiltration of macrophages and changes in hepatocyte histology including ballooning, termed steatohepatitis and referred to as non-alcoholic steatohepatitis (NASH). NASH may progress to fibrosis with interlobular bridging fibrosis or cirrhosis. As used herein, the term NASH may encompass steatohepatitis, hepatocellular ballooning and lobular inflammation.

The term “metabolic syndrome” refers to a cluster of risk factors that raises the risk for cardiovascular disease including coronary artery disease, heart failure with reduced ejection fraction, heart failure with preserved ejection fraction, cerebrovascular disease and peripheral vascular disease. These risk factors include: abdominal fat, high blood sugar (at least 110 milligrams per deciliter (mg/dl)) after fasting; high triglycerides (at least 150 mg/dL) in the bloodstream; low HDL (less than 40 mg/dl); and, blood pressure of 130/85 mmHg or higher (World Health Organization).

The term “cardiovascular diseases” refers to diseases related to the heart or blood vessels.

The term “peripheral arterial disease” refers to when a build-up of fatty deposits in the arteries restricts blood supply to leg muscles.

The term “stroke” refers to when the blood supply to part of the brain is cut off.

The term “heart failure” refers to when the heart has reduced ability to pump blood and can include heart failure with preserved ejection fraction (HFpEF), heart failure with reduced ejection fraction (HFrEF) and heart failure with mid-range ejection fraction (HFmrEF).

The term “coronary heart disease”, also called coronary artery disease, is a narrowing of the arteries that supply blood to the heart.

The term “arrhythmias” refers to abnormal heart rhythm and can include atrial arrhythmias, atrial fibrillation, and ventricular arrhythmias The term “neuropathy” refers to when nerves are damaged. The term includes peripheral neuropathy, which develops when nerves in the extremities such as hands, feet, and arms are damaged. Diabetes is a common cause of peripheral neuropathy.

The term “cardiomyopathies” is defined as acquired or congenital structural abnormalities of the atrial or ventricular myocardium that may affect cardiac function, or physiology, and conduction.

The pharmaceutical composition or combination provided for herein can be in a unit dosage of about 1-1000 mg of active ingredient(s) for a subject of about 50-70 kg. The therapeutically effective dosage of a compound, the pharmaceutical composition, or the combinations thereof, is dependent on the species of the subject, the body weight, age and individual condition, the disorder or disease or the severity thereof being treated.

A compound of the present invention (including, e.g., a compound of any of formula (Ia), (II), (IIa), (III), (IIIa), (IIIb), (IV), (IVa), (IVb), (V), (Va) and (Vb), or a pharmaceutically acceptable salt thereof) may be administered either simultaneously with, or before, or after one or more other therapeutic agent. A compound of the present invention may be administered separately, by the same or different route of administration, or together in the same pharmaceutical composition as the other agents. A therapeutic agent is, for example, a chemical compound, peptide, peptide conjugates and fusions, antibody, antibody fragment, or nucleic acid, which is therapeutically active or enhances the therapeutic activity when administered to a subject in combination with a compound of the present invention.

Thus, in another aspect, provided herein is a combination, in particular a pharmaceutical combination, comprising (e.g., a therapeutically effective amount of) a compound of formula (I), or a pharmaceutically acceptable salt thereof (including, e.g., a compound of any of formula (Ia), (II), (IIa), (III), (IIIa), (IIIb), (IV), (IVa), (IVb), (V), (Va) and (Vb), or a pharmaceutically acceptable salt thereof), and one or more other therapeutically active agents.

In one embodiment, provided herein is a product comprising a compound of formula (I) or a pharmaceutically acceptable salt thereof (including, e.g., a compound of any of formula (Ia), (II), (IIa), (III), (IIIa), (IIIb), (IV), (IVa), (IVb), (V), (Va) and (Vb), or a pharmaceutically acceptable salt thereof), and at least one other therapeutic agent as a combined preparation for simultaneous, separate or sequential use in therapy. In one embodiment, the therapy is the treatment of a disease, disorder or condition selected from the afore-mentioned lists, suitably type 2 diabetes, obesity, atherosclerosis and heart failure (in particular, heart failure with preserved ejection fraction), including where these present as co-morbidities, for example, in a subject with type 2 diabetes who is also obese, or a subject with type 2 diabetes who also has heart failure.

Products provided as a combined preparation include a composition comprising a compound of formula (I) or a pharmaceutically acceptable salt thereof (including, e.g., a compound of any of formula (Ia), (II), (IIa), (III), (IIIa), (IIIb), (IV), (Iva), (Ivb), (V), (Va) and (Vb), or a pharmaceutically acceptable salt thereof), and the other therapeutic agent(s) together in the same pharmaceutical composition, or the compound of the present invention and the other therapeutic agent(s) in separate form, e.g., in the form of a kit.

In one embodiment, the invention provides a pharmaceutical composition comprising a compound of formula (I) or a pharmaceutically acceptable salt thereof (including, e.g., a compound of any of formula (Ia), (II), (Iia), (III), (IIIa), (IIIb), (IV), (Iva), (Ivb), (V), (Va) and (Vb), or a pharmaceutically acceptable salt thereof) and another therapeutic agent(s). Optionally, the pharmaceutical composition may comprise a pharmaceutically acceptable carrier, as described above.

In one embodiment, provided herein is a kit comprising two or more separate pharmaceutical compositions, at least one of which contains a compound of the present invention. In one embodiment, the kit comprises means for separately retaining said compositions, such as a container, divided bottle, or divided foil packet. An example of such a kit is a blister pack, as typically used for the packaging of tablets, capsules and the like.

The kit may be used for administering different dosage forms, for example, oral and parenteral, for administering the separate compositions at different dosage intervals, or for titrating the separate compositions against one another. To assist compliance, the kit provided for herein typically comprises directions for administration.

In the combination therapies provided herein, the compound of formula (I) or a pharmaceutically acceptable salt thereof (including, e.g., a compound of any of formula (Ia), (II), (Iia), (III), (IIIa), (IIIb), (IV), (Iva), (Ivb), (V), (Va) and (Vb), or a pharmaceutically acceptable salt thereof), and the other therapeutic agent may be manufactured and/or formulated by the same or different manufacturers. Moreover, the compound of formula (I) or a pharmaceutically acceptable salt thereof, and the other therapeutic may be brought together into a combination therapy: (i) prior to release of the combination product to physicians (e.g. in the case of a kit comprising the compound of compound of formula (I) or a pharmaceutically acceptable salt thereof, and the other therapeutic agent); (ii) by the physician themselves (or under the guidance of the physician) shortly before administration; (iii) in the patient themselves, e.g. during sequential administration of the compound of formula (I), or a pharmaceutically acceptable salt thereof, and the other therapeutic agent.

Accordingly, the provided herein is a use of a compound of formula (I) or a pharmaceutically acceptable salt thereof (including, e.g., a compound of any of formula (Ia), (II), (Iia), (III), (IIIa), (IIIb), (IV), (Iva), (Ivb), (V), (Va) and (Vb), or a pharmaceutically acceptable salt thereof) in the preparation of medicament for treating a disease, disorder, or condition selected from the afore-mentioned lists, suitably type 2 diabetes, obesity, atherosclerosis, and heart failure (in particular, heart failure with preserved ejection fraction), including where these present as co-morbidities, for example, in a subject with type 2 diabetes who is also obese, or a subject with type 2 diabetes who also has heart failure, wherein the medicament is prepared for administration with another therapeutic agent.

Provided herein is the use of another therapeutic agent for treating a disease, disorder or condition selected from the afore-mentioned lists, suitably type 2 diabetes, obesity, atherosclerosis and heart failure (in particular, heart failure with preserved ejection fraction), including where these present as co-morbidities, for example, in a subject with type 2 diabetes who is also obese, or a subject with type 2 diabetes who also has heart failure, wherein the medicament is administered with a compound of the present invention.

Provided herein is a compound of formula (I) or a pharmaceutically acceptable salt thereof (including, e.g., a compound of any of formula (Ia), (II), (IIa), (III), (IIIa), (IIIb), (IV), (IVa), (IVb), (V), (Va) and (Vb), or a pharmaceutically acceptable salt thereof), for use in a method of treating a disease, disorder or condition selected from the afore-mentioned lists, suitably type 2 diabetes, obesity, atherosclerosis, and heart failure (in particular, heart failure with preserved ejection fraction), including where these present as co-morbidities, for example, in a subject with type 2 diabetes who is also obese, or a subject with type 2 diabetes who also has heart failure, wherein the compound of formula (I) or a pharmaceutically acceptable slat thereof, is prepared for administration with another therapeutic agent.

Also provided herein is another therapeutic agent for use in a method of treating a disease, disorder or condition selected from the afore-mentioned lists, suitably type 2 diabetes, obesity, atherosclerosis and heart failure (in particular, heart failure with preserved ejection fraction), including where these present as co-morbidities, for example, in a subject with type 2 diabetes who is also obese, or a subject with type 2 diabetes who also has heart failure, wherein the other therapeutic agent is prepared for administration with a compound of formula (I) or a pharmaceutically acceptable salt thereof (including, e.g., a compound of any of formula (Ia), (II), (IIa), (III), (IIIa), (IIIb), (IV), (IVa), (IVb), (V), (Va) and (Vb), or a pharmaceutically acceptable salt thereof).

Also provided herein is a compound of formula (I) or a pharmaceutically acceptable salt thereof (including, e.g., a compound of any of formula (Ia), (II), (IIa), (III), (IIIa), (IIIb), (IV), (IVa), (IVb), (V), (Va) and (Vb), or a pharmaceutically acceptable salt thereof), for use in a method of treating a disease, disorder or condition selected from the afore-mentioned lists, suitably type 2 diabetes, obesity, atherosclerosis and heart failure (in particular, heart failure with preserved ejection fraction), including where these present as co-morbidities, for example, in a subject with type 2 diabetes who is also obese, or a subject with type 2 diabetes who also has heart failure, wherein the compound of formula (I) or a pharmaceutically acceptable salt thereof, is administered with another therapeutic agent.

Also provided is another therapeutic agent for use in a method of treating a disease, disorder or condition selected from the afore-mentioned lists, suitably type 2 diabetes, obesity, atherosclerosis, and heart failure (in particular, heart failure with preserved ejection fraction), including where these present as co-morbidities, for example, in a subject with type 2 diabetes who is also obese, or a subject with type 2 diabetes who also has heart failure, wherein the other therapeutic agent is administered with a compound of formula (I), or a pharmaceutically acceptable salt thereof.

Also provided herein is a use of a compound of formula (I) or a pharmaceutically acceptable salt thereof (including, e.g., a compound of any of formula (Ia), (II), (IIa), (III), (IIIa), (IIIb), (IV), (IVa), (IVb), (V), (Va) and (Vb), or a pharmaceutically acceptable salt thereof), for treating a disease, disorder or condition selected from the afore-mentioned lists, suitably type 2 diabetes, obesity, atherosclerosis, and heart failure (in particular, heart failure with preserved ejection fraction), including where these present as co-morbidities, for example, in a subject with type 2 diabetes who is also obese, or a subject with type 2 diabetes who also has heart failure, wherein the patient has previously (e.g. within 24 hours) been treated with another therapeutic agent.

Also provided herein is a use of another therapeutic agent for treating a disease, disorder or condition selected from the afore-mentioned lists, suitably type 2 diabetes, obesity, atherosclerosis, and heart failure (in particular, heart failure with preserved ejection fraction), including where these present as co-morbidities, for example, in a subject with type 2 diabetes who is also obese, or a subject with type 2 diabetes who also has heart failure, wherein the patient has previously (e.g. within 24 hours) been treated with a compound of formula (I) or a pharmaceutically acceptable salt thereof (including, e.g., a compound of any of formula (Ia), (II), (IIa), (III), (IIIa), (IIIb), (IV), (IVa), (IVb), (V), (Va) and (Vb), or a pharmaceutically acceptable salt thereof).

In one embodiment, the other therapeutic agent is selected from:
1. Antidiabetic agents, such as insulin, insulin derivatives and mimetics; insulin secretagogues such as the sulfonylureas (e.g., chlorpropamide, tolazamide, acetohexamide, tolbutamide, glyburide, glimepiride, glipizide); glyburide and Amaryl; insulinotropic sulfonylurea receptor ligands such as meglitinides, e.g., nateglinide and repaglinide; thiazolidinediones (e.g., rosiglitazone (AVANDIA), troglitazone (REZULIN), pioglitazone (ACTOS), balaglitazone, rivoglitazone, netoglitazone, troglitazone, englitazone, ciglitazone, adaglitazone, darglitazone that enhance insulin action (e.g., by insulin sensitization), thus promoting glucose utilization in peripheral tissues; protein tyrosine phosphatase-1B (PTP-1B) inhibitors such as PTP-112; Cholesteryl ester transfer protein (CETP) inhibitors such as torcetrapib, GSK3 (glycogen synthase kinase-3) inhibitors such as SB-517955, SB-4195052, SB-216763, NN-57-05441 and NN-57-05445; RXR ligands such as GW-0791 and AGN-194204; sodium-dependent glucose cotransporter inhibitors such as canagliflozin, dapagliflozin, empagliflozin, ertugliflozin, ipragliflozin, luseogliflozin, remogliflozin etabonate, sotagliflozin, tofogliflozin; glycogen phosphorylase A inhibitors such as BAY R3401; biguanides such as metformin and other agents that act by promoting glucose utilization, reducing hepatic glucose production and/or diminishing intestinal glucose output; alpha-glucosidase inhibitors such as acarbose and migiitoi) and other agents that slow down carbohydrate digestion and consequently absorption from the gut and reduce postprandial hyperglycemia; GIPR modulators such as trizepatide, CT-868, CT-388, AMG133, HM15211, NN9423, TAK-094, LBT-6030, ZP-1-98, NN9709, RG7685, RG7697, SAR438335; and DPPIV (dipeptidyl peptidase IV) inhibitors such as vildagliptin;
2. Hypolipidemic agents such as 3-hydroxy-3-methyl-glutaryl coenzyme A (HMG-CoA) reductase inhibitors, e.g., lovastatin, pitavastatin, simvastatin, pravastatin, cerivastatin, mevastatin, velostatin, fluvastatin, dalvastatin, atorvastatin, rosuvastatin and rivastatin; squalene synthase inhibitors; FXR (farnesoid X receptor) and LXR (liver X receptor) ligands; bile acid sequenstrants, such as cholestyramine and colesevelam; fibrates; nicotinic acid and aspirin;
3. Anti-obesity agents such as orlistat, rimonabant, phentermine, topiramate, qunexa, and locaserin; GDF15 (such as variants, conjugates, fusions, analogs, mutants and fragments thereof, including other GFRAL modulators, such as NGM386, NGM395); the molecules described in PCT Publications WO2013/148117, WO2014/120619 and all related patent family members (including but not limited to U.S. Pat. No. 9,161,966B1), WO2012/138919, WO2013/113008, WO2015/017710, WO2015/200078, WO2015/197446, WO2015/198199 and WO2017/109706, in particular GDF15 conjugates with fatty acids (such as the conjugates described in PCT Publications WO2015/200078 and WO2017/109706) and GDF15 fusions (including for example GDF15 fusions with human serum albumin (HSA)) such as the fusions described in PCT Publications WO2015/197446, WO2015/198199 and WO2017/109706; FGF21 mimetics such as PEG-FGF21, Pegbelfermin; ActRII anatgonists such as ramatercept; and Alk7 antagonists;
4. Anti-hypertensive agents, e.g., loop diuretics such as ethacrynic acid, furosemide and torsemide; angiotensin converting enzyme (ACE) inhibitors such as benazepril, captopril, enalapril, fosinopril, lisinopril, moexipril, perinodopril, quinapril, ramipril and trandolapril; inhibitors of the Na—K-ATPase membrane pump such as digoxin; neutralendopeptidase (NEP) inhibitors; ACE/NEP inhibitors such as omapatrilat, sampatrilat and fasidotril; angiotensin II antagonists such as candesartan, eprosartan, irbesartan, losartan, telmisartan and valsartan, in particular valsartan; angiotensin receptor-neprilysin inhibitors (ARNi) such as sacubitril/valsartan (Entresto); renin inhibitors such as ditekiren, zankiren, terlakiren, aliskiren, RO 66-1132 and RO-66-1168; β-adrenergic receptor blockers such as acebutolol, atenolol, betaxolol, bisoprolol, metoprolol, nadolol, propranolol, sotalol and timolol; inotropic agents such as digoxin, dobutamine and milrinone; calcium channel blockers such as amlodipine, bepridil, diltiazem, felodipine, nicardipine, nimodipine, nifedipine, nisoldipine and verapamil; aldosterone receptor antagonists; and aldosterone synthase inhibitors;
5. Agonists of peroxisome proliferator-activator receptors, such as fenofibrate, pioglitazone, rosiglitazone, tesaglitazar, BMS-298585, L-796449, the compounds specifically described in the patent application WO 2004/103995 i.e. compounds of examples 1 to 35 or compounds specifically listed in claim 21, or the compounds specifically described in the patent application WO 03/043985 i.e. compounds of examples 1 to 7 or compounds specifically listed in claim 19 and especially (R)-1-{4-[5-methyl-2-(4-trifluoromethyl-phenyl)-oxazol-4-ylmethoxy]-benzenesulfonyl}-2,3-dihydro-1H-indole-2-carboxylic or a salt thereof;
6. The specific anti-diabetic compounds described in Expert Opin Investig Drugs 2003, 12(4): 623-633, FIGS. 1 to 7.
7. Compounds that bind the corticotropin-releasing hormone receptors, such as Urocortin 2.

Furthermore, the present invention provides combination therapy with agents and methods for promoting weight loss, such as agents that stimulate metabolism or decrease appetite, and modified diets and/or exercise regimens to promote weight loss.

### EXAMPLES

The following examples are intended to illustrate the invention and are not to be construed as being limitations thereon. Temperatures are given in degrees Celsius. If not mentioned otherwise, all evaporations are performed under reduced pressure, typically between about 15 mm Hg and 100 mm Hg (=20-133 mbar). The structure of final products, intermediates and starting materials is confirmed by standard analytical methods, e.g., microanalysis and spectroscopic characteristics, e.g., MS, IR, NMR. Abbreviations used are those conventional in the art.

All starting materials, building blocks, reagents, acids, bases, dehydrating agents, solvents, and catalysts utilized to synthesis the compounds of the present invention are either commercially available or can be produced by organic synthesis methods known to one of ordinary skill in the art. Further, the compounds of the present invention can be produced by organic synthesis methods known to one of ordinary skill in the art as shown in the following examples.

### Abbreviations Used in the Following Examples and Elsewhere Herein are

A₀ plateau value of Hill curve at low concentrations
AC₅₀ concentration at half-maximal compound effect
A_{inf} plateau value of Hill curve at high concentrations
ACN acetonitrile
BSA bovine serum albumin
BOC tertiary butyl carboxy
br broad
BSA bovine serum albumin
cAMP cyclic adenosine monophosphate
CDI carbonyldiimidazole
CO₂ carbon dioxide
d doublet
dd doublet of doublets
DBU diazabicycloundecene
DCM dichloromethane
DIEA/DIPEA diethylisopropylamine
DMA dimethylacetamide
DMEM Dulbecco's Modified Eagle Media
DMF N,N-dimethylformamide
DMI dimethylimidazolidinone
DMPU N,N-dimethylpropyleneurea
DMSO dimethylsulfoxide
EC effective concentration
EC₀ effective concentration of a compound that gives no response
EC₅₀ effective concentration of a compound that gives a half maximal response (AC₅₀ in μM)
EC₁₀₀ effective concentration of a compound that gives a maximal (100%) response
EDTA ethylenediaminetetraacetic acid
Eₘₐₓ efficacy: maximum response achievable from a dosed agent
ESI electrospray ionization
EtOAc ethyl acetate
ETOH ethanol
FA formic acid
FBS fetal bovine serum
G418 geneticin, a selection antibiotic
GLP1 glucagon-like peptide 1
GLP1R glucagon-like peptide 1 receptor
GPCR G-protein coupled receptor
h hour(s)
HATU (1-[bis(dimethylamino)methylene]-1H-1,2,3-triazolo[4,5-b]pyridinium 3-oxid hexafluorophosphate)
hGLP1R human glucagon-like peptide 1 receptor
HPLC high pressure liquid chromatography
HRMS high resolution mass spectrometry
HTRF homogenous time resolved fluorescence
IBMX 3-isobutyl-1-methylxanthine
KHMDS potassium bis(trimethylsilyl)amide
LCMS liquid chromatography and mass spectrometry
LDA lithium diisopropylamide
LiOH lithium hydroxide
MeCN acetonitrile
MeOH methanol
MS mass spectrometry
m multiplet
mg milligram
min minutes
mL milliliter
mM millimolar
mmol millimol
nM nanomolar
m/z mass to charge ratio
NMR nuclear magnetic resonance
ND not determined
PBS phosphate-buffered saline
Pd(OAc)₂ palladium (II) acetate
PdCl₂(dppf)-CH₂Cl₂ 1,1′-bis(diphenylphosphino)ferrocene-palladium(II)dichloride dichloromethane complex
Pd(OAc)₂ palladium(II) acetate
ppm parts per million
p-TsOH p-toluenesulfonic acid
PyBOP benzotriazol-1-yloxytripyrrolidinophosphonium hexafluorophosphate
PyBroP bromotripyrrolidinophosphonium hexafluorophosphate
rac racemic
Rₜ retention time
rt room temperature
s singlet
SFC superfluid carbon dioxide
SM starting material/starting materials
SEM-Cl 2-(trimethylsilyl)ethoxymethyl chloride
t triplet
TEA triethylamine
TFA trifluoroacetic acid
THF tetrahydrofuran
TMS trimethylsilyl
TMSCl trimethylsilyl chloride
Tol toluene
Turbo Grignard isopropylmagnesium chloride lithium chloride complex solution
v/v volume/volume
μL microliter
μM micromolar

### General Conditions:

### NMR

Unless otherwise noted, reagents and solvents were used as received from commercial suppliers. Proton nuclear magnetic resonance (¹H NMR) spectra were recorded on (a) Bruker AVANCE 400 MHz or 500 MHz NMR spectrometers using ICON-NMR, under TopSpin program control; (b) Bruker ASCEND 400 MHz NMR spectrometers using Console-Avance III 400, under TopSpin 3.2 program control; or (c) Bruker ASCEND 400 MHz NMR spectrometers using Console-Avance III HD, under TopSpin 3.2 program control. Spectra were measured at 293-298K, unless indicated otherwise, and were referenced relative to the solvent resonance. Spectra are given in ppm (δ) and coupling constants, J, are reported in Hertz. Tetramethylsilane (TMS) was used as an internal standard.

### NMR-01:

Unless otherwise noted, reagents and solvents were used as received from commercial suppliers. Proton nuclear magnetic resonance (1H NMR) spectra were recorded on Bruker ASCEND 400 MHz NMR spectrometers using Console-Avance III 400, under TopSpin 3.2 program control. Spectra were measured at 293K-298K, unless indicated otherwise, and were referenced relative to the solvent resonance. Spectra are given in ppm (δ) and coupling constants, J, are reported in Hertz. Tetramethylsilane (TMS) was used as an internal standard.

### NMR-02:

Unless otherwise noted, reagents and solvents were used as received from commercial suppliers. Proton nuclear magnetic resonance (1H NMR) spectra were recorded on Bruker ASCEND 400 MHz NMR spectrometers using Console-Avance III HD, under TopSpin 3.2 program control. Spectra were measured at 293K-298K, unless indicated otherwise, and were referenced relative to the solvent resonance. Spectra are given in ppm (δ) and coupling constants, J, are reported in Hertz. Tetramethylsilane (TMS) was used as an internal standard.

### LCMS Instrumentation

Mass spectra were acquired using Agilent 1100 HPLC systems with an Agilent 6110 Mass Spectrometer. [M+H]⁺ refers to protonated molecular ion of the chemical species. Column temperature was 50° C. Flow was 1.0 mL/min.

### LCMS Method 1 (Acidic):

Instrument: Waters Acquity UPLC, photodiode array detector; Column: AcQuity UPLC BEH C₁₈ 1.7 μm, 21×30 mm; 2 min run time, 2% solvent B from 0 to 0.1 min, 2→98% solvent B: solvent A from 0.1 to 1.8 min, 98% solvent B for 0.2 min. Solvents: Solvent A=0.1% formic acid in water (v/v), solvent B=0.1% formic acid in acetonitrile (v/v). Injection volume 2-5 uL; UV detection array 210-400, Mass detection 120-1250 (electrospray ionization); column at 50° C.; flow rate 1.0 mL/min.

### LCMS Method 2 (Basic):

Instrument: Waters Acquity UPLC, photodiode array detector; Column: AcQuity UPLC BEH C₁₈ 1.7 μm 21×50 mm; 2 min run time, 2% solvent B from 0 to 0.1 min, 2→98% solvent B: solvent A from 0.1 to 1.8 min, 98% solvent B for 0.2 min. Solvents: Solvent A=5 mM ammonium hydroxide in water, solvent B=5 mM ammonium hydroxide in acetonitrile. Injection volume 2-5 uL; UV detection array 210-400, Mass detection 120-1250 (electrospray ionization); column at 50° C.; flow rate 1.0 mL/min.

### LCMS Method 3 (Product Analysis-Acidic):

Instrument: Waters Acquity UPLC, photodiode array detector; Column AcQuity UPLC BEH C₁₈ 1.7 μm 21×30 mm; 5.2 min run time, 2→98% solvent B:solvent A from 0 to 5.15 min, 98% solvent B from 5.15 to 5.20 min. Solvents: Solvent A=0.1% formic acid in water (v/v), solvent B=0.1% formic acid in acetonitrile (v/v). Injection volume 2-5 uL; UV detection array 210-400, Mass detection 120-1600; column at 50° C., flow rate 1.0 mL/min.

### LCMS Method 4 (Product Analysis-Basic):

Instrument: Waters Acquity UPLC, photodiode array detector; Column AcQuity UPLC BEH C₁₈ 1.7 μm 21×30 mm; 5.2 min run time, 2→98% solvent B:solvent A from 0 to 5.15 min, 98% solvent B from 5.15 to 5.20 min. Solvents: Solvent A=5 mM ammonium hydroxide in water, solvent B=5 mM ammonium hydroxide in acetonitrile). Injection volume 2-5 uL; UV detection array 210-400, Mass detection 120-1600; column at 50° C., flow rate 1.0 mL/min.

### HRMS Method 5:

Instrument: Agilent 1200 LC/G1956A, diode array detector; Column: Chromolith Flash C₁₈, 1.6 micron 2×25 mm; 1.5 minute run time, 5→95% solvent B:solvent A from 0→1.2 minutes and then 95% solvent B from 1.21→1.5 minutes. Solvents: Solvent A=0.0375% TFA in Water (v/v), Solvent B=0.01875% TFA in acetonitrile (v/v). Injection volume 2-5 uL; UV detection 220 and 254 nM, Mass detection 100-1000 (electrospray ionization); column at 50° C.; flow rate 1.5 mL/min.

### Chiral Preparation HPLC Method:

Instrument: Shimadzu LC-20AP and UV detector.

Column: CHIRALPAK IG, (250×21.0) mm, 5 micron,

Column flow: 16.0 mL/min.

Mobile phase: (A) 0.1% DEA in methanol.

The UV spectra were recorded at 276.0 nm Lambdamax.

Isocratic ratio was:

| Time (min) | % A |
|---|---|
| 0.01 | 100 |
| 18.00 | 100 |

### Example 1: Synthesis of Intermediates

### Example 1-1: Synthesis of (S)-3-fluoro-4-(2-methyl-4-(piperidin-4-yl)benzo[d][1,3]dioxol-2-yl)benzonitrile 4-methylbenzenesulfonate (II.B1)

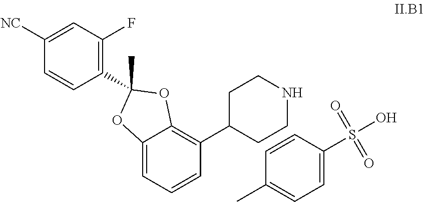

### Step 1: Synthesis of tert-butyl (S)-4-(2-(4-cyano-2-fluorophenyl)-2-methylbenzo[d][1,3]dioxol-4-yl)piperidine-1-carboxylate (II.1b)

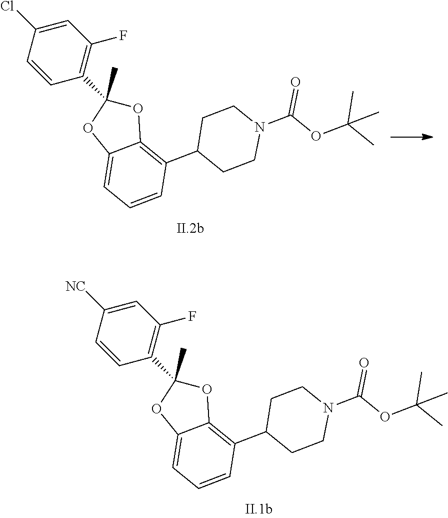 [SMILES: CC(C)(C)OC(=O)N1CCC(c2cccc3c2O[C@@](C)(c2ccc(Cl)cc2F)O3)CC1 ; CC(C)(C)OC(=O)N1CCC(c2cccc3c2O[C@@](C)(c2ccc(C#N)cc2F)O3)CC1]

Intermediate II.2b (0.500 g, 1.12 mmol), zinc cyanide (144 mg, 77.9 μL, 1.23 mmol), and (2-dicyclohexylphosphino-2′,6′-dimethoxybiphenyl)[2-(2′-amino-1,1′-biphenyl)]palladium(II) methanesulfonate (48.9 mg, 55.8 μmol) were dissolved in dry N,N-dimethylformamide (5 mL) under nitrogen. Water (0.1 mL) was added, and the mixture was degassed by bubbling nitrogen. The reaction was heated in the microwave to 150° C. for 25 minutes. By LCMS analysis it was found near complete conversion to the desired mass. The vial was opened and poured into a mixture of 1 N sodium hydroxide (30 mL) and ethyl acetate (75 mL). The mixture was strongly stirred for 10 minutes and then the phases was separated. The organic phase was dried with sodium sulfate and evaporated to dryness under reduced pressure. Purification using the ISCO (5-60% ethyl acetate in heptane gradient) gave the desired II.1b (445 mg, 1.02 mmol, 91% yield) as a white solid. ¹H NMR (400 MHz, CDCl₃) δ ppm 1.11-1.20 (m, 1H) 1.30-1.38 (m, 9H) 1.45-1.73 (m, 5H) 1.89-1.95 (m, 3H) 2.60-2.72 (m, 3H) 4.03-4.15 (m, 2H) 6.49-6.60 (m, 2H) 6.60-6.69 (m, 1H) 7.22-7.33 (m, 2H) 7.53-7.59 (m, 1H).

### Step 2: Synthesis of (S)-3-fluoro-4-(2-methyl-4-(piperidin-4-yl)benzo[d][1,3]dioxol-2-yl)benzonitrile 4-methylbenzenesulfonate (II.B1)

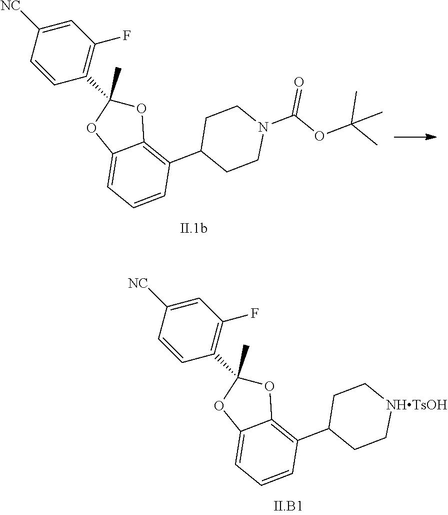

To a solution of II.1b (445 mg, 1.02 mmol) in EtOAc (7 mL) was added Ts-OH·H₂O (231.6 mg, 1.218 mmol) at 25° C. The mixture was heated to 45° C. for 16 h, when LCMS analysis showed full consumption of starting materials and formation of desired product. The crude product was evaporated to dryness under reduced pressure to obtain the tosylic acid salt of II.B1 (518 mg, 1.02 mmol, 100% yield) as a white solid. LCMS Method 1 (acidic): Rt=0.75 min, MS (ESI+) m/z 339.1 (M+1)⁺.

### Example 1-2: Synthesis of (S)-4-(2-(4-chloro-2-fluorophenyl)-2-methylbenzo[d][1,3]dioxol-4-yl)piperidine hydrochloride (II.A1) and (S)-4-(2-(4-chloro-2-fluorophenyl)-2-methylbenzo[d][1,3]dioxol-4-yl)piperidine 4-methylbenzenesulfonate (II.B2)

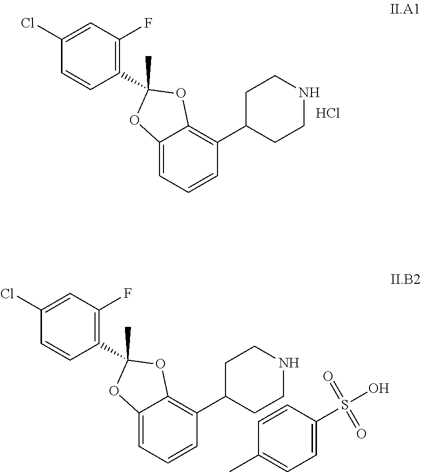

### Step 1: Synthesis of 4-bromo-2-(4-chloro-2-fluorophenyl)-2-methylbenzo[d][1,3]dioxole (II.2a)

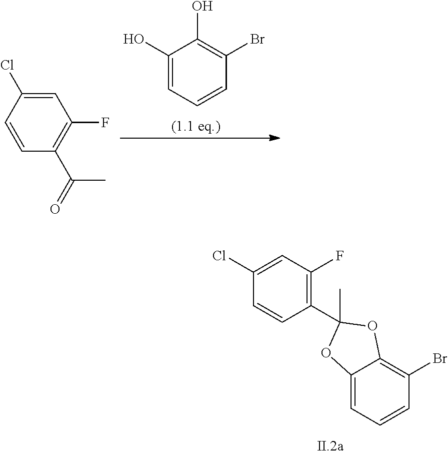 [SMILES: CC(=O)c1ccc(Cl)cc1F ; Oc1cccc(Br)c1O ; CC1(c2ccc(Cl)cc2F)Oc2cccc(Br)c2O1]

To a solution of 1-(4-chloro-2-fluorophenyl)ethan-1-one (150 g, 793.61 mmol) in toluene (1500 mL) was added 3-bromobenzene-1,2-diol (123.27 g, 714.25 mmol) and p-TsOH (27.33 g, 158.72 mmol) at 25° C. The mixture was stirred at 140° C. for 48 h. TLC (petroleum ether: ethyl acetate=30:1) showed 1-(4-chloro-2-fluorophenyl)ethan-1-one was consumed completely and a new spot (Rf=0.4) was detected. The mixture was concentrated to give the crude product. The crude product was purified by silica gel column chromatography (SiO₂, petroleum ether) to get II.2a (175 g, crude) as a black oil. It was used for next step without further purification. ¹H-NMR (400 MHz, CDCl₃) δ 2.04 (s, 3H), 7.47 (t, J=8.38 Hz, 1H), 7.05-7.09 (m, 2H), 6.88 (dd, J=7.88, 1.13 Hz, 1H), 6.57-6.71 (m, 2H), 2.04 (s, 3H).

### Step 2: Synthesis of tert-butyl 4-(2-(4-chloro-2-fluorophenyl)-2-methylbenzo[d][1,3]dioxol-4-yl)-3,6-dihydropyridine-1(2H)-carboxylate (II.3a)

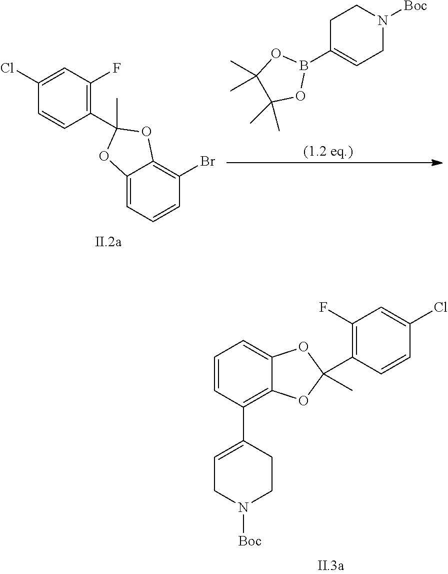 [SMILES: CC1(c2ccc(Cl)cc2F)Oc2cccc(Br)c2O1 ; CC(C)(C)OC(=O)N1CC=C(B2OC(C)(C)C(C)(C)O2)CC1 ; CC(C)(C)OC(=O)N1CC=C(c2cccc3c2OC(C)(c2ccc(Cl)cc2F)O3)CC1]

To a solution of II.2a (150 g, 436.58 mmol) in dioxane (1500 mL) was added tert-butyl 4-(4,4,5,5-tetramethyl-1,3,2-dioxaborolan-2-yl)-3,6-dihydropyridine-1(2H)-carboxylate (162.00 g, 523.90 mmol), Na₂CO₃ (254.50 g, 2401.19 mmol) and Pd(dppf)Cl₂ (31.99 g, 43.66 mmol) at 25° C. The mixture was stirred at 90° C. for 16 h. The reaction mixture was filtered and concentrated to give the crude product. The crude product was purified by silica gel column chromatography (SiO₂, petroleum ether: ethyl acetate=10:1) to get II.3a (50 g, 112.11 mmol, 96% purity, 44% yield) as a yellow oil. ¹H NMR (400 MHz, CDCl₃) δ 7.52 (t, J=8.3 Hz, 1H), 7.19-7.10 (m, 2H), 6.85-6.73 (m, 3H), 6.37 (br s, 1H), 4.22-4.03 (m, 2H), 3.79-3.55 (m, 2H), 2.56 (br d, J=14.3 Hz, 2H), 2.08 (d, J=0.6 Hz, 3H), 1.51 (s, 9H).

### Step 3: Synthesis of tert-butyl 4-(2-(4-chloro-2-fluorophenyl)-2-methylbenzo[d][1,3]dioxol-4-yl)piperidine-1-carboxylate (II.4a)

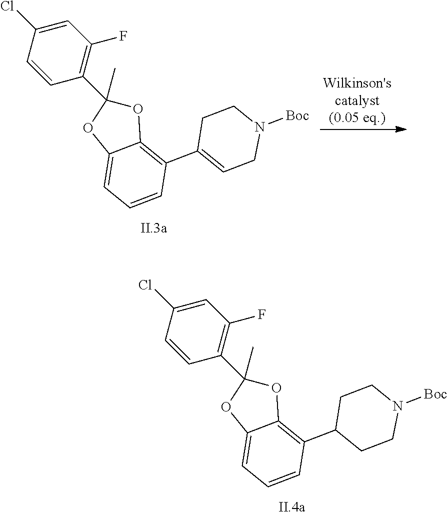 [SMILES: CC(C)(C)OC(=O)N1CC=C(c2cccc3c2OC(C)(c2ccc(Cl)cc2F)O3)CC1 ; CC(C)(C)OC(=O)N1CCC(c2cccc3c2OC(C)(c2ccc(Cl)cc2F)O3)CC1]

To a solution of II.3a (50 g, 112.13 mmol) in MeOH (200 mL) was added ((C₆H₅)₃P)₃RhCl (5.19 g, 5.61 mmol) at 25° C. The mixture was stirred at 50° C. under H₂ for 16 h. The reaction mixture was filtered and concentrated to give the crude product. The crude product was purified by silica gel column chromatography (SiO₂, PE:EA=10:1) to get II.4a (37.5 g, 83.71 mmol, 98% purity, 75% yield) as a yellow oil. ¹H NMR (400 MHz, CDCl₃) δ 7.52 (t, J=8.3 Hz, 1H), 7.19-7.08 (m, 2H), 6.81-6.75 (m, 1H), 6.74-6.70 (m, 1H), 6.67 (dd, J=0.9, 7.8 Hz, 1H), 4.25 (br s, 2H), 2.83 (tt, J=3.7, 11.9 Hz, 3H), 2.06 (d, J=0.7 Hz, 3H), 1.86-1.64 (m, 4H), 1.50 (s, 9H).

### Step 4: SFC Separation of II.6a and II.6b

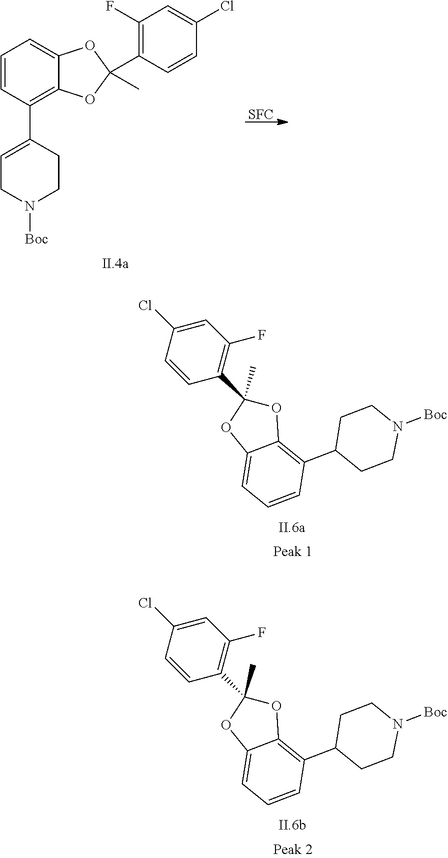 [SMILES: CC(C)(C)OC(=O)N1CC=C(c2cccc3c2OC(C)(c2ccc(Cl)cc2F)O3)CC1 ; "SFC" ; CC(C)(C)OC(=O)N1CCC(c2cccc3c2O[C@](C)(c2ccc(Cl)cc2F)O3)CC1 ; CC(C)(C)OC(=O)N1CCC(c2cccc3c2O[C@@](C)(c2ccc(Cl)cc2F)O3)CC1]

II.4a (170 g, 379.52 mmol, 96% purity) was purified by SFC (CAS-WH-ANA-SFC-A(Agilent-1260), Column: Chiralpak AD-3 50×4.6 mm I.D., 3 um; Mobile phase: Phase A for CO₂, and Phase B for IPA (0.05% DEA); Gradient elution: B in A from 5% to 40%; Flow rate: 3 mL/min; Detector: DAD; Column Temp: 35° C.; Back Pressure: 100 Bar) to get II.6a (77 g, 171.90 mmol, 100% e.e.) and II.6b (75 g, 167.44 mmol, 100% e.e.). SFC: Rt=0.884 min, II.6a Peak 1. SFC: Rt=1.118 min, II.6b Peak 2.

### Step 5a: Synthesis of (S)-4-(2-(4-chloro-2-fluorophenyl)-2-methylbenzo[d][1,3]dioxol-4-yl)piperidine 4-methylbenzenesulfonate (II.B2)

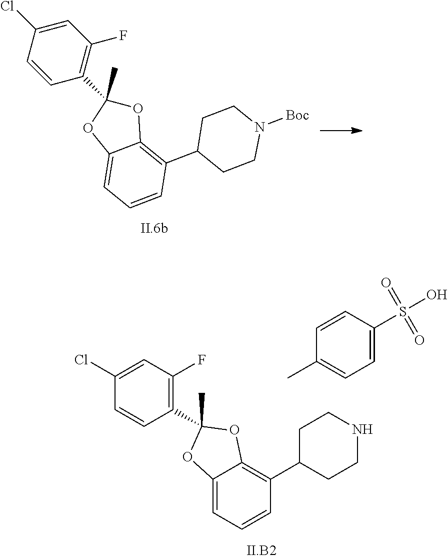

To a solution of compound II.6b (19.4 g, 43.3 mmol) in EtOAc (200 mL) was added TsOH·H₂O (9.88 g, 51.9 mmol, 1.20 eq). After addition, the light yellow suspension was stirred at 45° C. for 12 h. The reaction mixture was poured into petroleum ether (400 mL), filtered, collected and dried. II.B2 was obtained as white solid (20.0 g, 36.9 mmol, 85.2% yield, 96.0% purity). ¹H NMR (400 MHz, DMSO-d₆) δ) 8.58 (br d, J=8.63 Hz, 1H), 8.31 (br d, J=8.25 Hz, 1H), 7.49 (d, J=8.00 Hz, 2H), 7.56-7.65 (m, 2H), 7.34 (dd, J=8.38, 1.75 Hz, 1H), 7.12 (d, J=7.88 Hz, 2H), 6.82-6.87 (m, 2H), 6.68-6.74 (m, 1H), 3.37 (br d, J=12.51 Hz, 2H), 2.94-3.07 (m, 3H), 2.03 (s, 3H), 2.29 (s, 3H), 1.84-1.94 (m, 4H).

### Step 5b: Synthesis of (S)-4-(2-(4-chloro-2-fluorophenyl)-2-methylbenzo[d][1,3]dioxol-4-yl)piperidine hydrochloride (II.A1)

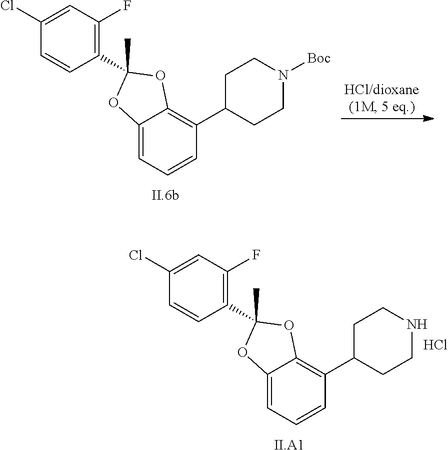

To a solution of II.6b (67 g, 149.58 mmol) in dioxane (670 mL) was added HCl (670 mL, 1M in dioxane) at 25° C. The mixture was stirred at 25° C. for 16 h. The mixture was concentrated to give the crude product. The crude product was triturated with MTBE:MeOH=10:1 (700 mL) to get II.A1 (71 g, 204.13 mmol, 98% purity, 99.27% e.e. quant) as a white solid. ¹H NMR (400 MHz, CDCl₃) δ 9.98-9.43 (m, 2H), 7.57 (t, J=8.3 Hz, 1H), 7.19-7.09 (m, 2H), 6.87-6.65 (m, 3H), 3.96 (s, 1H), 3.64 (br d, J=10.9 Hz, 2H), 3.50 (s, 2H), 3.22 (s, 1H), 3.12-2.90 (m, 3H), 2.39-2.19 (m, 2H), 2.15-1.98 (m, 5H), 1.20 (s, 1H).

### Example 1-3: Synthesis of 1-((4-bromo-5-methyl-1-(((S)-oxetan-2-yl)methyl)-1H-imidazol-2-yl)methyl)-4-((S)-2-(4-chloro-2-fluorophenyl)-2-methylbenzo[d][1,3]dioxol-4-yl)piperidine (I.A1)

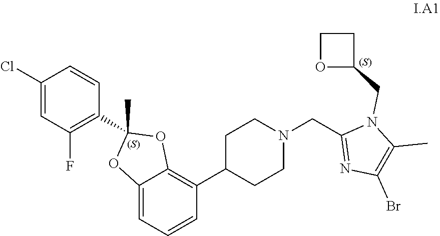

### Step 1: Synthesis of (S)-4-bromo-5-methyl-1-(oxetan-2-ylmethyl)-1H-imidazole (2a) and (S)-5-bromo-4-methyl-1-(oxetan-2-ylmethyl)-1H-imidazole (III.2b)

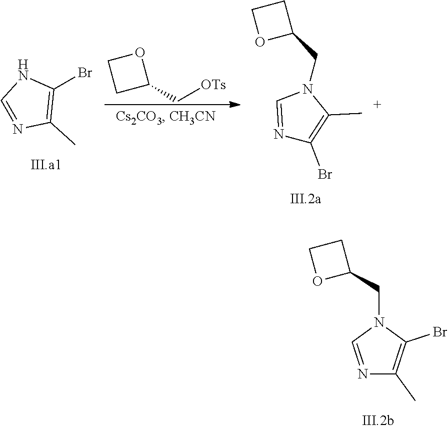

To a solution of 4-bromo-5-methyl-1H-imidazole (III.a1) (2.000 g, 12.4 mmol) in acetonitrile (50 mL) at room temperature was added (S)-oxetan-2-ylmethyl 4-methylbenzenesulfonate (3.33 g, 13.0 mmol) and cesium carbonate (10.1 g, 31.1 mmol). The mixture was stirred at 80° C. for 16 hours using a findenser, and then cooled down to room temperature. Water was added and the mixture was extracted with ethyl acetate twice, washed with brine, dried over magnesium sulfate, filtered and concentrated. The residue was purified via chromatography (0-100% EtOAc/heptane, 0-5% MeOH/EtOAc) to separate the isomers to afford (S)-4-bromo-5-methyl-1-(oxetan-2-ylmethyl)-1H-imidazole (III.2a) (1.2 g, 42%) (LCMS Method 2 (Basic): Rt=0.67 min, MS (ESI+) m/z 233.0 [M+1]⁺), and (S)-5-bromo-4-methyl-1-(oxetan-2-ylmethyl)-1H-imidazole (III.2b) (0.35 g, 12%) (LCMS Method 2 (Basic): Rt=0.68 min, MS (ESI+) m/z 231.3 (M+1)⁺).

### Step 2: Synthesis of (S)-4-bromo-5-methyl-1-(oxetan-2-ylmethyl)-1H-imidazole-2-carbaldehyde (III.3)

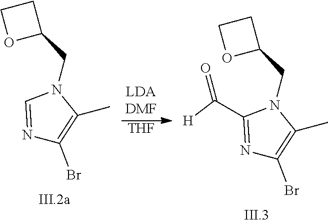

To a solution of (S)-4-bromo-5-methyl-1-(oxetan-2-ylmethyl)-1H-imidazole (III.2a) (580 mg, 2.51 mmol) in THF (12 mL) at −78° C., LDA in THF/heptane/ethylbenzene (2.51 mL, 2 M, 5.02 mmol) was added slowly. The reaction was stirred at −78° C. for 40 min, then N, N-dimethylformamide (972 μL, 12.5 mmol) was added. The reaction was stirred from −78° C. to rt for 2 h. The reaction was quenched with a saturated aqueous solution of NH₄Cl and extracted with ethyl ether three times. The combined organic layer was washed with brine, dried over Na₂SO₄, filtered and concentrated to afford (S)-4-bromo-5-methyl-1-(oxetan-2-ylmethyl)-1H-imidazole-2-carbaldehyde (III.3) (588.9 mg, 90.6% yield) as brownish solid. The crude was used for next step w/o further purification. LCMS Method 2 (Basic): Rt=1.02 min, MS (ESI+) m/z 259.1 (M+H)⁺.

### Step 3: Synthesis of 1-((4-bromo-5-methyl-1-(((S)-oxetan-2-yl)methyl)-1H-imidazol-2-yl)methyl)-4-((S)-2-(4-chloro-2-fluorophenyl)-2-methylbenzo[d][1,3]dioxol-4-yl)piperidine (I.A1)

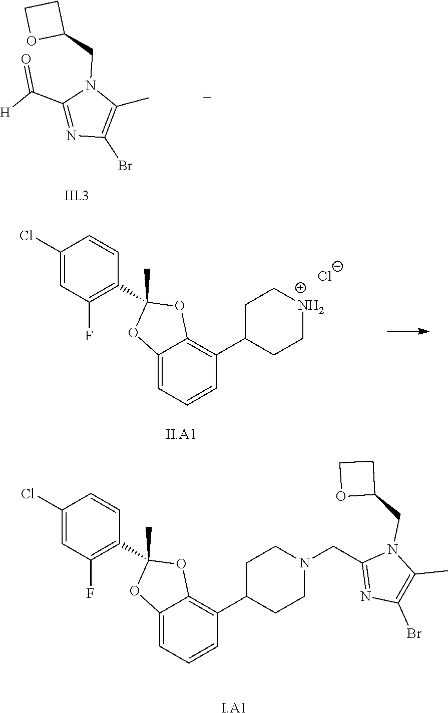

To a solution of (S)-4-bromo-5-methyl-1-(oxetan-2-ylmethyl)-1H-imidazole-2-carbaldehyde (1.6 g, 6.18 mmol) (III.3) in DCM (40 mL) was added (S)-4-(2-(4-chloro-2-fluorophenyl)-2-methylbenzo[d][1,3]dioxol-4-yl)piperidin-1-ium chloride (II.A1) (1.9 g, 4.94 mmol) and TEA (1.72 mL, 12.4 mmol). The reaction was stirred at rt for 15-25 min, then sodium triacetoxyborohydride (1.36 g, 6.43 mmol) was added. The reaction was stirred at rt for another 2 h. The reaction mixture was cooled to 0° C. and quenched with a saturated aqueous solution of NaHCO₃. The reaction mixture was extracted with DCM twice. The combined organic layer was dried over Na₂SO₄, filtered and concentrated to give an off-white foam solid. This crude was further purified by normal phase chromatography (0-100% EtOAc/heptane) to afford 1-((4-bromo-5-methyl-1-(((S)-oxetan-2-yl)methyl)-1H-imidazol-2-yl)methyl)-4-((S)-2-(4-chloro-2-fluorophenyl)-2-methylbenzo[d][1,3]dioxol-4-yl)piperidine (I.A1) as a white foam solid (1.7 g, 58.2% yield). LCMS Method 4: (Product analysis-basic: Rt=3.42 min, MS (ESI+) m/z 592.3 (M+H)⁺. ¹H NMR (400 MHz, CD₂Cl₂) δ 7.58 (t, J=8.4 Hz, 1H), 7.24-7.13 (m, 2H), 6.85-6.77 (m, 1H), 6.74 (s, 2H), 5.07 (qd, J=7.3, 2.9 Hz, 1H), 4.64 (td, J=8.0, 5.8 Hz, 1H), 4.53-4.37 (m, 2H), 4.29 (d, J=15.0 Hz, 1H), 3.67 (d, J=13.5 Hz, 1H), 3.56 (d, J=13.7 Hz, 1H), 3.07-2.84 (m, 2H), 2.76 (dtd, J=11.4, 8.0, 5.9 Hz, 2H), 2.46 (ddt, J=11.3, 9.3, 7.2 Hz, 1H), 2.23 (s, 3H), 2.21 (br,s, 2H), 2.08 (d, J=1.2 Hz, 3H), 1.84 (d, J=23.4 Hz, 4H).

### Example 2: Synthesis of 2-(2-((4-((S)-2-(4-chloro-2-fluorophenyl)-2-methylbenzo[d][1,3]dioxol-4-yl)piperidin-1-yl)methyl)-4-methyl-1-(((S)-oxetan-2-yl)methyl)-1H-imidazol-5-yl)-5-methyloxazole-4-carboxylic acid (C-1)

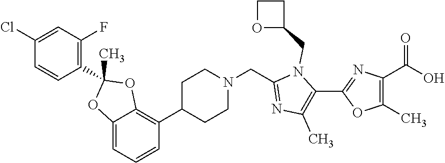

### Step 1: Synthesis of (S)-4-iodo-5-methyl-1-(oxetan-2-ylmethyl)-1H-imidazole (III.3a) and (S)-5-iodo-4-methyl-1-(oxetan-2-ylmethyl)-1H-imidazole (III.3b)

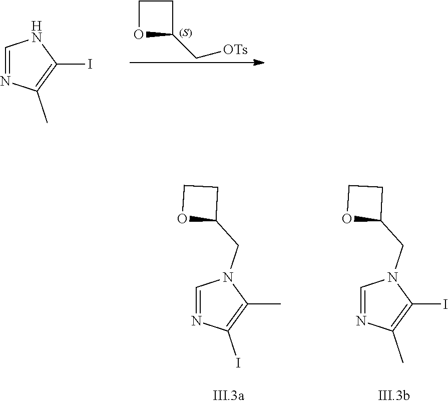

To a solution of (S)-oxetan-2-ylmethyl 4-methylbenzenesulfonate (2.17 g, 8.96 mmol) and 5-iodo-4-methyl-1H-imidazole (CAS #15813-07-72, 2.049 g, 9.85 mmol) in ACN (50 mL), Cs₂CO₃ (2.92 g, 8.96 mmol) was added. The reaction mixture was heated to reflux for 16 h. Reaction mixture was cooled to rt and filtered. The filtrate was concentrated, the residue was purified by FCC (MeOH/ethyl acetate=0 to 20%) to separate the regioisomers and afford intermediates III.3a and III.3b. (S)-4-iodo-5-methyl-1-(oxetan-2-ylmethyl)-1H-imidazole (III.3a): ¹H NMR (400 MHz, CDCl3) δ 7.51 (s, 1H), 5.03-4.95 (m, 1H), 4.64-4.56 (m, 1H), 4.32 (td, J=6.0, 9.2 Hz, 1H), 4.11-4.05 (m, 2H), 2.68 (dtd, J=6.0, 8.0, 11.6 Hz, 1H), 2.34-2.26 (m, 1H), 2.20 (s, 3H). (S)-5-iodo-4-methyl-1-(oxetan-2-ylmethyl)-1H-imidazole (III.3b): ¹H NMR (400 MHz, CDCl3) δ 7.80 (s, 1H), 5.12-4.98 (m, 1H), 4.74-4.58 (m, 1H), 4.37 (td, J=6.0, 9.2 Hz, 1H), 4.18-4.06 (m, 2H), 2.69 (dtd, J=6.0, 8.0, 11.6 Hz, 1H), 2.35 (tdd, J=7.2, 9.2, 11.2 Hz, 1H), 2.26 (s, 3H).

### Step 2: Synthesis of (S)-5-iodo-4-methyl-1-(oxetan-2-ylmethyl)-1H-imidazole-2-carbaldehyde (III.4)

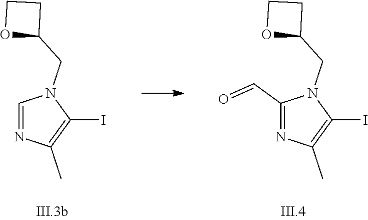

To a solution of III.3b (0.222 g, 0.8 mmol) in THF (4 mL) at −78° C., LDA (1.600 mL, 1.600 mmol) was added slowly. The reaction was stirred at −78° C. for 30 min, then DMF (0.310 mL, 4.00 mmol) was added. The reaction was stirred from −78° C. to rt for 2 h. After which, the reaction was quenched with a saturated NH₄Cl aqueous solution and extracted with ether three times. The combined organic layers were washed with brine and dried over Na₂SO₄. After filtration and concentration, the residue of III.4 was used to next step without purification. LCMS Method 2 (Basic): Rt=0.69 min, MS (ESI+) m/z=306.9 (M+1)⁺.

### Step 3: Synthesis of 4-((S)-2-(4-chloro-2-fluorophenyl)-2-methylbenzo[d][1,3]dioxol-4-yl)-1-((5-iodo-4-methyl-1-(((S)-oxetan-2-yl)methyl)-1H-imidazol-2-yl)methyl)piperidine (I.B1)

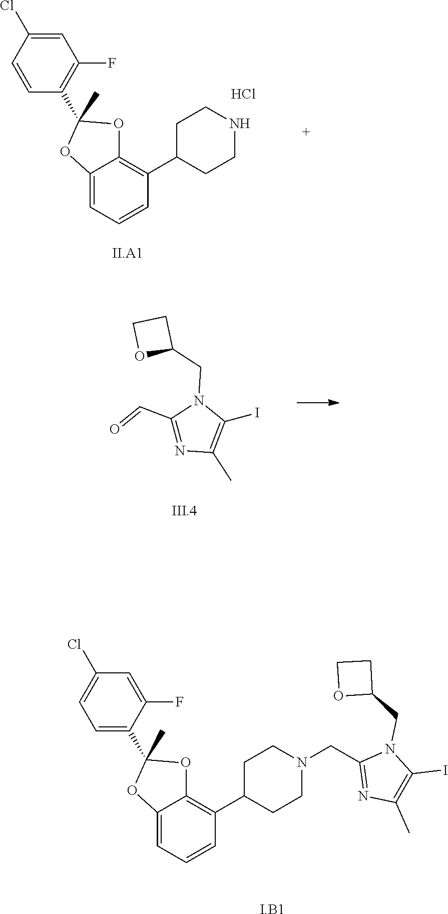

To a solution of III.4 (245 mg, 0.800 mmol) and intermediate II.A1 (215 mg, 0.560 mmol) in DCM (5 mL), pyridine (64.7 μl, 0.800 mmol) was added. The reaction was stirred at rt for 15 min, then sodium triacetoxyborohydride (170 mg, 0.800 mmol) was added. The reaction was stirred at rt for 1.5 h before being cooled to 0° C. and quenched with a saturated NaHCO₃ aqueous solution and extracted with DCM twice. The combined organic layers were concentrated, the residue was purified by FCC (EA/Hep=0 to 100%) to afford I.B1. LCMS Method 1 (Acidic): Rt=1.23 min, MS (ESI+) m/z 638.2 (M+H)⁺.

### Step 4: Synthesis of ethyl 2-(2-((4-((S)-2-(4-chloro-2-fluorophenyl)-2-methylbenzo[d][1,3]dioxol-4-yl)piperidin-1-yl)methyl)-4-methyl-1-(((S)-oxetan-2-yl)methyl)-1H-imidazol-5-yl)-5-methyloxazole-4-carboxylate (I.1)

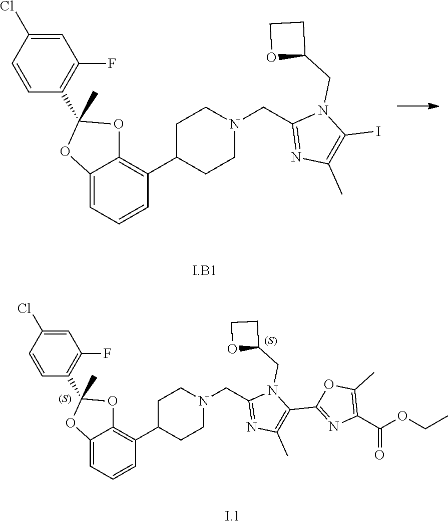

To a solution of I.B1 (2.3 g, 3.61 mmol) and ethyl 5-methyloxazole-4-carboxylate (1.12 g, 7.21 mmol) in toluene (16 mL) under nitrogen atmosphere, Xphos Pd G2 (283.7 mg, 360.5 μmol) and Cs₂CO₃ (2.35 g, 7.21 mmol) were added. Reaction was heated to 120° C. for 16 h, when LCMS analysis shows presence of desired product. Reaction mixture was quenched with a saturated NaHCO₃ aqueous solution and extracted with EtOAc three times. Combined organic layers were dried over Na₂SO₄. After filtration and concentration, the residue was purified by FCC (DCM/MeOH=0 to 10%) to afford 1.1 (1.6 g, 2.4 mmol, 66.7% yield). LCMS Method 2 (Basic): Rt=1.41 min, MS (ESI+) m/z 665.5 (M+H)⁺.

### Step 5: Synthesis of 2-(2-((4-((S)-2-(4-chloro-2-fluorophenyl)-2-methylbenzo[d][1,3]dioxol-4-yl)piperidin-1-yl)methyl)-4-methyl-1-(((S)-oxetan-2-yl)methyl)-1H-imidazol-5-yl)-5-methyloxazole-4-carboxylic acid (C-1)

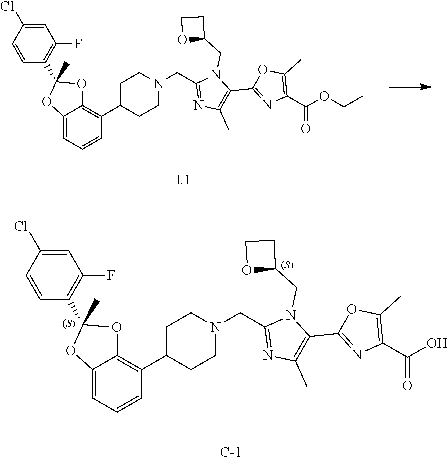

To a solution of 1.1 (1.6 g, 2.405 mmol) in mixed solvents of THF-MeOH (7 mL/4 mL), LiOH 1M in H₂O (9.622 mL, 9.622 mmol) was added. The reaction mixture was stirred overnight, when LCMS analysis showed full consumption of starting ester and formation of desired product. Reaction was then acidified with 1M citric acid to pH=4 and extracted with ethyl acetate three times. Combined organic layers were washed with brine and dried over Na₂SO₄. After filtration and concentration, the residue was purified by RP ISCO (2×150 g C18 column, 10-70% ACN in water, 0.1% conc. NH₄OH as the modifier) to afford compound C-1 (155.0 mg, 0.24 mmol, 10% yield) as a white solid after lyophilization.

C-1: ¹H NMR (400 MHz, DMSO) δ 7.57-7.39 (m, 2H), 7.27 (dd, J=8.4, 2.1 Hz, 1H), 6.74-6.59 (m, 3H), 4.93-4.80 (m, 2H), 4.72-4.57 (m, 1H), 4.38-4.29 (m, 1H), 4.23 (dt, J=8.9, 6.0 Hz, 1H), 3.69 (d, J=13.4 Hz, 1H), 3.49 (d, J=13.4 Hz, 1H), 2.90 (d, J=11.2 Hz, 1H), 2.79 (d, J=11.3 Hz, 1H), 2.62-2.48 (m, 5H), 2.32-2.21 (m, 4H), 2.16-1.99 (m, 2H), 1.95 (s, 3H), 1.66 (d, J=27.9 Hz, 4H). LCMS Method 2 (Basic): Rt=0.79 min, MS (ESI+) m/z 637.5 (M+H)⁺. HRMS calculated for C₃₃H₃₅ClFN₄O₆ (M+H)⁺637.2229, found 637.2401.

### Example 2-1: Synthesis of 2-(2-((4-((S)-2-(4-chloro-2-fluorophenyl)-2-methylbenzo[d][1,3]dioxol-4-yl)piperidin-1-yl)methyl)-4-methyl-1-(((R)-oxetan-2-yl)methyl)-1H-imidazol-5-yl)oxazole-5-carboxylic acid (C-2)

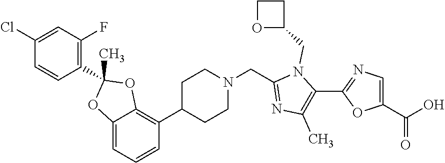

Compound C-2 was synthesized using same procedures as set forth in Example 2 but using (R)-oxetan-2-ylmethyl 4-methylbenzenesulfonate in step 1, and ethyl oxazole-5-carboxylate in step 4.

C-2: ¹H NMR (400 MHz, MeOD) δ 7.53-7.45 (m, 2H), 7.18 (dd, J=11.0, 2.1 Hz, 1H), 7.11 (dd, J=8.4, 2.1 Hz, 1H), 6.76-6.60 (m, 3H), 5.17-5.06 (m, 1H), 4.90-4.80 (m, 2H), 4.60 (td, J=8.1, 5.8 Hz, 1H), 4.47-4.36 (m, 2H), 4.36-4.28 (m, 1H), 3.64-3.51 (m, 2H), 3.11-2.95 (m, 2H), 2.90-2.85 (m, 1H), 2.72-2.59 (m, 1H), 2.48 (s, 3H), 2.44-2.32 (m, 1H), 2.07-1.95 (m, 4H), 1.93 (s, 3H). HRMS calculated for C₃₂H₃₃ClFN₄O₆ (M+H)⁺623.2073, found 623.2139.

### Example 2-2: Synthesis of 2-(2-((4-((S)-2-(4-chloro-2-fluorophenyl)-2-methylbenzo[d][1,3]dioxol-4-yl)piperidin-1-yl)methyl)-4-methyl-1-(((R)-oxetan-2-yl)methyl)-1H-imidazol-5-yl)oxazole-4-carboxylic acid (C-3)

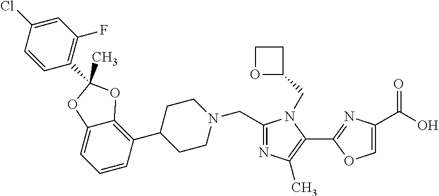 [SMILES: Cc1nc(CN2CCC(c3cccc4c3O[C@@](C)(c3ccc(Cl)cc3F)O4)CC2)n(C[C@H]2CCO2)c1-c1nc(C(=O)O)co1]

Compound C-3 was synthesized using same procedures as set forth in Example 2 but using (R)-oxetan-2-ylmethyl 4-methylbenzenesulfonate in step 1, and ethyl oxazole-4-carboxylate in step 4.

C-3: ¹H NMR (400 MHz, MeOD) δ 8.23 (s, 1H), 7.49 (t, J=8.3 Hz, 1H), 7.19 (dd, J=10.9, 2.1 Hz, 1H), 7.11 (dd, J=8.4, 2.1 Hz, 1H), 6.75-6.68 (m, 1H), 6.68-6.59 (m, 2H), 5.15-5.05 (m, 1H), 4.95-4.90 (m, 2H), 4.61-4.51 (m, 1H), 4.44-4.34 (m, 1H), 4.20 (s, 2H), 3.43-3.35 (m, 2H), 2.83 (d, J=14.9 Hz, 3H), 2.74-2.61 (m, 1H), 2.43 (s, 3H), 2.41-2.30 (m, 1H), 1.98-1.86 (m, 7H). HRMS calculated for C32H33ClFN4O6 (M+H)⁺ 623.2073, found 623.2103.

### Example 2-3: Synthesis of 2-(2-((4-((S)-2-(4-chloro-2-fluorophenyl)-2-methylbenzo[d][1,3]dioxol-4-yl)piperidin-1-yl)methyl)-1-(((S)-oxetan-2-yl)methyl)-4-(trifluoromethyl)-1H-imidazol-5-yl)oxazole-4-carboxylic acid (C-4)

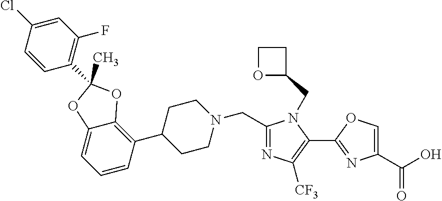 [SMILES: C[C@]1(c2ccc(Cl)cc2F)Oc2cccc(C3CCN(Cc4nc(C(F)(F)F)c(-c5nc(C(=O)O)co5)n4C[C@@H]4CCO4)CC3)c2O1]

Compound C-4 was synthesized using same procedures as set forth in Example 2 but using 5-iodo-4-(trifluoromethyl)-1H-imidazole in step 1, and ethyl oxazole-4-carboxylate in step 4.

C-4: ¹H NMR (400 MHz, METHANOL-d₄) δ ppm 1.90-2.13 (m, 7H) 2.31-2.57 (m, 1H) 2.62-3.04 (m, 4H) 3.32-3.46 (m, 2H) 4.02-4.33 (m, 2H) 4.34-4.49 (m, 1H) 4.53-4.68 (m, 1H) 4.88-4.98 (m, 1H) 5.02-5.30 (m, 2H) 6.66-6.76 (m, 2H) 6.76-6.84 (m, 1H) 7.17-7.24 (m, 1H) 7.24-7.32 (m, 1H) 7.53-7.62 (m, 1H) 8.10-8.79 (m, 1H). HRMS calculated for C₃₂H₃₀ClF₄N₄O₆ (M+H)⁺ 677.1790, found 677.1899.

### Example 2-4: Synthesis of 2-(2-((4-((S)-2-(4-chloro-2-fluorophenyl)-2-methylbenzo[d][1,3]dioxol-4-yl)piperidin-1-yl)methyl)-4-ethyl-1-(((S)-oxetan-2-yl)methyl)-1H-imidazol-5-yl)oxazole-4-carboxylic acid (C-5)

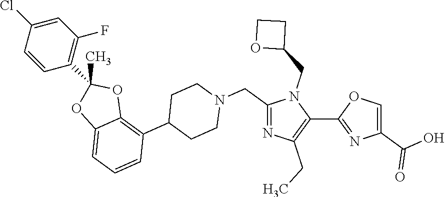 [SMILES: CCc1nc(CN2CCC(c3cccc4c3O[C@@](C)(c3ccc(Cl)cc3F)O4)CC2)n(C[C@@H]2CCO2)c1-c1nc(C(=O)O)co1]

Compound C-5 was synthesized using same procedures as set forth in Example 2 but using 4-ethyl-5-iodo-1H-imidazole in step 1, and ethyl oxazole-4-carboxylate in step 4.

C-5: ¹H NMR (400 MHz, METHANOL-d₄) δ ppm 1.27 (t, J=7.52 Hz, 3H) 1.91-2.14 (m, 8H) 2.41-2.51 (m, 1H) 2.73-2.81 (m, 1H) 2.87-2.96 (m, 5H) 3.44-3.52 (m, 2H) 4.28-4.33 (m, 2H) 4.46-4.53 (m, 1H) 4.64-4.70 (m, 1H) 4.99-5.08 (m, 2H) 5.17-5.24 (m, 1H) 6.72-6.82 (m, 3H) 7.18-7.24 (m, 1H) 7.25-7.32 (m, 1H) 7.54-7.63 (m, 1H) 8.27-8.34 (m, 1H). HRMS calculated for C₃₃H₃₅ClFN₄O₆ (M+H)⁺ 637.2229, found 637.2405.

### Example 2-5: Synthesis of 2-(2-((4-((S)-2-(4-chloro-2-fluorophenyl)-2-methylbenzo[d][1,3]dioxol-4-yl)piperidin-1-yl)methyl)-4-ethyl-1-(((S)-oxetan-2-yl)methyl)-1H-imidazol-5-yl)oxazole-5-carboxylic acid (C-6)

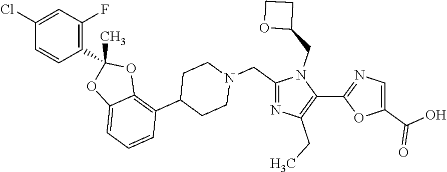 [SMILES: CCc1nc(CN2CCC(c3cccc4c3O[C@@](C)(c3ccc(Cl)cc3F)O4)CC2)n(C[C@@H]2CCO2)c1-c1ncc(C(=O)O)o1]

Compound C-6 was synthesized using same procedures as set forth in Example 2 but using 4-ethyl-5-iodo-1H-imidazole in step 1, and ethyl oxazole-5-carboxylate in step 4.

C-6: ¹H NMR (400 MHz, DMSO-d₆) δ ppm 1.15-1.23 (m, 3H) 1.65-1.82 (m, 4H) 2.02 (s, 3H) 2.07-2.40 (m, 4H) 2.62-2.68 (m, 2H) 2.79-2.90 (m, 3H) 2.96-3.02 (m, 1H) 3.59 (br d, J=13.45 Hz, 1H) 3.76-3.87 (m, 1H) 4.27-4.46 (m, 2H) 4.53-4.78 (m, 1H) 4.79-5.06 (m, 2H) 6.73-6.81 (m, 3H) 7.31-7.37 (m, 1H) 7.51-7.61 (m, 2H). HRMS calculated for C₃₃H₃₅ClFN₄O₆ (M+H)⁺ 637.2229, found 637.2264.

### Example 2-6: Synthesis of 2-(2-((4-((S)-2-(4-chloro-2-fluorophenyl)-2-methylbenzo[d][1,3]dioxol-4-yl)piperidin-1-yl)methyl)-4-ethyl-1-(((R)-oxetan-2-yl)methyl)-1H-imidazol-5-yl)oxazole-4-carboxylic acid (C-7)

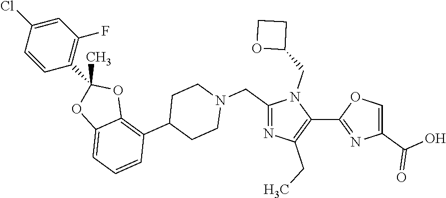 [SMILES: CCc1nc(CN2CCC(c3cccc4c3O[C@@](C)(c3ccc(Cl)cc3F)O4)CC2)n(C[C@H]2CCO2)c1-c1nc(C(=O)O)co1]

Compound C-7 was synthesized using same procedures as set forth in Example 2 but using 4-ethyl-5-iodo-1H-imidazole and (R)-oxetan-2-ylmethyl 4-methylbenzenesulfonate in step 1, and ethyl oxazole-4-carboxylate in step 4.

C-7: ¹H NMR (400 MHz, DMSO-d₆) δ ppm 1.12-1.22 (m, 3H) 1.68-1.82 (m, 4H) 1.98-2.04 (m, 3H) 2.06-2.25 (m, 2H) 2.25-2.37 (m, 1H) 2.57-2.68 (m, 2H) 2.72-2.82 (m, 2H) 2.82-2.91 (m, 1H) 2.93-3.02 (m, 1H) 3.54-3.65 (m, 1H) 3.72-3.87 (m, 1H) 4.23-4.33 (m, 1H) 4.34-4.44 (m, 1H) 4.63-4.76 (m, 1H) 4.86-5.03 (m, 2H) 6.70-6.82 (m, 3H) 7.30-7.37 (m, 1H) 7.51-7.61 (m, 2H) 8.74-8.79 (m, 1H). HRMS calculated for C₃₃H₃₅ClFN₄O₆ (M+H)⁺ 637.2229, found 637.2377.

### Example 2-7: Synthesis of 2-(2-((4-((S)-2-(4-chloro-2-fluorophenyl)-2-methylbenzo[d][1,3]dioxol-4-yl)piperidin-1-yl)methyl)-4-ethyl-1-(((R)-oxetan-2-yl)methyl)-1H-imidazol-5-yl)oxazole-5-carboxylic acid (C-8)

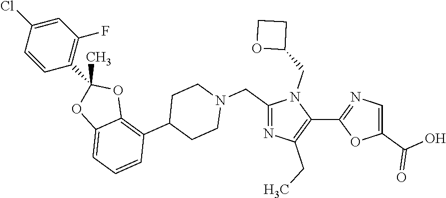 [SMILES: CCc1nc(CN2CCC(c3cccc4c3O[C@@](C)(c3ccc(Cl)cc3F)O4)CC2)n(C[C@H]2CCO2)c1-c1ncc(C(=O)O)o1]

Compound C-8 was synthesized using same procedures as set forth in Example 2 but using 4-ethyl-5-iodo-1H-imidazole and (R)-oxetan-2-ylmethyl 4-methylbenzenesulfonate in step 1, and ethyl oxazole-5-carboxylate in step 4.

C-8: ¹H NMR (400 MHz, DMSO-d₆) δ ppm 1.11-1.21 (m, 3H) 1.69-1.80 (m, 4H) 2.02 (s, 3H) 2.08-2.28 (m, 2H) 2.29-2.37 (m, 1H) 2.59-2.68 (m, 2H) 2.70 (br s, 3H) 2.95-3.05 (m, 1H) 3.59-3.65 (m, 1H) 3.77-3.86 (m, 1H) 4.27-4.46 (m, 2H) 4.56-4.75 (m, 1H) 4.75-5.06 (m, 2H) 6.72-6.82 (m, 3H) 7.31-7.37 (m, 1H) 7.52-7.60 (m, 2H) 7.76-8.03 (m, 1H). HRMS calculated for C₃₃H₃₅ClFN₄O₆ (M+H)⁺ 637.2229, found 637.2351.

### Example 2-8: Synthesis of 2-(2-((4-((S)-2-(4-chloro-2-fluorophenyl)-2-methylbenzo[d][1, 3]dioxol-4-yl)piperidin-1-yl)methyl)-4-methyl-1-(((S)-oxetan-2-yl)methyl)-1H-imidazol-5-yl)oxazole-4-carboxylic acid (C-9)

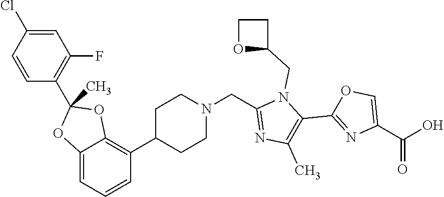 [SMILES: Cc1nc(CN2CCC(c3cccc4c3O[C@@](C)(c3ccc(Cl)cc3F)O4)CC2)n(C[C@@H]2CCO2)c1-c1nc(C(=O)O)co1]

Compound C-9 was synthesized using same procedures as set forth in Example 2 but using ethyl oxazole-4-carboxylate in step 4.

C-9: ¹H NMR (400 MHz, MeOD) δ 8.30 (s, 1H), 7.60-7.56 (m, 1H), 7.28 (dd, J=11.2 Hz, 2 Hz, 1H), 7.23-7.20 (m, 1H), 6.82-6.78 (m, 1H), 6.75-6.71 (m, 2H), 5.22-5.20 (m, 1H), 5.06-5.04 (m, 2H), 4.68-4.66 (m, 1H), 4.5-4.48 (m, 1H), 4.26 (s, 2H), 3.47-3.43 (m, 2H), 2.88-2.77 (m, 4H), 2.52 (s, 3H), 2.51-2.47 (m, 1H), 2.03 (s, 3H), 2.02-1.99 (m, 4H). LCMS Method 1 (Acidic): Rt=1.57 min, MS (ESI+) m/z 623.2 (M+H)⁺.

### Example 2-9: Synthesis of 2-(2-((4-((S)-2-(4-chloro-2-fluorophenyl)-2-methylbenzo[d][1,3]dioxol-4-yl)piperidin-1-yl)methyl)-4-methyl-1-(((S)-oxetan-2-yl)methyl)-1H-imidazol-5-yl)-5-ethyloxazole-4-carboxylic acid (C-10)

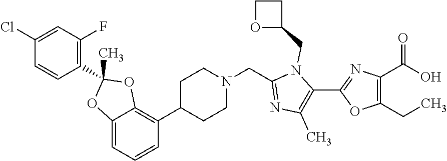

Compound C-10 was synthesized using same procedures as set forth in Example 2 but using ethyl 5-ethyloxazole-4-carboxylate in step 4.

C-10: ¹H NMR (400 MHz, DMSO) δ 7.56-7.43 (m, 2H), 7.27 (dd, J=8.5, 2.1 Hz, 1H), 6.76-6.63 (m, 3H), 4.86 (dd, J=10.2, 3.4 Hz, 2H), 4.73-4.56 (m, 1H), 4.34 (q, J=6.8 Hz, 1H), 4.24 (dt, J=8.9, 6.0 Hz, 1H), 3.69 (d, J=13.3 Hz, 1H), 3.49 (d, J=13.4 Hz, 1H), 2.99 (q, J=7.6 Hz, 2H), 2.90 (d, J=11.0 Hz, 1H), 2.79 (d, J=11.2 Hz, 1H), 2.56 (dd, J=16.3, 11.7 Hz, 2H), 2.31-2.20 (m, 4H), 2.15-1.98 (m, 2H), 1.95 (s, 3H), 1.77-1.53 (m, 4H), 1.13 (t, J=7.4 Hz, 3H). HRMS calculated for C₃₄H₃₇ClFN₄O₅ (M+H)⁺ 651.2386, found 651.2513.

### Example 2-10: Synthesis of 2-(2-((4-((S)-2-(4-cyano-2-fluorophenyl)-2-methylbenzo[d][1,3]dioxol-4-yl)piperidin-1-yl)methyl)-4-methyl-1-(((S)-oxetan-2-yl)methyl)-1H-imidazol-5-yl)-5-methyloxazole-4-carboxylic acid (C-11)

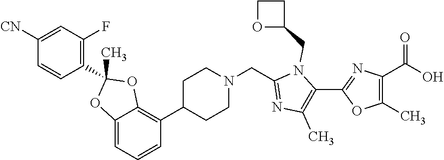

Compound C-11 was synthesized using same procedures as set forth in Example 2 but using intermediate II.B1 instead of II.A1 in step 3.

C-11: ¹H NMR (400 MHz, DMSO) δ 7.91 (d, J=11.2 Hz, 1H), 7.67 (d, J=6.5 Hz, 2H), 6.82-6.62 (m, 3H), 4.94-4.78 (m, 2H), 4.71-4.53 (m, 1H), 4.34 (dt, J=8.5, 6.3 Hz, 1H), 4.24 (dt, J=8.8, 6.0 Hz, 1H), 3.70 (d, J=13.5 Hz, 1H), 3.50 (d, J=13.5 Hz, 1H), 2.90 (d, J=11.1 Hz, 1H), 2.79 (d, J=11.0 Hz, 1H), 2.63-2.51 (m, 5H), 2.33-2.19 (m, 4H), 2.17-1.91 (m, 5H), 1.76-1.55 (m, 4H). HRMS calculated for C₃₄H₃₅FN₅O₆ (M+H)⁺ 628.2571, found 628.2587.

### Example 3: Synthesis of 2-(2-((4-((S)-2-(4-chloro-2-fluorophenyl)-2-methylbenzo[d][1,3]dioxol-4-yl)piperidin-1-yl)methyl)-5-methyl-1-(((S)-oxetan-2-yl)methyl)-1H-imidazol-4-yl)-5-methyloxazole-4-carboxylic acid (C-12)

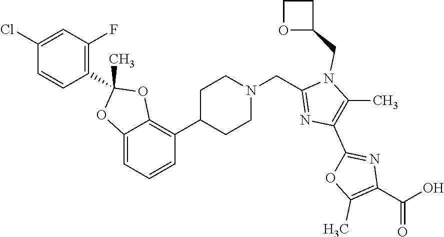

### Step 1: Synthesis of (S)-4-iodo-5-methyl-1-(oxetan-2-ylmethyl)-1H-imidazole-2-carbaldehyde (III.5)

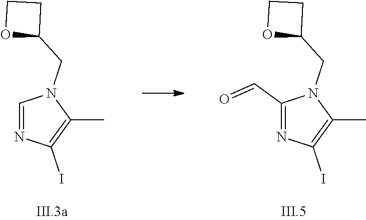

Intermediate 111.5 was synthesized using same procedures as set forth in step 2 of Example 2 but using III.3a instead of III.3b. LCMS Method 2 (Basic): Rt=0.69 min, MS (ESI+) m/z 306.9 (M+H)⁺.

### Step 2: Synthesis of 4-((S)-2-(4-chloro-2-fluorophenyl)-2-methylbenzo[d][1,3]dioxol-4-yl)-1-((4-iodo-5-methyl-1-(((S)-oxetan-2-yl)methyl)-1H-imidazol-2-yl)methyl)piperidine (I.A2)

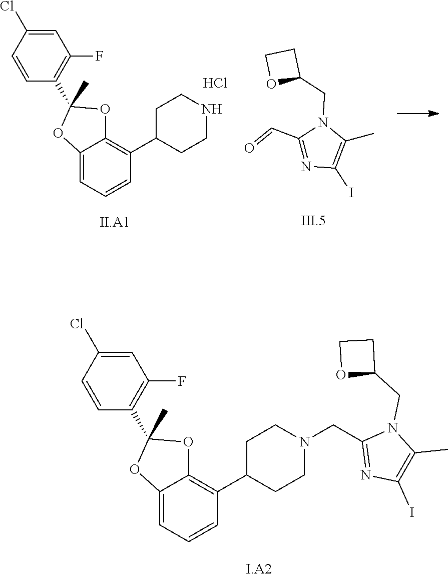

Intermediate I.A2 was synthesized using same procedures as set forth in step 3 of Example 2 but using 111.5 instead of III.4. LCMS Method 2 (Basic): Rt=1.43 min, MS (ESI+) m/z 633.0 (M+H)⁺.

### Step 3: Synthesis of ethyl 2-(2-((4-((S)-2-(4-chloro-2-fluorophenyl)-2-methylbenzo[d][1,3]dioxol-4-yl)piperidin-1-yl)methyl)-5-methyl-1-(((S)-oxetan-2-yl)methyl)-1H-imidazol-4-yl)-5-methyloxazole-4-carboxylate (I.2)

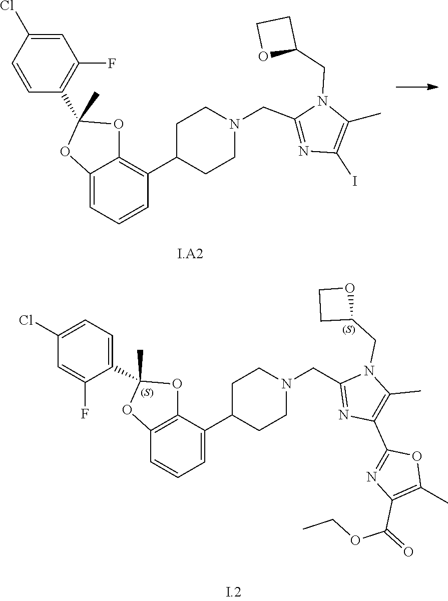

Intermediate 1.2 was synthesized using same procedures as set forth in step 4 of Example 2 but using I.A2 instead of I.B1. LCMS Method 2 (Basic): Rt=1.38 min, MS (ESI+) m/z 665.5 (M+H)⁺.

### Step 4: Synthesis of 2-(2-((4-((S)-2-(4-chloro-2-fluorophenyl)-2-methylbenzo[d][1,3]dioxol-4-yl)piperidin-1-yl)methyl)-5-methyl-1-(((S)-oxetan-2-yl)methyl)-1H-imidazol-4-yl)-5-methyloxazole-4-carboxylic acid (C-12)

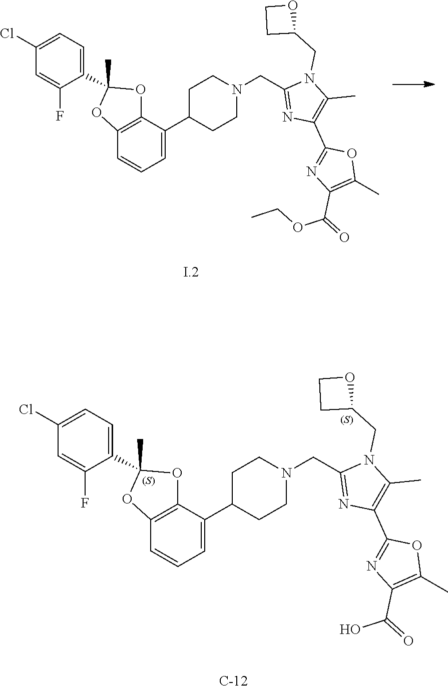

Compound C-12 was synthesized using same procedures as set forth in step 5 of Example 2 but using 1.2 instead of 1.1.

C-12: ¹H NMR (400 MHz, DMSO) δ 7.55-7.44 (m, 2H), 7.27 (dd, J=8.5, 2.1 Hz, 1H), 6.76-6.60 (m, 3H), 4.96 (dd, J=8.8, 6.3 Hz, 1H), 4.49-4.29 (m, 3H), 4.23 (dd, J=15.2, 2.8 Hz, 1H), 3.66 (d, J=13.5 Hz, 1H), 3.42 (d, J=13.4 Hz, 1H), 2.91 (d, J=11.1 Hz, 1H), 2.76 (d, J=11.3 Hz, 1H), 2.61 (td, J=17.4, 8.7 Hz, 2H), 2.51 (s, 3H), 2.49 (s, 3H), 2.39-2.31 (m, 1H), 2.13-1.99 (m, 2H), 1.96 (s, 3H), 1.75-1.47 (m, 4H). LCMS Method 2 (Basic): Rt=0.87 min, MS (ESI+) m/z 637.5 (M+H)⁺. HRMS calculated for C₃₃H₃₅ClFN₄O₆ (M+H)⁺ 637.2229, found 637.2288.

### Example 3-1: Synthesis of 2-(2-((4-((S)-2-(4-chloro-2-fluorophenyl)-2-methylbenzo[d][1,3]dioxol-4-yl)piperidin-1-yl)methyl)-5-methyl-1-(((S)-oxetan-2-yl)methyl)-1H-imidazol-4-yl)oxazole-4-carboxylic acid (C-13)

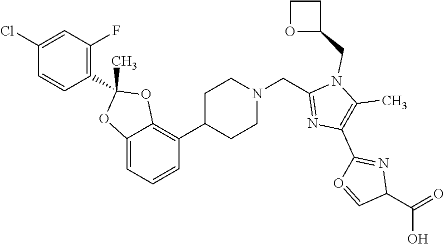

Compound C-13 was synthesized using same procedures as set forth in Example 3 but using ethyl oxazole-4-carboxylate in step 3.

C-13: ¹H NMR (400 MHz, MeOD) δ 8.64 (s, 1H), 7.58-7.53 (m, 2H), 7.33 (dd, J=8.4, 1.6 Hz, 1H), 6.79-6.78 (m, 2H), 6.76-6.73 (m, 1H), 5.05-5.03 (m, 1H), 4.51-4.42 (m, 3H), 4.33-4.29 (m, 1H), 3.74 (d, J=13.2 Hz, 1H), 3.50 (d, J=5 Hz, 1H), 3.0-2.97 (m, 1H), 2.85-2.83 (m, 1H), 2.69-2.67 (m, 2H), 2.58 (s, 3H), 2.49-2.32 (m, 1H), 2.18-2.01 (m, 3H), 2.02 (s, 3H), 1.76-1.66 (m, 4H). LCMS Method 1 (Acidic): Rt=1.12 min, MS (ESI+) m/z 623.2 (M+H)+.

### Example 3-2: Synthesis of 2-(2-((4-((S)-2-(4-chloro-2-fluorophenyl)-2-methylbenzo[d][1,3]dioxol-4-yl)piperidin-1-yl)methyl)-5-methyl-1-(((S)-oxetan-2-yl)methyl)-1H-imidazol-4-yl)-4-(trifluoromethyl)oxazole-5-carboxylic acid (C-14)

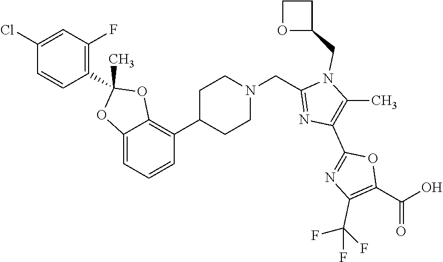

Compound C-14 was synthesized using same procedures as set forth in Example 3 but using ethyl 4-(trifluoromethyl)oxazole-5-carboxylate in step 3.

C-14: ¹H NMR (400 MHz, DMSO) δ 7.54-7.44 (m, 2H), 7.28 (dd, J=8.5, 2.1 Hz, 1H), 6.77-6.60 (m, 3H), 5.04-4.88 (m, 1H), 4.49-4.29 (m, 3H), 4.24 (d, J=14.4 Hz, 1H), 3.67 (d, J=13.6 Hz, 1H), 3.43 (d, J=13.3 Hz, 1H), 2.92 (d, J=11.1 Hz, 1H), 2.78 (d, J=11.3 Hz, 1H), 2.67-2.54 (m, 2H), 2.49 (s, 3H), 2.37-2.31 (m, 1H), 2.15-1.97 (m, 2H), 1.96 (s, 3H), 1.77-1.44 (m, 4H). HRMS calculated for C₃₃H₃₂ClF₄N₄O₆ (M+H)⁺ 691.1947, found 691.1964.

### Example 3-3: Synthesis of 2-(2-((4-((S)-2-(4-chloro-2-fluorophenyl)-2-methylbenzo[d][1,3]dioxol-4-yl)piperidin-1-yl)methyl)-5-methyl-1-(((S)-oxetan-2-yl)methyl)-1H-imidazol-4-yl)-4-methyloxazole-5-carboxylic acid (C-15)

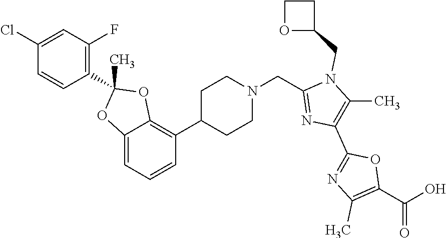

Compound C-15 was synthesized using same procedures as set forth in Example 3 but using ethyl 4-methyloxazole-5-carboxylate in step 3.

C-15: ¹H NMR (400 MHz, DMSO) δ 7.55-7.46 (m, 2H), 7.28 (dd, J=8.4, 2.1 Hz, 1H), 6.77-6.62 (m, 3H), 4.97 (qd, J=7.4, 2.9 Hz, 1H), 4.50-4.29 (m, 3H), 4.20 (dd, J=15.5, 2.9 Hz, 1H), 3.65 (d, J=13.3 Hz, 1H), 3.41 (d, J=13.3 Hz, 1H), 2.92 (d, J=11.2 Hz, 1H), 2.79 (d, J=11.2 Hz, 1H), 2.65-2.54 (m, 2H), 2.48 (s, 3H), 2.38-2.29 (m, 1H), 2.24 (s, 3H), 2.13-1.89 (m, 5H), 1.79-1.51 (m, 4H). HRMS calculated for C₃₃H₃₅ClFN₄O₆ (M+H)⁺637.2229, found 637.2372.

### Example 4: Synthesis of 5-(2-((4-((S)-2-(4-chloro-2-fluorophenyl)-2-methylbenzo[d][1,3]dioxol-4-yl)piperidin-1-yl)methyl)-5-methyl-1-(((S)-oxetan-2-yl)methyl)-1H-imidazol-4-yl)oxazole-2-carboxylic acid (C-16)

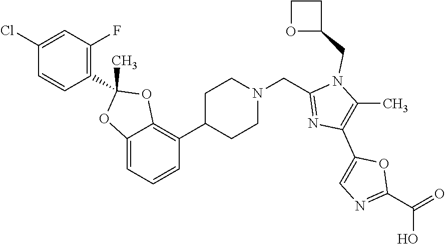

### Step 1: Synthesis of ethyl 5-(2-((4-((S)-2-(4-chloro-2-fluorophenyl)-2-methylbenzo[d][1,3]dioxol-4-yl)piperidin-1-yl)methyl)-5-methyl-1-(((S)-oxetan-2-yl)methyl)-1H-imidazol-4-yl)oxazole-2-carboxylate (I.3)

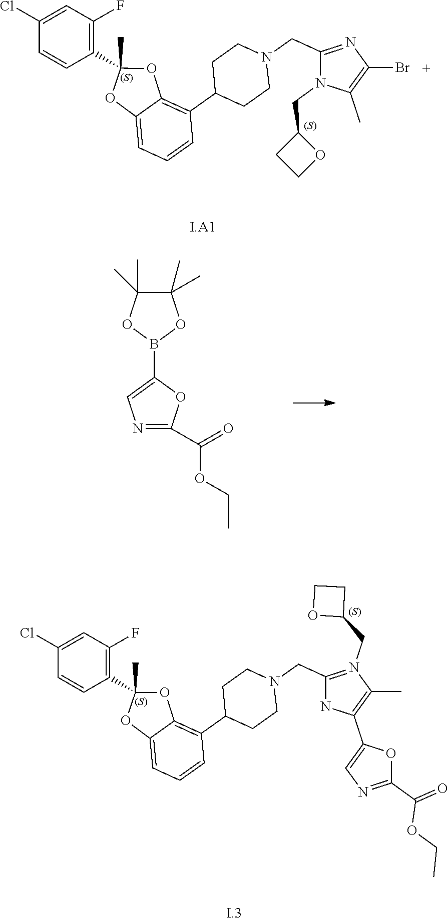

To a solution of 1-((4-bromo-5-methyl-1-(((S)-oxetan-2-yl)methyl)-1H-imidazol-2-yl)methyl)-4-((S)-2-(4-chloro-2-fluorophenyl)-2-methylbenzo[d][1,3]dioxol-4-yl)piperidine (I.A1) (50.00 mg, 84.61 μmol) in 1,4-dioxane (1.000 mL) was added ethyl 5-(4,4,5,5-tetramethyl-1,3,2-dioxaborolan-2-yl)oxazole-2-carboxylate (29.38 mg, 110.0 μmol), K₃PO₄ (126.9 μL, 2.000 M, 253.8 μmol) and Pd 118 (5.515 mg, 8.461 μmol). The reaction mixture was microwaved at 120° C., for 30 min. LCMS showed that the reaction was complete. The crude was diluted with DCM and washed with saturated NaHCO₃. The separated organic layer was filtered to remove water, concentrated via rotovap to afford a brown oil (I.3), which is used ‘as is’ in next step. LCMS Method 2 (Basic): Rt=1.37 min, MS (ESI+) m/z 651.2 (M+H)⁺.

### Step 2: Synthesis of 5-(2-((4-((S)-2-(4-chloro-2-fluorophenyl)-2-methylbenzo[d][1,3]dioxol-4-yl)piperidin-1-yl)methyl)-5-methyl-1-(((S)-oxetan-2-yl)methyl)-1H-imidazol-4-yl)oxazole-2-carboxylic acid (C-16)

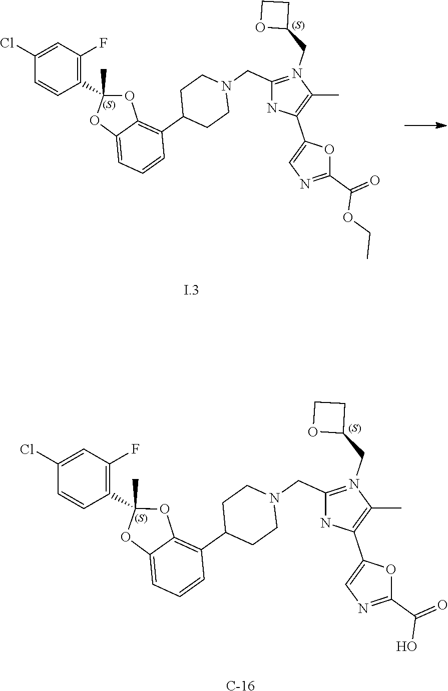

To a solution of ethyl 5-(2-((4-((S)-2-(4-chloro-2-fluorophenyl)-2-methylbenzo[d][1,3]dioxol-4-yl)piperidin-1-yl)methyl)-5-methyl-1-(((S)-oxetan-2-yl)methyl)-1H-imidazol-4-yl)oxazole-2-carboxylate (I.3) (55.00 mg, 84.47 μmol) in THF (0.500 mL) and methanol (0.25 mL) was added LiOH (0.337 mL, 1.000 M, 337.9 μmol). The reaction mixture was stirred at rt for 1 hr. LCMS showed that the reaction was complete. The crude was directly loaded onto a 50 g C18 column, and purified by ISCO (10-100% ACN in water, modified with 0.1% conc. NH₄OH) to afford 5-(2-((4-((S)-2-(4-chloro-2-fluorophenyl)-2-methylbenzo[d][1,3]dioxol-4-yl)piperidin-1-yl)methyl)-5-methyl-1-(((S)-oxetan-2-yl)methyl)-1H-imidazol-4-yl)oxazole-2-carboxylic acid lithium salt (C-16) (12.00 mg, 22%) after lyophilization.

C-16: LCMS Method 2 (Basic): Rt=0.89 min, MS (ESI+) m/z 623.2 (M+H)⁺. ¹H NMR (400 MHz, MeOD) δ 7.48 (t, J=8.3 Hz, 1H), 7.21-7.06 (m, 3H), 6.72-6.54 (m, 3H), 5.08 (qd, J=7.4, 2.5 Hz, 1H), 4.61-4.46 (m, 2H), 4.41 (dt, J=9.2, 5.9 Hz, 1H), 4.30 (dd, J=15.3, 2.6 Hz, 1H), 3.70 (d, J=13.6 Hz, 1H), 3.53 (d, J=13.6 Hz, 1H), 2.92 (d, J=11.3 Hz, 1H), 2.85-2.74 (m, 1H), 2.71 (ddd, J=11.5, 5.5, 2.7 Hz, 1H), 2.65-2.51 (m, 1H), 2.47 (s, 3H), 2.40 (dt, J=11.2, 8.0 Hz, 1H), 2.10 (dtd, J=30.6, 11.4, 2.9 Hz, 2H), 1.93 (s, 3H), 1.77 (dddd, J=25.3, 17.4, 12.7, 7.0 Hz, 4H).

### Example 4-1: Synthesis of 3-(2-((4-((S)-2-(4-chloro-2-fluorophenyl)-2-methylbenzo[d][1,3]dioxol-4-yl)piperidin-1-yl)methyl)-5-methyl-1-(((S)-oxetan-2-yl)methyl)-1H-imidazol-4-yl)-1-methyl-1H-pyrazole-5-carboxylic acid (C-17)

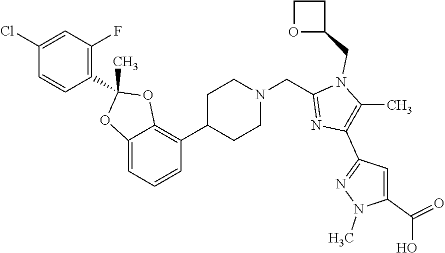

Compound C-17 was synthesized using same procedures as set forth in Example 4 but using methyl 1-methyl-3-(4,4,5,5-tetramethyl-1,3,2-dioxaborolan-2-yl)-1H-pyrazole-5-carboxylate in step 1.

C-17: ¹H NMR (400 MHz, MeOD) δ 7.48 (t, J=8.3 Hz, 1H), 7.18 (dd, J=11.0, 2.1 Hz, 1H), 7.11 (dd, J=8.3, 2.0 Hz, 1H), 6.76 (s, 1H), 6.71-6.53 (m, 3H), 5.07 (qd, J=7.3, 2.6 Hz, 1H), 4.59-4.33 (m, 3H), 4.28 (dd, J=15.4, 2.7 Hz, 1H), 4.05 (s, 3H), 3.67 (d, J=13.5 Hz, 1H), 3.54 (d, J=13.5 Hz, 1H), 2.93 (d, J=11.3 Hz, 1H), 2.83 (d, J=11.5 Hz, 1H), 2.69 (dtd, J=11.2, 8.0, 6.0 Hz, 1H), 2.58 (ddd, J=11.8, 7.6, 3.4 Hz, 1H), 2.47-2.38 (m, 1H), 2.36 (s, 3H), 2.10 (dtd, J=26.9, 11.4, 2.9 Hz, 2H), 1.93 (s, 3H), 1.87-1.63 (m, 4H). HRMS calculated for C₃₃H₃₆ClFN₅O₅ (M+H)⁺ 636.2389, found 636.2411.

### Example 4-2: Synthesis of 4-(2-((4-((S)-2-(4-chloro-2-fluorophenyl)-2-methylbenzo[d][1,3]dioxol-4-yl)piperidin-1-yl)methyl)-5-methyl-1-(((S)-oxetan-2-yl)methyl)-1H-imidazol-4-yl)oxazole-2-carboxylic acid (C-18)

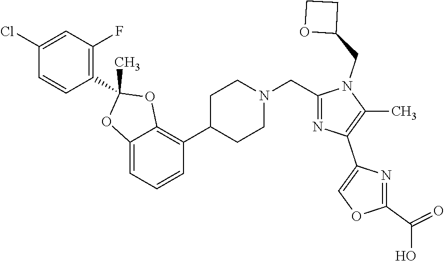

Compound C-18 was synthesized using same procedures as set forth in Example 4 but using ethyl 4-(4,4,5,5-tetramethyl-1,3,2-dioxaborolan-2-yl)oxazole-2-carboxylate in step 1.

C-18: ¹H NMR (400 MHz, MeOD) δ 7.92 (s, 1H), 7.48 (t, 1H), 7.18 (dd, J=10.8, 2.0 Hz, 1H), 7.11 (dd, J=8.5, 2.0 Hz, 1H), 6.73-6.53 (m, 3H), 5.13-4.98 (m, 1H), 4.59-4.44 (m, 2H), 4.40 (dt, J=9.2, 5.9 Hz, 1H), 4.28 (dd, J=15.3, 2.7 Hz, 1H), 3.69 (d, J=13.6 Hz, 1H), 3.53 (d, J=13.6 Hz, 1H), 2.93 (d, J=11.3 Hz, 1H), 2.82 (d, J=11.4 Hz, 1H), 2.77-2.65 (m, 1H), 2.63-2.53 (m, 1H), 2.50 (s, 3H), 2.46-2.34 (m, 1H), 2.21-1.99 (m, 2H), 1.93 (s, 3H), 1.86-1.61 (m, 4H). LCMS Method 2 (Basic): Rt=0.81 min, MS (ESI+) m/z 623.2 (M+H)⁺.

### Example 4-3: Synthesis of 5-(2-((4-((S)-2-(4-chloro-2-fluorophenyl)-2-methylbenzo[d][1,3]dioxol-4-yl)piperidin-1-yl)methyl)-5-methyl-1-(((S)-oxetan-2-yl)methyl)-1H-imidazol-4-yl)-3-methylfuran-2-carboxylic acid (C-19)

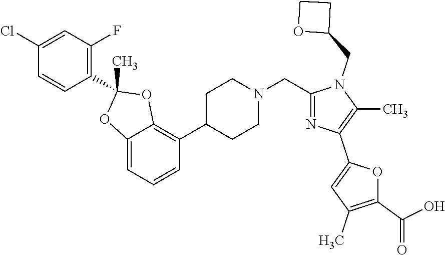

Compound C-19 was synthesized using same procedures as set forth in Example 4 but using methyl 3-methyl-5-(4,4,5,5-tetramethyl-1,3,2-dioxaborolan-2-yl)furan-2-carboxylate in step 1.

C-19: ¹H NMR (400 MHz, MeOD) δ 7.57 (t, J=8.3 Hz, 1H), 7.26 (dd, J=10.8, 2.0 Hz, 1H), 7.18 (dd, J=8.5, 2.1 Hz, 1H), 6.81-6.74 (m, 1H), 6.70 (dd, J=7.1, 5.5 Hz, 2H), 6.51 (s, 1H), 5.15 (qd, J=7.4, 2.4 Hz, 1H), 4.64 (td, J=7.8, 5.6 Hz, 1H), 4.59-4.51 (m, 1H), 4.48 (dt, J=9.1, 5.9 Hz, 1H), 4.37 (dd, J=15.6, 2.5 Hz, 1H), 3.90 (d, J=13.8 Hz, 1H), 3.79 (d, J=14.0 Hz, 1H), 3.24 (s, 1H), 3.11 (d, J=12.3 Hz, 1H), 2.86-2.66 (m, 2H), 2.53 (s, 3H), 2.57-2.32 (m, 3H), 2.36 (s, 3H), 2.02 (s, 3H), 2.05-1.75 (m, 4H). HRMS calculated for C₃₄H₃₆ClFN₃O₆ (M+H)⁺636.2277, found 636.2281.

### Example 5: Synthesis of 4-(2-((4-((S)-2-(4-chloro-2-fluorophenyl)-2-methylbenzo[d][1,3]dioxol-4-yl)piperidin-1-yl)methyl)-4-methyl-1-(((S)-oxetan-2-yl)methyl)-1H-imidazol-5-yl)oxazole-2-carboxylic acid (C-20)

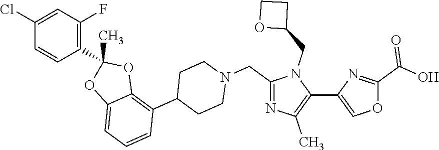 [SMILES: Cc1nc(CN2CCC(c3cccc4c3O[C@@](C)(c3ccc(Cl)cc3F)O4)CC2)n(C[C@@H]2CCO2)c1-c1coc(C(=O)O)n1]

### Step 1: Synthesis of ethyl 4-(2-((4-((S)-2-(4-chloro-2-fluorophenyl)-2-methylbenzo[d][1,3]dioxol-4-yl)piperidin-1-yl)methyl)-4-methyl-1-(((S)-oxetan-2-yl)methyl)-1H-imidazol-5-yl)oxazole-2-carboxylate (I.4)

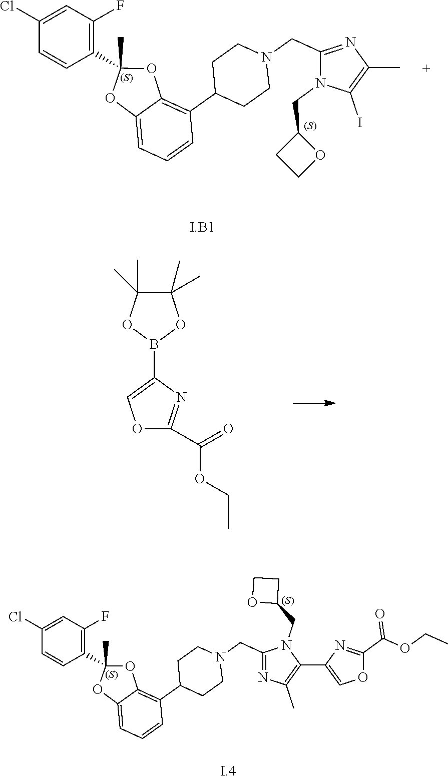

To a solution of 4-((S)-2-(4-chloro-2-fluorophenyl)-2-methylbenzo[d][1,3]dioxol-4-yl)-1-((5-iodo-4-methyl-1-(((S)-oxetan-2-yl)methyl)-1H-imidazol-2-yl)methyl)piperidine (I.B1) (60.00 mg, 94.06 μmol) in 1,4-dioxane (1.000 mL) was added ethyl 4-(4,4,5,5-tetramethyl-1,3,2-dioxaborolan-2-yl)oxazole-2-carboxylate (75.36 mg, 2 μmol), K₂CO₃ (211.6 μL, 2.000 M, 4423.3 μmol) and XPhos Pd G2 (7.400 mg, 9.406 μmol). The reaction mixture was purged with nitrogen for 5 min before being heated in microwave at 110° C. for 30 min. LCMS showed formation of ethyl 4-(2-((4-((S)-2-(4-chloro-2-fluorophenyl)-2-methylbenzo[d][1,3]dioxol-4-yl)piperidin-1-yl)methyl)-4-methyl-1-(((S)-oxetan-2-yl)methyl)-1H-imidazol-5-yl)oxazole-2-carboxylate (I.4). The hydrolyzed acid pdt (Rt=0.81 min) and fair amount of bis-coupling side pdt (Rt=1.27 min) were observed, too. The crude was acidified by 1M citric acid (to pH=4), and diluted with brine and extracted with EtOAc (2×). The separated organics were filtered, concentrated, and used in next step without further purifications. LCMS Method 2 (Basic): Rt=1.34 min, MS (ESI+) m/z 651.3 (M+H)⁺.

### Step 2: Synthesis of 4-(2-((4-((S)-2-(4-chloro-2-fluorophenyl)-2-methylbenzo[d][1,3]dioxol-4-yl)piperidin-1-yl)methyl)-4-methyl-1-(((S)-oxetan-2-yl)methyl)-1H-imidazol-5-yl)oxazole-2-carboxylic acid (C-20)

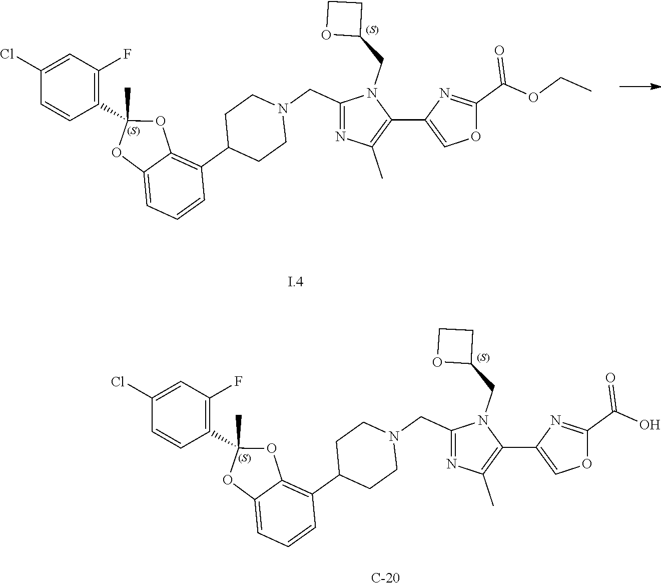

To a solution of ethyl 4-(2-((4-((S)-2-(4-chloro-2-fluorophenyl)-2-methylbenzo[d][1,3]dioxol-4-yl)piperidin-1-yl)methyl)-4-methyl-1-(((S)-oxetan-2-yl)methyl)-1H-imidazol-5-yl)oxazole-2-carboxylate (I.4) (61.00 mg, 93.68 μmol) in THF (0.500 mL) and MeOH (0.250 mL) was added LiOH (374.7 μL, 1.000 M, 374.7 μmol). The resulting mixture was stirred at rt for overnight. LCMS showed that the reaction was complete. The crude was directly purified with ISCO (10-100% ACN in water, 0.1% conc. NH₄OH as buffer) on 50 g C18 column, affording 4-(2-((4-((S)-2-(4-chloro-2-fluorophenyl)-2-methylbenzo[d][1,3]dioxol-4-yl)piperidin-1-yl)methyl)-4-methyl-1-(((S)-oxetan-2-yl)methyl)-1H-imidazol-5-yl)oxazole-2-carboxylic acid lithium salt (C-20) (7.000 mg, 12%) after lyophilization.

C-20: LCMS Method 2 (Basic): Rt=0.78 min, MS (ESI+) m/z 623.4 (M+H)⁺. ¹H NMR (400 MHz, MeOD) δ 7.94 (s, 1H), 7.48 (t, J=8.3 Hz, 1H), 7.18 (dd, J=11.0, 2.0 Hz, 1H), 7.11 (dd, J=8.3, 2.1 Hz, 1H), 6.72-6.54 (m, 3H), 4.95 (dt, J=7.4, 3.8 Hz, 1H), 4.85 (dd, J=15.0, 7.6 Hz, 1H), 4.56 (dd, J=15.1, 2.9 Hz, 1H), 4.45 (td, J=7.9, 5.8 Hz, 1H), 4.30 (dt, J=9.2, 5.9 Hz, 1H), 3.76 (d, J=13.6 Hz, 1H), 3.55 (d, J=13.6 Hz, 1H), 2.94 (d, J=11.2 Hz, 1H), 2.83 (d, J=11.4 Hz, 1H), 2.71-2.45 (m, 2H), 2.37-2.23 (m, 1H), 2.20-2.00 (m, 5H), 1.93 (s, 3H), 1.89-1.61 (m, 4H).

### Example 5-1: Synthesis of 3-(2-((4-((S)-2-(4-chloro-2-fluorophenyl)-2-methylbenzo[d][1,3]dioxol-4-yl)piperidin-1-yl)methyl)-4-methyl-1-(((S)-oxetan-2-yl)methyl)-1H-imidazol-5-yl)-1-methyl-1H-pyrazole-5-carboxylic acid (C-21)

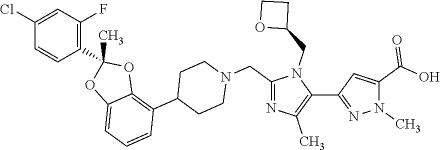 [SMILES: Cc1nc(CN2CCC(c3cccc4c3O[C@@](C)(c3ccc(Cl)cc3F)O4)CC2)n(C[C@@H]2CCO2)c1-c1cc(C(=O)O)n(C)n1]

Compound C-21 was synthesized using same procedures as set forth in Example 5 but using methyl 1-methyl-3-(4,4,5,5-tetramethyl-1,3,2-dioxaborolan-2-yl)-1H-pyrazole-5-carboxylate in step 1.

C:21: ¹H NMR (400 MHz, MeOD) δ 7.48 (t, J=8.3 Hz, 1H), 7.18 (dd, J=10.8, 2.0 Hz, 1H), 7.11 (dd, J=8.4, 2.1 Hz, 1H), 6.73-6.54 (m, 4H), 4.91 (qd, J=7.0, 3.3 Hz, 1H), 4.71 (dd, J=15.0, 6.8 Hz, 1H), 4.52-4.43 (m, 2H), 4.30 (dt, J=9.2, 5.9 Hz, 1H), 4.06 (s, 3H), 3.75 (d, J=13.7 Hz, 1H), 3.59 (d, J=13.5 Hz, 1H), 2.94 (d, J=11.6 Hz, 1H), 2.87 (d, J=11.5 Hz, 1H), 2.65-2.54 (m, 1H), 2.54-2.44 (m, 1H), 2.29-2.20 (m, 1H), 2.19-2.02 (m, 5H), 1.93 (s, 3H), 1.89-1.61 (m, 4H). HRMS calculated for C₃₃H₃₆ClFN₅O₅ (M+H)⁺636.2389, found 636.2394.

### Example 5-2: Synthesis of 5-(2-((4-((S)-2-(4-chloro-2-fluorophenyl)-2-methylbenzo[d][1,3]dioxol-4-yl)piperidin-1-yl)methyl)-4-methyl-1-(((S)-oxetan-2-yl)methyl)-1H-imidazol-5-yl)furan-2-carboxylic acid (C-22)

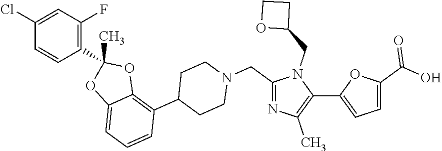 [SMILES: Cc1nc(CN2CCC(c3cccc4c3O[C@@](C)(c3ccc(Cl)cc3F)O4)CC2)n(C[C@@H]2CCO2)c1-c1ccc(C(=O)O)o1]

Compound C-22 was synthesized using same procedures as set forth in Example 5 but using (5-(ethoxycarbonyl)furan-2-yl)boronic acid in step 1.

C-22: ¹H NMR (400 MHz, MeOD) δ 7.48 (t, J=8.3 Hz, 1H), 7.18 (dd, J=10.9, 2.2 Hz, 1H), 7.11 (dd, J=8.3, 2.2 Hz, 1H), 6.93 (d, J=3.4 Hz, 1H), 6.74-6.54 (m, 3H), 6.43 (d, J=3.4 Hz, 1H), 4.93 (qd, J=7.1, 3.0 Hz, 1H), 4.84-4.73 (m, 1H), 4.52 (dd, J=15.1, 3.1 Hz, 1H), 4.45 (td, J=7.9, 5.7 Hz, 1H), 4.31 (dt, J=9.0, 6.0 Hz, 1H), 3.74 (d, J=13.7 Hz, 1H), 3.56 (d, J=13.6 Hz, 1H), 2.93 (d, J=11.3 Hz, 1H), 2.83 (d, J=11.4 Hz, 1H), 2.67-2.50 (m, 2H), 2.27 (ddt, J=11.4, 9.2, 7.0 Hz, 1H), 2.19-2.01 (m, 5H), 1.93 (s, 3H), 1.88-1.60 (m, 4H). HRMS calculated for C₃₃H₃₄ClFN₃O₆ (M+H)⁺622.21, found 622.2185.

### Example 5-3: Synthesis of 4-(2-((4-((S)-2-(4-chloro-2-fluorophenyl)-2-methylbenzo[d][1,3]dioxol-4-yl)piperidin-1-yl)methyl)-4-methyl-1-(((S)-oxetan-2-yl)methyl)-1H-imidazol-5-yl)thiazole-2-carboxylic acid (C-31)

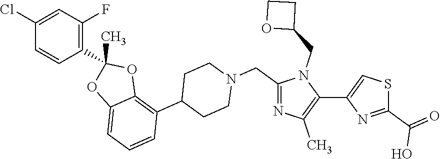 [SMILES: Cc1nc(CN2CCC(c3cccc4c3O[C@@](C)(c3ccc(Cl)cc3F)O4)CC2)n(C[C@@H]2CCO2)c1-c1csc(C(=O)O)n1]

Compound C-31 was synthesized using same procedures as set forth in Example 5 but using (2-(ethoxycarbonyl)thiazol-4-yl)boronic acid in step 1.

C-31: LCMS Method 2 (Basic): Rt=0.83 min, MS (ESI+) m/z 639.2 (M+H)⁺.

¹H NMR (400 MHz, DMSO) δ 7.91 (s, 1H), 7.63-7.53 (m, 2H), 7.34 (dd, J=8.5, 2.1 Hz, 1H), 6.80 (d, J=4.5 Hz, 2H), 6.78-6.72 (m, 1H), 4.89-4.68 (m, 2H), 4.52 (dd, J=14.6, 3.3 Hz, 1H), 4.44-4.34 (m, 1H), 4.34-4.20 (m, 1H), 3.31 (s, 6H), 2.74 (s, 1H), 2.54 (s, 1H), 2.20 (s, 4H), 2.03 (s, 3H), 1.94-1.68 (m, 4H).

### Example 6: Synthesis of 5-(2-((4-((S)-2-(4-chloro-2-fluorophenyl)-2-methylbenzo[d][1,3]dioxol-4-yl)piperidin-1-yl)methyl)-5-methyl-1-(((S)-oxetan-2-yl)methyl)-1H-imidazol-4-yl)nicotinic acid (C-23)

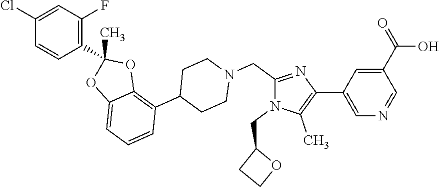 [SMILES: Cc1c(-c2cncc(C(=O)O)c2)nc(CN2CCC(c3cccc4c3O[C@@](C)(c3ccc(Cl)cc3F)O4)CC2)n1C[C@@H]1CCO1]

To a solution of 1-((4-bromo-5-methyl-1-(((S)-oxetan-2-yl)methyl)-1H-imidazol-2-yl)methyl)-4-((S)-2-(4-chloro-2-fluorophenyl)-2-methylbenzo[d][1,3]dioxol-4-yl)piperidine (I.A1) (39 mg, 66 μmol), prepared using same procedures as set forth in steps 1 to 3 of Example 1-3, in 1,4-dioxane (0.7 mL) was added 5-carboxypyridine-3-boronic acid (13 mg, 79 μmol), K₃PO₄ (0.30 mL, 1 M, 0.30 mmol) and Pd 118 (4.3 mg, 6.6 μmol). The mixture was degassed under N₂ and then microwaved at 120° C. for 20 min. The reaction mixture was diluted with water, filtered through a plug and purified on basic HPLC to afford 5-(2-((4-((S)-2-(4-chloro-2-fluorophenyl)-2-methylbenzo[d][1,3]dioxol-4-yl)piperidin-1-yl)methyl)-5-methyl-1-(((S)-oxetan-2-yl)methyl)-1H-imidazol-4-yl)nicotinic acid (C-23).

C-23: LCMS Method 4 (Product analysis-basic): Rt=1.79 min, MS (ESI+) m/z 633.5 (M+H)⁺.

¹H NMR (400 MHz, DMSO) δ 8.91 (d, J=2.3 Hz, 1H), 8.86 (d, J=2.0 Hz, 1H), 8.42 (t, J=2.1 Hz, 1H), 7.62-7.50 (m, 2H), 7.34 (dd, J=8.4, 2.0 Hz, 1H), 6.83-6.71 (m, 3H), 5.04 (qd, J=7.4, 2.8 Hz, 1H), 4.58-4.39 (m, 3H), 4.30 (dd, J=15.2, 2.9 Hz, 1H), 3.76 (d, J=13.4 Hz, 1H), 3.52 (d, J=13.3 Hz, 1H), 3.05-2.96 (m, 1H), 2.88 (dq, J=12.5, 4.2 Hz, 1H), 2.76-2.58 (m, 2H), 2.44 (s, 4H), 2.21-2.05 (m, 2H), 2.02 (s, 3H), 1.73 (dqd, J=28.3, 11.3, 3.5 Hz, 4H).

### Example 7: 5-(2-((4-((S)-2-(4-chloro-2-fluorophenyl)-2-methylbenzo[d][1,3]dioxol-4-yl)piperidin-1-yl)methyl)-5-methyl-1-(((S)-oxetan-2-yl)methyl)-1H-imidazol-4-yl)furan-2-carboxylicacid (C-24)

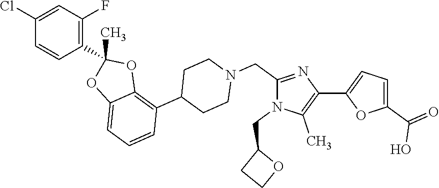 [SMILES: Cc1c(-c2ccc(C(=O)O)o2)nc(CN2CCC(c3cccc4c3O[C@@](C)(c3ccc(Cl)cc3F)O4)CC2)n1C[C@@H]1CCO1]

To 5-boronofuran-2-carboxylic acid (1.3 mg, 8.46 μmol) at room temperature was added a 0.1 M solution of 1-((4-bromo-5-methyl-1-(((S)-oxetan-2-yl)methyl)-1H-imidazol-2-yl)methyl)-4-((S)-2-(4-chloro-2-fluorophenyl)-2-methylbenzo[d][1,3]dioxol-4-yl)piperidine (I.A1) (5 mg, 85 μL, 8.46 μmol), prepared using same procedures as set forth in steps 1 to 3 of Example 1-3, containing PdCl₂(dtbpf) (1.4 mg, 2.1 μmol) in DMA. To this solution was added a 1 M aqueous solution of potassium phosphate tribasic (5.4 mg, 25.4 μL, 25.4 μmol). The mixture was stirred at 110° C. for 18 hours. After this time, solvent was removed under reduced pressure in a genevac. The crude product was dissolved in 7:2:1 acetonitrile:water:dimethyl sulfoxide, passed through a metal scavenger filter (SiliCycle SiliaPrep 96-well dimercaptotriazine, 40-63 μm, 60 Å), purified by HPLC preparation method MC-1 and immediately quantitated by CAD-equipped LCMS (LCMS method MC-1). Solvent was removed under a Porvair Sciences Ultravap Mistral evaporator, then the purified solid was immediately reconstituted in DMSO to afford 5-(2-((4-((S)-2-(4-chloro-2-fluorophenyl)-2-methylbenzo[d][1,3]dioxol-4-yl)piperidin-1-yl)methyl)-5-methyl-1-(((S)-oxetan-2-yl)methyl)-1H-imidazol-4-yl)furan-2-carboxylic acid (C-24) as a solution in DMSO. LCMS Method MC-1: Rt=1.23 min; MS m/z 622.8 [M+1]⁺. LCMS Method MC-2: Rt=1.16 min; MS m/z 622.0 [M+1]⁺.

### Biological Assays and Data

Compounds of formula (I) according to the invention were tested in the following cellular assays that measure the intracellular cAMP concentration, beta-arrestin recruitment and receptor internalization. The cAMP is generated by the activation of GLP1R. The cAMP data obtained is shown in Table 1. Beta-arrestin is recruited upon activation of GLP1R and the data obtained are shown in Table 2. The extent to which the GLP1R is internalized into the cell and away from the plasma membrane following activation is also shown in Table 2. EC₅₀ for each assay is defined as the concentration of the compound that leads to half of the maximum response (after baseline correction). Eₘₐₓ is defined as the maximum response observed for the test compound, normalized to the maximum response observed for the endogenous ligand (GLP1(7-36)) to GLP1R.

Human GLP1R cAMP Agonist Assay

The agonist activity of compounds was determined using the GloSensor™ cAMP Assay (Promega Corp.), which measures changes in the intracellular concentration of cAMP after ligand activation of GPCRs. The assay uses a biosensor encoded by pGloSensor™-22F cAMP plasmid (Promega, cat #E2301) with cAMP binding domains fused to a mutant form of Photinus pyralis luciferase. Binding to cAMP causes conformational changes that promote large increases in light output, which can be measured by a luminescence detector. HEK293-SNAP-hGLP1R-GloSensor cells stably overexpressing the human GLP1 receptor (hGLP1R) and pGloSensor™-22F were seeded in white 384-well poly-D-Lysine coated plates (Greiner Bio One, cat #781945) in CO₂-independent media (Gibco cat #18045-088 with 1.0% FBS, 2 mM L-glutamine, penicillin and streptomycin) and incubated overnight at 37° C., 5% CO₂ with humidity. The assay was started the following morning by adding an equal volume of CO₂-independent media containing 4% v/v dilution of the GloSensor substrate (Promega, cat #E1291) to all wells. The cell plate was incubated at rt for 2 h in the dark. The Biomek 7 (Beckman Coulter) instrument was used for the liquid handling steps. To generate duplicate dose response curves, 3-fold serially diluted compounds were added in to the cell assay plate to a final volume of 60 μL with final concentrations ranging from 30 μM through 0.06 μM in CO₂-independent media containing 0.1% BSA, 0.5 mM IBMX and 0.4% DMSO. In the same plate and assay buffer as the tested compounds, were EC₁₀₀ control wells containing GLP1 (7-36) peptide (Bachem, cat #H-6795) at a final concentration of 2 nM and also EC₀ control wells containing no peptide. This plate was incubated at rt in the dark for 12 min after adding the compounds to the cells. Luminescence was then measured with an Envision 2104 Multilabel reader with “TRF Light Unit, 337 nm” (PerkinElmer) using the Ultra-Sensitive protocol setting “384-well US luminescence detector” with the 384-well luminescence aperture, 0.1 seconds per well. cAMP activity was calculated as percent of the GLP1 (7-36) EC₁₀₀ control wells: [(sample signal−mean EC₀ signal)/(mean EC₁₀₀ of GLP1 (7-36) signal−mean EC₀ signal)}*100. Curve fitting for EC₅₀ determinations was performed in the Helios module of the software package DAVID. The 4-parameter logistic model, Hill slope was used: y=A_{inf}+(A₀−A_{inf})/(1+(x/AC₅₀)^{Hill Slope}) where y is the functional response; x is the compound concentration; A₀ is the minimum value (at 0 dose); A_{inf} is the maximum value (at infinite dose); AC₅₀ corresponds to the point of inflection (i.e. the point on the sigmoid shaped curve halfway between A₀ and A_{inf}). The EC₅₀ value was represented by the AC₅₀ value calculated from Helios in μM. Eₘₐₓ is the maximal activity detected within the concentration range, derived from the fitted curve.

### Generation of the HEK293-SNAP-hGLP1R Cell Line

327 μL of Opti-MEM medium (Gibco, cat #31985-062) were mixed with 12 μL of FuGENE® HD (Promega, cat #E2311) and incubated at rt for 5 min. Then 8.2 μL (4 μg, 0.485 μg/μL solution) of pSNAP-hGLP1R plasmid (Cisbio, cat #PSNAP-GLP1) encoding human GLP1R (NCBI Reference Sequence: NM_002062.3) fused with Cisbio's SNAP tag was added in to the Fugene HD/Opti-MEM mix, and incubated at rt for 20 min. A suspension of HEK293 cells (ATCC® CRL-1573™) was prepared at 800,000 cells/mL. Then, the plasmid/FuGene HD mixture was added to 8 mL of cells and mixed gently. 2 mL of the new mix were added to 4 wells in a 6-well plate and 2 mL of un-transfected cells were added to two wells as control. The plate was incubated at 37° C. until 100% confluence. The antibiotic selection [800 μg/mL G418 (Geneticin, Gibco, cat #10131-035)] was done after cell trypsinization at a dilution of 2500 cells/mL. 1 mL cell suspension was added to 20 mL selection medium in a 10 cm culture dish (2500 cells in total) and in parallel, 4 mL diluted cell suspension were added to 20 mL selection medium in a 10 cm culture dish (10000 cells in total). The rest of the cells were cultured in a T150 flask. In addition, HEK293 cells were cultured in a T75 flask in selection medium as negative control. Finally, single clones were picked from a 10 cm culture dish and continued culture until there were enough cells for gene expression analysis and HTRF cAMP assay. Clone 2 showed the highest GLP1R-dependent cAMP response and was expanded for the generation of the GloSensor stable cell line.

### Generation of HEK293-SNAP-hGLP1R-GloSensor Stable Cell Line

The HEK293 cells stably overexpressing SNAP-hGLP1R (described above) were plated at a density of 3 million cells in a 10 cm dish containing 17 mL of DMEM complete growth medium (Gibco, cat #11965-092)+10% Fetal Bovine Serum (FBS, Gibco, cat #16140-071). The following day, cells were transfected as follows. The DNA complex was prepared as 0.020 μg/μL pGloSensor™-22F cAMP plasmid (Promega, cat #E2301; GenBank® accession is GU174434) by adding 37 μg of plasmid DNA in 1758 μL Opti-MEM solution. Then, 112 μL of FuGENE® HD reagent were added to that by mixing carefully. After 5-10 min incubation at RT, 850 μL of complex per well were added to the cells, and mixed thoroughly. After 24 h incubation at 37° C., 5% CO₂ with humidity, media was removed and cells were rinsed with PBS. Then, selection medium [600 μg/mL G418 and 600 μg/mL hygromycin B (Gibco, cat #10687010)], was added. The medium was changed twice a week until no more dead cells were observed. Once cell clones were visible, single cells were isolated. For that, 10 μL of 0.05% Trypsin-EDTA solution was added to single cells by pipetting up and down. These single cell-derived clones were then cultured in six well plates with selection medium (600 μg/mL G418+600 μg/mL hygromycin B) until enough cells were available to be tested for cAMP agonist response in the GloSensor luminescence assay. The HEK293-SNAP-hGLP1R stable cell clone that yielded the desired response was used for human GLP1R cAMP agonist assay.

TABLE 1

| Compound | EC₅₀ mean | |
|---|---|---|
| No. | (μM) | Eₘₐₓ mean (%) |
| C-1 | 5.60E−05 | 110 |
| C-2 | 1.49E−04 | 108 |
| C-3 | 4.84E−05 | 105 |
| C-4 | 2.98E−04 | 120 |
| C-5 | 4.18E−05 | 123 |
| C-6 | 4.64E−04 | 123 |
| C-7 | 3.51E−05 | 118 |
| C-8 | 3.53E−04 | 101 |
| C-9 | 1.03E−04 | 107 |
| C-10 | 1.42E−04 | 108 |
| C-11 | 4.28E−05 | 105 |
| C-12 | 5.75E−04 | 114 |
| C-13 | 3.76E−04 | 102 |
| C-14 | 4.92E−04 | 104 |
| C-15 | 1.12E−04 | 103 |
| C-16 | 1.71E−04 | 108 |
| C-17 | 7.54E−04 | 113 |
| C-18 | 1.88E−04 | 115 |
| C-19 | 3.77E−04 | 109 |
| C-20 | 2.40E−04 | 112 |
| C-21 | 8.36E−04 | 117 |
| C-22 | 3.50E−04 | 119 |
| C-23 | 6.13E−04 | 106 |
| C-24 | 1.02E−04 | 113 |

### Human GLP1R β-Arrestin Recruitment Assay

The extent to which agonists recruited β-arrestin was measured using the PathHunter® β-arrestin assay (DiscoverX). This assay measures binding of β-arrestin to the receptor using an enzyme complementation approach. Two inactive portions of a β-galactosidase enzyme (termed Prolink and Enzyme Acceptor, or ‘EA’) are tagged so that the human GLP1R (hGLP1R) contains the Prolink portion and β-arrestin contains the EA portion. When β-arrestin is recruited to the receptor the enzyme becomes active and generates luminescence in the presence of a chemiluminescent substrate (PathHunter® Detection Kit, DiscoverX cat #93-0001). Luminescence can be measured on a relevant detector. CHO-hGLP1R-β-arrestin cells stably overexpressing hGLP1R with Prolink tag and β-arrestin with EA tag were seeded at 20 μL per well in white 384-well poly-D-Lysine coated plates (Greiner Bio One, cat #781945) in Plating Reagent 2 (DiscoverX, cat #93-0563R^{2A}), and incubated overnight at 37° C., 5% CO₂ with humidity. The following day, agonists were prepared at 5 times the final required concentration. To generate triplicate dose response curves, compounds were serially diluted 3-fold in assay buffer (HBSS, 10 mM Hepes and 0.1% BSA), then added to the cell assay plate to a final volume of 25 μL and final top concentrations starting at 30 μM. In the same plate and assay buffer as the tested compounds, were EC₁₀₀ control wells containing GLP1 (7-36) peptide (Bachem, cat #H-6795) at a final concentration of 1 μM and also EC₀ control wells containing no compound. The plate was incubated at 37° C., 5% CO₂ with humidity for 2 h after adding the compounds to the cells. Then the detection reagent was prepared (19 parts cell assay buffer, 5 parts substrate reagent 1 and 1 part substrate reagent 2 as per manufacturers recommendations, DiscoverX cat #93-0001), and 12 μL were added per well to the cell assay plate. The plate was incubated for an additional hour in the dark at RT. Luminescence was then measured with an Envision 2104 Multilabel reader with “TRF Light Unit, 337 nm” (Perkin Elmer) using the Ultra-Sensitive protocol setting “384-well US luminescence detector” with the 384-well luminescence aperture, 0.1 sec per well. β-arrestin recruitment was calculated and expressed as percent of the GLP1 (7-36) EC₁₀₀ control wells: [(sample signal−mean EC₀ signal)/(mean EC₁₀₀ of GLP1 (7-36) signal−mean EC₀ signal)]*100 using Microsoft Excel. Curve fitting for EC₅₀ determinations was performed using GraphPad Prism. The 4-parameter logistic model, Hill slope was used: Y=Bottom+(Top−Bottom)/(1+10\hat{ }((Log EC₅₀−X)*Hill Slope)), where Y is the functional response; X is the compound concentration; bottom is A₀ or the minimum value (at 0 dose); top is A_{inf} or the maximum value (at infinite dose); EC₅₀ is the point of inflection (i.e. the point on the sigmoid shaped curve halfway between A₀ and Ainf). The EC₅₀ value was calculated in μM. Eₘₐₓ is the maximal activity detected within the concentration range, derived from the fitted curve relative to GLP1(7-36). The reference compound is 2-((4-((S)-2-(4-chloro-2-fluorophenyl)-2-methylbenzo[d][1,3]dioxol-4-yl)piperidin-1-yl)methyl)-1-(((S)-oxetan-2-yl)methyl)-1H-benzo[d]imidazole-6-carboxylic acid (WO 2019/239319, Example 7)

### Generation of CHO-hGLP1R-β-Arrestin Cell Line

PathHunter CHO-K1-EA parental cells (DiscoverX, cat #93-0164) were plated at a density of 2×10⁶ cells per T75 cm² flask in 22 mL of complete medium (AssayComplete Cell Culture kit 107, DiscoverX, cat #92-3107G). The following day, the medium was replaced with 22 mL of fresh medium with no antibiotics and cells were transfected as follows. Plasmid/Fugene® HD Transfection mix was prepared in Opti-MEM media (3:1 Ratio of Reagent:DNA). 25 μg (34 μL) of pCMV-PK1-GLP1R plasmid [(DiscoverX pCMV PK vector bundle, cat #93-0491 with sequence inserted encoding full-length human GLP1R—NCBI Reference Sequence: NM_002062, synthesized by GeneArt (Thermo Fisher Scientific)], was added to 1129 μL Opti-MEM for a total volume of 1163 μL. Then, 74 μL of FuGENE® HD Reagent was added by mixing carefully. After 5-10 min incubation at RT, 1125 μL of complex solution were added to the cells and incubated for 48 h at 37° C. Then, medium was removed and selection medium containing 300 μg/mL hygromycin (Gibco, cat #10687010) and 500 μg/mL geneticin (Gibco, cat #10131035) was added. The medium was changed every 2-3 days until no more dead cells were observed. Cells were detached, re-suspended at 300000 cells/mL and strained with 40 μm strainer. The cells were then FACS sorted using Aria G instrument into single cells in black, clear bottom poly-D-lysine coated 96-well plates in 100 μL medium. Medium was changed every 2-3 days by removing up to 80 μL and adding fresh medium containing selection antibiotics. Surviving single clones were expanded and tested. Single clone 1 was selected for the β-arrestin assay based on optimal signal and curve profile.

### Human GLP1R DERET Internalization Assay

The extent to which agonists internalize the human GLP1R was determined based on an optimized version of a RealTime FRET-based ‘DERET’ (Dissociation Enhanced Resonance Energy Transfer) assay. The technology relies on labeling of the SNAP-tagged GPCR with a SNAP-Lumi-Terbium (donor fluorophore, Cisbio, cat #SSNPTBD). The compounds are incubated with the cells over-expressing the GPCR of interest in the presence of an excess of fluorescein (acceptor fluorophore). When the GPCR is on the cell surface, the donor signal is quenched by the acceptor and the donor/acceptor ratio is low. As the GPCR internalizes, the donor signal is no longer quenched, and the acceptor is no longer excited so the donor/acceptor ratio increases.

HEK293-SNAP-hGLP1R-GloSensor cells (stably overexpressing SNAP-tagged hGLP1R) were seeded overnight in white 384-well poly-D-Lysine coated plates (Greiner Bio One, cat #781945) in regular DMEM growth medium (Gibco, cat #11965-092, 10% heat-inactivated FBS, 10 mM HEPES, 1× penicillin/streptomycin, 0.5 mg/mL geneticin (Gibco, cat #10131-035) and 0.25 mg/mL hygromycin B (Invitrogen, cat #10687010). On the assay day, cell medium was removed and 100 nM SNAP-Lumi-Tb reagent was added in Opti-MEM solution. The cells were incubated at 37° C. for 1 h. Cells were washed using a plate washer in assay buffer [1×HBSS (10× Gibco, cat #14065-056), 20 mM Hepes (Gibco, cat #15630-080), 1 mM CaCl₂ (Fluka, cat #21114-1L), 1 mM MgCl₂ (Ambion, cat #AM9530G) pH7.4], and 20 μL buffer with 0.1% BSA was added to each well. After leaving cells to equilibrate for −15 min at 37° C., 10 μL of Fluorescein (sodium salt, Sigma, cat #F6377, diluted in buffer) was added at 25 μM final concentration. To generate triplicate dose response curves, compounds were serially diluted 3-fold in assay buffer, then added to the cell assay plate to a final volume of 40 μL and final top concentrations starting at 30 μM. In the same plate and assay buffer as the tested compounds, a GLP1 (7-36) peptide (Bachem, cat #H-6795) control curve was included at a final top concentration of 1 μM in order to establish EC₁₀₀. EC₀ wells with buffer only were also included. The plate FRET fluorescence was measured immediately using a Perkin Elmer Envision with LANCE/DELFIA D400 single mirror, excitation filter X320, and emission filters M615_203 (donor emission) and M515 (acceptor emission), and then measured every 30 min. Peak Internalization was reached at 120 min. Plates were kept at 37° C. between reads. Data was expressed as the ratio of donor/acceptor emissions using Microsoft Excel and plotted in GraphPad Prism. In order to determine EC₅₀ and Eₘₐₓ for internalization, data was calculated and expressed as percent of the GLP1 (7-36) EC₁₀₀ control wells: [(sample signal−mean EC₀ signal)/(mean EC₁₀₀ of GLP1 (7-36) signal−mean EC₀ signal)]*100 using Microsoft Excel. Curve fitting for EC₅₀ determinations was performed using GraphPad Prism. The 4-parameter logistic model, Hill slope was used:

$$Y = Bottom + ( Top - Bottom ) / ( 1 + 10 \hat{} ( {( Log {EC}_{50} - X )}^{*} Hill Slope ) ) ,$$

where Y is the functional response; X is the compound concentration; bottom is A₀ or the minimum value (at 0 dose); top is A_{inf} or the maximum value (at infinite dose); EC₅₀ is the point of inflection (i.e. the point on the sigmoid shaped curve halfway between A₀ and A_{inf}). The EC₅₀ value was calculated in μM. Eₘₐₓ is the maximal activity that was measured within the concentration range, derived from the fitted curve relative to GLP1(7-36).

TABLE 2

| | Beta-Arrestin assay | | DERET Internalization assay | |
|---|---|---|---|---|
| Compound | EC₅₀ Mean | | EC₅₀ Mean | |
| No. | (μM) | Eₘₐₓ (%) | (uM) | Eₘₐₓ (%) |
| C-1 | >30 | 2 | >30 | 8 |
| C-4 | >30 | 0 | >30 | 1 |
| C-5 | >30 | 0 | >30 | 6 |
| C-7 | >30 | 5 | 0.03 | 51 |
| C-9 | >30 | 0 | >30 | 11 |
| C-10 | >30 | 0 | >30 | 7 |
| C-12 | >30 | 10 | >30 | 45 |
| C-13 | >30 | 12 | 1.279 | 42 |
| C-14 | >30 | 0 | >30 | 20 |
| C-16 | >30 | 11 | 2.943 | 50 |
| C-17 | >30 | 2 | >30 | 6 |
| C-18 | >30 | 16 | 2.376 | 86 |
| C-19 | >30 | 7 | >30 | 34 |
| C-20 | >30 | 0 | >30 | 5 |
| C-21 | >30 | 0 | >30 | 2 |
| C-23 | ND | ND | >30 | 10 |
| C-24 | >30 | 1 | 0.49 | 19 |
| Reference | 0.123 | 27 | 0.015 | 87 |
| compound | | | | |

1. A compound of formula (I): 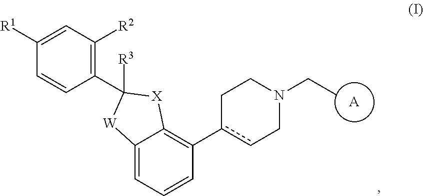
or a pharmaceutically acceptable salt thereof, wherein [symbol]
is a single or double bond; 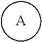
is 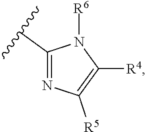
wherein [symbol]
indicates the point of attachment to the rest of the molecule;
W is O or CH₂;
X is O or CH₂;
R¹ and R² are each independently selected from H, C₁₋₃-alkyl, halo and CN;
R³ is selected from H and C₁₋₃-alkyl;
R⁴ and R⁵ are selected from:
(aa) R⁴ is selected from H, C₁₋₆-alkyl, C₂₋₆ alkenyl, C₂₋₆ alkynyl, C₃₋₆-cycloalkyl, 4-10 membered heterocycloalkyl, phenyl, C₃₋₆-cycloalkyl-C₁₋₃-alkyl-, (4-10 membered heterocycloalkyl)-C₁₋₃-alkyl-, phenyl-C₁₋₃-alkyl-, halo, CN, NO₂, OR^{4a}, SR^{4a}, C(O)R^{4b}, C(O)NR^{4c}R^{4d}, C(O)NR^{4c}(OR^{4a}), C(O)NR^{4c}(S(O)₂R^{4b}), C(O)NR^{4c}(S(O)₂NR^{4c}R^{4d}), NR^{4c}OR^{4a}, NR^{4c}R^{4d}, NR^{4c}(C(O)R^{4b}), NR^{4c}(C(O)OR^{4a}), N(OR^{4a})(C(O)R^{4b}), NR^{4c}(C(O)NR^{4c}R^{4d}), NR^{4c}(C(O)NR^{4c}(C(O)R^{4b})), NR^{4c}(S(O)₂R⁴), NR^{4c}(S(O)₂NR^{4c}R^{4d}), NR^{4c}(C(O)NR^{4c}(S(O)₂R^{4b})), OC(O)R^{4b}, OC(O)NR^{4c}R^{4d}, ONR^{4c}(C(O)R^{4b}), OS(O)₂R^{4b}, OP(O)(OR^{4e})(OR^{4f}), S(O)OR^{4a}, S(O)R^{4b}, S(O)₂R^{4b}, S(O)₂NR^{4c}R^{4d}, S(O)₂OR^{4a}, S(═NR^{4g})(O)R^{4b}, S(═NR^{4g})(O)NR^{4c}NR^{4d}, P(O)(OR^{4e})(OR^{4f}), and P(O)(OR^{4e})(R^{4f}), wherein the C₁₋₆-alkyl, C₂₋₆ alkenyl, C₂₋₆ alkynyl, C₃₋₆-cycloalkyl, 4-10 membered heterocycloalkyl, phenyl, C₃₋₆-cycloalkyl-C₁₋₃-alkyl-, (4-10 membered heterocycloalkyl)-C₁₋₃-alkyl-, and phenyl-C₁₋₃-alkyl- of R⁴ are each optionally substituted with 1, 2, or 3 groups independently selected from halo, CN, NO₂, OR^{4A}, SR^{4A}, C(O)R^{4B}, C(O)NR^{4C}R^{4D}, C(O)NR^{4C}(OR^{4A}), C(O)NR^{4C}(S(O)₂R^{4B}), C(O)NR^{4C}(S(O)₂NR^{4C}R^{4D}), NR^{4C}OR^{4A}, NR^{4C}R^{4D}, NR^{4C}(C(O)R^{4B}), NR^{4C}(C(O)OR^{4A}), N(OR^{4A})(C(O)R^{4B}), NR^{4C}(C(O)NR^{4C}R^{4D}), NR^{4C}(C(O)NR^{4C}(C(O)R^{4B})), NR^{4C}(S(O)₂R^{4B}), NR^{4C}(S(O)₂NR^{4C}R^{4D}), NR^{4C}(C(O)NR^{4C}(S(O)₂R^{4B})), OC(O)R^{4B}, OC(O)NR^{4C}R^{4D}, ONR^{4C}(C(O)R^{4B}), OS(O)₂R^{4B}, OP(O)(OR^{4E})(OR^{4F}), S(O)OR^{4A}, S(O)R^{4B}, S(O)₂R^{4B}, S(O)₂NR^{4C}R^{4D}, S(O)₂OR^{4A}, S(═NR^{4G})(O)R^{4B}, S(═NR^{4G})(O)NR^{4C}NR^{4D}, P(O)(OR^{4E})(OR^{4F}), and P(O)(OR^{4E})(R^{4F}), and
R⁵ is 5-10 membered heteroaryl or (5-10 membered heteroaryl)-C₁₋₃-alkyl-, wherein the 5-10 membered heteroaryl and (5-10 membered heteroaryl)-C₁₋₃-alkyl- are each substituted with C(O)OR^{5A} or with a carboxylic acid isostere, and optionally with 1, 2, or 3 groups independently selected from halo, CN, NO₂, OR^{5A}, SR^{5A}, C₁₋₃-alkyl, C₁₋₃-alkyl substituted with 1, 2 or 3 halo, C(O)R^{5B}, C(O)NR^{5C}R^{5D}, C(O)NR^{5C}(OR^{5A}), C(O)NR^{5C}(S(O)₂R^{5B}), C(O)NR^{5C}(S(O)₂NR^{5C}R^{5D}), NR^{5C}OR^{5A}, NR^{5C}R^{5D}, NR^{5C}(C(O)R^{5B}), NR^{5C}(C(O)OR^{5A}), N(OR^{5A})(C(O)R^{5B}), NR^{5C}(C(O)NR^{5C}R^{5D}), NR^{5C}(C(O)NR^{5C}(C(O)R^{5B})), NR^{5C}(S(O)₂R^{5B}), NR^{5C}(S(O)₂NR^{5C}R^{5D}), NR^{5C}(C(O)NR^{5C}(S(O)₂R^{5B})), OC(O)R^{5B}, OC(O)NR^{5C}R^{5D}, ONR^{5C}(C(O)R^{5B}), OS(O)₂R^{5B}, OP(O)(OR^{5E})(OR^{5F}), S(O)OR^{5A}, S(O)R^{5B}, S(O)₂R^{5B}, S(O)₂NR^{5C}R^{5D}, S(O)₂OR^{5A}, S(═NR^{5G})(O)R^{5B}, S(═NR^{5G})(O)NR^{5C}NR^{5D}, P(O)(OR^{5E})(OR^{5F}), and P(O)(OR^{5E})(R^{5F}); or
(bb) R⁴ is 5-10 membered heteroaryl or (5-10 membered heteroaryl)-C₁₋₃-alkyl-, wherein the 5-10 membered heteroaryl and (5-10 membered heteroaryl)-C₁₋₃-alkyl- are each substituted with C(O)OR^{4A} or with a carboxylic acid isostere, and optionally with 1, 2, or 3 groups independently selected from halo, CN, NO₂, OR^{4A}, SR^{4A}, C₁₋₃-alkyl, C₁₋₃-alkyl substituted with 1, 2 or 3 halo, C(O)R^{4B}, C(O)NR^{4C}R^{4D}, C(O)NR^{4C}(OR^{4A}), C(O)NR^{4C}(S(O)₂R^{4B}), C(O)NR^{4C}(S(O)₂NR^{4C}R^{4D}), NR^{4C}OR^{4A}, NR^{4C}R^{4D}, NR^{4C}(C(O)R^{4B}), NR^{4C}(C(O)OR^{4A}), N(OR^{4A})(C(O)R^{4B}), NR^{4C}(C(O)NR^{4C}R^{4D}), NR^{4C}(C(O)NR^{4C}(C(O)R^{4B})), NR^{4C}(S(O)₂R^{4B}), NR^{4C}(S(O)₂NR^{4C}R^{4D}), NR^{4C}(C(O)NR^{4C}(S(O)₂R^{4B})), OC(O)R^{4B}, OC(O)NR^{4C}R^{4D}, ONR⁴(C(O)R^{4B}), OS(O)₂R^{4B}, OP(O)(OR^{4E})(OR^{4F}), S(O)OR^{4A}, S(O)R^{4B}, S(O)₂R^{4B}, S(O)₂NR^{4C}R^{4D}, S(O)₂OR^{4A}, S(═NR^{4G})(O)R^{4B}, S(═NR^{4G})(O)NR^{4C}NR^{4D}, P(O)(OR^{4E})(OR^{4F}), and P(O)(OR^{4E})(R^{4F}), and
R⁵ is selected from H, C₁₋₆-alkyl, C₂₋₆ alkenyl, C₂₋₆ alkynyl, C₃₋₆-cycloalkyl, 4-10 membered heterocycloalkyl, phenyl, C₃₋₆-cycloalkyl-C₁₋₃-alkyl, (4-10 membered heterocycloalkyl)-C₁₋₃-alkyl-, phenyl-C₁₋₃-alkyl-, halo, CN, NO₂, OR^{5a}, SR^{5a}, C(O)R^{5b}, C(O)NR^{5c}R^{5d}, C(O)NR^{5c}(OR^{5a}), C(O)NR^{5c}(S(O)₂R^{5b}), C(O)NR^{5c}(S(O)₂NR^{5c}R^{5d}), NR^{5c}OR^{5a}, NR^{5c}R^{5d}, NR^{5c}(C(O)R^{5b}), NR^{5c}(C(O)OR^{5a}), N(OR^{5a})(C(O)R^{5b}), NR^{5c}(C(O)NR^{5c}R^{5d}), NR^{5c}(C(O)NR^{5c}(C(O)R^{5b})), NR^{5c}(S(O)₂R^{5b}), NR^{5c}(S(O)₂NR^{5c}R^{5d}), NR^{5c}(C(O)NR^{5c}(S(O)₂R^{5b})), OC(O)R^{5b}, OC(O)NR^{5c}R^{5d}, ONR^{5c}(C(O)R^{5b}), OS(O)₂R^{5b}, OP(O)(OR^{5e})(OR^{5f}), S(O)OR^{5a}, S(O)R^{5b}, S(O)₂R^{5b}, S(O)₂NR^{5c}R^{5d}, S(O)₂OR^{5a}, S(═NR^{5g})(O)R^{5b}, S(═NR^{5g})(O)NR^{5c}NR^{5d}, P(O)(OR^{5e})(OR⁵), and P(O)(OR^{5e})(R⁵), wherein the C₁₋₆-alkyl, C₂₋₆ alkenyl, C₂₋₆ alkynyl, C₃₋₆-cycloalkyl, 4-10 membered heterocycloalkyl, phenyl, C₃₋₆-cycloalkyl-C₁₋₃-alkyl-, (4-10 membered heterocycloalkyl)-C₁₋₃-alkyl-, and phenyl-C₁₋₃-alkyl- of R⁵ are each optionally substituted with 1, 2, or 3 groups independently selected from halo, CN, NO₂, OR^{5A}, SR^{5A}, C(O)R^{5B}, C(O)NR^{5C}R^{5D}, C(O)NR^{5C}(OR^{5A}), C(O)NR^{5C}(S(O)₂R^{5B}), C(O)NR^{5C}(S(O)₂NR^{5C}R^{5D}), NR^{5C}OR^{5A}, NR^{5C}R^{5D}, NR^{5C}(C(O)R^{5B}), NR^{5C}(C(O)OR^{5A}), N(OR^{5A})(C(O)R^{5B}), NR^{5C}(C(O)NR^{5C}R^{5D}), NR^{5C}(C(O)NR^{5C}(C(O)R^{5B})), NR^{5C}(S(O)₂R^{5B}), NR^{5C}(S(O)₂NR^{5C}R^{5D}), NR^{5C}(C(O)NR^{5C}(S(O)₂R^{5B})), OC(O)R^{5B}, OC(O)NR^{5C}R^{5D}, ONR^{5C}(C(O)R^{5B}), OS(O)₂R^{5B}, OP(O)(OR^{5E})(OR^{5F}), S(O)OR^{5A}, S(O)R^{5B}, S(O)₂R^{5B}, S(O)₂NR^{5C}R^{5D}, S(O)₂OR^{5A}, S(═NR^{5G})(O)R^{5B}, S(═NR^{5G})(O)NR^{5C}NR^{5D}, P(O)(OR^{5E})(OR^{5F}), and P(O)(OR^{5E})(R^{5F}); R⁶ is selected from (4-10 membered heterocycloalkyl)-C₁₋₃-alkyl- and (5-10 membered heteroaryl)-C₁₋₃-alkyl-, wherein the (4-10 membered heterocycloalkyl)-C₁₋₃-alkyl- and (5-10 membered heteroaryl)-C₁₋₃-alkyl- of R⁶ are each optionally substituted with 1, 2, or 3 groups independently selected from C₁₋₆-alkyl, —OH, and halo;
R^{4a}, R^{4b}, R^{4c}, and R^{4d} are each independently selected from H, C₁₋₆-alkyl, C₂₋₆ alkenyl, C₂₋₆ alkynyl, C₃₋₆-cycloalkyl, 4-10 membered heterocycloalkyl, 5-10 membered heteroaryl, and phenyl, wherein the C₁₋₆-alkyl, C₂₋₆ alkenyl, and C₂₋₆ alkynyl of R^{4a}, R^{4b}, R^{4c}, and R^{4d} are each optionally substituted with 1, 2, or 3 groups independently selected from —OH and halo; and
the C₃₋₆-cycloalkyl, 4-10 membered heterocycloalkyl, 5-10 membered heteroaryl, and phenyl of R^{4a}, R^{4b}, R^{4c}, and R^{4d} are each optionally substituted with 1, 2, or 3 groups independently selected from C₁₋₆-alkyl, —OH, and halo;
R^{4c}, R^{4f}, and R^{4g} are each independently selected from H and C₁₋₆-alkyl;
R^{4A}, R^{4B}, R^{4C}, and R^{4D} are each optionally substituted with 1, 2, or 3 groups independently selected from —OH, halo and —OC(O)—C₁₋₁₅-alkyl; and the C₃₋₆-cycloalkyl, 4-10 membered heterocycloalkyl, 4-10 membered heterocycloalkyl-C₁₋₃-alkyl-, 5-10 membered heteroaryl, and phenyl of R^{4A}, R^{4B}, R^{4C}, and R^{4D} are each optionally substituted with 1, 2, or 3 groups independently selected from C₁₋₆-alkyl, —OH, and halo;
R^{4E}, R^{4F}, and R^{4G} are each independently selected from H and C₁₋₆-alkyl;
R^{5a}, R^{5b}, R^{5c}, and R^{5d} are each independently selected from H, C₁₋₆-alkyl, C₂₋₆ alkenyl, C₂₋₆ alkynyl, C₃₋₆-cycloalkyl, 4-10 membered heterocycloalkyl, 5-10 membered heteroaryl, and phenyl, wherein the C₁₋₆-alkyl, C₂₋₆ alkenyl, and C₂₋₆ alkynyl of R^{5a}, R^{5b}, R^{5c}, and R^{5d} are each optionally substituted with 1, 2, or 3 groups independently selected from —OH and halo; and
the C₃₋₆-cycloalkyl, 4-10 membered heterocycloalkyl, 5-10 membered heteroaryl, and phenyl of R^{5a}, R^{5b}, R^{5c}, and R^{5d} are each optionally substituted with 1, 2, or 3 groups independently selected from C₁₋₆-alkyl, —OH, and halo;
R^{5c}, R^{5f}, and R^{5g} are each independently selected from H and C₁₋₆-alkyl;
R^{5A}, R^{5B}, R^{5C}, and R^{5D} are each optionally substituted with 1, 2, or 3 groups independently selected from —OH, halo and —OC(O)—C₁₋₁₅-alkyl; and the C₃₋₆-cycloalkyl, 4-10 membered heterocycloalkyl, 4-10 membered heterocycloalkyl-C₁₋₃-alkyl-, 5-10 membered heteroaryl, and phenyl of R^{5A}, R^{5B}, R^{5C}, and R^{5D} are each optionally substituted with 1, 2, or 3 groups independently selected from C₁₋₆-alkyl, —OH, and halo; and
R^{5E}, R^{5F}, and R^{5G} are each independently selected from H and C₁₋₆-alkyl.

2. The compound according to claim 1, or a pharmaceutically acceptable salt thereof, wherein R¹ is chloro, fluoro or CN, R² is chloro or fluoro, and R³ is —CH₃.

3. The compound according to claim 1, or a pharmaceutically acceptable salt thereof, wherein R⁴ is selected from C₁₋₃-alkyl, C₂₋₄ alkenyl, C₂₋₄ alkynyl, C₃₋₆-cycloalkyl, 4-10 membered heterocycloalkyl, phenyl, and halo, wherein the C₁₋₃-alkyl, C₂₋₄ alkenyl, C₂₋₄ alkynyl, C₃₋₆-cycloalkyl, 4-10 membered heterocycloalkyl, and phenyl of R⁴ are each optionally substituted with 1, 2, or 3 groups independently selected from halo and C₁₋₃-alkyl, and R⁵ is 5-10 membered heteroaryl or (5-10 membered heteroaryl)-C₁₋₃-alkyl-, wherein the 5-10 membered heteroaryl and (5-10 membered heteroaryl)-C₁₋₃-alkyl- are each substituted with C(O)OR^{5A} or with a carboxylic acid isostere, and optionally with 1, 2, or 3 groups independently selected from halo, C₁₋₃-alkyl and C₁₋₃-alkyl substituted with 1, 2 or 3 halo; wherein R^{5A} is selected from H and C₁₋₃-alkyl.

4. The compound according to claim 1, or a pharmaceutically acceptable salt thereof, wherein R⁴ is C₁₋₃-alkyl or C₁₋₃-alkyl substituted with 1, 2 or 3 halo.

5. The compound according to claim 1, or a pharmaceutically acceptable salt thereof, wherein R⁴ is 5-10 membered heteroaryl or (5-10 membered heteroaryl)-C₁₋₃-alkyl-, wherein the 5-10 membered heteroaryl and (5-10 membered heteroaryl)-C₁₋₃-alkyl- are each substituted with C(O)OR^{4A} or with a carboxylic acid isostere, and optionally with 1, 2, or 3 groups independently selected from halo, C₁₋₃-alkyl and C₁₋₃-alkyl substituted with 1, 2 or 3 halo; wherein R^{4A} is selected from H and C₁₋₃-alkyl, and R⁵ is selected from C₁₋₃-alkyl, C₂₋₄ alkenyl, C₂₋₄ alkynyl, C₃₋₆-cycloalkyl, 4-10 membered heterocycloalkyl, phenyl, and halo, wherein the C₁₋₃-alkyl, C₂₋₄ alkenyl, C₂₋₄ alkynyl, C₃₋₆-cycloalkyl, 4-10 membered heterocycloalkyl, and phenyl are each optionally substituted with 1, 2, or 3 groups independently selected from halo and C₁₋₃-alkyl.

6. The compound according to claim 5, or a pharmaceutically acceptable salt thereof, wherein R⁵ is C₁₋₃-alkyl or C₁₋₃-alkyl substituted with 1, 2 or 3 halo.

7. The compound according to claim 5, or a pharmaceutically acceptable salt thereof, wherein R⁴ is 5-10 membered heteroaryl substituted with C(O)OR^{4A}, and optionally with 1, 2, or 3 groups independently selected from halo, C₁₋₃-alkyl and C₁₋₃-alkyl substituted with 1, 2 or 3 halo; wherein R^{4A} is selected from H and C₁₋₃-alkyl.

8. The compound according to claim 5, or a pharmaceutically acceptable salt thereof, wherein R⁴ has the structure of Formula H1a or H2a: 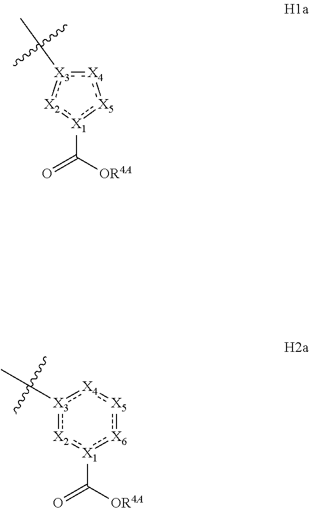
wherein the ring is aromatic; wherein
X₁ is C, and each of X₂, X₃, X₄, X₅, and X₆ is independently C═O, CR^{4h}, NR⁴ⁱ, O, or S, wherein at least one of X₂-X₆ is N, O, or S;
R^{4A} is selected from H and C₁₋₃-alkyl; and
if present, R^{4h} is halo, C₁₋₃-alkyl or C₁₋₃-alkyl substituted with 1, 2 or 3 halo, and R⁴ⁱ is C₁₋₃-alkyl.

9. The compound according to claim 5, or a pharmaceutically acceptable salt thereof, wherein R⁴ is selected from the group consisting of: 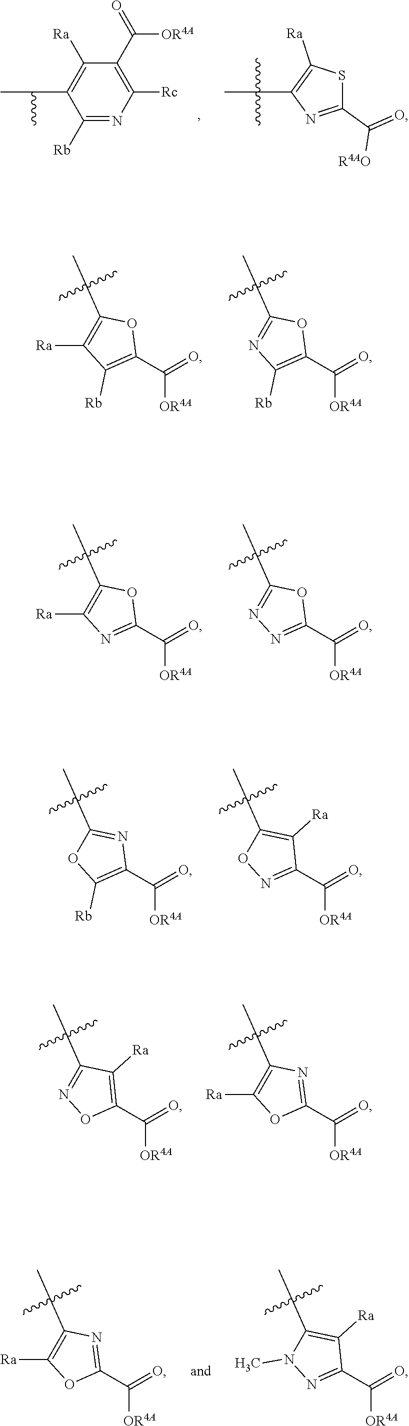
wherein Ra, Rb and Rc are independently selected from H, C₁₋₃-alkyl and C₁₋₃-alkyl substituted with 1, 2 or 3 halo, and R^{4A} is selected from H and C₁₋₃-alkyl.

10. The compound according to claim 1, or a pharmaceutically acceptable salt thereof, wherein R⁵ is 5-10 membered heteroaryl substituted with C(O)OR^{5A}, and optionally with 1, 2, or 3 groups independently selected from halo, C₁₋₃-alkyl and C₁₋₃-alkyl substituted with 1, 2 or 3 halo; wherein R^{5A} is selected from H and C₁₋₃-alkyl.

11. The compound according to claim 10, or a pharmaceutically acceptable salt thereof, wherein R⁵ has the structure of Formula H11a or H12a: 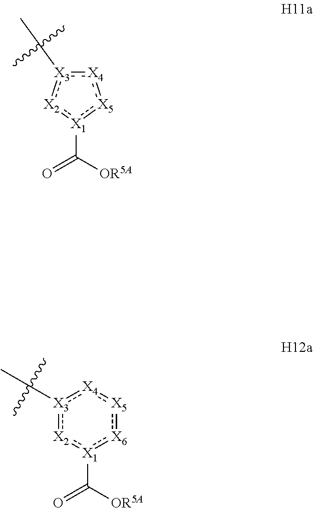
wherein the ring is aromatic; wherein
X₁ is C, and each of X₂, X₃, X₄, X₅, and X₆ is independently C═O, CR^{5h}, NR⁵ⁱ, O, or S, wherein at least one of X₂-X₆ is N, O, or S;
R^{5A} is selected from H and C₁₋₃-alkyl; and
if present, R^{5h} is halo, C₁₋₃-alkyl or C₁₋₃-alkyl substituted with 1, 2 or 3 halo, and R⁵ⁱ is C₁₋₃-alkyl.

12. The compound according to claim 10, or a pharmaceutically acceptable salt thereof, wherein R⁵ is selected from the group consisting of 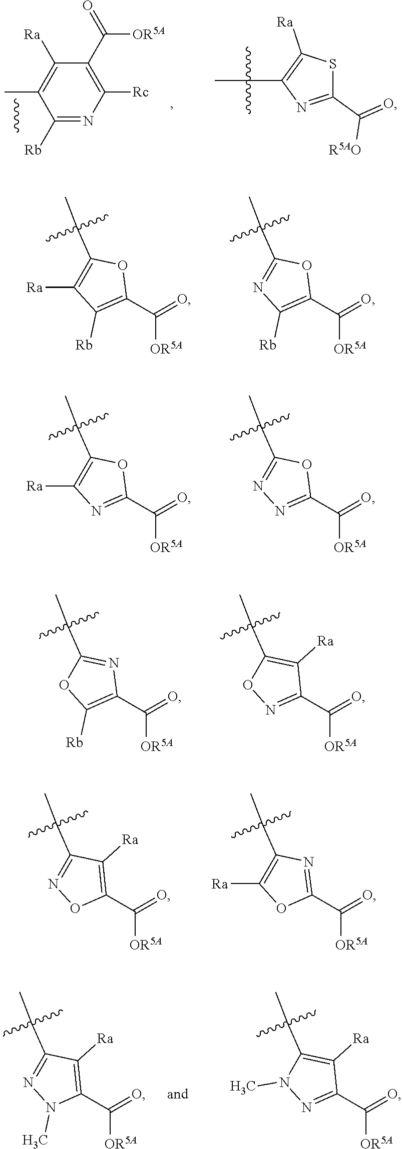
wherein Ra, Rb and Rc are independently selected from H, C₁₋₃-alkyl and C₁₋₃-alkyl substituted with 1, 2 or 3 halo, and R^{5A} is selected from H and C₁₋₃-alkyl.

13. The compound according to claim 1, or a pharmaceutically acceptable salt thereof, wherein R⁶ is selected from (4-6 membered heterocycloalkyl)-CH₂— and (5-6 membered heteroaryl)-CH₂—, wherein the (4-6 membered heterocycloalkyl)-CH₂— and (5-6 membered heteroaryl)-CH₂— of R⁶ are each optionally substituted with 1, 2, or 3 groups independently selected from C₁₋₆-alkyl, —OH, and halo.

14. The compound according to claim 13, or a pharmaceutically acceptable salt thereof, wherein R⁶ is of formula: 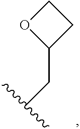 [SMILES: *CC1CCO1]
wherein [symbol] indicates the point of attachment to the rest of the molecule.

15. The compound according to claim 1, which is a compound of formula (IIa): 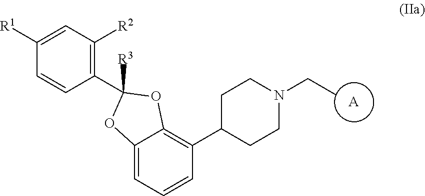
or a pharmaceutically acceptable salt thereof.

16. The compound according to claim 1, which is a compound of formula (IIIa): 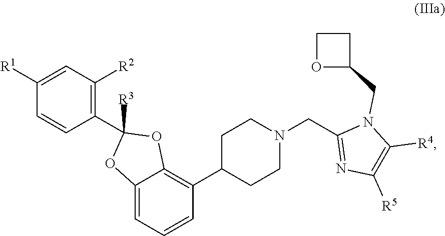
or a pharmaceutically acceptable salt thereof.

17. The compound according to claim 1, which is selected from the group consisting of: 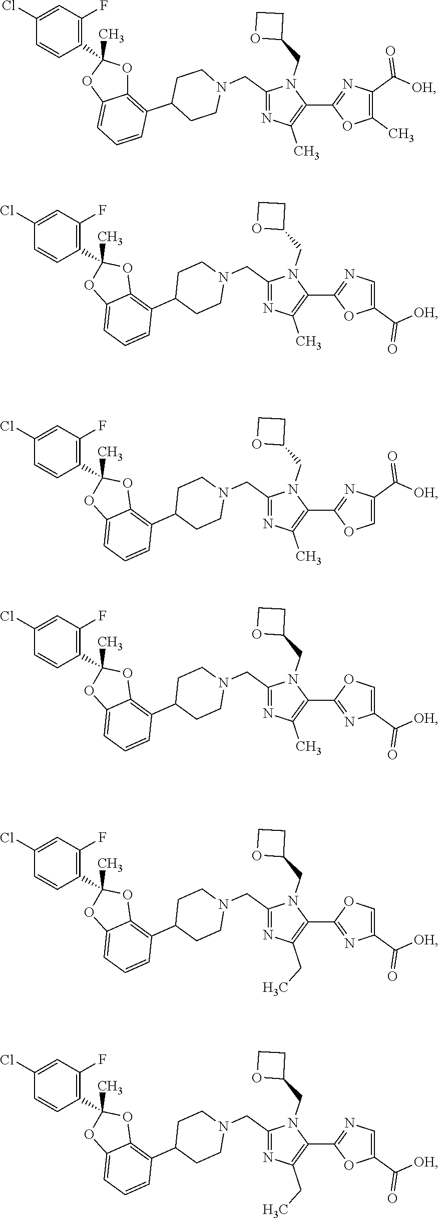 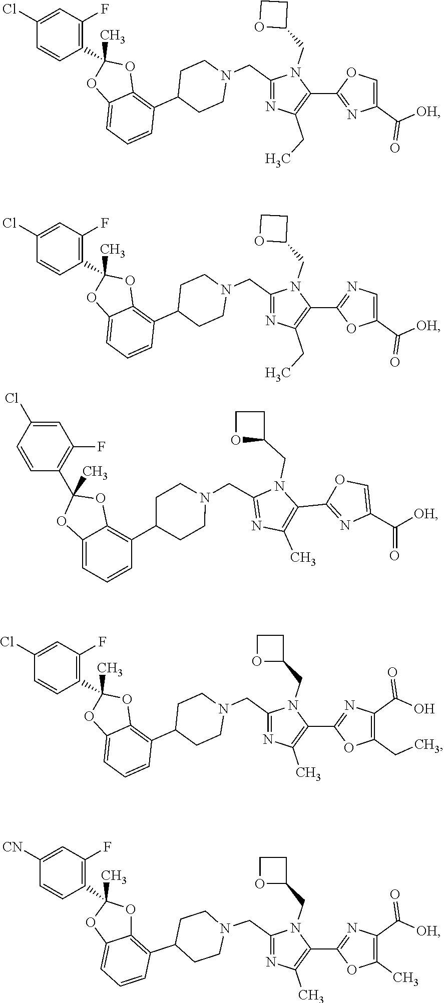 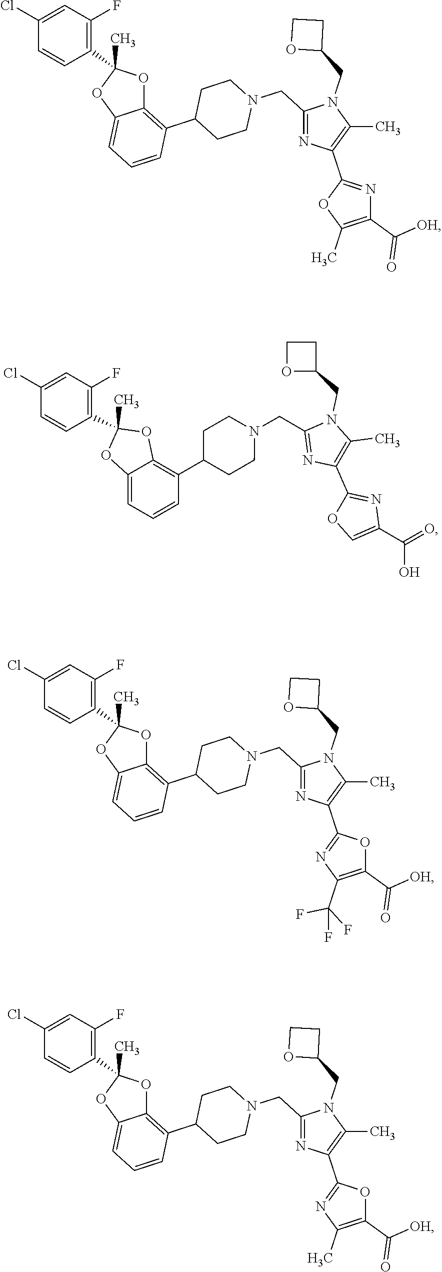 [SMILES: Cc1oc(-c2nc(CN3CCC(c4cccc5c4O[C@@](C)(c4ccc(Cl)cc4F)O5)CC3)n(C[C@@H]3CCO3)c2C)nc1C(=O)O ; Cc1c(-c2nc(C(=O)O)co2)nc(CN2CCC(c3cccc4c3O[C@@](C)(c3ccc(Cl)cc3F)O4)CC2)n1C[C@@H]1CCO1 ; Cc1c(-c2nc(C(F)(F)F)c(C(=O)O)o2)nc(CN2CCC(c3cccc4c3O[C@@](C)(c3ccc(Cl)cc3F)O4)CC2)n1C[C@@H]1CCO1 ; Cc1nc(-c2nc(CN3CCC(c4cccc5c4O[C@@](C)(c4ccc(Cl)cc4F)O5)CC3)n(C[C@@H]3CCO3)c2C)oc1C(=O)O] 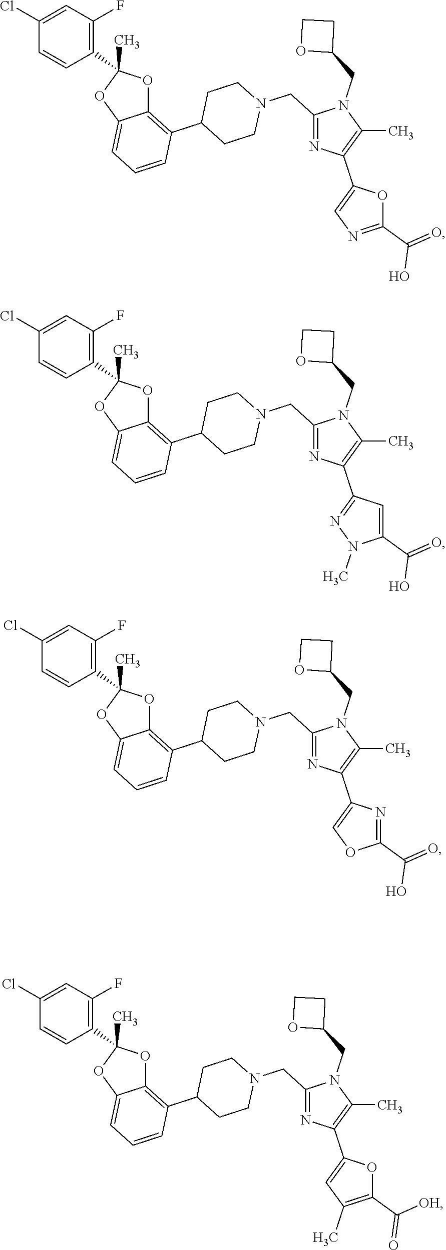 [SMILES: Cc1c(-c2cnc(C(=O)O)o2)nc(CN2CCC(c3cccc4c3O[C@@](C)(c3ccc(Cl)cc3F)O4)CC2)n1C[C@@H]1CCO1 ; Cc1c(-c2cc(C(=O)O)n(C)n2)nc(CN2CCC(c3cccc4c3O[C@@](C)(c3ccc(Cl)cc3F)O4)CC2)n1C[C@@H]1CCO1 ; Cc1c(-c2coc(C(=O)O)n2)nc(CN2CCC(c3cccc4c3O[C@@](C)(c3ccc(Cl)cc3F)O4)CC2)n1C[C@@H]1CCO1 ; Cc1cc(-c2nc(CN3CCC(c4cccc5c4O[C@@](C)(c4ccc(Cl)cc4F)O5)CC3)n(C[C@@H]3CCO3)c2C)oc1C(=O)O] 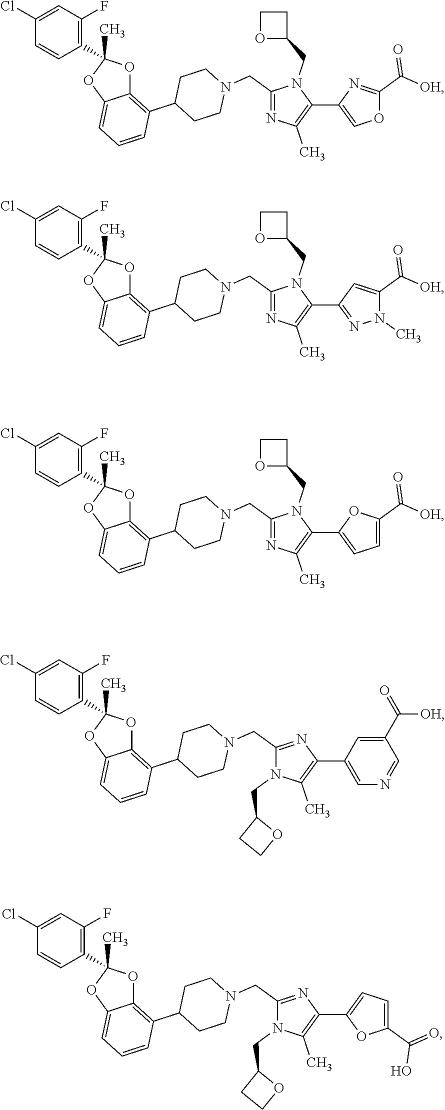 [SMILES: Cc1nc(CN2CCC(c3cccc4c3O[C@@](C)(c3ccc(Cl)cc3F)O4)CC2)n(C[C@@H]2CCO2)c1-c1coc(C(=O)O)n1 ; Cc1nc(CN2CCC(c3cccc4c3O[C@@](C)(c3ccc(Cl)cc3F)O4)CC2)n(C[C@@H]2CCO2)c1-c1cc(C(=O)O)n(C)n1 ; Cc1nc(CN2CCC(c3cccc4c3O[C@@](C)(c3ccc(Cl)cc3F)O4)CC2)n(C[C@@H]2CCO2)c1-c1ccc(C(=O)O)o1 ; Cc1c(-c2cncc(C(=O)O)c2)nc(CN2CCC(c3cccc4c3O[C@@](C)(c3ccc(Cl)cc3F)O4)CC2)n1C[C@@H]1CCO1 ; Cc1c(-c2ccc(C(=O)O)o2)nc(CN2CCC(c3cccc4c3O[C@@](C)(c3ccc(Cl)cc3F)O4)CC2)n1C[C@@H]1CCO1] 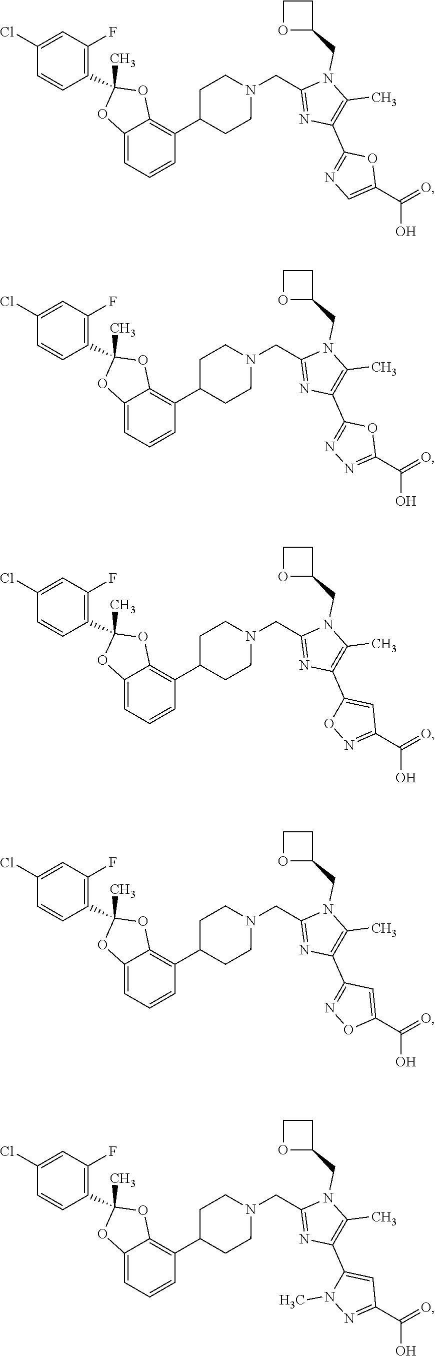 [SMILES: Cc1c(-c2ncc(C(=O)O)o2)nc(CN2CCC(c3cccc4c3O[C@@](C)(c3ccc(Cl)cc3F)O4)CC2)n1C[C@@H]1CCO1 ; Cc1c(-c2nnc(C(=O)O)o2)nc(CN2CCC(c3cccc4c3O[C@@](C)(c3ccc(Cl)cc3F)O4)CC2)n1C[C@@H]1CCO1 ; Cc1c(-c2cc(C(=O)O)no2)nc(CN2CCC(c3cccc4c3O[C@@](C)(c3ccc(Cl)cc3F)O4)CC2)n1C[C@@H]1CCO1 ; Cc1c(-c2cc(C(=O)O)on2)nc(CN2CCC(c3cccc4c3O[C@@](C)(c3ccc(Cl)cc3F)O4)CC2)n1C[C@@H]1CCO1 ; Cc1c(-c2cc(C(=O)O)nn2C)nc(CN2CCC(c3cccc4c3O[C@@](C)(c3ccc(Cl)cc3F)O4)CC2)n1C[C@@H]1CCO1] 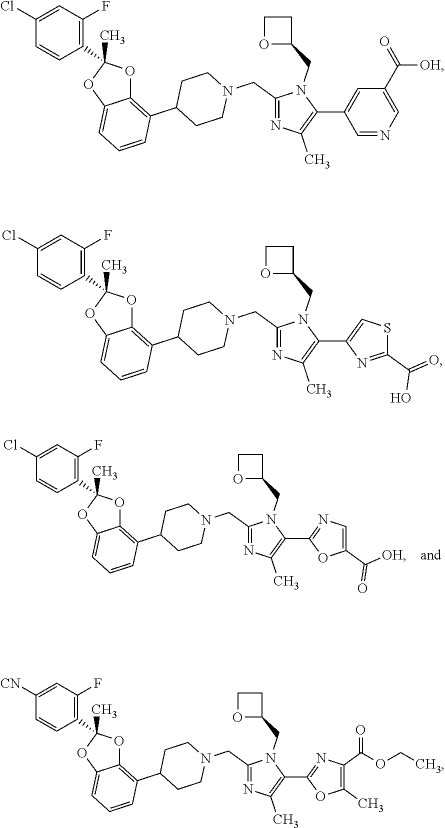
or a pharmaceutically acceptable salt thereof.

18. The compound according to claim 1, or a pharmaceutically acceptable salt thereof, selected from the group consisting of:
2-(2-((4-((S)-2-(4-chloro-2-fluorophenyl)-2-methylbenzo[d][1,3]dioxol-4-yl)piperidin-1-yl)methyl)-4-methyl-1-(((S)-oxetan-2-yl)methyl)-1H-imidazol-5-yl)-5-methyloxazole-4-carboxylic acid,
2-(2-((4-((S)-2-(4-chloro-2-fluorophenyl)-2-methylbenzo[d][1,3]dioxol-4-yl)piperidin-1-yl)methyl)-4-methyl-1-(((R)-oxetan-2-yl)methyl)-1H-imidazol-5-yl)oxazole-5-carboxylic acid,
2-(2-((4-((S)-2-(4-chloro-2-fluorophenyl)-2-methylbenzo[d][1,3]dioxol-4-yl)piperidin-1-yl)methyl)-4-methyl-1-(((R)-oxetan-2-yl)methyl)-1H-imidazol-5-yl)oxazole-4-carboxylic acid,
2-(2-((4-((S)-2-(4-chloro-2-fluorophenyl)-2-methylbenzo[d][1,3]dioxol-4-yl)piperidin-1-yl)methyl)-1-(((S)-oxetan-2-yl)methyl)-4-(trifluoromethyl)-1H-imidazol-5-yl)oxazole-4-carboxylic acid,
2-(2-((4-((S)-2-(4-chloro-2-fluorophenyl)-2-methylbenzo[d][1,3]dioxol-4-yl)piperidin-1-yl)methyl)-4-ethyl-1-(((S)-oxetan-2-yl)methyl)-1H-imidazol-5-yl)oxazole-4-carboxylic acid,
2-(2-((4-((S)-2-(4-chloro-2-fluorophenyl)-2-methylbenzo[d][1,3]dioxol-4-yl)piperidin-1-yl)methyl)-4-ethyl-1-(((S)-oxetan-2-yl)methyl)-1H-imidazol-5-yl)oxazole-5-carboxylic acid,
2-(2-((4-((S)-2-(4-chloro-2-fluorophenyl)-2-methylbenzo[d][1,3]dioxol-4-yl)piperidin-1-yl)methyl)-4-ethyl-1-(((R)-oxetan-2-yl)methyl)-1H-imidazol-5-yl)oxazole-4-carboxylic acid,
2-(2-((4-((S)-2-(4-chloro-2-fluorophenyl)-2-methylbenzo[d][1,3]dioxol-4-yl)piperidin-1-yl)methyl)-4-ethyl-1-(((R)-oxetan-2-yl)methyl)-1H-imidazol-5-yl)oxazole-5-carboxylic acid,
2-(2-((4-((S)-2-(4-chloro-2-fluorophenyl)-2-methylbenzo[d][1,3]dioxol-4-yl)piperidin-1-yl)methyl)-4-methyl-1-(((S)-oxetan-2-yl)methyl)-1H-imidazol-5-yl)oxazole-4-carboxylic acid,
2-(2-((4-((S)-2-(4-chloro-2-fluorophenyl)-2-methylbenzo[d][1,3]dioxol-4-yl)piperidin-1-yl)methyl)-4-methyl-1-(((S)-oxetan-2-yl)methyl)-1H-imidazol-5-yl)-5-ethyloxazole-4-carboxylic acid,
2-(2-((4-((S)-2-(4-cyano-2-fluorophenyl)-2-methylbenzo[d][1,3]dioxol-4-yl)piperidin-1-yl)methyl)-4-methyl-1-(((S)-oxetan-2-yl)methyl)-1H-imidazol-5-yl)-5-methyloxazole-4-carboxylic acid,
2-(2-((4-((S)-2-(4-chloro-2-fluorophenyl)-2-methylbenzo[d][1,3]dioxol-4-yl)piperidin-1-yl)methyl)-5-methyl-1-(((S)-oxetan-2-yl)methyl)-1H-imidazol-4-yl)-5-methyloxazole-4-carboxylic acid,
2-(2-((4-((S)-2-(4-chloro-2-fluorophenyl)-2-methylbenzo[d][1,3]dioxol-4-yl)piperidin-1-yl)methyl)-5-methyl-1-(((S)-oxetan-2-yl)methyl)-1H-imidazol-4-yl)oxazole-4-carboxylic acid,
2-(2-((4-((S)-2-(4-chloro-2-fluorophenyl)-2-methylbenzo[d][1,3]dioxol-4-yl)piperidin-1-yl)methyl)-5-methyl-1-(((S)-oxetan-2-yl)methyl)-1H-imidazol-4-yl)-4-(trifluoromethyl)oxazole-5-carboxylic acid,
2-(2-((4-((S)-2-(4-chloro-2-fluorophenyl)-2-methylbenzo[d][1,3]dioxol-4-yl)piperidin-1-yl)methyl)-5-methyl-1-(((S)-oxetan-2-yl)methyl)-1H-imidazol-4-yl)-4-methyloxazole-5-carboxylic acid,
5-(2-((4-((S)-2-(4-chloro-2-fluorophenyl)-2-methylbenzo[d][1,3]dioxol-4-yl)piperidin-1-yl)methyl)-5-methyl-1-(((S)-oxetan-2-yl)methyl)-1H-imidazol-4-yl)oxazole-2-carboxylic acid,
3-(2-((4-((S)-2-(4-chloro-2-fluorophenyl)-2-methylbenzo[d][1,3]dioxol-4-yl)piperidin-1-yl)methyl)-5-methyl-1-(((S)-oxetan-2-yl)methyl)-1H-imidazol-4-yl)-1-methyl-1H-pyrazole-5-carboxylic acid,
4-(2-((4-((S)-2-(4-chloro-2-fluorophenyl)-2-methylbenzo[d][1,3]dioxol-4-yl)piperidin-1-yl)methyl)-5-methyl-1-(((S)-oxetan-2-yl)methyl)-1H-imidazol-4-yl)oxazole-2-carboxylic acid,
5-(2-((4-((S)-2-(4-chloro-2-fluorophenyl)-2-methylbenzo[d][1,3]dioxol-4-yl)piperidin-1-yl)methyl)-5-methyl-1-(((S)-oxetan-2-yl)methyl)-1H-imidazol-4-yl)-3-methylfuran-2-carboxylic acid,
4-(2-((4-((S)-2-(4-chloro-2-fluorophenyl)-2-methylbenzo[d][1,3]dioxol-4-yl)piperidin-1-yl)methyl)-4-methyl-1-(((S)-oxetan-2-yl)methyl)-1H-imidazol-5-yl)oxazole-2-carboxylic acid,
3-(2-((4-((S)-2-(4-chloro-2-fluorophenyl)-2-methylbenzo[d][1,3]dioxol-4-yl)piperidin-1-yl)methyl)-4-methyl-1-(((S)-oxetan-2-yl)methyl)-1H-imidazol-5-yl)-1-methyl-1H-pyrazole-5-carboxylic acid,
5-(2-((4-((S)-2-(4-chloro-2-fluorophenyl)-2-methylbenzo[d][1,3]dioxol-4-yl)piperidin-1-yl)methyl)-4-methyl-1-(((S)-oxetan-2-yl)methyl)-1H-imidazol-5-yl)furan-2-carboxylic acid,
5-(2-((4-((S)-2-(4-chloro-2-fluorophenyl)-2-methylbenzo[d][1,3]dioxol-4-yl)piperidin-1-yl)methyl)-5-methyl-1-(((S)-oxetan-2-yl)methyl)-1H-imidazol-4-yl)nicotinic acid,
5-(2-((4-((S)-2-(4-chloro-2-fluorophenyl)-2-methylbenzo[d][1,3]dioxol-4-yl)piperidin-1-yl)methyl)-5-methyl-1-(((S)-oxetan-2-yl)methyl)-1H-imidazol-4-yl)furan-2-carboxylic acid,
2-(2-((4-((S)-2-(4-chloro-2-fluorophenyl)-2-methylbenzo[d][1,3]dioxol-4-yl)piperidin-1-yl)methyl)-5-methyl-1-(((S)-oxetan-2-yl)methyl)-1H-imidazol-4-yl)oxazole-5-carboxylic acid,
5-(2-((4-((S)-2-(4-chloro-2-fluorophenyl)-2-methylbenzo[d][1,3]dioxol-4-yl)piperidin-1-yl)methyl)-5-methyl-1-(((S)-oxetan-2-yl)methyl)-1H-imidazol-4-yl)-1,3,4-oxadiazole-2-carboxylic acid,
5-(2-((4-((S)-2-(4-chloro-2-fluorophenyl)-2-methylbenzo[d][1,3]dioxol-4-yl)piperidin-1-yl)methyl)-5-methyl-1-(((S)-oxetan-2-yl)methyl)-1H-imidazol-4-yl)isoxazole-3-carboxylic acid,
3-(2-((4-((S)-2-(4-chloro-2-fluorophenyl)-2-methylbenzo[d][1,3]dioxol-4-yl)piperidin-1-yl)methyl)-5-methyl-1-(((S)-oxetan-2-yl)methyl)-1H-imidazol-4-yl)isoxazole-5-carboxylic acid,
5-(2-((4-((S)-2-(4-chloro-2-fluorophenyl)-2-methylbenzo[d][1,3]dioxol-4-yl)piperidin-1-yl)methyl)-5-methyl-1-(((S)-oxetan-2-yl)methyl)-1H-imidazol-4-yl)-1-methyl-1H-pyrazole-3-carboxylic acid,
5-(2-((4-((S)-2-(4-chloro-2-fluorophenyl)-2-methylbenzo[d][1,3]dioxol-4-yl)piperidin-1-yl)methyl)-4-methyl-1-(((S)-oxetan-2-yl)methyl)-1H-imidazol-5-yl)nicotinic acid,
4-(2-((4-((S)-2-(4-chloro-2-fluorophenyl)-2-methylbenzo[d][1,3]dioxol-4-yl)piperidin-1-yl)methyl)-4-methyl-1-(((S)-oxetan-2-yl)methyl)-1H-imidazol-5-yl)thiazole-2-carboxylic acid, and
2-(2-((4-((S)-2-(4-chloro-2-fluorophenyl)-2-methylbenzo[d][1,3]dioxol-4-yl)piperidin-1-yl)methyl)-4-methyl-1-(((S)-oxetan-2-yl)methyl)-1H-imidazol-5-yl)oxazole-5-carboxylic acid, and
ethyl 2-(2-((4-((S)-2-(4-cyano-2-fluorophenyl)-2-methylbenzo[d][1,3]dioxol-4-yl)piperidin-1-yl)methyl)-4-methyl-1-(((S)-oxetan-2-yl)methyl)-1H-imidazol-5-yl)-5-methyloxazole-4-carboxylate,
or a pharmaceutically acceptable salt thereof.

19. A pharmaceutical composition comprising a compound according to claim 1, or a pharmaceutically acceptable salt thereof, and one or more pharmaceutically acceptable carriers.

20. A combination comprising a compound according to claim 1, or a pharmaceutically acceptable salt thereof, and one or more therapeutically active agents.

21-23. (canceled)

24. A method of agonizing GLP1R activity in a subject, wherein the method comprises administering to the subject a therapeutically effective amount of the compound according to claim 1, or a pharmaceutically acceptable salt thereof.

25. A method of treating a disorder or disease selected from obesity, type 2 diabetes mellitus, insulin resistance, hyperinsulinemia, glucose intolerance, hyperglycemia, one or more diabetic complications, diabetic nephropathy, dyslipidemia, non-alcoholic fatty liver disease (NAFLD), non-alcoholic steatohepatitis (NASH), hypertension, atherosclerosis, peripheral arterial disease, stroke, cardiomyopathy, atrial fibrillation, heart failure, coronary heart disease, and neuropathy in a subject, comprising administering to the subject a therapeutically effective amount of the compound according to claim 1, or a pharmaceutically acceptable salt thereof.
